(19) United States
(12) Patent Application Publication
(10) Pub. No.: US 2006/0134768 A1
(43) Pub. Date: Jun. 22, 2006

(54) Erbb4 co-crystal

(76) Inventors: Lisa Marie Shewchuk, Durham, NC (US); Anne Moore Hassell, Durham, NC (US); Perry Scott Brignola, Alpharetta, GA (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/543,046

(22) PCT Filed: Jan. 20, 2004
(86) PCT No.: PCT/US04/01291

§ 371 Date: Jul. 21, 2005

(30) Foreign Application Priority Data
Jan. 21, 2003 (US) 60441443

(51) Int. Cl. C12N 9/12; G06F 19/00

(52) U.S. Cl. 435/194; 702/19

(57) ABSTRACT

A crystal structure of the ErbB4 kinase domain (ErbB4K), specifically the ErbB4K in liganded form as well as methods of using the same in the discovery of ErbB4 inhibitors. Disclosed further are methods of treatment of diseases mediated by inappropriate ErbB4 activity utilizing ErbB4 inhibitors identified by the methods disclosed herein.

### BACKGROUND OF THE INVENTION

The present invention relates to the crystal structure of the ErbB4 kinase domain (ErbB4K), specifically the ErbB4K in liganded form as well as methods of using the same in the discovery of ErbB4 inhibitors and in the treatment of diseases mediated by inappropriate ErbB4 activity.

Abberant protein tyrosine kinase (PTK) activity has been implicated in a variety of disorders including psoriasis, rheumatoid arthritis, bronchitis, as well as cancer. Development of effective treatments for such disorders is a constant and ongoing enterprise in the medical field. The ErbB family of PTKs, which includes c-ErbB-2, EGFR, and ErbB-4, is one group of PTKs that has attracted interest as a therapeutic target. Currently, of special interest, is the role of ErbB family PTKs in hyperproliferative disorders, particularly human malignancies. Consequently, inhibition of ErbB family PTKs should provide for disorders characterized by aberrant ErbB family PTK activity. The biological role of ErbB family PTKs and their implication in various disease states is discussed, for instance in U.S. Pat. No. 5,773,476; International patent application WO99/35146; M. C Hung et al, Seminars in Oncology, 26: 4, Suppl. 12 (August) 1999, 51-59; Ulrich et al, Cell, 61:203-212, Apr. 20, 1990; Modjtahedi et al, Int'l J. of Oncology, 13: 335-342, 1998; and J. R. Woodburn, Pharmacol. Ther., 82: 2-3, 241-250, 1999.

Polypeptides, including ErbB4, have a three-dimensional structure determined by the primary amino acid sequence and the environment surrounding the polypeptide. This three-dimensional structure establishes the polypeptide's activity, stability, binding affinity, binding specificity, and other biochemical attributes. Thus, knowledge of a protein's three-dimensional structure can provide much guidance in designing agents that mimic, inhibit, or improve its biological activity in soluble or membrane bound forms.

The three-dimensional structure of a polypepetide can be determined in a number of ways. Many of the most precise methods employ X-ray crystallography (See e.g., Van Holde, (1971) Physical Biochemistry, Prentice-Hall, N.J., 221-239). This technique relies on the ability of crystalline lattices to diffract X-rays or other forms of radiation. Diffraction experiments suitable for determining the three-dimensional structure of macromolecules typically require high-quality crystals. Since such crystals have been unavailable for ErbB4, a three-dimensional structure of ErbB4 has proven difficult to elucidate.

The present inventors have developed such crystals and have now determined the crystal structure of the nonphosphorylated human ErbB4K complexed with an irreversible inhibitor to 2.5 Å resolution. Such a crystal structure is useful in discovering compounds suitable for inhibiting ErbB4 and for treating diseases characterized by aberrant ErbB4 activity.

### BRIEF SUMMARY OF THE INVENTION

In one aspect of the present invention, there is provided an ErbB4 kinase domain in liganded crystalline form, comprising the amino acid sequence of SEQ ID NO: 1 and having the structural coordinates of Table 2.

In a second aspect of the present invention, there is provided a method of ErbB4 inhibitor design, comprising:

generating a three dimensional computer model which represents ErbB4 kinase domain in liganded form, said kinase domain described by the amino acid sequence of SEQ ID NO: 1 and having the structural coordinates of Table 2;

evaluating compounds as potential ErbB4 inhibitors using said model; and

selecting compounds for further testing based on said evaluation.

In a third aspect of the present invention, there is provided a method of ErbB4 inhibitor design, comprising:

generating a three dimensional computer model which represents an ErbB4 kinase domain in liganded form, said kinase domain described by the amino acid sequence of SEQ ID NO: 1 and having the structural coordinates of Table 2;

evaluating compounds as potential ErbB4 inhibitors using said model; wherein said evaluation comprises identifying compounds capable of at least one of the following ErbB4 kinase domain/compound interactions:
(i) one or more interactions with amino acid residues of the ErbB4 kinase domain hinge region;
(ii) one or more interactions with amino acid residues of the ErbB4 kinase domain adenine pocket,
(iii) one or more interactions with amino acid residues of the ErbB4 kinase sugar pocket,
(iv) one or more interactions with amino acid residues of the ErbB4 kinase domain back pocket, and
(v) one or more interactions with amino acid residues of the ErbB4 kinase domain solvent interface; and
selecting compounds for further testing based on said evaluation.

In a fourth aspect of the present invention, there is provided a method of ErbB4 inhibitor design, comprising:

generating a three dimensional computer model which represents a ErbB4 kinase domain in liganded form, said kinase domain described by the amino acid sequence of SEQ ID NO: 1 and having the structural coordinates of Table 2;

evaluating compounds as potential ErbB4 inhibitors using said model; wherein said evaluation comprises identifying compounds capable of at least one of the following ErbB4 kinase domain/compound interactions:

(i) one or more interactions with amino acid residues 796, 797, 798, 799, and 800;

(ii) one or more interactions with amino acid residues 724, 749, and 850;

(iii) one or more interactions with amino acid residues 848, 860, 803, 847, 732, and 725;

(iv) one or more interactions with amino acid residues 732, 749, 751, 796, 861, 860, 772, 781, 783, 794, 796, and 862; and

(v) one or more interactions with residues 801, 802, 803, 806, and 810; and

selecting compounds for further testing based on said evaluation.

In a fifth aspect of the present invention, there is provided a method of treating a disorder characterized by inappropriate ErbB4 activity in a mammal, comprising: administering to said mammal a therapeutically effective amount of a compound that can form a complex with an ErbB4 kinase domain thereby resulting in a ErbB4 kinase domain in liganded form, said kinase domain in liganded form being described by the amino acid sequence of SEQ ID NO: 1 and the structural coordinates of Table 2, wherein said complex is characterized by at least one of the following ErbB4 kinase domain/compound interactions:

(i) one or more interactions with amino acid residues of the ErbB4 kinase domain hinge region;

(ii) one or more interactions with amino acid residues of the ErbB4 kinase domain adenine pocket,

(iii) one or more interactions with amino acid residues of the ErbB4 kinase sugar pocket and phosphate region,

(iv) one or more interactions with amino acid residues of the ErbB4 kinase domain back pocket, and

(v) one or more interactions with amino acid residues of the ErbB4 kinase domain solvent interface.

In a sixth aspect of the present invention, there is provided a method of inhibiting ErbB4 in a mammal, comprising: administering to said mammal a therapeutically effective amount of a compound that can form a complex with a ErbB4 kinase domain thereby resulting in an ErbB4 kinase domain in liganded form, said kinase domain in liganded form being described by the amino acid sequence of SEQ ID NO: 1 and the structural coordinates of Table 2, wherein said complex is characterized by at least one of the following ErbB4 kinase domain/compound interactions:

(i) one or more interactions with amino acid residues of the ErbB4 kinase domain hinge region;

(ii) one or more interactions with amino acid residues of the ErbB4 kinase domain adenine pocket,

(iii) one or more interactions with amino acid residues of the ErbB4 kinase sugar pocket and phosphate region,

(iv) one or more interactions with amino acid residues of the ErbB4 kinase domain back pocket, and

(v) one or more interactions with amino acid residues of the ErbB4 kinase domain solvent interface.

In a seventh aspect of the present invention, there is provided an ErbB4 kinase domain/inhibitor complex, comprising: an ErbB4 kinase domain form being described by the amino acid sequence of SEQ ID NO: 1 and the structural coordinates of Table 2 and a compound that can form a complex with the ErbB4 kinase domain said complex is characterized by at least one of the following ErbB4 kinase domain/compound interactions:

(i) one or more interactions with amino acid residues of the ErbB4 kinase domain hinge region;

(ii) one or more interactions with amino acid residues of the ErbB4 kinase domain adenine pocket,

(iii) one or more interactions with amino acid residues of the ErbB4 kinase sugar pocket and phosphate region,

(iv) one or more interactions with amino acid residues of the ErbB4 kinase domain back pocket, and

(v) one or more interactions with amino acid residues of the ErbB4 kinase domain solvent interface.

### BRIEF DESCRIPTION OF THE DRAWINGS

FIG. 1 depicts the structure of two thienopyrimidine inhibitors before reaction with the protein.

FIG. 2 depicts a ribbon representation of the ErbB4K complexed with an irreversible inhibitor. A loop that is disordered in the crystal structure is shown as a dashed line. The figure was prepared with RIBBONS.

FIG. 3 depicts the binding of a thienopyrimidine inhibitor in the ATP binding site of ErbB4K. The inhibitor is highlighted with thick black lines. The hydrogen bond between the inhibitor and the backbone NH of Met799 is shown as a grey dashed line. The covalent bond between the inhibitor and Cys803 is shown as a black dashed line. The figure was created with QUANTA.

### DETAILED DESCRIPTION OF THE INVENTION

Table 1 is a table summarizing the crystal and data statistics obtained from ErbB4K crystal forms. Data on the unit cell is presented, including data on the crystal space group, unit cell dimensions, molecules per asymmetric cell and crystal resolution.

Table 2 is a table of the atomic structure coordinate data obtained from X-ray diffraction from the liganded ErbB4K crystal form.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “mutation” carries its traditional connotation and means a change, inherited, naturally occurring or introduced, in a nucleic acid or polypeptide sequence, and is used in its sense as generally known to those of skill in the art.

As used herein, the term “labeled” means the attachment of a moiety, capable of detection by spectroscopic, radiologic or other methods, to a probe molecule.

As used herein, the term “target cell” refers to a cell, into which it is desired to insert a nucleic acid sequence or polypeptide, or to otherwise effect a modification from conditions known to be standard in the unmodified cell. A nucleic acid sequence introduced into a target cell can be of variable length. Additionally, a nucleic acid sequence can enter a target cell as a component of a plasmid or other vector or as a naked sequence.

As used herein, the term “transcription” means a cellular process involving the interaction of an RNA polymerase with a gene that directs the expression as RNA of the structural information present in the coding sequences of the gene. The process includes, but is not limited to, the following steps: (a) the transcription initiation, (b) transcript elongation, (c) transcript splicing, (d) transcript capping, (e) transcript termination, (f) transcript polyadenylation, (g) nuclear export of the transcript, (h) transcript editing, and (i) stabilizing the transcript.

As used herein, the term “expression” generally refers to the cellular processes by which a biologically active polypeptide is produced from RNA.

As used herein, the term “transcription factor” means a cytoplasmic or nuclear protein which binds to a gene, or binds to an RNA transcript of such gene, or binds to another protein which binds to such gene or such RNA transcript or another protein which in turn binds to such gene or such RNA transcript, so as to thereby modulate expression of the gene. Such modulation can additionally be achieved by other mechanisms; the essence of “transcription factor for a gene” is that the level of transcription of the gene is altered in some way.

As used herein, the term “hybridization” means the binding of a probe molecule, a molecule to which a detectable moiety has been bound, to a target sample.

As used herein, the term “detecting” means confirming the presence of a target entity by observing the occurrence of a detectable signal, such as a radiologic or spectroscopic signal that will appear exclusively in the presence of the target entity.

As used herein, the term “sequencing” means determining the ordered linear sequence of nucleic acids or amino acids of a DNA or protein target sample, using conventional manual or automated laboratory techniques.

As used herein, the term “isolated” means for example oligonucleotides substantially free of other nucleic acids, proteins, lipids, carbohydrates or other materials with which they can be associated, such association being either in cellular material or in a synthesis medium. The term can also be applied to other molecule types including polypeptides, in which case the polypeptide will be substantially free of nucleic acids, carbohydrates, lipids and other undesired polypeptides.

As used herein, the term “substantially pure” means that the polynucleotide or polypeptide is substantially free of the sequences and molecules with which it is associated in its natural state, and those molecules used in the isolation procedure. The term “substantially free” means that the sample is at least 50%, preferably at least 70%, more preferably 80% and most preferably 90% free of the materials and compounds with which it is associated in nature.

As used herein, the term “primer” means a sequence comprising two or more deoxyribonucleotides or ribonucleotides, preferably more than three, and more preferably more than eight and most preferably at least about 20 nucleotides of an exonic or intronic region. Such oligonucleotides are preferably between ten and thirty bases in length.

As used herein, the term “DNA segment” means a DNA molecule that has been isolated free of total genomic DNA of a particular species. For example, a DNA segment encoding a erbB4 or erbB4K polypeptide refers to a DNA segment that encodes SEQ ID NO: 1 yet is isolated away from, or purified free from, total genomic DNA of a source species, such as Homo sapiens. Included within the term “DNA segment” are DNA segments and smaller fragments of such segments, and also recombinant vectors, including, for example, plasmids, cosmids, phages, viruses, and the like.

As used herein, the phrase “enhancer-promoter” means a composite unit that contains both enhancer and promoter elements. An enhancer-promoter is operatively linked to a coding sequence that encodes at least one gene product.

As used herein, the phrase “operatively linked” means that an enhancer-promoter is connected to a coding sequence in such a way that the transcription of that coding sequence is controlled and regulated by that enhancer-promoter. Techniques for operatively linking an enhancer-promoter to a coding sequence are well known in the art; the precise orientation and location relative to a coding sequence of interest is dependent, inter alia, upon the specific nature of the enhancer-promoter.

As used herein, the term “inhibitor candidate” means a substance that is believed to interact with another moiety, for example a given ligand that is believed to interact to at least partially inhibit the activity of a complete enzyme or enzyme polypeptide, or fragment thereof, and which can be subsequently evaluated for such an interaction and activity inhibition. In a like manner, the term “ErbB4 inhibitor candidate” means a substance that is believed to interact with another moiety, for example a given ligand that is believed to interact to at least partially inhibit the activity of a complete ErbB4 or ErbB4 polypeptide, or fragment thereof, and which can be subsequently evaluated for such an interaction and activity inhibition. Representative candidate compounds or substrates include xenobiotics such as drugs and other therapeutic agents, carcinogens and environmental pollutants, natural products and extracts, as well as endobiotics such as steroids, fatty acids and prostaglandins. Other examples of candidate substances that can be investigated using the methods of the present invention include, but are not restricted to, agonists and antagonists of a ErbB4 or ErbB4 polypeptide, toxins and venoms, viral epitopes, hormones (e.g., opioid peptides, steroids, etc.), hormone receptors, peptides, enzymes, enzyme substrates, co-factors, lectins, sugars, oligonucleotides or nucleic acids, oligosaccharides, proteins, small molecules and monoclonal antibodies.

As used herein, the term “modified” means an alteration from an entity's normally occurring state. An entity can be modified by removing discrete chemical units or by adding discrete chemical units. The term “modified” encompasses detectable labels as well as those entities added as aids in purification.

As used herein, the term “interaction” means any relationship between atoms or molecules whereby atomic and/or molecular conditions or forces exist which promote binding equilibrium between such atoms or molecules. Suitable examples include, but are not limited to covalent, electrostatic, hydrophobic, hydrophilic, hydrogen, and van der Waals bonding. The nature of such bonding relationships is known in the art and is described for instance in Mathews et al (1990) Biochemistry, Chapter 2, pgs 30-54.

As used herein, the terms “structure coordinates” and “structural coordinates” are interchangeable and mean mathematical coordinates derived from mathematical equations related to the patterns obtained on diffraction of a monochromatic beam of X-rays by the atoms (scattering centers) of a molecule, for instance ErbB4K, in crystal form. The diffraction data are used to calculate an electron density map of the repeating unit of the crystal. The electron density maps are used to establish the positions of the individual atoms within the unit cell of the crystal. Those of skill in the art understand that a set of structure coordinates determined by X-ray crystallography is not without standard error. For the purpose of this invention, any set of structure coordinates for ErbB4 or a ErbB4K mutant that have a root mean square (RMS) deviation from ideal of no more than 1.5 Å, when superimposed using the polypeptide backbone atoms on the structure coordinates listed in Table 2, shall be considered identical, except that for the activation loop and nucleotide binding loop such deviation from ideal have a RMS of no more than 10 Å.

As used herein, the term “asymmetric unit” means part of a symmetric object from which the whole is built up by repeats. Thus, it is the smallest unit from which the object can be generated by the symmetry operations of its point group.

As used herein, the term “molecular replacement” means a method that involves generating a preliminary model of ErbB4 or ErbB4K mutant crystal whose structure coordinates are unknown, by orienting and positioning a molecule whose structure coordinates are known within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal (Lattman, (1985) in Methods in Enzymology, t15: 55-77). Using the structure coordinates of erbB4K and erbB4K in liganded form provided by this invention, molecular replacement can be used to determine the structure coordinates of a crystalline mutant or homologue of ErbB4K or of a different crystal form of ErbBK.

As used herein, the terms “α-sheet” and “beta sheet” are interchangeable and mean the conformation of a polypeptide chain stretched into an extended zig-zig conformation. Portions of polypeptide chains that run “parallel” all run in the same direction. Polypeptide chains that are “antiparallel” run in the opposite direction from the parallel chains.

As used herein, the terms “α-helix” and “alpha helix” are interchangeable and mean the conformation of a polypeptide chain wherein the polypeptide backbone is wound around the long axis of the molecule in a left-handed or right-handed direction. The substituent groups of the amino acids protrude outward from the helical backbone, wherein the repeating unit of the structure is a single turn of the helix, which extends about 0.56 nm along the long axis.

As used herein, the term “mutant” means a polypeptide which is obtained by replacing at least one amino acid residue in a native erbB4 or erbB4K polypeptide with a different amino acid residue and/or by adding and/or deleting amino acid residues within the native polypeptide or at the N- and/or C-terminus of a polypeptide corresponding to a native erbB4 or erbB4K and which has substantially the same three-dimensional structure as the native erbB4 or erbB4K from which it is derived. By having substantially the same three-dimensional structure is meant having a set of atomic structure coordinates that have a root mean square deviation (RMS deviation) of less than or equal to about 1.5 Å, (10 Å for the activation loop and nucleotide binding loop) when superimposed with the atomic structure coordinates of the native erbB4 or erbB4K from which the mutant is derived when at least about 50% to 100% of the Cα atoms of the native erbB4 or erbB4K are included in the superposition. A mutant can have, but need not have, autophosphorylation activity.

As used herein, the term “space group” means a group or array of operations consistent with an infinitely extended regularly repeating pattern. It is the symmetry of a three-dimensional structure, or the arrangement of symmetry elements of a crystal. There are 230 space group symmetries possible; however, there are only 65 space group symmetries available for biological structures.

As used herein, the term “symmetry” means some spatial manipulation of an object resulting in an indistinguishable object. A symmetric object can, therefore, be superimposed on itself by some operation.

As used herein, the term “unit cell” means the fundamental portion of a crystal structure that is repeated infinitely by translation in three dimensions. A unit cell is characterized by three vectors a, b, and c, not located in one plane, which form the edges of a parallelepiped. Angles α, β and γ define the angles between the vectors: angle α is the angle between vectors b and c; angle β is the angle between vectors a and c; and angle γ is the angle between vectors a and b. The entire volume of a crystal can be constructed by regular assembly of unit cells; each unit cell comprises a complete representation of the unit of pattern, the repetition of which builds up the crystal.

As used herein, “monoclinic unit cell” means a unit cell wherein a≠b≠c and α=γ=90° and β≠90°. The vectors a, b and c describe the unit cell edges and the angles α, β, and γ describe the unit cell angles.

As used herein, “orthorhombic unit cell” means a unit cell wherein a≠b≠c; and α=β=γ=90°. The vectors a, b and c describe the unit cell edges and the angles α, β, and γ describe the unit cell angles.

As used herein, the term “crystal lattice” means the array of points defined by the vertices of packed unit cells.

As used herein, the term “active site” means that site in the erbB4K domain where substrate peptide binding, ATP binding and catalysis occur. For erbB4, the active site comprises at least the activation loop and the nucleotide binding loop.

As used herein, the term “activation loop” refers to a loop in tyrosine kinase domains between the conserved AspPheGly sequence and the conserved AlaProGlu sequence that is believed to act as a regulatory loop.

As used herein the terms “nucleotide-binding loop” and “glycine-rich loop” are synonomous and mean a loop in an RTK which contains the protein kinase-conserved glycine-rich consensus sequence.

As used herein, the term “autophosphorylation site” means a residue or residues in erbB4K that is phosphorylated by a domain of erbB4 itself.

As used herein the term “juxtamembrane region” means that portion of erbB4K located between the transmembrane helix and the tyrosine kinase domain.

As used herein, the terms “kinase insert” and “kinase insert domain” are synonymous and mean an additional domain not found in non-receptor tyrosine kinases or serine/threonine kinases. It is found between helices αD and αE in the C-terminal domain of receptor tyrosine kinases and can vary greatly in sequence and length.

As used herein, the term “C-terminal tail” means that region of an RTK that extends beyond the final helix of the C-terminal domain of the RTK.

As used herein, the term “N-terminal domain” means that region of an RTK that has a defined structure and precedes in sequence the hinge region.

As used herein, the term “modulate” means an increase, decrease, or other alteration of any or all chemical and biological activities or properties of a wild-type or mutant erbB4 or erbB4K polypeptide.

Description of ErbB4K Structure

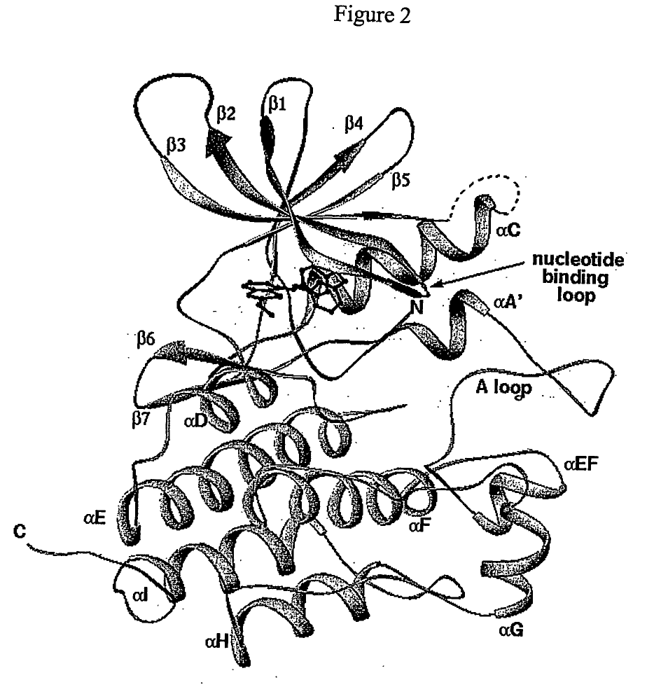

The overall architecture of ErbB4K was analogous to structures reported previously for both serine/threonine and tyrosine protein kinases (Johnson et al, Cell, 85: 149-158; Cox et al, Curr. Opin. Struct. Biol., 4: 893-901). A C_{α} trace of ErbB4 is shown in FIG. 2, where kinase secondary structural elements are labeled according to the convention originally given for cAPK (Knighton et al, Science, 253:407-413). ErbB4K folds into two domains, with catalysis occurring in a cleft between the two domains. Residues in the N-terminal domain are primarily responsible for ligating ATP, while residues in the C-terminal domain are involved in catalysis and substrate binding.

The N-terminal domain (residues 690-801) folds into a twisted β-sheet and one α-helix. The larger C-terminal domain (residues 802-999) contains eight α-helices (αD-αI) and a set of anti-parallel β-strands (β6/β7). Strands 6 and 7 are positioned at the interdomain interface adjacent to the N-terminal β-sheet. Like other kinases, ErbB4K also contains functionally important loop regions: the glycine-rich nucleotide binding loop (residues 725-730), the catalytic loop (residues 841-848) and the activation loop (residues 861-890), which will be described in further detail below.

Activation Loop

Protein kinases contain a large flexible loop, called the activation loop or A-loop, whose conformation is believed to regulate kinase activity. In many kinases, the conformation of the A-loop is controlled by the phosphorylation of specific residues within this region (Johnson et al). The activation loop generally begins with a conserved AspPheGly sequence (ErbB4K 861) and ends at a conserved AlaProGlu (ErbB4K 890, AlaLeuGlu in ErbB4K) (Johnson et al). In structures of inactive kinases, portions of this loop are often disordered. In those structures where the A loop is ordered, it often blocks either the substrate or ATP binding sites (Mohammadi et al; Wybenga-Groot et al; Hubbard et al—1997; Hubbard et al—1994; McTigue et al; and Xu et al). Upon phosphorylation, the A-loop is repositioned to contact residues in the C-terminal domain (Hubbard et al—1997). The activating phosphate can then interact with a cluster of basic residues, which includes a conserved arginine (ErbB4K R⁸⁴²), that precedes the catalytic aspartate (ErbB4K D843). The aspartyl residue of the AspPheGly motif ligates a Mg²⁺ ion, which, in turn contacts the β and γ phosphates of ATP.

In ErbB4K, the activation loop corresponds to residues 861-890 and contains a single tyrosine at position 875. In the ErbB4K structure presented, the A-loop is completely ordered and does not significantly block either the ATP or substrate binding sites. Unlike other A loops observed to date, the A loop in ErbB4 contains a short helix (helix A) immediately following the AspPheGly motif.

In ErbB4, TyR⁸⁷⁵ is found in a similar position within the A-loop as tyrosines required for activation of other tyrosine kinases. The side chain of this residue in the ErbB4K crystal structure is pointing towards the interior of the protein rather than solvent and its OH forms a hydrogen bond to the backbone carbonyl of Cys891. It is not clear whether phosphorylation of this tyrosine in ErbB4 is required for full activity of the kinase.

Nucleotide Binding Loop

The nucleotide binding loop (NB loop) contains residues responsible for binding the triphosphate moiety of ATP in the correct position for catalysis (Johnson etal and Cox et al). This glycine-rich loop is believed to be quite flexible and is often either disordered or has high b-factors in many unliganded kinase structures sites (Mohammadi et al; Wybenga-Groot et al; Hubbard et al—1997; Hubbard et al—1994; McTigue et al; and Xu et al). In ErbB4K, this loop is ordered and occupies a similar position to that seen in other kinase structures.

Catalytic Loop

The catalytic loop of protein kinases lies between αE and β7 and contains an invariant aspartic acid (D843 in ErbB4) that serves as the catalytic base in the phosphotransfer reaction (Johnson et al). The sequence (HRDLAARN), as well as the backbone and side chain positions of this loop are similar to those in the unliganded EphB2, FGFR¹, Tie2, IRK and VEGFR² and in the ternary phosphorylated IRK complex structures sites (Mohammadi et al; Wybenga-Groot et al; Hubbard et al—1997; Hubbard et al—1994; and McTigue et al).

Inhibitor Binding Site

The ATP binding site can be broken down into several regions: hinge, adenine pocket, solvent interface, back pocket and sugar pocket. ATP is modeled into ErbB4 based on the activated IR structure. The hinge region runs from thR⁷⁹⁶ to pro800 and would be expected to form hydrogen bonds with the adenine base of ATP. The adenine pocket would be formed by the hinge residues on the side and ala749 and leu724 on top and leu850 on the bottom. The back pocket in ErbB4 is an elongated channel that can be divided by 2 regions defined by (1) val732, ala749, lys751, thR⁷⁹⁶, asp861, and thR⁸⁶⁰, and (2) met772, val781, leu783, leu794, thR⁷⁹⁶, and phe862. Typically, potential inhibitors bind in region (1) and do not reach back into region (2). A surface at the solvent interface formed by residues his801, gly802, cys803, glu806, and glu810 could form interactions with inhibitors. The ribose or sugar pocket is defined by asn848, thR⁸⁶⁰, cys803, arg847, val732, and gly725.

Inhibitor/ErbB4K Complex Structure

The structure of nonphosphorylated ErbB4 was solved in the presence of a thienopyrimidine inhibitor (formula Ia):

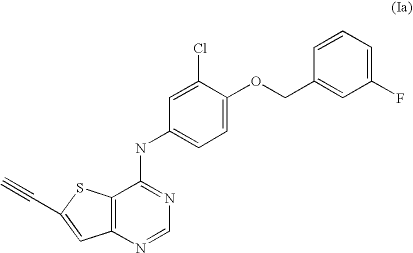

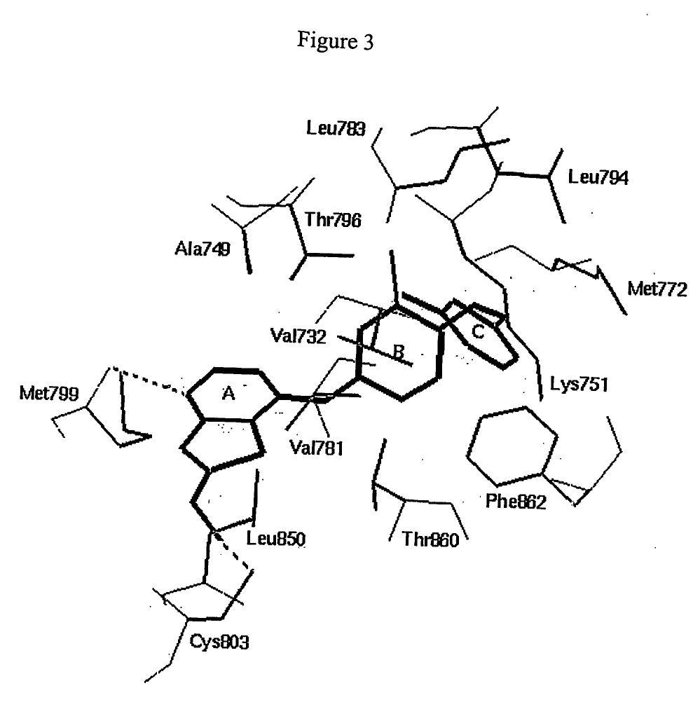

The inhibitor binds in the ATP binding site tunneling into the back of the pocket. The thienopyrimidine group, like the adenosine base of ATP, hydrogen bonds to the hinge region between the N and C-terminal domains (FIG. 3). The inhibitor makes a single hydrogen bond to the protein. N1 of the inhibitor hydrogen bonds to the backbone NH of Met799. The thienopyrimidine ring is sandwiched from the top and bottom by the side chains of Ala749 and Leu850, respectively. The side chain of cys803 adds into the alkynyl group forming a cis double bond. The B and C rings of the inhibitor lie deep in the back of the ATP binding site. The B ring packs against the side chains of Val732, Ala749, Lys751, ThR⁷⁹⁶, and ThR⁸⁶⁰, while the C ring sits in a hydrophobic pocket formed by Met772, Val781, Leu783, Leu794, ThR⁷⁹⁶, and Phe862.

Co-crystal structures were also solved using the thienopyrimidine compound of formula (Ib):

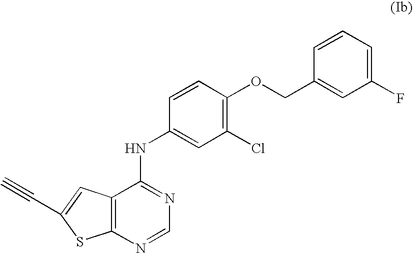

Further compounds which may be co-crystallized with the ErbB4 kinase domain include compounds of formula (I): 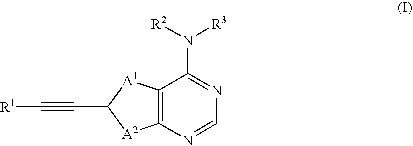
wherein:
one of A¹ and A² is S and the other is CH;
R¹ is H or —(CR¹¹R¹¹)ₙ—R⁵;
R² is H or C₁₋₆alkyl;
R³ is selected from the group consisting of aryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰, and heteroaryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴ is selected from the group consisting of H, C₁₋₆alkyl, —(CH₂)ₙNR⁹R¹⁰, —(CH₂)ₙheterocyclyl, —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, arylC₁₋₆alkenylene in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkenylene in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of heterocyclyl, —N(R⁶)—C(O)—N(R⁶)(R⁷)—N(R⁶)—C(S)—N(R⁶)(R⁷), —N(R⁶)—C(O)—OR⁷, —N(R⁶)—C(O)—(CH₂)ₙ—R⁷—N(R⁶)—SO₂R⁶, —(CH₂)ₙNR⁶R⁷₁—(CH₂)ₙOR⁷—(CH₂)ₙSR⁸—(CH₂)ₙS(O)R⁸, —(CH₂)ₙS(O)₂R⁸, —OC(O)R⁸, —OC(O)OR⁸, —C(O)NR⁶R⁷, heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙC(O)R⁸, —C(O)₂R⁸, —(CH₂)ₙSR⁸, —(CH₂)ₙS(O)R⁸, —(CH₂)ₙS(O)₂R⁸, —(CH₂)ₙR⁸, —(CH₂)ₙCN, aryl optionally substituted with one or more substituents selected from the group consisting of halo, —CF₃, C₁₋₆alkoxy, —NO₂, C₁₋₆alkyl, —CN, —(CH₂)ₙOR⁸, —(CH₂)ₙheterocyclyl, —(CH₂)ₙheteroaryl, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —(CH₂)ₙOR⁸, —(CH₂)ₙheterocyclyl, —(CH₂)ₙheteroaryl, —SO₂R⁹, and —(CH₂)ₙNR⁹R¹⁰, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H, C₁₋₆alkyl, C₃₋₈cycloalkyl, and —C(O)R¹¹ or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

Such thienopyrimidines are described in U.S. Provisional Patent Application Ser. No. 60/342,207 filed Dec. 19, 2001 which was filed Dec. 13, 2002 as PCT Patent Application No. PCT/US02/39872 and published as WO 03/053446 on Jul. 3, 2003. Such applications are incorporated herein by reference to the extent they disclose and describe such thienopyrimidines as well as the making and use thereof.

As recited above, the present invention provides an ErbB4 kinase domain in liganded crystalline form. Such ErbB4 liganded kinase domain is described by the amino acid sequence of SEQ ID NO: 1 and the structural coordinates of Table 2. SEQ ID NO: 1 is encoded by the DNA sequence of SEQ ID NO: 2. In another embodiment, is an ErbB4 liganded kinase domain described by the amino acid sequence encoded by the DNA sequence of SEQ ID NO: 2 and the structural coordinates of Table 2. In a further embodiment, is a substantially pure and isolated ErbB4 liganded kinase domain described by the amino acid sequence of SEQ ID NO: 1 and the structural coordinates of Table 2. In another embodiment, is a substantially pure and isolated ErbB4 liganded kinase domain described by the amino acid sequence encoded by the DNA sequence of SEQ ID NO: 2 and the structural coordinates of Table 2.

In one embodiment the liganded ErbB4 kinase domain in crystalline form has lattice constants of a=63.95 Å, b=63.95 Å, c=163.95 Å, a=90°, β=90°, and γ=90°. In one embodiment, the liganded ErbB4 kinase domain in crystalline form has a space group of P4₃. In another embodiment, the liganded ErbB4 kinase in crystalline form has an entire NT region which is ordered. In still another embodiment, the liganded ErbB4 kinase in crystalline form has structural coordinates having a deviation from ideal with a RMS of no more than 1.5 Å except that the activation loop and/or a nucleotide binding loop have structural coordinates having a deviation from ideal with a RMS of no more than 10 Å. In a further embodiment, the liganded ErbB4 kinase in crystalline form has an activation loop and/or a nucleotide binding loop have structural coordinates having a deviation from ideal with a RMS of no more than 10 Å.

In another embodiment, there is provided an ErbB4 kinase domain/inhibitor complex which includes an ErbB4 liganded kinase domain described by the amino acid sequence of SEQ ID NO: 1 or 2 and the structural coordinates of Table 2 and a compound capable of at least one of the following interactions with the cFMS kinase domain:

(i) one or more interactions with amino acid residues of the ErbB4 kinase domain hinge region;

(ii) one or more interactions with amino acid residues of the ErbB4 kinase domain adenine pocket;

(iii) one or more interactions with amino acid residues of the ErbB4 kinase sugar pocket;

(iv) one or more interactions with amino acid residues of the ErbB4 kinase domain back pocket; and

(v) one or more interactions with amino acid residues of the ErbB4 kinase domain solvent interface;

preferably

(i) one or more interactions with amino acid residues 796, 797, 798, 799, and 800;

(ii) one or more interactions with amino acid residues 724, 749, and 850;

(iii) one or more interactions with amino acid residues 848, 860, 803, 847, 732, and 725;

(iv) one or more interactions with amino acid residues 732, 749, 751, 796, 861, 860, 772, 781, 783, 794, 796, and 862; and

(v) one or more interactions with residues 801, 802, 803, 806, and 810.

More preferred embodiments of interactions (i), (ii), (iii), (iv), and (v) are described following.

The amino acid region referred to in the interaction described in (i), which include amino acid residues 796-800, is typically referred to as the hinge region. In one embodiment, there are one or more kinase domain/compound bonding interactions with at least one of amino acid residues 796 to 800, preferably at least one of the bonding interactions is a hydrogen bonding interaction. In another embodiment, there are two or more bonding interactions with at least one of amino acid residues 796 to 800, preferably at least one of the bonding interactions is a hydrogen bonding interaction. In a further embodiment, there are three or more bonding interactions with at least one of amino acid residues 796 to 800, preferably at least one of the bonding interactions is a hydrogen bonding interaction. In a still further embodiment, there are four or more bonding interactions with at least one of amino acid residues 796 to 800, preferably at least one of the bonding interactions is a hydrogen bonding interaction.

In a preferred embodiment, there are one or more kinase domain/compound hydrogen bonding interactions with at least one of amino acid residues 796 to 800, alternatively two or more hydrogen bonding interactions with at least one of amino acid residues 796 to 800, in a further alternative embodiment three or more hydrogen bonding interactions with at least one of amino acid residues 796 to 800, and in a still further embodiment four or more bonding interactions with at least one of amino acid residues 796 to 800.

In a more preferred embodiment, there is a kinase domain/compound hydrogen bonding interaction with methionine 799, preferably one hydrogen bonding interaction with the backbone NH of methionine 799. Typically, this hydrogen bond is at a distance of 2.5 to 3.5, preferably 2.6 to 3.3, more preferably 2.8 to 3.0 Å.

In another embodiment, there is a kinase domain/compound hydrogen bonding interaction with methionine 799 and there are one or more kinase domain/compound bonding interactions with at least one of amino acid residues 796 to 800. In another embodiment, there is a kinase domain/compound hydrogen bonding interaction with methionine 799 and there are two or more bonding interactions with at least one of amino acid residues 796 to 800. In a further embodiment, there is a kinase domain/compound hydrogen bonding interaction with methionine 799 and there are three or more bonding interactions with at least one of amino acid residues 796 to 800. Finally, in a still further embodiment, there is a kinase domain/compound hydrogen bonding interaction with methionine 799 and there are four or more bonding interactions with at least one of amino acid residues 796 to 800. Preferably, the one of the hydrogen bonding interactions with methionine 799 is with the backbone NH of methionine 799. Typically, this hydrogen bond is at a distance of 2.5 to 3.5, preferably 2.6 to 3.3, more preferably 2.8 to 3.0.

The amino acid region referred to in the interaction described in (ii), which includes amino acid residues 749, 724, and 850, is commonly termed the adenine pocket. In one embodiment, there are one or more kinase domain/compound interactions with at least one of amino acid residues 749, 724, and 850, preferably two or more interactions with at least two of amino acid residues 749, 724, and 850, more preferably three or more interactions with at least three of amino acid residues 749, 724, and 850, most preferably four or more bonding interactions with amino acid residues 749, 724, and 850.

In one embodiment, there is a kinase domain/compound hydrophobic interaction with alanine 749, preferably an interaction with the side chain of alanine 749. In another embodiment, there is a kinase domain/compound interaction with leucine 724. In a further embodiment, there is a kinase domain/compound interaction with leucine 850, preferably an interaction with the side chain of leucine 850. In a more preferred embodiment, there is one kinase domain/compound interaction with at least one of alanine 749, leucine 724, and leucine 850. In a most preferred embodiment, there are two kinase domain/compound bonding interactions: (i) an interaction with the side chain of alanine 749 and ii) an interaction with the side chain of leucine 850.

The amino acid region referred to in the interactions described in (iii) describe what is commonly termed the sugar (ribose) pocket and is defined by amino acid residues 848, 860, 803, 847, 732, and 725. In one embodiment, there are one or more kinase domain/compound interactions with at least one of amino acid residues 848, 860, 803, 847, 732, and 725, preferably two or more interactions with amino acid residues 848, 860, 803, 847, 732, and 725.

In one embodiment, there is a kinase domain/compound interaction with asparagine 848. In another embodiment, there is a kinase domain/compound interaction with threonine 860. In another embodiment, there is a kinase domain/compound interaction with cysteine 803, preferably a covalent interaction between cysteine 803 and the compound, more preferably a covalent interaction between cysteine 803 and the alkynyl group of the compound forming a cis double bond. In another embodiment, there is a kinase domain/compound interaction with arginine 847. In another embodiment, there is a kinase domain/compound interaction with valine 732. In another embodiment, there is a kinase domain/compound interaction with glycine 725.

The amino acid region referred to in the interactions in (iv) described what is commonly termed the back pocket, which is formed by residues 732, 749, 751, 796, 861, 860, 772, 781, 783, 794, 796, and 862. In one embodiment, there are one or more kinase domain/compound interactions with at least one of amino acid residues 732, 749, 751, 796, 861, 860, 772, 781, 783, 794, 796, and 862, preferably two or more interactions with at least two of amino acid residues 732, 749, 751, 796, 861, 860, 772, 781, 783, 794, 796, and 862, more preferably three or more interactions with at least three of amino acid residues 732, 749, 751, 796, 861, 860, 772, 781, 783, 794, 796, and 862, still more preferably four or more bonding interactions with at least four of amino acid residues 732, 749, 751, 796, 861, 860, 772, 781, 783, 794, 796, and 862.

In one embodiment, there is a kinase domain/compound interaction with valine 732, preferably a hydrophobic interaction with valine 732. In another embodiment, there is a kinase domain/compound interaction with alanine 749, preferably a hydrophobic bonding interaction with alanine 749. In another embodiment, there is a kinase domain/compound interaction with lysine 751, preferably a hydrophobic interaction with lysine 751. In a further embodiment, there is a kinase domain/compound interaction with threonine 796, preferably a hydrophobic interaction with threonine 796. In another further embodiment, there is a kinase domain/compound hydrophobic interaction with aspartic acid 861, preferably a hydrophobic interaction with aspartic acid 861. In another embodiment, there is a kinase domain/compound interaction with threonine 860, preferably a hydrophobic interaction with threonine 860. In another embodiment, there is a kinase domain/compound interaction with methionine 772, preferably a hydrophobic interaction with methionine 772. In another embodiment, there is a kinase domain/compound interaction with valine 781, preferably a hydrophobic interaction with valine 781. In another embodiment, there is a kinase domain/compound interaction with leucine 783, preferably a hydrophobic interaction with leucine 783. In another embodiment, there is a kinase domain/compound interaction with leucine 794, preferably a hydrophobic interaction with leucine 794. In another embodiment, there is a kinase domain/compound interaction with threonine 796, preferably a hydrophobic interaction with threonine 796. In another embodiment, there is a kinase domain/compound interaction with phenylalanine 862, preferably a hydrophobic interaction with phenylalanine 862.

The amino acid region referred to in the interactions in (v) describe what is commonly termed the solvent interface, which is formed by residues 801, 802, 803, 806, and 810. In one embodiment, there are one or more kinase domain/compound interactions with at least one of amino acid residues 801, 802, 803, 806, and 810, preferably two or more interactions with at least two of amino acid residues 801, 802, 803, 806, and 810, more preferably three or more interactions with at least two of amino acid residues 801, 802, 803, 806, and 810.

In one embodiment, there is a kinase domain/compound interaction with histidine 801. In another embodiment, there is a kinase domain/compound interaction with glycine 802. In one embodiment, there is a kinase domain/compound interaction with cysteine 803. In another embodiment, there is a kinase domain/compound interactions glutamic acid 806. In a further embodiment, there is a kinase domain/compound interaction with glutamic acid 810.

The method of ErbB4 inhibitor design of the present invention includes as a first step: generating a three dimensional computer model which represents a ErbB4 kinase domain in liganded form, said kinase domain being described by the amino acid sequence of SEQ ID NO: 1 and having the structural coordinates of Table 2. Typically, such a computer model of SEQ ID NO: 1 and the structural coordinates of Table 2 is constructed utilizing a commercially available software program. Software programs for generating three-dimensional graphical representations of molecules or portions thereof from a set of structural coordinates are well known and used in the art. Suitable examples of such computer programs for viewing or otherwise manipulating protein structures include, but are not limited to, the following: Midas (University of California, San Francisco), MidasPlus (University of California, San Francisco),MOIL (Univeristy of Illinois), Yummie (Yale University), Sybyl (Tripos, Inc.), Insight/Discover (Biosym Technologies), MacroModel (Columbia University), Quanta (Molecular Simulations, Inc.), CNS (Molecular Simulations, Inc.), Cerius (Molucular Simulations, Inc.), Alchemy (Tripos, Inc.), LabVision (Tripos, Inc.), Rasmol (Glaxo Research and Development), Ribbon (University of Alabama), NAOMI (Oxford University), Explorer Eyechem (Silicon Graphics, Inc.), Univision (Cray Research), Molscript (Uppsala University), Chem-3D (Cambridge Scientific), Chain (Baylor College of Medicine), O (Uppsala University), GRASP (Columbia University), X-Plor (Molecular Simulations, Inc., Yale University), Spartan (Wavefunction, Inc.), Catalyst (Molecular Simulations, Inc.), Molcadd (Tripos, Inc.), VMD (University of Illinois/Beckman Institute), Sculpt (Interactive Simulations, Inc.), Procheck (Brookhaven National Laboratory), DGEOM (QCPE), RE_VIEW (Brunel University), Modeller (Birbeck College, University of London), Xmol (Minnesota Supercomputing Center), Protein Expert (Cambridge Scientific), HyperChem (Hypercube), MD Display (University of Washington), PKB (National Center for Biotechnology Information, NIH), ChemX (Chemical Design, Ltd.), Cameleon (Oxford Molecular, Inc.), and Iditis (Oxford Molecular, Inc.).

Once the three dimensional model of the ErbB4 kinase domain is established candidate inhibitor compounds may be evaluated utilizing the model and the selected software application. Initially, it is understood that the term “evaluate” includes within its scope, without limitation, de novo inhibitor molecular design, computer-aided optimization of known candidate inhibitors, as well as computer-based selection of candidate inhibitors. Various computational analysis methods are known in the art for the evaluation of potential binding interactions between a polypeptide binding pocket and a candidate inhibitor molecule. Such methods typically utilize at least one of the software packages recited above and are known in the art. Computational and other evaluation methods are described for instance in U.S. Pat. Nos. 6,251,620 and 6,356,845, such patents being incorporated herein by reference to the extent that they disclose computational and other evaluation methods for drug design, selection and/or optimization.

Examples of protein-inhibitor interactions which are screened for include potential covalent, electrostatic, hydrophobic, hydrophilic, van der Waals, and hydrogen bonding between the ErbB4 kinase molecule and candidate inhibitors as well as favorable candidate inhibitor conformations within the ErbB4 kinase binding pocket.

In one embodiment, evaluation of compounds as potential ErbB4 inhibitors using said model comprises identifying compounds capable of at least one of the following ErbB4 kinase domain/compound interactions:

(i) one or more interactions with amino acid residues of the ErbB4 kinase domain hinge region;

(ii) one or more interactions with amino acid residues of the ErbB4 kinase domain adenine pocket,

(iii) one or more interactions with amino acid residues of the ErbB4 kinase sugar pocket and phosphate region,

(iv) one or more interactions with amino acid residues of the ErbB4 kinase domain back pocket, and

(vi) one or more interactions with amino acid residues of the ErbB4 kinase domain solvent interface;

preferably

(i) one or more interactions with amino acid residues 796, 797, 798, 799, and 800;

(iii) one or more interactions with amino acid residues 724, 749, and 850;

(iii) one or more interactions with amino acid residues 848, 860, 803, 847, 732, and 725;

(iv) one or more interactions with amino acid residues 732, 749, 751, 796, 861, 860, 772, 781, 783, 794, 796, and 862; and

(v) one or more interactions with residues 801, 802, 803, 806, and 810.

Further preferred embodments of the interactions (i), (ii), (iii), (iv), and (v) are as described above.

If evaluation indicates that a compound shows promise as a candidate inhibitor the compounds are selected for further testing based on said evaluation. An inhibitor candidate is generally sought which can exist in a conformation, which appears to be structurally compatible with at least a part of the ErbB4 kinase domain binding pocket. Such conformation will be sterically and energetically compatible with the ErbB4 kinase domain. Typically, the above listed non-covalent or secondary bonding interactions will be important in the interaction of the candidate inhibitor and the ErbB4 kinase domain. In addition, other conformational factors include the overall three dimensional structure and orientation of the candidate inhibitor within the protein structure, especially the binding pocket as well as spacial and energetic relationships of the various functional groups of the candidate inhibitor and ErbB4 kinase domain which have potential for interaction. The further testing done typically is to evaluate the inhibitory effect on the kinase activity of ErbB4 and may take the form of enzyme or cell based assays as well as other assays known in the art for measuring the inhibitory effect on the kinase activity of ErbB4.

The present invention also provides a method of inhibiting ErbB4 in a mammal, which includes administering to said mammal a therapeutically effective amount of a compound that can form a complex with a ErbB4 kinase domain thereby resulting in a ErbB4 kinase domain in liganded form. Also provided is a method of treating a disorder characterized by inappropriate ErbB4 activity in a mammal which includes administering to said mammal a therapeutically effective amount of a compound that can form a complex with a ErbB4 kinase domain thereby resulting in a ErbB4 kinase domain in liganded form.

Compounds useful in the treatment methods of the present invention include those having interactions (i), (ii), (iii), (iv), and (v) with the ErbB4 kinase domain. Such interactions are as described above.

The inappropriate ErbB4 activity referred to herein is any ErbB4 activity that deviates from the normal ErbB4 activity expected in a particular mammalian subject. Inappropriate ErbB4 activity may take the form of, for instance, an abnormal increase in activity, or an aberration in the timing and or control of ErbB4 activity. Such inappropriate activity may result then, for example, from overexpression or mutation of the protein kinase leading to inappropriate or uncontrolled activation. Furthermore, it is also understood that unwanted ErbB4 activity may reside in an abnormal source, such as a malignancy. That is, the level of ErbB4 activity does not have to be abnormal to be considered inappropriate, rather the activity derives from an abnormal source.

While it is possible that, for use in therapy, therapeutically effective amounts of the compounds described in the present invention, as well as salts, solvates and physiologically functional derivatives thereof, may be administered as the raw chemical, it is possible to present the active ingredient as a pharmaceutical composition. Accordingly, the invention further provides pharmaceutical compositions, which include therapeutically effective amounts of the compound described herein and salts, solvates and physiological functional derivatives thereof, and one or more pharmaceutically acceptable carriers, diluents, or excipients. The compounds of the formula (I) and salts, solvates and physiological functional derivatives thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the present invention or salts, solvates and physiological functional derivatives thereof, with one or more pharmaceutically acceptable carriers, diluents or excipients.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the present invention, depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compounds of the present invention, and salts, solvates and physiological functional derivatives thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of the present invention and salts, solvates and physiological functional derivatives thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamidephenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

A therapeutically effective amount of a compound of the present invention will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of a compound of the present invention for the treatment of neoplastic growth, for example colon or breast carcinoma, will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, or physiologically functional derivative thereof, may be determined as a proportion of the effective amount of the compound of the present invention per se. It is envisaged that similar dosages would be appropriate for treatment of the other conditions referred to above.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification.

Structure Determination

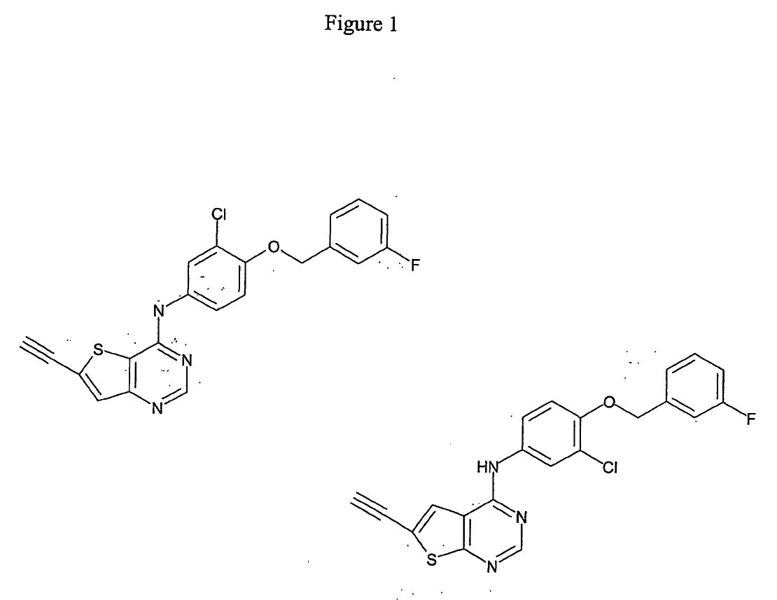

Modeling and limited proteolysis of the entire intracellular domain of ErbB4 were used to define a construct suitable for structural studies (residues 690-999). A 6×-His tag was added at the N-terminus to aid in the purification. The construct was expressed in baculovirus-infected insect cells and purified by standard chromatographic procedures. Crystallization screens were performed using nonphosphorylated protein complexed with an irreversible inhibitor (FIG. 1). Crystals were obtained in the tetragonal space group P4₃ with two molecules in the asymmetric unit.

The structure was solved by molecular replacement using the structure of the FGFR¹ as a search model [molecule 1 of PDB entry 1FGK]. The structure was refined to an R-factor of 21% at 2.5 Å resolution (Table 1). 8 residues at the N-terminus, 7 residues at the C-terminus and 6 residues within a surface exposed loop (residues 754-761) were disordered and could not be modeled. The structure of the two molecules in the asymmetric unit was essentially identical with a C_{α} rmsd of 0.20 Å.

Certain embodiments of the present invention will now be illustrated by way of example only.

Materials and Methods

Construct Generation

A combination of limited proteolysis and modeling was used to define the construct for structural studies. First, the cytoplasmic domain of ErbB4 (residues 690-1309) was ligated in frame behind a 6×His tag (MKKGHHHHHHG) in a pFastBac1 vector (Invitrogen). The cloned sequence was identical to that reported in GENBANK (L07868).

Limited proteolysis was performed on purified protein from the 6×His-EphB4_{—}690-1309 construct to define a smaller catalytic domain (see below for more details). Proteolysis suggested that both the C-terminus could be truncated. Therefore, a second construct was generated corresponding to residues 690-999 fused to a 6×his tag (MKKGHHHHHHG). The His-tagged kinase domain was cloned by PCR from the pFastBac1-His-EphB4_{—}690-1309 construct and ligated into a pFastBac1 vector (Invitrogen).

Both constructs were transfected into Spodoptera frugiperda (sf-9) cells, single plaques were isolated, and high titer stocks were generated. The proteins were expressed and purified as described below.

Limited Proteolysis

Purified 6×His-ErbB4_{—}690-1309 was digested with a panel of 8 proteases in a 96 well plate. 5 ug of 6×His-ErbB4 (5 uL at 1 mg/mL) was added to 5 uL of 10 mg/mL protease in 20 uL of reaction buffer (56 mM Tris-HCl, pH 8.0, 100 mM NaCl). Reactions were stopped at 0.75, 2, and 18 hours with 10 uL of 4×SDS-PAGE sample buffer. All digests where analyzed by SDS-PAGE (NuPAGE Novex 10% Bis-Tris gel, MES running buffer). Bands of interest were electroblotted on PVDF membrane and subjected to Edman sequencing.

| | Source (Boehringer Mannheim |
|---|---|
| Proteases used | unless otherwise noted) |
| 1.) Trypsin | catalogue # 1418475 |
| 2.) Chymotrypsin | catalogue # 1418467 |
| 3.) Lys C | catalogue # 1047825 |
| 4.) Glu C | catalogue # 1047817 |
| 5.) Asp N | catalogue # 1054589 |
| 6.) Arg C | catalogue # 1370529 |
| 7.) Thermolysin | catalogue # 161586 |
| 8.) Subtilisin | catalogue # 572908 (Calbiochem) |

Protein Fermentation/Purification

Fermentation: Large-scale (2L) virus preparations for fermentation were made by infecting Sf-9 cells growing in Grace's Supplemented medium (GIBCO/Life Technologies)+0.1% capluronic® F-68 (GIBCO/Life Technologies)+10% FBS (HyClone Laboratories) at a multiplicity of infection (MOI) of 0.1 in 6L shake flasks at 27.5° C. and 120 RPM. Viral supernatants were harvested at 72 hours post-infection via centrifugation at 2500 RPM for 20 minutes. Viral titers were determined via ELISA. A 36L stirred bioreactor (University Research Glassware) was outfitted with external overhead stirrer & water bath and internal dip tubes, heat-transfer coil, paddle-style impeller and dO₂ probe. The bioreactor was inoculated with Trichoplusia ni (T. ni) cells [kindly obtained from JRH BioSciences (Woodland, Calif.)] at ˜0.5×10⁶/mL. The culture was grown in Ex-Cell™ 405 insect cell medium (JRH BioSciences). Temperature was maintained at 27.5° C. using an external water bath and an internal temperature probe & heat-transfer coil. Agitation was maintained at 30 RPM using an external overhead drive and an internal paddle-type impeller. Dissolved oxygen was maintained at 50% via sparging under the control of an internal dO₂ probe. Cells were allowed to double overnight at the above parameters, and the culture was then infected at a density of ˜1×10⁶/mL at MOI=1. The culture was monitored daily for pH, glucose, lactate and glutamine levels as well as cell count and viability via trypan blue exclusion. Infection was allowed to proceed at the above parameters, and cells were harvested at 48 hours post-infection using a Centritech® 100 continuous flow centrifuge (DuPont). Concentrated cells were subsequently centrifuged at 2000 RPM for 20 minutes and washed with protease inhibitor buffer [1× Dulbecco's PBS (GIBCO/Life Technologies), 1 mM EDTA (Sigma), 1 mM p-aminobenzamidine (Sigma), 1 μg/mL aprotinin (Boehringer Mannheim), 1 μg/mL leupeptin (Boehringer Mannheim)]. Cells were centrifuged again at 2000 RPM for 20 minutes. The supernatant was decanted, and the cells were flash frozen in a dry ice/ethanol bath and stored at −80° C. until further purification.

Purification:

All operations were carried out at 4° C. Insect cells were resuspended and thawed in buffer A (25 mM HEPES pH 7.5, 750 mM NaCl, 10% glycerol, 25 mM imidazole) supplemented with a protease inhibitor cocktail (Sigma), 1 mM MgCl₂ and 5 μg/ml of DNAse I and RNAse. The cells were lysed with a Polytron homogenizer (Brinkmann) and then centrifuged for 1 hour at 30,000 g (14,000 rpm) in a Sorvall SLA 1500 rotor. The pelleted material was discarded, and the supernatant was filtered through a 4.5μ filter (PALL Corp.). The lysate was directly loaded onto a Ni-Chelating Sepharose FF column (Amersham Pharmacia). Before sample loading, the column was equilibrated with 5 column volumes (CV's) of buffer A. After sample loading, the column was washed for 5 CV's with buffer A. The protein was eluted with a 20 CV linear gradient from 50 to 500 mM imidazole in buffer A. Fractions containing ErbB4K protein were analyzed by polyacrylamide gel electrophoresis and pooled. The pool was diluted 8 fold in Buffer B (20 mM HEPES, 20 mM NaH₂PO₄, pH 6.8, 10% glycerol) and loaded onto Ceramic HA (Bio-Rad) column previously equilibrated in buffer B. Active ErbB4 flows through and does not bind. The flow-through fraction was brought to 0.6M (NH₄)₂SO₄ by addition of a 2.5M (NH₄)₂SO₄ stock solution and the sample applied to a Phenyl HIC column previously equilibrated in buffer E (20 mM Tris-HCl, pH 7.5, 0.6M (NH₄)₂SO₄). A reverse linear gradient to 100% buffer F (20 mM Tris-HCl, pH 7.5, 10% glycerol) was used to elute protein. Fractions containing pure ErbB4 were pooled, aliquoted and stored at −80° C.

Preparation of Inhibitor Candidate Compounds

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

g (grams); mg (milligrams);

L (liters); mL (milliliters);

μL (microliters); psi (pounds per square inch);

M (molar); mM (millimolar);

mol (moles); mmol (millimoles);

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted.

¹H NMR spectra were recorded on a Varian VXR-300, a Varian Unity-300, a Varian Unity-400 instrument, a Brucker AVANCE-400, or a General Electric QE-300. Chemical shifts are expressed in parts per million (ppm, 8 units). Coupling constants are in units of hertz (Hz). Splitting patterns describe apparent multiplicities and are designated as s (singlet), d (doublet), t (triplet), q (quartet), quint (quintet), m (multiplet), br (broad).

HPLC were recorded on a Gilson HPLC or Shimazu HPLC system by the following conditions. Column: 50×4.6 mm (id) stainless steel packed with 5 μm Phenomenex Luna C-18; Flow rate: 2.0 mL/min; Mobile phase: A phase=50 mM ammonium acetate (pH 7.4), B phase=acetonitrile, 0-0.5 min (A: 100%, B: 0%), 0.5-3.0 min (A:100-0%, B:0-100%), 3.0-3.5 min (A: 0%, B: 100%), 3.5-3.7 min (A: 0-100%, B: 100-0%), 3.7-4.5 min (A: 100%, B: 0%); Detection: UV 254 nm; Injection volume: 3 μL.

Low-resolution mass spectra (MS) were recorded on a JOEL JMS-ΔX505HA, JOEL SX-102, or a SCIEX-APIiii spectrometer; LC-MS were recorded on a micromass 2MD and Waters 2690; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (1R) spectra were obtained on a Nicolet 510 FT-IR spectrometer using a 1-mm NaCl cell. Most of the reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck).

Compounds of Formula I(a) and I(b) can be prepared according to the synthetic sequence detailed in the Examples section following.

### Example 1

### N-{3-chloro-4-[(3-fluorobenzyl)oxy]phenyl}-6-ethynylthieno[3,2-d]pyrimidin-4-amine

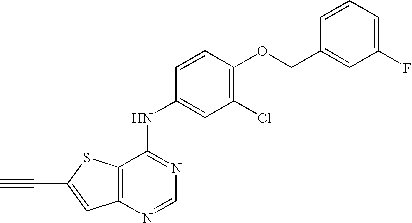 Step A

### Preparation of 6-Bromo-N-{3-chloro-4-[(3-fluorobenzyl)oxy]phenyl}thieno[3,2-d]pyrimidin-4-amine hydrochloride

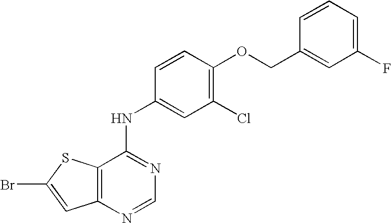

6-Bromo-4-chlorothieno[3,2-d]pyrimidine (2) (1.05 g, 4 mmol) and 3-chloro-4-[(3-fluorobenzyl)oxy]aniline (986 mg, 3.9 mmol) were heated at 60° C. for 3 h in isopropanol (30 mL). The mixture was concentrated and the resulting material was triturated with ethyl ether and collected by suction filtration to yield the product (1.7 g) as a white solid.

Step B

### Preparation of N-{3-chloro-4-[(3-fluorobenzyl)oxy]phenyl}-6-ethynylthieno[3,2-d]pyrimidin-4-amine

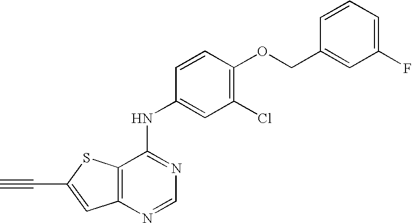

6-Bromo-N-{3-chloro-4-[(3-fluorobenzyl)oxy]phenyl}thieno[3,2-d]pyrimidin-4-amine hydrochloride (1.0 g, 2.0 mmol) was combined with CuI (45 mg, 0.24 mmol), dichlorobis(triphenylphosphine)palladium(II) (57 mg, 0.08 mmol), THF (14 mL), triethylamine (0.74 mL, 5.3 mmol), and trimethylsilylacetylene (0.37 mL, 2.62 mmol). The mixture stirred at room temperature for 6 h, concentrated and purified by silica gel chromatography (eluting with 3:1 to 2:1 hexane/ethyl acetate). The resulting silyl acetylene intermediate (618 mg) was dissolved in THF (17 mL) and cooled to 0° C. A 1.0 M solution of TBAF in THF (1.4 mL, 1.4 mmol) was added and the mixture was stirred 1 h. The reaction was partitioned between ethyl acetate and water, the organic layer was separated and dried (Na₂SO₄) filtered and concentrated. The resulting solid was purified by silica gel chromatography (eluting with 7:3 to 6:4 hexane/ethyl acetate) to give the title compound (400 mg) as an orange solid. ESI MS (positive ion): (M-H) 410.2 ¹H NMR (300 MHz, DMSO) δ 5.03 (s, 1H), 5.25 (s, 2H), 7.14-7.21 (m, 1H), 7.23-7.22 (m, 3H), 7.43-7.50 (d, 1H), 7.61 (dd, J=8.9, 2.6 Hz, 1H), 7.71 (s, 1H), 7.92 (d, J=2.5 Hz, 1H), 8.59 (s, 1H), 9.78 (s, 1H).

### Example 2

### N-{3-Chloro-4-[(3-fluorobenzyl)oxy]phenyl}-6-ethynylthieno[2,3-d]pyrimidin-4-amine

Step A

### Preparation of 6-bromothieno[2,3-d]pyrimidin-4(3H)-one

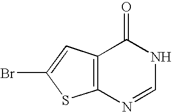

To a slurry of commercially available thieno[2,3-d]pyrimidin-4(3H)-one (1.5 g, 9.86 mmol) in glacial acetic acid (26 mL) was added dropwise bromine (1.0 mL, 20 mmol). The dark brown mixture was heated at 80° C. for 1.5 h. The mixture was allowed to cool to ambient temperature and was poured onto a mixture of saturated aqueous NaHCO₃ and ice. The resulting solid was collected by suction filtration, washed with water and dried in vacuo to afford 2.09 g of the title compound. ¹H NMR (400 MHz, DMSO-d₆) δ 7.54 (s, 1H), 8.13 (d, 2H, J=3.7 Hz), 12.6 (bs, 1H).

Step B

### Preparation of 6-Bromo-4-chlorothieno[2,3-d]pyrimidine

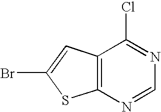

6-Bromothieno[2,3-d]pyrimidin-4(3H)-one (2.09 g, 9.05 mmol) was covered with phorphorous oxychloride (4.0 mL, 42.9 mmol) and the mixture was heated at 118-120° C. for 2 h. The mixture was allowed to cool to ambient temperature and was poured onto a mixture of saturated aqueous NaHCO₃ and ice. The resulting precipitate was collected by suction filtration and washed with water. The resulting solid was dried in vacuo to afford 2.07 g of the title compound. ¹H NMR (400 MHz, DMSO-d₆) δ 7.88 (s, 1H), 8.93 (s, 1H).

Step C

### Preparation of 6-Bromo-N-{3-chloro-4-[(3-fluorobenzyl)oxy]phenyl}thieno[2,3-d]pyrimidin-4-amine

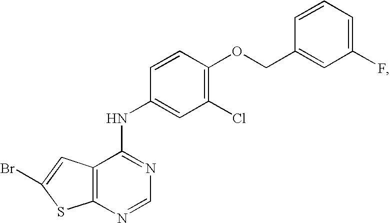

A mixture of 6-bromo-4-chlorothieno[2,3-d]pyrimidine (2.07 g, 8.29 mmol), 3-chloro-4-[(3-fluorobenzyl)oxy]aniline (2.09 g, 8.29 mmol), triethylamine (2.31 mL, 16.57 mmol) and isopropanol (40 mL) was heated at 85° C. for 16 h. The mixture was allowed to cool to ambient temperature and concentrated to leave a brown residue. The mixture was triturated with ether to afford the title compound (3.34 g) as a tan solid. ¹H NMR (400 MHz, DMSO-d₆) δ 5.24 (s, 2H), 7.18 (m, 1H), 7.26 (d, 1H, J=9.1 Hz), 7.32 (m, 1H), 7.64 (dd, 1H, J=12.1, 2.7 Hz), 8.00 (d, 1H, J=2.5 Hz), 8.02 (s, 1H), 8.48 (s, 1H), 9.63 (s, 1H).

Step D

### Preparation of N-{3-Chloro-4-[(3-fluorobenzyl)oxy]phenyl}-6-[(trimethylsilyl)ethynyl]-4,4a-dihydrothieno[2,3-d]pyrimidin-4-amine

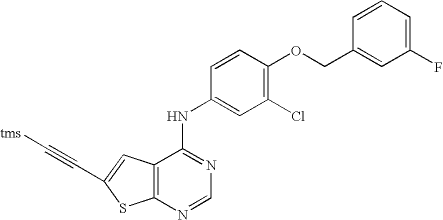

An N₂-flushed flask was charged with 6-bromo-N-{3-chloro-4-[(3-fluorobenzyl)oxy]phenyl}thieno[2,3-d]pyrimidin-4-amine (1.0 g, 2.15 mmol), Cu(I)I (46 mg, 0.24 mmol), dichlorobis(triphenylphosphino)palladium(II) (57 mg, 0.081 mmol), anhydrous THF (13.5 mL), triethylamine (600 μL, 4.3 mmol) and trimethylsilyl acetylene (370 μL, 2.62 mmol) and the resulting mixture was heated at 40° C. for 5 h. The mixture was concentrated with a rotary evaporator and the residue was purified by flash silica gel chromatography (eluting with 5:1 hexanes/ethyl acetate) to afford 623.4 mg of the title compound as a yellow solid. ¹H NMR (400 MHz, DMSO-d₆) δ 0.25 (s, 9H), 5.22 (s, 2H), 7.16 (dt, 1H, J=8.9, 2.5 Hz), 7.24 (d, 1H, J=9.0), 7.29 (m, 1H). 7.43 (m, 1H), 7.62 (dd, 1H, J=8.9, 2.5 Hz), 8.01 (d, 1H, J=2.8 Hz), 8.09 (s, 1H), 8.52 (s, 1H), 9.63 (s, 1H).

Step E

### Preparation of N-{3-Chloro-4-[(3-fluorobenzyl)oxy]phenyl}-6-ethynylthieno[2,3-d]pyrimidin-4-amine

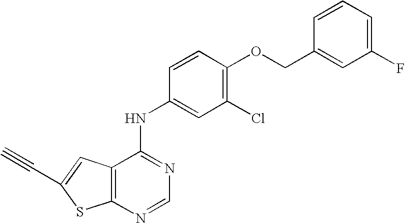

To a 0° C. solution of N-{3-chloro-4-[(3-fluorobenzyl)oxy]phenyl}-6-[(trimethylsilyl)ethynyl]-4,4a-dihydrothieno[2,3-a]pyrimidin-4-amine (623.4 mg, 1.29 mmol) in anhydrous THF (17 mL) was added 1.0 M TBAF in THF (1.41 mL, 1.41 mmol). The mixture was stirred at 0° C. for 30 min, then partitioned between ethyl acetate and water. The organic layer was separated, dried over Na₂SO₄, filtered and concentrated to give a residue that was purified by silica gel chromatography (eluting with 5:1 hexanes/ethyl acetate) to give 519.3 mg of the title compound as a pale yellow solid, mp 197° C. ¹H NMR (400 MHz, DMSO-d₆) δ 4.89 (s, 1H), 5.27 (s, 2H), 7.21 (t, 1H, J=9.2 Hz), 7.31 (d, 1H, J=9.2 Hz), 7.34-7.36 (m, 1H). 7.45-7.50 (m, 1H), 7.67 (dd, 1H, J=9.0, 2.4 Hz), 8.02 (d, 1H, J=2.4 Hz), 8.10 (s, 1H), 8.56 (s, 1H), 9.75 (s, 1H). ms (MH)+=382.3.

ErbB4 Enzyme Assays:

Compounds of the present invention may be tested for ErbB-4 protein tyrosine kinase inhibitory activity in substrate phosphorylation assays using enzymes purified from a baculovirus expression system. Reagent production and assay methodology were conducted essentially as described (Brignola, P. S., et al, (2002) J. Biol. Chem. v. 277 in press).

The method measures the ability of the isolated enzyme to catalyse the transfer of the y-phosphate from ATP onto tyrosine residues in a biotinylated synthetic peptide (biotin-Ahx-RAHEEIYHFFFAKKK-amide). Reactions were performed in 96-well polystyrene round-bottom plates in a final volume of 45 μL. Reaction mixtures contained 50 mM MOPS (pH 7.5), 2 mM MnCl₂, 10 μM ATP, 0.125 μCi [γ-³³P] ATP per reaction, 2 μM peptide substrate, and 1 mM dithiothreitol. Reactions were initiated by adding 1 pmol (20 nM) per reaction of the indicated enzyme. The reaction was allowed to proceed for 15 minutes, terminated and quantified using a scintillation proximity assay procedure as described in McDonald, O. B., Antonsson, B., Arkinstal, S., Marshall, C. J., and Wood, E. R. (1999) Analytical Biochemistry, 268, 318-329.

Compounds under analysis were dissolved in Me₂SO to 0.5 mM and serially diluted 1 to 3 with Me₂SO through eleven columns of a 96 well plate. 1 μL of each concentration was transferred to the corresponding well of the assay plate. This creates a final compound concentration range from 0.00019 to 11.1 μM.

The data for dose responses were plotted as % Control calculated with the data reduction formula 100*(U1−C2)/(C1−C₂) versus concentration of compound and fitted to the curve described by:
γ=((Vmax*x)/(K+x))
where Vmax is the upper asymptote and K is the IC₅₀. Typically, promising ErbB4 inhibitors will illicit a pIC50 >7.0.
Crystallization and Data Collection

Crystals were obtained by the hanging drop vapor diffusion method. Protein (˜4 mg/ml in 20 mM HEPES pH 7.5, 300 mM NaCl, 5 mM DTT, 1 mM CHAPS) was mixed with an equal volume of reservoir (50 mM cacodylate pH 6.5, 100 mM ammonium acetate, 10 mM Mg acetate, 30% PEG8000) and incubated at 22° C. Crystals belonged to the tetragonal space group P43 with two molecules in the asymmetric unit and the following cell dimensions: a=63.95 Å, b=63.95 Å, c=163.39 Å, α=90°, β=90°, γ=90°.

Prior to data collection, glycerol and PEG400 was added to a final concentration of 25% and 5%, respectively, and the crystals were flash frozen in liquid N₂. Data was collected at beamline 17-BM on a MAR-CCD detector in the facilities of the Industrial Macromolecular Crystallography Association Collaborative Access Team (IMCA-CAT) at the Advanced Photon Source, Argonne National Laboratory. These facilities are supported by the companies of the Industrial Macromolecular Crystallography Association through a contract with Illinois Institute of Technology (IIT), executed through the IIT's Center for Synchrotron Radiation Research and Instrumentation. The data were processed using HKL2000.

Structure Determination and Refinement

The structure was solved by molecular replacement using CNX and FGFR¹ as a search model (molecule 1 of PDB entry 1FGK). The search model contained FGFR¹ residues 464-485, 491-500, 506-578, 592-647 and 651-761. Residues not conserved between FGFR¹ and ErbB4 were truncated to alanine in the model. The correct solutions were the top two peaks in both the rotation and translation functions. Rigid body refinement gave an initial R-factor of 48%. Multiple rounds of model building and refinement were carried out with QUANTA and CNX. The overall structure was confirmed by a composite omit map calculated with CNX. Analysis of the structure with PROCHECK indicated that all main chain torsions fall within the allowed regions of the Ramachandran plot.

The results are depicted in Table 1 and 2 following.

(1) Amino Acid Sequence:

(SEQ ID NO: 1)

MKKGHHHHHHGLETELVEPLTPSGTAPNQAQLRILKETELKRVKVLGSGA
FGTVYKGIWVPEGETVKIPVAIKILNETTGPKANVEFMDEALIMASMDHP
HLVRLLGVCLSPTIQLVTQLMPHGCLLEYVHEHKDNIGSQLLLNWCVQIA
KGMMYLEERRLVHRDLAARNVLVKSPNHVKITDFGLARLLEGDEKEYNAD
GGKMPIKWMALECIHYRKFTHQSDVWSYGVTIWELMTFGGKPYDGIPTRE
IPDLLEKGERLPQPPICTIDVYMVMVKCWMIDADSRPKFKELAAEFSRMA
RDPQRYLVIQGDDRMKLPSPNDSKFFQNLLDEEDLEDMMDAEEYLVPQAF
NIPPPIYTSRARID

DNA Sequence 1: His-ErbB4Amino Acids 690-999 Nucleotide Sequence:

| SEQ ID No : 2 | |
|---|---|
| 1 | ATGAAAAAAG GTCATGATCA TCATCATCAT GGTTTGGAAA CAGAGTTGGT |
| 51 | GGAACCATTA AGTCCCAGTG GCACAGCACC CAATCAAGCT CAACTTCGTA |
| 101 | TTTTGAAAGA AACTGAGCTG AAGAGGGTAA AAGTCC1TGG CTCAGGTGCT |
| 151 | TTTGGAACGG TTTATAAAGG TATTTGGGTA CCTGAAGGAG AAACTGTGAA |
| 201 | GATTCCTGTG GCTATTAAGA TTCTTAATGA GACAACTGGT CCCAAGGCAA |
| 251 | ATGTGGAGTT GATGGATGAA GCTCTGATCA TGGCAAGTAT GGATCATCCA |
| 301 | CACCTAGTCC GG1TGCTGGG TGTGTGTCTG AGCCCAACCA TCCAGCTGGT |
| 351 | TACTCAACTT ATGCCCCATG GGTGCGTGTT GGAGTATGTC GAGGAGGACA |
| 401 | AGGATAACAT TGGATCACAA CTGCTGCTTA ACTGGTGTGT CCAGATAGCT |
| 451 | AAGGGAATGA TGTACCTGGA AGAAAGACGA CTCGTTCATC GGGATTTGGC |
| 501 | AGCCCGTAAT GTCTTAGTGA AATCTCCAAA CCATGTGAAA ATCACAGATT |
| 551 | TTGGGCTAGC CAGACTCTTG GAAGGAGATG AAAAAGAGTA CAATGCTGAT |
| 601 | GGAGGAAAGA TGCCAATTAA ATGGATGGCT CTGGAGTGTA TACATTACAG |
| 651 | GAAATTCACC CATCAGAGTG ACGTTTGGAG CTATGGAGTT ACTATATGGG |
| 701 | AACTGATGAC CTTTGGAGGA AAACCCTATG ATGGAATTCC AACGCGAGAA |
| 751 | ATCCCTGATT TATTAGAGAA AGGAGAACGT TTGCCTCAGG CTCCCATCTG |
| 801 | CACTATTGAC GTTTACATGG TCATGGTCAA ATGTTGGATG ATTGATGCTG |
| 851 | ACAGTAGACC TAAATTTAAG GAACTGGCTG GTGAGTTTTC AAGGATGGCT |
| 901 | CGAGACCCTC AAAGATACCT AGTTATTCAG GGTGATGATC GTATGAAGCT |
| 951 | TCCCAGTCCA AATTGA. |

TABLE 1
DATA STATISTICS

| Space group | P4₃ |
|---|---|
| Unit Cell a (Å) | 63.95 |
| Unit Cell b (Å) | 63.95 |
| Unit Cell c (Å) | 163.39 |
| Unit Cell α = β = γ (°) | 90 |
| Mol/asu | 2 |
| Resolution (Å) | 2.5 |
| R_{sym} (%) | 8.8 |
| Completeness (%) | 91.9 |
| R_{factor} (%) | 21 |
| R_{free} (%) | 27 |
| Rmsd from ideal | |
| Bond lengths (Å) | 0.011 |
| Bond angles (°) | 1.48 |

TABLE 2
erbB4(690-999) thienopyrimidine cocrystal
resolution: 500.0-2.5 A
sg = P4(3) a = 63.954 b = 63.954 c = 163.391 alpha = 90 beta = 90 gamma = 90
final r = 0.2130 free_r = 0.2762
rmsd bonds = 0.011188 rmsd angles = 1.48513
data completeness 91.9% Rmerge 8.8%
data collected on a marCCD at IMCA, −180 C., processed with HKL2000, solved
with CNX

| | | | | | | | | | | |
|---|---|---|---|---|---|---|---|---|---|---|
| ATOM | 1 | N | LEU | A | 698 | −53.411 | 26.468 | 10.538 | 1.00 | 81.54 |
| ATOM | 2 | CA | LEU | A | 698 | −53.129 | 25.423 | 9.504 | 1.00 | 81.33 |
| ATOM | 3 | C | LEU | A | 698 | −53.233 | 26.015 | 8.103 | 1.00 | 81.09 |
| ATOM | 4 | O | LEU | A | 698 | −54.198 | 26.709 | 7.791 | 1.00 | 81.42 |
| ATOM | 5 | CB | LEU | A | 698 | −54.120 | 24.260 | 9.636 | 1.00 | 81.21 |
| ATOM | 6 | CG | LEU | A | 698 | −53.757 | 22.974 | 8.884 | 1.00 | 81.79 |
| ATOM | 7 | CD1 | LEU | A | 698 | −52.552 | 22.311 | 9.555 | 1.00 | 81.75 |
| ATOM | 8 | CD2 | LEU | A | 698 | −54.949 | 22.024 | 8.877 | 1.00 | 81.23 |
| ATOM | 9 | N | THR | A | 699 | −52.236 | 25.740 | 7.265 | 1.00 | 81.31 |
| ATOM | 10 | CA | THR | A | 699 | −52.210 | 26.243 | 5.890 | 1.00 | 82.23 |
| ATOM | 11 | C | THR | A | 699 | −51.171 | 25.519 | 5.018 | 1.00 | 82.37 |
| ATOM | 12 | O | THR | A | 699 | −50.003 | 25.905 | 4.971 | 1.00 | 82.32 |
| ATOM | 13 | CB | THR | A | 699 | −51.933 | 27.781 | 5.852 | 1.00 | 82.76 |
| ATOM | 14 | OG1 | THR | A | 699 | −50.977 | 28.138 | 6.863 | 1.00 | 81.44 |
| ATOM | 15 | CG2 | THR | A | 699 | −53.226 | 28.567 | 6.062 | 1.00 | 82.15 |
| ATOM | 16 | N | PRO | A | 700 | −51.593 | 24.452 | 4.315 | 1.00 | 82.54 |
| ATOM | 17 | CA | PRO | A | 700 | −50.743 | 23.642 | 3.435 | 1.00 | 82.57 |
| ATOM | 18 | C | PRO | A | 700 | −49.901 | 24.432 | 2.446 | 1.00 | 82.12 |
| ATOM | 19 | O | PRO | A | 700 | −50.175 | 25.596 | 2.159 | 1.00 | 82.75 |
| ATOM | 20 | CB | PRO | A | 700 | −51.747 | 22.741 | 2.724 | 1.00 | 82.43 |
| ATOM | 21 | CG | PRO | A | 700 | −52.758 | 22.504 | 3.777 | 1.00 | 82.66 |
| ATOM | 22 | CD | PRO | A | 700 | −52.958 | 23.898 | 4.353 | 1.00 | 83.18 |
| ATOM | 23 | N | ALA | A | 701 | −48.873 | 23.772 | 1.926 | 1.00 | 82.09 |
| ATOM | 24 | CA | ALA | A | 701 | −47.958 | 24.364 | 0.957 | 1.00 | 81.38 |
| ATOM | 25 | C | ALA | A | 701 | −47.697 | 23.318 | −0.117 | 1.00 | 80.59 |
| ATOM | 26 | O | ALA | A | 701 | −47.769 | 22.118 | 0.149 | 1.00 | 81.14 |
| ATOM | 27 | CB | ALA | A | 701 | −46.646 | 24.755 | 1.643 | 1.00 | 81.49 |
| ATOM | 28 | N | GLY | A | 702 | −47.399 | 23.766 | −1.328 | 1.00 | 79.29 |
| ATOM | 29 | CA | GLY | A | 702 | −47.139 | 22.814 | −2.389 | 1.00 | 79.40 |
| ATOM | 30 | C | GLY | A | 702 | −46.042 | 21.833 | −2.016 | 1.00 | 78.74 |
| ATOM | 31 | O | GLY | A | 702 | −46.226 | 20.612 | −2.070 | 1.00 | 78.53 |
| ATOM | 32 | N | THR | A | 703 | −44.902 | 22.387 | −1.615 | 1.00 | 77.41 |
| ATOM | 33 | CA | THR | A | 703 | −43.720 | 21.621 | −1.238 | 1.00 | 75.67 |
| ATOM | 34 | C | THR | A | 703 | −43.960 | 20.300 | −0.510 | 1.00 | 73.00 |
| ATOM | 35 | O | THR | A | 703 | −44.742 | 20.222 | 0.432 | 1.00 | 73.21 |
| ATOM | 36 | CB | THR | A | 703 | −42.780 | 22.481 | −0.378 | 1.00 | 77.78 |
| ATOM | 37 | OG1 | THR | A | 703 | −42.497 | 23.708 | −1.065 | 1.00 | 78.99 |
| ATOM | 38 | CG2 | THR | A | 703 | −41.473 | 21.740 | −0.108 | 1.00 | 78.77 |
| ATOM | 39 | N | ALA | A | 704 | −43.259 | 19.264 | −0.960 | 1.00 | 70.01 |
| ATOM | 40 | CA | ALA | A | 704 | −43.361 | 17.942 | −0.366 | 1.00 | 66.51 |
| ATOM | 41 | C | ALA | A | 704 | −42.270 | 17.799 | 0.688 | 1.00 | 65.00 |
| ATOM | 42 | O | ALA | A | 704 | −41.318 | 18.588 | 0.737 | 1.00 | 63.04 |
| ATOM | 43 | CB | ALA | A | 704 | −43.201 | 16.872 | −1.435 | 1.00 | 66.82 |
| ATOM | 44 | N | PRO | A | 705 | −42.395 | 16.788 | 1.552 | 1.00 | 62.77 |
| ATOM | 45 | CA | PRO | A | 705 | −41.399 | 16.577 | 2.597 | 1.00 | 61.58 |
| ATOM | 46 | C | PRO | A | 705 | −40.009 | 16.258 | 2.067 | 1.00 | 60.23 |
| ATOM | 47 | O | PRO | A | 705 | −39.843 | 15.521 | 1.095 | 1.00 | 61.36 |
| ATOM | 48 | CB | PRO | A | 705 | −41.993 | 15.430 | 3.410 | 1.00 | 61.65 |
| ATOM | 49 | CG | PRO | A | 705 | −42.736 | 14.656 | 2.391 | 1.00 | 63.07 |
| ATOM | 50 | CD | PRO | A | 705 | −43.418 | 15.730 | 1.581 | 1.00 | 63.22 |
| ATOM | 51 | N | ASN | A | 706 | −39.017 | 16.843 | 2.724 | 1.00 | 57.06 |
| ATOM | 52 | CA | ASN | A | 706 | −37.621 | 16.649 | 2.392 | 1.00 | 54.07 |
| ATOM | 53 | C | ASN | A | 706 | −37.112 | 15.494 | 3.264 | 1.00 | 53.97 |
| ATOM | 54 | O | ASN | A | 706 | −36.701 | 15.707 | 4.404 | 1.00 | 52.73 |
| ATOM | 55 | CB | ASN | A | 706 | −36.846 | 17.926 | 2.715 | 1.00 | 52.73 |
| ATOM | 56 | CG | ASN | A | 706 | −35.377 | 17.816 | 2.374 | 1.00 | 52.89 |
| ATOM | 57 | OD1 | ASN | A | 706 | −34.883 | 16.732 | 2.070 | 1.00 | 51.75 |
| ATOM | 58 | ND2 | ASN | A | 706 | −34.664 | 18.940 | 2.434 | 1.00 | 51.00 |
| ATOM | 59 | N | GLN | A | 707 | −37.137 | 14.274 | 2.740 | 1.00 | 51.97 |
| ATOM | 60 | CA | GLN | A | 707 | −36.679 | 13.147 | 3.536 | 1.00 | 53.28 |
| ATOM | 61 | C | GLN | A | 707 | −35.202 | 12.775 | 3.376 | 1.00 | 51.56 |
| ATOM | 62 | O | GLN | A | 707 | −34.813 | 11.628 | 3.611 | 1.00 | 50.32 |
| ATOM | 63 | CB | GLN | A | 707 | −37.563 | 11.925 | 3.280 | 1.00 | 52.48 |
| ATOM | 64 | CG | GLN | A | 707 | −38.988 | 12.125 | 3.736 | 1.00 | 57.13 |
| ATOM | 65 | CD | GLN | A | 707 | −39.850 | 10.906 | 3.491 | 1.00 | 60.12 |
| ATOM | 66 | OE1 | GLN | A | 707 | −39.900 | 9.992 | 4.313 | 1.00 | 62.36 |
| ATOM | 67 | NE2 | GLN | A | 707 | −40.523 | 10.876 | 2.338 | 1.00 | 60.48 |
| ATOM | 68 | N | ALA | A | 708 | −34.378 | 13.742 | 2.990 | 1.00 | 49.29 |
| ATOM | 69 | CA | ALA | A | 708 | −32.950 | 13.483 | 2.843 | 1.00 | 48.15 |
| ATOM | 70 | C | ALA | A | 708 | −32.400 | 13.031 | 4.202 | 1.00 | 48.57 |
| ATOM | 71 | O | ALA | A | 708 | −32.871 | 13.477 | 5.248 | 1.00 | 47.60 |
| ATOM | 72 | CB | ALA | A | 708 | −32.239 | 14.741 | 2.390 | 1.00 | 45.11 |
| ATOM | 73 | N | GLN | A | 709 | −31.416 | 12.138 | 4.187 | 1.00 | 49.32 |
| ATOM | 74 | CA | GLN | A | 709 | −30.824 | 11.662 | 5.430 | 1.00 | 49.62 |
| ATOM | 75 | C | GLN | A | 709 | −29.474 | 12.323 | 5.665 | 1.00 | 50.85 |
| ATOM | 76 | O | GLN | A | 709 | −28.692 | 12.528 | 4.736 | 1.00 | 52.52 |
| ATOM | 77 | CB | GLN | A | 709 | −30.627 | 10.145 | 5.388 | 1.00 | 48.60 |
| ATOM | 78 | CG | GLN | A | 709 | −31.897 | 9.345 | 5.182 | 1.00 | 47.70 |
| ATOM | 79 | CD | GLN | A | 709 | −32.848 | 9.427 | 6.353 | 1.00 | 47.21 |
| ATOM | 80 | OE1 | GLN | A | 709 | −32.529 | 8.975 | 7.450 | 1.00 | 47.89 |
| ATOM | 81 | NE2 | GLN | A | 709 | −34.027 | 10.004 | 6.126 | 1.00 | 44.74 |
| ATOM | 82 | N | LEU | A | 710 | −29.212 | 12.668 | 6.913 | 1.00 | 51.76 |
| ATOM | 83 | CA | LEU | A | 710 | −27.945 | 13.268 | 7.283 | 1.00 | 54.69 |
| ATOM | 84 | C | LEU | A | 710 | −27.458 | 12.404 | 8.424 | 1.00 | 55.52 |
| ATOM | 85 | O | LEU | A | 710 | −28.112 | 12.334 | 9.461 | 1.00 | 54.54 |
| ATOM | 86 | CB | LEU | A | 710 | −28.129 | 14.702 | 7.782 | 1.00 | 56.03 |
| ATOM | 87 | CG | LEU | A | 710 | −26.833 | 15.259 | 8.384 | 1.00 | 57.05 |
| ATOM | 88 | CD1 | LEU | A | 710 | −25.863 | 15.544 | 7.264 | 1.00 | 56.49 |
| ATOM | 89 | CD2 | LEU | A | 710 | −27.102 | 16.510 | 9.198 | 1.00 | 57.87 |
| ATOM | 90 | N | ARG | A | 711 | −26.321 | 11.742 | 8.247 | 1.00 | 56.82 |
| ATOM | 91 | CA | ARG | A | 711 | −25.821 | 10.877 | 9.302 | 1.00 | 58.00 |
| ATOM | 92 | C | ARG | A | 711 | −24.737 | 11.545 | 10.114 | 1.00 | 56.44 |
| ATOM | 93 | O | ARG | A | 711 | −23.756 | 12.036 | 9.577 | 1.00 | 55.70 |
| ATOM | 94 | CB | ARG | A | 711 | −25.327 | 9.556 | 8.710 | 1.00 | 60.89 |
| ATOM | 95 | CG | ARG | A | 711 | −26.351 | 8.925 | 7.763 | 1.00 | 67.44 |
| ATOM | 96 | CD | ARG | A | 711 | −25.971 | 7.525 | 7.317 | 1.00 | 71.94 |
| ATOM | 97 | NE | ARG | A | 711 | −26.098 | 6.561 | 8.406 | 1.00 | 76.65 |
| ATOM | 98 | CZ | ARG | A | 711 | −25.853 | 5.258 | 8.283 | 1.00 | 78.71 |
| ATOM | 99 | NH1 | ARG | A | 711 | −25.465 | 4.760 | 7.111 | 1.00 | 78.46 |
| ATOM | 100 | NH2 | ARG | A | 711 | −26.002 | 4.455 | 9.331 | 1.00 | 78.34 |
| ATOM | 101 | N | ILE | A | 712 | −24.946 | 11.584 | 11.422 | 1.00 | 57.82 |
| ATOM | 102 | CA | ILE | A | 712 | −23.988 | 12.181 | 12.342 | 1.00 | 59.70 |
| ATOM | 103 | C | ILE | A | 712 | −23.017 | 11.065 | 12.730 | 1.00 | 59.96 |
| ATOM | 104 | O | ILE | A | 712 | −23.401 | 10.089 | 13.363 | 1.00 | 59.83 |
| ATOM | 105 | CB | ILE | A | 712 | −24.727 | 12.748 | 13.562 | 1.00 | 59.41 |
| ATOM | 106 | CG1 | ILE | A | 712 | −25.841 | 13.673 | 13.057 | 1.00 | 60.67 |
| ATOM | 107 | CG2 | ILE | A | 712 | −23.760 | 13.512 | 14.469 | 1.00 | 57.78 |
| ATOM | 108 | CD1 | ILE | A | 712 | −26.793 | 14.166 | 14.119 | 1.00 | 62.84 |
| ATOM | 109 | N | LEU | A | 713 | −21.761 | 11.215 | 12.328 | 1.00 | 60.81 |
| ATOM | 110 | CA | LEU | A | 713 | −20.753 | 10.191 | 12.566 | 1.00 | 62.74 |
| ATOM | 111 | C | LEU | A | 713 | −19.812 | 10.411 | 13.736 | 1.00 | 63.70 |
| ATOM | 112 | O | LEU | A | 713 | −19.297 | 11.512 | 13.939 | 1.00 | 63.91 |
| ATOM | 113 | CB | LEU | A | 713 | −19.907 | 10.007 | 11.307 | 1.00 | 62.56 |
| ATOM | 114 | CG | LEU | A | 713 | −20.661 | 9.698 | 10.016 | 1.00 | 62.99 |
| ATOM | 115 | CD1 | LEU | A | 713 | −19.656 | 9.255 | 8.970 | 1.00 | 64.29 |
| ATOM | 116 | CD2 | LEU | A | 713 | −21.714 | 8.620 | 10.254 | 1.00 | 62.63 |
| ATOM | 117 | N | ALA | A | 714 | −19.581 | 9.343 | 14.494 | 1.00 | 64.16 |
| ATOM | 118 | CA | ALA | A | 714 | −18.669 | 9.405 | 15.618 | 1.00 | 64.73 |
| ATOM | 119 | C | ALA | A | 714 | −17.294 | 9.443 | 14.978 | 1.00 | 65.80 |
| ATOM | 120 | O | ALA | A | 714 | −17.011 | 8.687 | 14.047 | 1.00 | 65.21 |
| ATOM | 121 | CB | ALA | A | 714 | −18.816 | 8.168 | 16.494 | 1.00 | 65.59 |
| ATOM | 122 | N | GLU | A | 715 | −16.461 | 10.352 | 15.470 | 1.00 | 67.13 |
| ATOM | 123 | CA | GLU | A | 715 | −15.092 | 10.556 | 15.000 | 1.00 | 68.35 |
| ATOM | 124 | C | GLU | A | 715 | −14.346 | 9.234 | 14.778 | 1.00 | 68.80 |
| ATOM | 125 | O | GLU | A | 715 | −13.419 | 9.145 | 13.965 | 1.00 | 67.36 |
| ATOM | 126 | CB | GLU | A | 715 | −14.370 | 11.404 | 16.046 | 1.00 | 70.35 |
| ATOM | 127 | CG | GLU | A | 715 | −12.937 | 11.784 | 15.762 | 1.00 | 74.38 |
| ATOM | 128 | CD | GLU | A | 715 | −12.337 | 12.568 | 16.925 | 1.00 | 76.58 |
| ATOM | 129 | OE1 | GLU | A | 715 | −11.155 | 12.966 | 16.837 | 1.00 | 79.37 |
| ATOM | 130 | OE2 | GLU | A | 715 | −13.051 | 12.785 | 17.932 | 1.00 | 75.16 |
| ATOM | 131 | N | THR | A | 716 | −14.779 | 8.211 | 15.509 | 1.00 | 68.91 |
| ATOM | 132 | CA | THR | A | 716 | −14.189 | 6.880 | 15.465 | 1.00 | 68.54 |
| ATOM | 133 | C | THR | A | 716 | −14.486 | 6.062 | 14.214 | 1.00 | 67.91 |
| ATOM | 134 | O | THR | A | 716 | −13.757 | 5.117 | 13.901 | 1.00 | 67.89 |
| ATOM | 135 | CB | THR | A | 716 | −14.643 | 6.049 | 16.675 | 1.00 | 69.38 |
| ATOM | 136 | OG1 | THR | A | 716 | −14.187 | 4.700 | 16.521 | 1.00 | 72.15 |
| ATOM | 137 | CG2 | THR | A | 716 | −16.163 | 6.049 | 16.782 | 1.00 | 69.99 |
| ATOM | 138 | N | GLU | A | 717 | −15.551 | 6.408 | 13.500 | 1.00 | 66.43 |
| ATOM | 139 | CA | GLU | A | 717 | −15.910 | 5.661 | 12.302 | 1.00 | 65.47 |
| ATOM | 140 | C | GLU | A | 717 | −15.111 | 6.104 | 11.085 | 1.00 | 64.70 |
| ATOM | 141 | O | GLU | A | 717 | −15.137 | 5.440 | 10.047 | 1.00 | 63.74 |
| ATOM | 142 | CB | GLU | A | 717 | −17.408 | 5.806 | 12.035 | 1.00 | 65.65 |
| ATOM | 143 | CG | GLU | A | 717 | −18.276 | 5.259 | 13.166 | 1.00 | 65.53 |
| ATOM | 144 | CD | GLU | A | 717 | −19.658 | 5.892 | 13.215 | 1.00 | 65.95 |
| ATOM | 145 | OE1 | GLU | A | 717 | −20.471 | 5.662 | 12.291 | 1.00 | 62.49 |
| ATOM | 146 | OE2 | GLU | A | 717 | −19.922 | 6.635 | 14.187 | 1.00 | 67.09 |
| ATOM | 147 | N | LEU | A | 718 | −14.382 | 7.211 | 11.223 | 1.00 | 64.90 |
| ATOM | 148 | CA | LEU | A | 718 | −13.591 | 7.752 | 10.119 | 1.00 | 65.63 |
| ATOM | 149 | C | LEU | A | 718 | −12.082 | 7.598 | 10.274 | 1.00 | 67.11 |
| ATOM | 150 | O | LEU | A | 718 | −11.521 | 7.846 | 11.345 | 1.00 | 66.91 |
| ATOM | 151 | CB | LEU | A | 718 | −13.909 | 9.236 | 9.917 | 1.00 | 64.23 |
| ATOM | 152 | CG | LEU | A | 718 | −15.353 | 9.609 | 9.575 | 1.00 | 64.11 |
| ATOM | 153 | CD1 | LEU | A | 718 | −15.478 | 11.119 | 9.521 | 1.00 | 63.98 |
| ATOM | 154 | CD2 | LEU | A | 718 | −15.757 | 8.988 | 8.251 | 1.00 | 62.29 |
| ATOM | 155 | N | ALA | A | 719 | −11.435 | 7.204 | 9.178 | 1.00 | 67.97 |
| ATOM | 156 | CA | ALA | A | 719 | −9.986 | 7.027 | 9.139 | 1.00 | 67.89 |
| ATOM | 157 | C | ALA | A | 719 | −9.394 | 7.806 | 7.968 | 1.00 | 67.48 |
| ATOM | 158 | O | ALA | A | 719 | −9.687 | 7.518 | 6.805 | 1.00 | 67.27 |
| ATOM | 159 | CB | ALA | A | 719 | −9.634 | 5.544 | 9.004 | 1.00 | 66.89 |
| ATOM | 160 | N | ARG | A | 720 | −8.572 | 8.801 | 8.284 | 1.00 | 67.63 |
| ATOM | 161 | CA | ARG | A | 720 | −7.918 | 9.605 | 7.264 | 1.00 | 67.22 |
| ATOM | 162 | C | ARG | A | 720 | −6.859 | 8.725 | 6.599 | 1.00 | 69.63 |
| ATOM | 163 | O | ARG | A | 720 | −6.763 | 7.534 | 6.890 | 1.00 | 70.38 |
| ATOM | 164 | CB | ARG | A | 720 | −7.230 | 10.810 | 7.901 | 1.00 | 65.71 |
| ATOM | 165 | CG | ARG | A | 720 | −8.103 | 11.668 | 8.793 | 1.00 | 65.13 |
| ATOM | 166 | CD | ARG | A | 720 | −7.881 | 13.135 | 8.465 | 1.00 | 65.92 |
| ATOM | 167 | NE | ARG | A | 720 | −8.268 | 14.063 | 9.529 | 1.00 | 67.39 |
| ATOM | 168 | CZ | ARG | A | 720 | −9.421 | 14.033 | 10.197 | 1.00 | 70.61 |
| ATOM | 169 | NH1 | ARG | A | 720 | −10.342 | 13.103 | 9.939 | 1.00 | 70.23 |
| ATOM | 170 | NH2 | ARG | A | 720 | −9.670 | 14.966 | 11.112 | 1.00 | 70.99 |
| ATOM | 171 | N | VAL | A | 721 | −6.060 | 9.315 | 5.716 | 1.00 | 71.34 |
| ATOM | 172 | CA | VAL | A | 721 | −4.988 | 8.599 | 5.018 | 1.00 | 71.66 |
| ATOM | 173 | C | VAL | A | 721 | −3.988 | 9.622 | 4.478 | 1.00 | 73.14 |
| ATOM | 174 | O | VAL | A | 721 | −3.027 | 10.001 | 5.157 | 1.00 | 73.20 |
| ATOM | 175 | CB | VAL | A | 721 | −5.533 | 7.764 | 3.833 | 1.00 | 70.82 |
| ATOM | 176 | CG1 | VAL | A | 721 | −4.386 | 7.253 | 2.984 | 1.00 | 70.96 |
| ATOM | 177 | CG2 | VAL | A | 721 | −6.344 | 6.592 | 4.347 | 1.00 | 70.37 |
| ATOM | 178 | N | ALA | A | 722 | −4.225 | 10.063 | 3.249 | 1.00 | 73.97 |
| ATOM | 179 | CA | ALA | A | 722 | −3.381 | 11.060 | 2.611 | 1.00 | 73.28 |
| ATOM | 180 | C | ALA | A | 722 | −4.262 | 12.283 | 2.405 | 1.00 | 72.83 |
| ATOM | 181 | O | ALA | A | 722 | −5.476 | 12.231 | 2.627 | 1.00 | 73.18 |
| ATOM | 182 | CB | ALA | A | 722 | −2.873 | 10.545 | 1.262 | 1.00 | 72.83 |
| ATOM | 183 | N | VAL | A | 723 | −3.640 | 13.381 | 1.996 | 1.00 | 71.49 |
| ATOM | 184 | CA | VAL | A | 723 | −4.356 | 14.615 | 1.728 | 1.00 | 68.53 |
| ATOM | 185 | C | VAL | A | 723 | −4.620 | 14.680 | 0.231 | 1.00 | 68.55 |
| ATOM | 186 | O | VAL | A | 723 | −3.765 | 14.319 | −0.576 | 1.00 | 67.71 |
| ATOM | 187 | CB | VAL | A | 723 | −3.527 | 15.842 | 2.138 | 1.00 | 67.22 |
| ATOM | 188 | CG1 | VAL | A | 723 | −4.174 | 17.111 | 1.605 | 1.00 | 66.63 |
| ATOM | 189 | CG2 | VAL | A | 723 | −3.410 | 15.900 | 3.649 | 1.00 | 66.06 |
| ATOM | 190 | N | LEU | A | 724 | −5.815 | 15.120 | −0.138 | 1.00 | 68.80 |
| ATOM | 191 | CA | LEU | A | 724 | −6.164 | 15.243 | −1.542 | 1.00 | 68.52 |
| ATOM | 192 | C | LEU | A | 724 | −5.904 | 16.689 | −1.969 | 1.00 | 69.72 |
| ATOM | 193 | O | LEU | A | 724 | −5.843 | 17.008 | −3.156 | 1.00 | 71.11 |
| ATOM | 194 | CB | LEU | A | 724 | −7.630 | 14.859 | −1.739 | 1.00 | 66.79 |
| ATOM | 195 | CG | LEU | A | 724 | −7.933 | 13.385 | −1.456 | 1.00 | 65.65 |
| ATOM | 196 | CD1 | LEU | A | 724 | −9.426 | 13.156 | −1.423 | 1.00 | 65.45 |
| ATOM | 197 | CD2 | LEU | A | 724 | −7.287 | 12.516 | −2.526 | 1.00 | 66.07 |
| ATOM | 198 | N | GLY | A | 725 | −5.721 | 17.554 | −0.979 | 1.00 | 70.85 |
| ATOM | 199 | CA | GLY | A | 725 | −5.467 | 18.954 | −1.244 | 1.00 | 72.13 |
| ATOM | 200 | C | GLY | A | 725 | −6.098 | 19.804 | −0.160 | 1.00 | 73.79 |
| ATOM | 201 | O | GLY | A | 725 | −6.806 | 19.290 | 0.707 | 1.00 | 73.67 |
| ATOM | 202 | N | SER | A | 726 | −5.842 | 21.106 | −0.204 | 1.00 | 75.09 |
| ATOM | 203 | CA | SER | A | 726 | −6.400 | 22.022 | 0.780 | 1.00 | 76.38 |
| ATOM | 204 | C | SER | A | 726 | −6.234 | 23.459 | 0.316 | 1.00 | 76.97 |
| ATOM | 205 | O | SER | A | 726 | −5.127 | 23.899 | 0.005 | 1.00 | 77.58 |
| ATOM | 206 | CB | SER | A | 726 | −5.707 | 21.846 | 2.133 | 1.00 | 77.43 |
| ATOM | 207 | OG | SER | A | 726 | −4.369 | 22.314 | 2.090 | 1.00 | 77.62 |
| ATOM | 208 | N | GLY | A | 727 | −7.346 | 24.183 | 0.265 | 1.00 | 77.88 |
| ATOM | 209 | CA | GLY | A | 727 | −7.309 | 25.570 | −0.151 | 1.00 | 77.49 |
| ATOM | 210 | C | GLY | A | 727 | −7.857 | 26.428 | 0.962 | 1.00 | 77.71 |
| ATOM | 211 | O | GLY | A | 727 | −7.950 | 25.984 | 2.107 | 1.00 | 76.78 |
| ATOM | 212 | N | ALA | A | 728 | −8.222 | 27.658 | 0.633 | 1.00 | 78.59 |
| ATOM | 213 | CA | ALA | A | 728 | −8.772 | 28.569 | 1.623 | 1.00 | 79.53 |
| ATOM | 214 | C | ALA | A | 728 | −10.094 | 28.003 | 2.130 | 1.00 | 80.29 |
| ATOM | 215 | O | ALA | A | 728 | −10.689 | 28.515 | 3.081 | 1.00 | 80.59 |
| ATOM | 216 | CB | ALA | A | 728 | −8.991 | 29.934 | 0.999 | 1.00 | 80.42 |
| ATOM | 217 | N | PHE | A | 729 | −10.533 | 26.924 | 1.495 | 1.00 | 80.52 |
| ATOM | 218 | CA | PHE | A | 729 | −11.789 | 26.283 | 1.845 | 1.00 | 80.77 |
| ATOM | 219 | C | PHE | A | 729 | −11.635 | 25.114 | 2.815 | 1.00 | 80.36 |
| ATOM | 220 | O | PHE | A | 729 | −12.611 | 24.430 | 3.122 | 1.00 | 81.24 |
| ATOM | 221 | CB | PHE | A | 729 | −12.480 | 25.821 | 0.563 | 1.00 | 81.06 |
| ATOM | 222 | CG | PHE | A | 729 | −12.521 | 26.879 | −0.503 | 1.00 | 82.69 |
| ATOM | 223 | CD1 | PHE | A | 729 | −11.372 | 27.197 | −1.230 | 1.00 | 83.00 |
| ATOM | 224 | CD2 | PHE | A | 729 | −13.690 | 27.601 | −0.744 | 1.00 | 82.96 |
| ATOM | 225 | CE1 | PHE | A | 729 | −11.385 | 28.222 | −2.180 | 1.00 | 82.28 |
| ATOM | 226 | CE2 | PHE | A | 729 | −13.712 | 28.625 | −1.690 | 1.00 | 82.16 |
| ATOM | 227 | CZ | PHE | A | 729 | −12.555 | 28.936 | −2.408 | 1.00 | 81.83 |
| ATOM | 228 | N | GLY | A | 730 | −10.417 | 24.895 | 3.307 | 1.00 | 78.97 |
| ATOM | 229 | CA | GLY | A | 730 | −10.176 | 23.800 | 4.234 | 1.00 | 75.86 |
| ATOM | 230 | C | GLY | A | 730 | −9.385 | 22.674 | 3.591 | 1.00 | 74.02 |
| ATOM | 231 | O | GLY | A | 730 | −9.022 | 22.756 | 2.418 | 1.00 | 74.18 |
| ATOM | 232 | N | THR | A | 731 | −9.124 | 21.613 | 4.347 | 1.00 | 71.57 |
| ATOM | 233 | CA | THR | A | 731 | −8.366 | 20.489 | 3.821 | 1.00 | 69.93 |
| ATOM | 234 | C | THR | A | 731 | −9.233 | 19.271 | 3.521 | 1.00 | 69.30 |
| ATOM | 235 | O | THR | A | 731 | −10.110 | 18.911 | 4.310 | 1.00 | 68.76 |
| ATOM | 236 | CB | THR | A | 731 | −7.255 | 20.078 | 4.795 | 1.00 | 70.50 |
| ATOM | 237 | OG1 | THR | A | 731 | −6.360 | 21.180 | 4.985 | 1.00 | 69.99 |
| ATOM | 238 | CG2 | THR | A | 731 | −6.479 | 18.893 | 4.244 | 1.00 | 69.46 |
| ATOM | 239 | N | VAL | A | 732 | −8.971 | 18.639 | 2.378 | 1.00 | 68.82 |
| ATOM | 240 | CA | VAL | A | 732 | −9.720 | 17.462 | 1.944 | 1.00 | 69.33 |
| ATOM | 241 | C | VAL | A | 732 | −8.852 | 16.202 | 1.933 | 1.00 | 70.00 |
| ATOM | 242 | O | VAL | A | 732 | −7.975 | 16.057 | 1.087 | 1.00 | 70.94 |
| ATOM | 243 | CB | VAL | A | 732 | −10.299 | 17.658 | 0.517 | 1.00 | 69.44 |
| ATOM | 244 | CG1 | VAL | A | 732 | −11.149 | 16.457 | 0.143 | 1.00 | 69.31 |
| ATOM | 245 | CG2 | VAL | A | 732 | −11.123 | 18.942 | 0.439 | 1.00 | 67.78 |
| ATOM | 246 | N | TYR | A | 733 | −9.112 | 15.291 | 2.867 | 1.00 | 70.94 |
| ATOM | 247 | CA | TYR | A | 733 | −8.361 | 14.040 | 2.980 | 1.00 | 70.76 |
| ATOM | 248 | C | TYR | A | 733 | −9.094 | 12.875 | 2.331 | 1.00 | 71.20 |
| ATOM | 249 | O | TYR | A | 733 | −10.322 | 12.862 | 2.263 | 1.00 | 70.51 |
| ATOM | 250 | CB | TYR | A | 733 | −8.164 | 13.661 | 4.449 | 1.00 | 73.12 |
| ATOM | 251 | CG | TYR | A | 733 | −7.466 | 14.679 | 5.318 | 1.00 | 75.05 |
| ATOM | 252 | CD1 | TYR | A | 733 | −6.080 | 14.646 | 5.498 | 1.00 | 75.57 |
| ATOM | 253 | CD2 | TYR | A | 733 | −8.193 | 15.665 | 5.984 | 1.00 | 75.38 |
| ATOM | 254 | CE1 | TYR | A | 733 | −5.439 | 15.571 | 6.326 | 1.00 | 74.72 |
| ATOM | 255 | CE2 | TYR | A | 733 | −7.564 | 16.594 | 6.809 | 1.00 | 75.15 |
| ATOM | 256 | CZ | TYR | A | 733 | −6.191 | 16.541 | 6.973 | 1.00 | 75.27 |
| ATOM | 257 | OH | TYR | A | 733 | −5.578 | 17.472 | 7.774 | 1.00 | 77.22 |
| ATOM | 258 | N | LYS | A | 734 | −8.340 | 11.888 | 1.857 | 1.00 | 72.36 |
| ATOM | 259 | CA | LYS | A | 734 | −8.955 | 10.688 | 1.294 | 1.00 | 72.08 |
| ATOM | 260 | C | LYS | A | 734 | −9.168 | 9.821 | 2.534 | 1.00 | 71.34 |
| ATOM | 261 | O | LYS | A | 734 | −8.445 | 9.984 | 3.524 | 1.00 | 69.91 |
| ATOM | 262 | CB | LYS | A | 734 | −8.005 | 9.974 | 0.333 | 1.00 | 72.27 |
| ATOM | 263 | CG | LYS | A | 734 | −8.519 | 8.605 | −0.097 | 1.00 | 73.95 |
| ATOM | 264 | CD | LYS | A | 734 | −7.471 | 7.804 | −0.857 | 1.00 | 73.38 |
| ATOM | 265 | CE | LYS | A | 734 | −7.982 | 6.400 | −1.146 | 1.00 | 73.91 |
| ATOM | 266 | NZ | LYS | A | 734 | −7.047 | 5.599 | −1.976 | 1.00 | 72.92 |
| ATOM | 267 | N | GLY | A | 735 | −10.139 | 8.913 | 2.512 | 1.00 | 70.61 |
| ATOM | 268 | CA | GLY | A | 735 | −10.338 | 8.104 | 3.702 | 1.00 | 71.08 |
| ATOM | 269 | C | GLY | A | 735 | −11.390 | 7.015 | 3.719 | 1.00 | 71.04 |
| ATOM | 270 | O | GLY | A | 735 | −12.152 | 6.839 | 2.767 | 1.00 | 71.51 |
| ATOM | 271 | N | ILE | A | 736 | −11.422 | 6.281 | 4.829 | 1.00 | 71.26 |
| ATOM | 272 | CA | ILE | A | 736 | −12.368 | 5.188 | 5.005 | 1.00 | 72.22 |
| ATOM | 273 | C | ILE | A | 736 | −13.364 | 5.496 | 6.107 | 1.00 | 72.21 |
| ATOM | 274 | O | ILE | A | 736 | −13.003 | 6.023 | 7.161 | 1.00 | 71.05 |
| ATOM | 275 | CB | ILE | A | 736 | −11.673 | 3.867 | 5.415 | 1.00 | 73.36 |
| ATOM | 276 | CG1 | ILE | A | 736 | −10.379 | 3.663 | 4.621 | 1.00 | 74.33 |
| ATOM | 277 | CG2 | ILE | A | 736 | −12.627 | 2.698 | 5.186 | 1.00 | 71.11 |
| ATOM | 278 | CD1 | ILE | A | 736 | −9.552 | 2.470 | 5.102 | 1.00 | 75.22 |
| ATOM | 279 | N | TRP | A | 737 | −14.618 | 5.146 | 5.858 | 1.00 | 72.87 |
| ATOM | 280 | CA | TRP | A | 737 | −15.674 | 5.346 | 6.832 | 1.00 | 73.66 |
| ATOM | 281 | C | TRP | A | 737 | −16.373 | 4.014 | 7.085 | 1.00 | 75.42 |
| ATOM | 282 | O | TRP | A | 737 | −16.835 | 3.358 | 6.147 | 1.00 | 75.21 |
| ATOM | 283 | CB | TRP | A | 737 | −16.681 | 6.383 | 6.328 | 1.00 | 71.21 |
| ATOM | 284 | CG | TRP | A | 737 | −17.960 | 6.395 | 7.106 | 1.00 | 69.09 |
| ATOM | 285 | CD1 | TRP | A | 737 | −18.096 | 6.373 | 8.466 | 1.00 | 68.36 |
| ATOM | 286 | CD2 | TRP | A | 737 | −19.285 | 6.432 | 6.571 | 1.00 | 68.26 |
| ATOM | 287 | NE1 | TRP | A | 737 | −19.426 | 6.391 | 8.810 | 1.00 | 67.91 |
| ATOM | 288 | CE2 | TRP | A | 737 | −20.179 | 6.430 | 7.668 | 1.00 | 67.63 |
| ATOM | 289 | CE3 | TRP | A | 737 | −19.806 | 6.470 | 5.271 | 1.00 | 67.83 |
| ATOM | 290 | CZ2 | TRP | A | 737 | −21.565 | 6.463 | 7.506 | 1.00 | 67.13 |
| ATOM | 291 | CZ3 | TRP | A | 737 | −21.189 | 6.503 | 5.107 | 1.00 | 68.96 |
| ATOM | 292 | CH2 | TRP | A | 737 | −22.053 | 6.500 | 6.223 | 1.00 | 68.91 |
| ATOM | 293 | N | VAL | A | 738 | −16.437 | 3.619 | 8.355 | 1.00 | 76.80 |
| ATOM | 294 | CA | VAL | A | 738 | −17.085 | 2.374 | 8.746 | 1.00 | 78.00 |
| ATOM | 295 | C | VAL | A | 738 | −18.328 | 2.714 | 9.563 | 1.00 | 79.76 |
| ATOM | 296 | O | VAL | A | 738 | −18.226 | 3.062 | 10.736 | 1.00 | 80.23 |
| ATOM | 297 | CB | VAL | A | 738 | −16.146 | 1.511 | 9.600 | 1.00 | 78.37 |
| ATOM | 298 | CG1 | VAL | A | 738 | −16.738 | 0.125 | 9.772 | 1.00 | 77.43 |
| ATOM | 299 | CG2 | VAL | A | 738 | −14.761 | 1.446 | 8.957 | 1.00 | 77.28 |
| ATOM | 300 | N | PRO | A | 739 | −19.518 | 2.605 | 8.948 | 1.00 | 81.79 |
| ATOM | 301 | CA | PRO | A | 739 | −20.837 | 2.887 | 9.531 | 1.00 | 83.53 |
| ATOM | 302 | C | PRO | A | 739 | −21.013 | 2.624 | 11.025 | 1.00 | 85.81 |
| ATOM | 303 | O | PRO | A | 739 | −20.216 | 1.912 | 11.643 | 1.00 | 85.32 |
| ATOM | 304 | CB | PRO | A | 739 | −21.768 | 2.042 | 8.676 | 1.00 | 82.99 |
| ATOM | 305 | CG | PRO | A | 739 | −21.149 | 2.186 | 7.327 | 1.00 | 83.21 |
| ATOM | 306 | CD | PRO | A | 739 | −19.671 | 1.980 | 7.621 | 1.00 | 82.26 |
| ATOM | 307 | N | ALA | A | 740 | −22.076 | 3.207 | 11.585 | 1.00 | 87.67 |
| ATOM | 308 | CA | ALA | A | 740 | −22.407 | 3.091 | 13.008 | 1.00 | 89.23 |
| ATOM | 309 | C | ALA | A | 740 | −22.747 | 1.670 | 13.453 | 1.00 | 90.19 |
| ATOM | 310 | O | ALA | A | 740 | −23.730 | 1.456 | 14.170 | 1.00 | 90.44 |
| ATOM | 311 | CB | ALA | A | 740 | −23.571 | 4.031 | 13.345 | 1.00 | 88.58 |
| ATOM | 312 | N | GLY | A | 741 | −21.929 | 0.708 | 13.034 | 1.00 | 90.71 |
| ATOM | 313 | CA | GLY | A | 741 | −22.156 | −0.680 | 13.396 | 1.00 | 91.14 |
| ATOM | 314 | C | GLY | A | 741 | −22.167 | −1.612 | 12.199 | 1.00 | 91.59 |
| ATOM | 315 | O | GLY | A | 741 | −23.100 | −2.396 | 12.025 | 1.00 | 91.52 |
| ATOM | 316 | N | GLU | A | 742 | −21.130 | −1.528 | 11.369 | 1.00 | 91.74 |
| ATOM | 317 | CA | GLU | A | 742 | −21.033 | −2.374 | 10.185 | 1.00 | 91.44 |
| ATOM | 318 | C | GLU | A | 742 | −19.579 | −2.705 | 9.875 | 1.00 | 91.13 |
| ATOM | 319 | O | GLU | A | 742 | −18.667 | −2.139 | 10.475 | 1.00 | 90.38 |
| ATOM | 320 | CB | GLU | A | 742 | −21.688 | −1.680 | 8.986 | 1.00 | 91.24 |
| ATOM | 321 | CG | GLU | A | 742 | −23.106 | −1.209 | 9.276 | 1.00 | 91.68 |
| ATOM | 322 | CD | GLU | A | 742 | −23.905 | −0.905 | 8.027 | 1.00 | 92.00 |
| ATOM | 323 | OE1 | GLU | A | 742 | −23.483 | −0.040 | 7.232 | 1.00 | 91.88 |
| ATOM | 324 | OE2 | GLU | A | 742 | −24.966 | −1.538 | 7.844 | 1.00 | 91.68 |
| ATOM | 325 | N | ALA | A | 743 | −19.370 | −3.625 | 8.936 | 1.00 | 91.28 |
| ATOM | 326 | CA | ALA | A | 743 | −18.024 | −4.048 | 8.559 | 1.00 | 91.22 |
| ATOM | 327 | C | ALA | A | 743 | −17.472 | −3.312 | 7.335 | 1.00 | 91.06 |
| ATOM | 328 | O | ALA | A | 743 | −16.276 | −3.011 | 7.273 | 1.00 | 90.46 |
| ATOM | 329 | CB | ALA | A | 743 | −18.010 | −5.559 | 8.307 | 1.00 | 90.34 |
| ATOM | 330 | N | VAL | A | 744 | −18.346 | −3.025 | 6.371 | 1.00 | 90.80 |
| ATOM | 331 | CA | VAL | A | 744 | −17.950 | −2.344 | 5.135 | 1.00 | 89.78 |
| ATOM | 332 | C | VAL | A | 744 | −17.025 | −1.146 | 5.353 | 1.00 | 88.68 |
| ATOM | 333 | O | VAL | A | 744 | −17.191 | −0.380 | 6.301 | 1.00 | 89.33 |
| ATOM | 334 | CB | VAL | A | 744 | −19.191 | −1.867 | 4.323 | 1.00 | 89.22 |
| ATOM | 335 | CG1 | VAL | A | 744 | −20.073 | −3.053 | 3.983 | 1.00 | 89.59 |
| ATOM | 336 | CG2 | VAL | A | 744 | −19.975 | −0.831 | 5.111 | 1.00 | 88.82 |
| ATOM | 337 | N | LYS | A | 745 | −16.042 | −0.999 | 4.469 | 1.00 | 86.57 |
| ATOM | 338 | CA | LYS | A | 745 | −15.098 | 0.111 | 4.542 | 1.00 | 84.05 |
| ATOM | 339 | C | LYS | A | 745 | −15.364 | 1.094 | 3.401 | 1.00 | 82.42 |
| ATOM | 340 | O | LYS | A | 745 | −14.580 | 1.174 | 2.460 | 1.00 | 81.96 |
| ATOM | 341 | CB | LYS | A | 745 | −13.655 | −0.399 | 4.439 | 1.00 | 83.71 |
| ATOM | 342 | CG | LYS | A | 745 | −13.093 | −1.055 | 5.696 | 1.00 | 82.67 |
| ATOM | 343 | CD | LYS | A | 745 | −11.580 | −1.229 | 5.574 | 1.00 | 81.20 |
| ATOM | 344 | CE | LYS | A | 745 | −10.962 | −1.742 | 6.864 | 1.00 | 81.60 |
| ATOM | 345 | NZ | LYS | A | 745 | −9.473 | −1.807 | 6.792 | 1.00 | 80.27 |
| ATOM | 346 | N | ILE | A | 746 | −16.469 | 1.834 | 3.486 | 1.00 | 80.20 |
| ATOM | 347 | CA | ILE | A | 746 | −16.829 | 2.804 | 2.451 | 1.00 | 77.59 |
| ATOM | 348 | C | ILE | A | 746 | −15.753 | 3.867 | 2.234 | 1.00 | 75.28 |
| ATOM | 349 | O | ILE | A | 746 | −15.354 | 4.565 | 3.165 | 1.00 | 75.06 |
| ATOM | 350 | CB | ILE | A | 746 | −18.153 | 3.521 | 2.784 | 1.00 | 78.03 |
| ATOM | 351 | CG1 | ILE | A | 746 | −19.299 | 2.508 | 2.844 | 1.00 | 78.83 |
| ATOM | 352 | CG2 | ILE | A | 746 | −18.451 | 4.568 | 1.726 | 1.00 | 79.05 |
| ATOM | 353 | CD1 | ILE | A | 746 | −20.671 | 3.125 | 3.096 | 1.00 | 78.81 |
| ATOM | 354 | N | PRO | A | 747 | −15.268 | 4.000 | 0.990 | 1.00 | 74.01 |
| ATOM | 355 | CA | PRO | A | 747 | −14.235 | 4.985 | 0.654 | 1.00 | 71.83 |
| ATOM | 356 | C | PRO | A | 747 | −14.866 | 6.370 | 0.601 | 1.00 | 70.14 |
| ATOM | 357 | O | PRO | A | 747 | −15.882 | 6.574 | −0.077 | 1.00 | 68.69 |
| ATOM | 358 | CB | PRO | A | 747 | −13.753 | 4.510 | −0.709 | 1.00 | 71.63 |
| ATOM | 359 | CG | PRO | A | 747 | −15.018 | 4.016 | −1.336 | 1.00 | 72.44 |
| ATOM | 360 | CD | PRO | A | 747 | −15.673 | 3.237 | −0.207 | 1.00 | 74.00 |
| ATOM | 361 | N | VAL | A | 748 | −14.269 | 7.322 | 1.311 | 1.00 | 67.54 |
| ATOM | 362 | CA | VAL | A | 748 | −14.820 | 8.669 | 1.346 | 1.00 | 65.96 |
| ATOM | 363 | C | VAL | A | 748 | −13.797 | 9.791 | 1.302 | 1.00 | 64.98 |
| ATOM | 364 | O | VAL | A | 748 | −12.584 | 9.565 | 1.370 | 1.00 | 63.82 |
| ATOM | 365 | CB | VAL | A | 748 | −15.659 | 8.902 | 2.622 | 1.00 | 66.10 |
| ATOM | 366 | CG1 | VAL | A | 748 | −16.839 | 7.953 | 2.666 | 1.00 | 65.30 |
| ATOM | 367 | CG2 | VAL | A | 748 | −14.776 | 8.731 | 3.846 | 1.00 | 64.29 |
| ATOM | 368 | N | ALA | A | 749 | −14.325 | 11.008 | 1.179 | 1.00 | 62.57 |
| ATOM | 369 | CA | ALA | A | 749 | −13.525 | 12.221 | 1.172 | 1.00 | 61.68 |
| ATOM | 370 | C | ALA | A | 749 | −14.004 | 12.942 | 2.411 | 1.00 | 61.42 |
| ATOM | 371 | O | ALA | A | 749 | −15.204 | 13.026 | 2.656 | 1.00 | 62.73 |
| ATOM | 372 | CB | ALA | A | 749 | −13.814 | 13.058 | 0.060 | 1.00 | 60.54 |
| ATOM | 373 | N | ILE | A | 750 | −13.070 | 13.432 | 3.212 | 1.00 | 60.76 |
| ATOM | 374 | CA | ILE | A | 750 | −13.425 | 14.143 | 4.423 | 1.00 | 59.53 |
| ATOM | 375 | C | ILE | A | 750 | −12.830 | 15.519 | 4.312 | 1.00 | 58.84 |
| ATOM | 376 | O | ILE | A | 750 | −11.650 | 15.667 | 4.014 | 1.00 | 58.42 |
| ATOM | 377 | CB | ILE | A | 750 | −12.855 | 13.456 | 5.671 | 1.00 | 59.88 |
| ATOM | 378 | CG1 | ILE | A | 750 | −13.315 | 11.999 | 5.710 | 1.00 | 61.77 |
| ATOM | 379 | CG2 | ILE | A | 750 | −13.312 | 14.193 | 6.917 | 1.00 | 59.44 |
| ATOM | 380 | CD1 | ILE | A | 750 | −12.707 | 11.180 | 6.836 | 1.00 | 64.53 |
| ATOM | 381 | N | LYS | A | 751 | −13.654 | 16.529 | 4.532 | 1.00 | 59.29 |
| ATOM | 382 | CA | LYS | A | 751 | −13.188 | 17.897 | 4.458 | 1.00 | 60.16 |
| ATOM | 383 | C | LYS | A | 751 | −13.373 | 18.529 | 5.814 | 1.00 | 61.42 |
| ATOM | 384 | O | LYS | A | 751 | −14.406 | 18.347 | 6.463 | 1.00 | 60.51 |
| ATOM | 385 | CB | LYS | A | 751 | −13.982 | 18.676 | 3.410 | 1.00 | 59.82 |
| ATOM | 386 | CG | LYS | A | 751 | −13.519 | 20.109 | 3.247 | 1.00 | 59.02 |
| ATOM | 387 | CD | LYS | A | 751 | −14.138 | 20.745 | 2.021 | 1.00 | 58.49 |
| ATOM | 388 | CE | LYS | A | 751 | −13.435 | 22.035 | 1.690 | 1.00 | 57.20 |
| ATOM | 389 | NZ | LYS | A | 751 | −13.915 | 22.606 | 0.411 | 1.00 | 58.47 |
| ATOM | 390 | N | ILE | A | 752 | −12.365 | 19.271 | 6.242 | 1.00 | 63.64 |
| ATOM | 391 | CA | ILE | A | 752 | −12.420 | 19.935 | 7.528 | 1.00 | 66.22 |
| ATOM | 392 | C | ILE | A | 752 | −12.349 | 21.438 | 7.329 | 1.00 | 67.39 |
| ATOM | 393 | O | ILE | A | 752 | −11.394 | 21.945 | 6.749 | 1.00 | 67.55 |
| ATOM | 394 | CB | ILE | A | 752 | −11.256 | 19.473 | 8.422 | 1.00 | 66.68 |
| ATOM | 395 | CG1 | ILE | A | 752 | −11.241 | 17.943 | 8.481 | 1.00 | 66.85 |
| ATOM | 396 | CG2 | ILE | A | 752 | −11.404 | 20.065 | 9.818 | 1.00 | 67.28 |
| ATOM | 397 | CD1 | ILE | A | 752 | −10.147 | 17.356 | 9.336 | 1.00 | 66.47 |
| ATOM | 398 | N | ALA | A | 753 | −13.372 | 22.146 | 7.799 | 1.00 | 70.53 |
| ATOM | 399 | CA | ALA | A | 753 | −13.419 | 23.602 | 7.673 | 1.00 | 74.64 |
| ATOM | 400 | C | ALA | A | 753 | −12.365 | 24.214 | 8.594 | 1.00 | 76.96 |
| ATOM | 401 | O | ALA | A | 753 | −11.877 | 23.534 | 9.500 | 1.00 | 77.82 |
| ATOM | 402 | CB | ALA | A | 753 | −14.806 | 24.110 | 8.038 | 1.00 | 73.77 |
| ATOM | 403 | N | VAL | A | 754 | −12.020 | 25.485 | 8.372 | 1.00 | 79.45 |
| ATOM | 404 | CA | VAL | A | 754 | −10.997 | 26.157 | 9.189 | 1.00 | 81.01 |
| ATOM | 405 | CB | VAL | A | 754 | −10.273 | 27.279 | 8.378 | 1.00 | 80.88 |
| ATOM | 406 | CG1 | VAL | A | 754 | −8.977 | 27.694 | 9.096 | 1.00 | 78.71 |
| ATOM | 407 | CG2 | VAL | A | 754 | −9.989 | 26.799 | 6.945 | 1.00 | 79.23 |
| ATOM | 408 | C | VAL | A | 754 | −11.541 | 26.769 | 10.494 | 1.00 | 82.00 |
| ATOM | 409 | OT1 | VAL | A | 754 | −11.212 | 27.942 | 10.798 | 1.00 | 82.05 |
| ATOM | 410 | OT2 | VAL | A | 754 | −12.277 | 26.057 | 11.216 | 1.00 | 82.11 |
| ATOM | 411 | N | ALA | A | 761 | −22.557 | 30.871 | 14.717 | 1.00 | 87.31 |
| ATOM | 412 | CA | ALA | A | 761 | −22.100 | 30.438 | 13.364 | 1.00 | 87.94 |
| ATOM | 413 | C | ALA | A | 761 | −22.469 | 28.982 | 13.071 | 1.00 | 88.76 |
| ATOM | 414 | O | ALA | A | 761 | −23.035 | 28.682 | 12.017 | 1.00 | 89.19 |
| ATOM | 415 | CB | ALA | A | 761 | −20.589 | 30.632 | 13.236 | 1.00 | 86.83 |
| ATOM | 416 | N | ASN | A | 762 | −22.147 | 28.080 | 13.995 | 1.00 | 88.98 |
| ATOM | 417 | CA | ASN | A | 762 | −22.459 | 26.666 | 13.812 | 1.00 | 89.06 |
| ATOM | 418 | C | ASN | A | 762 | −23.933 | 26.471 | 13.493 | 1.00 | 89.09 |
| ATOM | 419 | O | ASN | A | 762 | −24.339 | 25.428 | 12.975 | 1.00 | 89.26 |
| ATOM | 420 | CB | ASN | A | 762 | −22.083 | 25.865 | 15.060 | 1.00 | 89.85 |
| ATOM | 421 | CG | ASN | A | 762 | −20.608 | 25.519 | 15.102 | 1.00 | 90.47 |
| ATOM | 422 | OD1 | ASN | A | 762 | −19.749 | 26.403 | 15.132 | 1.00 | 89.73 |
| ATOM | 423 | ND2 | ASN | A | 762 | −20.306 | 24.225 | 15.098 | 1.00 | 90.01 |
| ATOM | 424 | N | VAL | A | 763 | −24.735 | 27.481 | 13.811 | 1.00 | 88.96 |
| ATOM | 425 | CA | VAL | A | 763 | −26.160 | 27.425 | 13.533 | 1.00 | 88.35 |
| ATOM | 426 | C | VAL | A | 763 | −26.274 | 27.447 | 12.020 | 1.00 | 88.39 |
| ATOM | 427 | O | VAL | A | 763 | −26.950 | 26.609 | 11.427 | 1.00 | 88.27 |
| ATOM | 428 | CB | VAL | A | 763 | −26.899 | 28.657 | 14.076 | 1.00 | 88.18 |
| ATOM | 429 | CG1 | VAL | A | 763 | −28.380 | 28.348 | 14.200 | 1.00 | 87.36 |
| ATOM | 430 | CG2 | VAL | A | 763 | −26.302 | 29.086 | 15.406 | 1.00 | 88.46 |
| ATOM | 431 | N | GLU | A | 764 | −25.598 | 28.420 | 11.408 | 1.00 | 87.88 |
| ATOM | 432 | CA | GLU | A | 764 | −25.599 | 28.574 | 9.956 | 1.00 | 86.88 |
| ATOM | 433 | C | GLU | A | 764 | −25.164 | 27.260 | 9.339 | 1.00 | 84.91 |
| ATOM | 434 | O | GLU | A | 764 | −25.826 | 26.724 | 8.448 | 1.00 | 85.31 |
| ATOM | 435 | CB | GLU | A | 764 | −24.609 | 29.654 | 9.507 | 1.00 | 88.52 |
| ATOM | 436 | CG | GLU | A | 764 | −24.708 | 30.982 | 10.231 | 1.00 | 90.41 |
| ATOM | 437 | CD | GLU | A | 764 | −23.821 | 32.047 | 9.600 | 1.00 | 91.24 |
| ATOM | 438 | OE1 | GLU | A | 764 | −22.685 | 31.714 | 9.190 | 1.00 | 92.50 |
| ATOM | 439 | OE2 | GLU | A | 764 | −24.254 | 33.218 | 9.523 | 1.00 | 91.08 |
| ATOM | 440 | N | PHE | A | 765 | −24.037 | 26.751 | 9.823 | 1.00 | 81.83 |
| ATOM | 441 | CA | PHE | A | 765 | −23.491 | 25.507 | 9.322 | 1.00 | 78.37 |
| ATOM | 442 | C | PHE | A | 765 | −24.495 | 24.367 | 9.297 | 1.00 | 75.51 |
| ATOM | 443 | O | PHE | A | 765 | −24.687 | 23.722 | 8.269 | 1.00 | 74.23 |
| ATOM | 444 | CB | PHE | A | 765 | −22.280 | 25.080 | 10.141 | 1.00 | 79.06 |
| ATOM | 445 | CG | PHE | A | 765 | −21.729 | 23.762 | 9.717 | 1.00 | 80.47 |
| ATOM | 446 | CD1 | PHE | A | 765 | −22.371 | 22.577 | 10.079 | 1.00 | 81.96 |
| ATOM | 447 | CD2 | PHE | A | 765 | −20.638 | 23.701 | 8.861 | 1.00 | 80.75 |
| ATOM | 448 | CE1 | PHE | A | 765 | −21.945 | 21.353 | 9.586 | 1.00 | 81.90 |
| ATOM | 449 | CE2 | PHE | A | 765 | −20.201 | 22.482 | 8.360 | 1.00 | 82.39 |
| ATOM | 450 | CZ | PHE | A | 765 | −20.861 | 21.302 | 8.722 | 1.00 | 82.56 |
| ATOM | 451 | N | MET | A | 766 | −25.117 | 24.092 | 10.433 | 1.00 | 72.71 |
| ATOM | 452 | CA | MET | A | 766 | −26.083 | 23.014 | 10.472 | 1.00 | 70.93 |
| ATOM | 453 | C | MET | A | 766 | −27.205 | 23.255 | 9.468 | 1.00 | 69.55 |
| ATOM | 454 | O | MET | A | 766 | −27.946 | 22.338 | 9.120 | 1.00 | 70.42 |
| ATOM | 455 | CB | MET | A | 766 | −26.638 | 22.849 | 11.887 | 1.00 | 71.33 |
| ATOM | 456 | CG | MET | A | 766 | −25.603 | 22.336 | 12.873 | 1.00 | 71.61 |
| ATOM | 457 | SD | MET | A | 766 | −24.622 | 20.959 | 12.199 | 1.00 | 74.04 |
| ATOM | 458 | CE | MET | A | 766 | −25.720 | 19.631 | 12.348 | 1.00 | 69.51 |
| ATOM | 459 | N | ASP | A | 767 | −27.331 | 24.489 | 8.995 | 1.00 | 66.54 |
| ATOM | 460 | CA | ASP | A | 767 | −28.355 | 24.795 | 8.011 | 1.00 | 65.26 |
| ATOM | 461 | C | ASP | A | 767 | −27.783 | 24.538 | 6.629 | 1.00 | 63.58 |
| ATOM | 462 | O | ASP | A | 767 | −28.415 | 23.896 | 5.795 | 1.00 | 64.00 |
| ATOM | 463 | CB | ASP | A | 767 | −28.807 | 26.249 | 8.131 | 1.00 | 67.20 |
| ATOM | 464 | CG | ASP | A | 767 | −29.615 | 26.504 | 9.390 | 1.00 | 68.51 |
| ATOM | 465 | OD1 | ASP | A | 767 | −30.624 | 25.794 | 9.594 | 1.00 | 68.20 |
| ATOM | 466 | OD2 | ASP | A | 767 | −29.242 | 27.408 | 10.172 | 1.00 | 68.59 |
| ATOM | 467 | N | GLU | A | 768 | −26.577 | 25.034 | 6.395 | 1.00 | 61.36 |
| ATOM | 468 | CA | GLU | A | 768 | −25.917 | 24.841 | 5.118 | 1.00 | 60.21 |
| ATOM | 469 | C | GLU | A | 768 | −25.744 | 23.350 | 4.846 | 1.00 | 58.77 |
| ATOM | 470 | O | GLU | A | 768 | −25.834 | 22.908 | 3.704 | 1.00 | 58.90 |
| ATOM | 471 | CB | GLU | A | 768 | −24.555 | 25.531 | 5.123 | 1.00 | 62.67 |
| ATOM | 472 | CG | GLU | A | 768 | −23.938 | 25.709 | 3.745 | 1.00 | 68.86 |
| ATOM | 473 | CD | GLU | A | 768 | −24.809 | 26.548 | 2.806 | 1.00 | 72.06 |
| ATOM | 474 | OE1 | GLU | A | 768 | −24.346 | 26.888 | 1.696 | 1.00 | 75.67 |
| ATOM | 475 | OE2 | GLU | A | 768 | −25.960 | 26.866 | 3.168 | 1.00 | 73.63 |
| ATOM | 476 | N | ALA | A | 769 | −25.515 | 22.574 | 5.902 | 1.00 | 56.78 |
| ATOM | 477 | CA | ALA | A | 769 | −25.329 | 21.134 | 5.764 | 1.00 | 53.94 |
| ATOM | 478 | C | ALA | A | 769 | −26.602 | 20.437 | 5.328 | 1.00 | 53.30 |
| ATOM | 479 | O | ALA | A | 769 | −26.547 | 19.423 | 4.626 | 1.00 | 55.21 |
| ATOM | 480 | CB | ALA | A | 769 | −24.840 | 20.544 | 7.065 | 1.00 | 53.65 |
| ATOM | 481 | N | LEU | A | 770 | −27.749 | 20.963 | 5.751 | 1.00 | 51.12 |
| ATOM | 482 | CA | LEU | A | 770 | −29.029 | 20.373 | 5.370 | 1.00 | 50.39 |
| ATOM | 483 | C | LEU | A | 770 | −29.127 | 20.440 | 3.851 | 1.00 | 49.87 |
| ATOM | 484 | O | LEU | A | 770 | −29.584 | 19.502 | 3.194 | 1.00 | 48.92 |
| ATOM | 485 | CB | LEU | A | 770 | −30.206 | 21.136 | 6.019 | 1.00 | 49.49 |
| ATOM | 486 | CG | LEU | A | 770 | −31.615 | 20.629 | 5.648 | 1.00 | 50.99 |
| ATOM | 487 | CD1 | LEU | A | 770 | −32.640 | 20.997 | 6.711 | 1.00 | 51.18 |
| ATOM | 488 | CD2 | LEU | A | 770 | −32.026 | 21.204 | 4.295 | 1.00 | 48.88 |
| ATOM | 489 | N | ILE | A | 771 | −28.683 | 21.561 | 3.298 | 1.00 | 49.87 |
| ATOM | 490 | CA | ILE | A | 771 | −28.714 | 21.751 | 1.860 | 1.00 | 50.49 |
| ATOM | 491 | C | ILE | A | 771 | −27.728 | 20.805 | 1.175 | 1.00 | 50.51 |
| ATOM | 492 | O | ILE | A | 771 | −28.092 | 20.059 | 0.271 | 1.00 | 51.88 |
| ATOM | 493 | CB | ILE | A | 771 | −28.352 | 23.196 | 1.491 | 1.00 | 49.76 |
| ATOM | 494 | CG1 | ILE | A | 771 | −29.258 | 24.175 | 2.249 | 1.00 | 51.25 |
| ATOM | 495 | CG2 | ILE | A | 771 | −28.509 | 23.394 | −0.006 | 1.00 | 48.11 |
| ATOM | 496 | CD1 | ILE | A | 771 | −30.729 | 24.104 | 1.846 | 1.00 | 52.39 |
| ATOM | 497 | N | MET | A | 772 | −26.479 | 20.830 | 1.616 | 1.00 | 49.68 |
| ATOM | 498 | CA | MET | A | 772 | −25.469 | 19.976 | 1.023 | 1.00 | 49.48 |
| ATOM | 499 | C | MET | A | 772 | −25.925 | 18.544 | 1.041 | 1.00 | 49.90 |
| ATOM | 500 | O | MET | A | 772 | −25.504 | 17.748 | 0.216 | 1.00 | 53.68 |
| ATOM | 501 | CB | MET | A | 772 | −24.152 | 20.078 | 1.784 | 1.00 | 50.02 |
| ATOM | 502 | CG | MET | A | 772 | −23.670 | 21.485 | 1.974 | 1.00 | 50.14 |
| ATOM | 503 | SD | MET | A | 772 | −22.079 | 21.532 | 2.743 | 1.00 | 51.82 |
| ATOM | 504 | CE | MET | A | 772 | −21.416 | 23.034 | 1.966 | 1.00 | 54.26 |
| ATOM | 505 | N | ALA | A | 773 | −26.797 | 18.210 | 1.975 | 1.00 | 49.25 |
| ATOM | 506 | CA | ALA | A | 773 | −27.265 | 16.841 | 2.078 | 1.00 | 49.85 |
| ATOM | 507 | C | ALA | A | 773 | −28.569 | 16.559 | 1.339 | 1.00 | 49.63 |
| ATOM | 508 | O | ALA | A | 773 | −28.963 | 15.405 | 1.189 | 1.00 | 49.98 |
| ATOM | 509 | CB | ALA | A | 773 | −27.403 | 16.467 | 3.554 | 1.00 | 50.21 |
| ATOM | 510 | N | SER | A | 774 | −29.243 | 17.597 | 0.867 | 1.00 | 50.09 |
| ATOM | 511 | CA | SER | A | 774 | −30.507 | 17.385 | 0.172 | 1.00 | 51.17 |
| ATOM | 512 | C | SER | A | 774 | −30.366 | 17.304 | −1.333 | 1.00 | 50.49 |
| ATOM | 513 | O | SER | A | 774 | −31.325 | 16.996 | −2.027 | 1.00 | 53.51 |
| ATOM | 514 | CB | SER | A | 774 | −31.503 | 18.496 | 0.514 | 1.00 | 50.00 |
| ATOM | 515 | OG | SER | A | 774 | −31.733 | 18.568 | 1.903 | 1.00 | 53.58 |
| ATOM | 516 | N | MET | A | 775 | −29.181 | 17.578 | −1.850 | 1.00 | 51.19 |
| ATOM | 517 | CA | MET | A | 775 | −28.991 | 17.536 | −3.296 | 1.00 | 51.57 |
| ATOM | 518 | C | MET | A | 775 | −29.005 | 16.115 | −3.879 | 1.00 | 50.46 |
| ATOM | 519 | O | MET | A | 775 | −28.128 | 15.298 | −3.603 | 1.00 | 49.82 |
| ATOM | 520 | CB | MET | A | 775 | −27.701 | 18.277 | −3.657 | 1.00 | 52.20 |
| ATOM | 521 | CG | MET | A | 775 | −27.711 | 19.720 | −3.185 | 1.00 | 52.73 |
| ATOM | 522 | SD | MET | A | 775 | −29.349 | 20.470 | −3.403 | 1.00 | 58.26 |
| ATOM | 523 | CE | MET | A | 775 | −29.044 | 21.567 | −4.684 | 1.00 | 55.94 |
| ATOM | 524 | N | ASP | A | 776 | −30.022 | 15.836 | −4.688 | 1.00 | 49.69 |
| ATOM | 525 | CA | ASP | A | 776 | −30.190 | 14.532 | −5.309 | 1.00 | 47.92 |
| ATOM | 526 | C | ASP | A | 776 | −30.278 | 14.653 | −6.819 | 1.00 | 46.24 |
| ATOM | 527 | O | ASP | A | 776 | −31.354 | 14.609 | −7.411 | 1.00 | 43.09 |
| ATOM | 528 | CB | ASP | A | 776 | −31.442 | 13.853 | −4.762 | 1.00 | 51.21 |
| ATOM | 529 | CG | ASP | A | 776 | −31.736 | 12.519 | −5.434 | 1.00 | 54.67 |
| ATOM | 530 | OD1 | ASP | A | 776 | −30.795 | 11.880 | −5.970 | 1.00 | 54.64 |
| ATOM | 531 | OD2 | ASP | A | 776 | −32.919 | 12.105 | −5.407 | 1.00 | 56.04 |
| ATOM | 532 | N | HIS | A | 777 | −29.110 | 14.797 | −7.428 | 1.00 | 45.15 |
| ATOM | 533 | CA | HIS | A | 777 | −28.977 | 14.929 | −8.862 | 1.00 | 43.19 |
| ATOM | 534 | C | HIS | A | 777 | −27.687 | 14.220 | −9.231 | 1.00 | 44.58 |
| ATOM | 535 | O | HIS | A | 777 | −26.690 | 14.324 | −8.524 | 1.00 | 43.08 |
| ATOM | 536 | CB | HIS | A | 777 | −28.899 | 16.401 | −9.239 | 1.00 | 41.16 |
| ATOM | 537 | CG | HIS | A | 777 | −28.776 | 16.641 | −10.705 | 1.00 | 42.58 |
| ATOM | 538 | ND1 | HIS | A | 777 | −27.601 | 16.439 | −11.397 | 1.00 | 42.84 |
| ATOM | 539 | CD2 | HIS | A | 777 | −29.684 | 17.063 | −11.616 | 1.00 | 40.33 |
| ATOM | 540 | CE1 | HIS | A | 777 | −27.791 | 16.728 | −12.672 | 1.00 | 42.28 |
| ATOM | 541 | NE2 | HIS | A | 777 | −29.047 | 17.109 | −12.830 | 1.00 | 41.77 |
| ATOM | 542 | N | PRO | A | 778 | −27.690 | 13.484 | −10.350 | 1.00 | 46.58 |
| ATOM | 543 | CA | PRO | A | 778 | −26.485 | 12.763 | −10.768 | 1.00 | 47.00 |
| ATOM | 544 | C | PRO | A | 778 | −25.241 | 13.622 | −10.969 | 1.00 | 47.70 |
| ATOM | 545 | O | PRO | A | 778 | −24.113 | 13.150 | −10.775 | 1.00 | 48.99 |
| ATOM | 546 | CB | PRO | A | 778 | −26.930 | 12.047 | −12.046 | 1.00 | 45.89 |
| ATOM | 547 | CG | PRO | A | 778 | −28.008 | 12.925 | −12.575 | 1.00 | 45.82 |
| ATOM | 548 | CD | PRO | A | 778 | −28.773 | 13.323 | −11.338 | 1.00 | 45.42 |
| ATOM | 549 | N | HIS | A | 779 | −25.415 | 14.890 | −11.318 | 1.00 | 46.96 |
| ATOM | 550 | CA | HIS | A | 779 | −24.231 | 15.701 | −11.533 | 1.00 | 46.65 |
| ATOM | 551 | C | HIS | A | 779 | −23.869 | 16.697 | −10.426 | 1.00 | 46.00 |
| ATOM | 552 | O | HIS | A | 779 | −23.195 | 17.709 | −10.640 | 1.00 | 43.82 |
| ATOM | 553 | CB | HIS | A | 779 | −24.318 | 16.330 | −12.922 | 1.00 | 45.35 |
| ATOM | 554 | CG | HIS | A | 779 | −24.475 | 15.309 | −14.009 | 1.00 | 46.09 |
| ATOM | 555 | ND1 | HIS | A | 779 | −23.614 | 14.242 | −14.153 | 1.00 | 45.56 |
| ATOM | 556 | CD2 | HIS | A | 779 | −25.423 | 15.156 | −14.966 | 1.00 | 46.11 |
| ATOM | 557 | CE1 | HIS | A | 779 | −24.025 | 13.476 | −15.148 | 1.00 | 44.45 |
| ATOM | 558 | NE2 | HIS | A | 779 | −25.121 | 14.008 | −15.658 | 1.00 | 43.34 |
| ATOM | 559 | N | LEU | A | 780 | −24.307 | 16.364 | −9.222 | 1.00 | 45.67 |
| ATOM | 560 | CA | LEU | A | 780 | −23.985 | 17.141 | −8.038 | 1.00 | 48.21 |
| ATOM | 561 | C | LEU | A | 780 | −23.651 | 16.086 | −6.994 | 1.00 | 48.90 |
| ATOM | 562 | O | LEU | A | 780 | −24.192 | 14.979 | −7.029 | 1.00 | 48.98 |
| ATOM | 563 | CB | LEU | A | 780 | −25.176 | 17.986 | −7.572 | 1.00 | 48.42 |
| ATOM | 564 | CG | LEU | A | 780 | −25.561 | 19.240 | −8.367 | 1.00 | 49.03 |
| ATOM | 565 | CD1 | LEU | A | 780 | −26.749 | 19.886 | −7.683 | 1.00 | 49.91 |
| ATOM | 566 | CD2 | LEU | A | 780 | −24.408 | 20.229 | −8.432 | 1.00 | 47.36 |
| ATOM | 567 | N | VAL | A | 781 | −22.746 | 16.398 | −6.081 | 1.00 | 49.42 |
| ATOM | 568 | CA | VAL | A | 781 | −22.413 | 15.420 | −5.060 | 1.00 | 51.41 |
| ATOM | 569 | C | VAL | A | 781 | −23.365 | 15.572 | −3.882 | 1.00 | 53.85 |
| ATOM | 570 | O | VAL | A | 781 | −24.077 | 16.572 | −3.762 | 1.00 | 53.60 |
| ATOM | 571 | CB | VAL | A | 781 | −20.969 | 15.592 | −4.564 | 1.00 | 49.34 |
| ATOM | 572 | CG1 | VAL | A | 781 | −19.998 | 15.434 | −5.730 | 1.00 | 48.15 |
| ATOM | 573 | CG2 | VAL | A | 781 | −20.809 | 16.942 | −3.910 | 1.00 | 48.13 |
| ATOM | 574 | N | ARG | A | 782 | −23.386 | 14.565 | −3.018 | 1.00 | 57.31 |
| ATOM | 575 | CA | ARG | A | 782 | −24.238 | 14.593 | −1.834 | 1.00 | 58.51 |
| ATOM | 576 | C | ARG | A | 782 | −23.429 | 14.365 | −0.560 | 1.00 | 57.98 |
| ATOM | 577 | O | ARG | A | 782 | −22.467 | 13.589 | −0.537 | 1.00 | 58.11 |
| ATOM | 578 | CB | ARG | A | 782 | −25.343 | 13.539 | −1.943 | 1.00 | 59.06 |
| ATOM | 579 | CG | ARG | A | 782 | −26.213 | 13.466 | −0.718 | 1.00 | 64.02 |
| ATOM | 580 | CD | ARG | A | 782 | −27.508 | 12.723 | −0.986 | 1.00 | 69.63 |
| ATOM | 581 | NE | ARG | A | 782 | −28.200 | 12.383 | 0.259 | 1.00 | 74.15 |
| ATOM | 582 | CZ | ARG | A | 782 | −29.459 | 11.955 | 0.325 | 1.00 | 77.34 |
| ATOM | 583 | NH1 | ARG | A | 782 | −30.173 | 11.821 | −0.793 | 1.00 | 76.96 |
| ATOM | 584 | NH2 | ARG | A | 782 | −29.999 | 11.642 | 1.504 | 1.00 | 76.96 |
| ATOM | 585 | N | LEU | A | 783 | −23.817 | 15.063 | 0.497 | 1.00 | 57.15 |
| ATOM | 586 | CA | LEU | A | 783 | −23.146 | 14.930 | 1.776 | 1.00 | 57.64 |
| ATOM | 587 | C | LEU | A | 783 | −23.644 | 13.666 | 2.470 | 1.00 | 57.90 |
| ATOM | 588 | O | LEU | A | 783 | −24.824 | 13.568 | 2.811 | 1.00 | 58.39 |
| ATOM | 589 | CB | LEU | A | 783 | −23.454 | 16.147 | 2.649 | 1.00 | 56.99 |
| ATOM | 590 | CG | LEU | A | 783 | −22.693 | 16.228 | 3.970 | 1.00 | 55.50 |
| ATOM | 591 | CD1 | LEU | A | 783 | −21.204 | 16.354 | 3.679 | 1.00 | 54.23 |
| ATOM | 592 | CD2 | LEU | A | 783 | −23.189 | 17.409 | 4.771 | 1.00 | 53.18 |
| ATOM | 593 | N | LEU | A | 784 | −22.755 | 12.698 | 2.671 | 1.00 | 57.71 |
| ATOM | 594 | CA | LEU | A | 784 | −23.137 | 11.456 | 3.341 | 1.00 | 57.94 |
| ATOM | 595 | C | LEU | A | 784 | −23.412 | 11.744 | 4.809 | 1.00 | 57.07 |
| ATOM | 596 | O | LEU | A | 784 | −24.453 | 11.360 | 5.353 | 1.00 | 57.68 |
| ATOM | 597 | CB | LEU | A | 784 | −22.020 | 10.416 | 3.249 | 1.00 | 58.52 |
| ATOM | 598 | CG | LEU | A | 784 | −21.758 | 9.742 | 1.905 | 1.00 | 61.56 |
| ATOM | 599 | CD1 | LEU | A | 784 | −20.493 | 8.905 | 2.013 | 1.00 | 62.06 |
| ATOM | 600 | CD2 | LEU | A | 784 | −22.950 | 8.881 | 1.499 | 1.00 | 60.90 |
| ATOM | 601 | N | GLY | A | 785 | −22.472 | 12.433 | 5.446 | 1.00 | 55.42 |
| ATOM | 602 | CA | GLY | A | 785 | −22.635 | 12.742 | 6.848 | 1.00 | 54.66 |
| ATOM | 603 | C | GLY | A | 785 | −21.732 | 13.831 | 7.380 | 1.00 | 54.77 |
| ATOM | 604 | O | GLY | A | 785 | −20.882 | 14.378 | 6.677 | 1.00 | 53.15 |
| ATOM | 605 | N | VAL | A | 786 | −21.933 | 14.152 | 8.649 | 1.00 | 55.12 |
| ATOM | 606 | CA | VAL | A | 786 | −21.147 | 15.177 | 9.300 | 1.00 | 55.18 |
| ATOM | 607 | C | VAL | A | 786 | −20.685 | 14.649 | 10.638 | 1.00 | 56.04 |
| ATOM | 608 | O | VAL | A | 786 | −21.445 | 13.976 | 11.337 | 1.00 | 57.35 |
| ATOM | 609 | CB | VAL | A | 786 | −21.983 | 16.449 | 9.546 | 1.00 | 55.04 |
| ATOM | 610 | CG1 | VAL | A | 786 | −21.148 | 17.477 | 10.282 | 1.00 | 54.87 |
| ATOM | 611 | CG2 | VAL | A | 786 | −22.471 | 17.024 | 8.226 | 1.00 | 54.17 |
| ATOM | 612 | N | CYS | A | 787 | −19.432 | 14.927 | 10.982 | 1.00 | 56.35 |
| ATOM | 613 | CA | CYS | A | 787 | −18.900 | 14.517 | 12.274 | 1.00 | 56.07 |
| ATOM | 614 | C | CYS | A | 787 | −18.624 | 15.800 | 13.043 | 1.00 | 55.27 |
| ATOM | 615 | O | CYS | A | 787 | −17.881 | 16.661 | 12.579 | 1.00 | 53.31 |
| ATOM | 616 | CB | CYS | A | 787 | −17.608 | 13.718 | 12.128 | 1.00 | 57.61 |
| ATOM | 617 | SG | CYS | A | 787 | −16.830 | 13.406 | 13.747 | 1.00 | 65.10 |
| ATOM | 618 | N | LEU | A | 788 | −19.216 | 15.918 | 14.222 | 1.00 | 56.11 |
| ATOM | 619 | CA | LEU | A | 788 | −19.062 | 17.115 | 15.032 | 1.00 | 59.11 |
| ATOM | 620 | C | LEU | A | 788 | −17.748 | 17.221 | 15.816 | 1.00 | 62.37 |
| ATOM | 621 | O | LEU | A | 788 | −17.417 | 18.290 | 16.337 | 1.00 | 62.61 |
| ATOM | 622 | CB | LEU | A | 788 | −20.250 | 17.220 | 15.987 | 1.00 | 56.56 |
| ATOM | 623 | CG | LEU | A | 788 | −21.630 | 17.156 | 15.312 | 1.00 | 56.06 |
| ATOM | 624 | CD1 | LEU | A | 788 | −22.708 | 17.085 | 16.375 | 1.00 | 53.74 |
| ATOM | 625 | CD2 | LEU | A | 788 | −21.851 | 18.363 | 14.409 | 1.00 | 53.03 |
| ATOM | 626 | N | SER | A | 789 | −16.991 | 16.130 | 15.893 | 1.00 | 65.37 |
| ATOM | 627 | CA | SER | A | 789 | −15.732 | 16.142 | 16.641 | 1.00 | 68.05 |
| ATOM | 628 | C | SER | A | 789 | −14.485 | 15.926 | 15.791 | 1.00 | 68.52 |
| ATOM | 629 | O | SER | A | 789 | −14.522 | 15.191 | 14.805 | 1.00 | 69.20 |
| ATOM | 630 | CB | SER | A | 789 | −15.767 | 15.078 | 17.739 | 1.00 | 68.67 |
| ATOM | 631 | OG | SER | A | 789 | −14.457 | 14.804 | 18.207 | 1.00 | 72.14 |
| ATOM | 632 | N | PRO | A | 790 | −13.365 | 16.587 | 16.154 | 1.00 | 69.26 |
| ATOM | 633 | CA | PRO | A | 790 | −13.256 | 17.506 | 17.299 | 1.00 | 69.34 |
| ATOM | 634 | C | PRO | A | 790 | −13.945 | 18.796 | 16.889 | 1.00 | 69.49 |
| ATOM | 635 | O | PRO | A | 790 | −14.528 | 19.515 | 17.702 | 1.00 | 69.32 |
| ATOM | 636 | CB | PRO | A | 790 | −11.750 | 17.677 | 17.458 | 1.00 | 68.09 |
| ATOM | 637 | CG | PRO | A | 790 | −11.257 | 17.571 | 16.042 | 1.00 | 68.60 |
| ATOM | 638 | CD | PRO | A | 790 | −12.051 | 16.403 | 15.507 | 1.00 | 68.85 |
| ATOM | 639 | N | THR | A | 791 | −13.844 | 19.065 | 15.595 | 1.00 | 69.16 |
| ATOM | 640 | CA | THR | A | 791 | −14.461 | 20.211 | 14.964 | 1.00 | 69.04 |
| ATOM | 641 | C | THR | A | 791 | −15.183 | 19.583 | 13.787 | 1.00 | 67.87 |
| ATOM | 642 | O | THR | A | 791 | −14.762 | 18.547 | 13.266 | 1.00 | 67.60 |
| ATOM | 643 | CB | THR | A | 791 | −13.425 | 21.225 | 14.447 | 1.00 | 70.57 |
| ATOM | 644 | OG1 | THR | A | 791 | −12.586 | 20.599 | 13.466 | 1.00 | 70.47 |
| ATOM | 645 | CG2 | THR | A | 791 | −12.577 | 21.743 | 15.601 | 1.00 | 72.25 |
| ATOM | 646 | N | ILE | A | 792 | −16.275 | 20.208 | 13.380 | 1.00 | 65.93 |
| ATOM | 647 | CA | ILE | A | 792 | −17.081 | 19.707 | 12.288 | 1.00 | 63.52 |
| ATOM | 648 | C | ILE | A | 792 | −16.307 | 19.199 | 11.082 | 1.00 | 61.83 |
| ATOM | 649 | O | ILE | A | 792 | −15.436 | 19.882 | 10.551 | 1.00 | 60.85 |
| ATOM | 650 | CB | ILE | A | 792 | −18.091 | 20.771 | 11.853 | 1.00 | 63.29 |
| ATOM | 651 | CG1 | ILE | A | 792 | −19.080 | 21.014 | 13.001 | 1.00 | 63.31 |
| ATOM | 652 | CG2 | ILE | A | 792 | −18.790 | 20.323 | 10.607 | 1.00 | 63.07 |
| ATOM | 653 | CD1 | ILE | A | 792 | −20.228 | 21.943 | 12.680 | 1.00 | 66.80 |
| ATOM | 654 | N | GLN | A | 793 | −16.642 | 17.983 | 10.666 | 1.00 | 61.24 |
| ATOM | 655 | CA | GLN | A | 793 | −15.999 | 17.350 | 9.526 | 1.00 | 61.88 |
| ATOM | 656 | C | GLN | A | 793 | −17.060 | 16.918 | 8.522 | 1.00 | 60.80 |
| ATOM | 657 | O | GLN | A | 793 | −18.049 | 16.272 | 8.885 | 1.00 | 59.39 |
| ATOM | 658 | CB | GLN | A | 793 | −15.196 | 16.122 | 9.968 | 1.00 | 63.66 |
| ATOM | 659 | CG | GLN | A | 793 | −14.283 | 16.345 | 11.166 | 1.00 | 64.96 |
| ATOM | 660 | CD | GLN | A | 793 | −13.435 | 15.123 | 11.493 | 1.00 | 66.60 |
| ATOM | 661 | OE1 | GLN | A | 793 | −13.341 | 14.190 | 10.695 | 1.00 | 68.16 |
| ATOM | 662 | NE2 | GLN | A | 793 | −12.824 | 15.119 | 12.676 | 1.00 | 67.52 |
| ATOM | 663 | N | LEU | A | 794 | −16.841 | 17.282 | 7.260 | 1.00 | 59.86 |
| ATOM | 664 | CA | LEU | A | 794 | −17.759 | 16.946 | 6.181 | 1.00 | 58.57 |
| ATOM | 665 | C | LEU | A | 794 | −17.334 | 15.628 | 5.571 | 1.00 | 58.29 |
| ATOM | 666 | O | LEU | A | 794 | −16.141 | 15.357 | 5.441 | 1.00 | 58.99 |
| ATOM | 667 | CB | LEU | A | 794 | −17.747 | 18.051 | 5.128 | 1.00 | 59.01 |
| ATOM | 668 | CG | LEU | A | 794 | −18.194 | 19.414 | 5.668 | 1.00 | 58.65 |
| ATOM | 669 | CD1 | LEU | A | 794 | −18.223 | 20.438 | 4.546 | 1.00 | 58.33 |
| ATOM | 670 | CD2 | LEU | A | 794 | −19.573 | 19.276 | 6.298 | 1.00 | 57.68 |
| ATOM | 671 | N | VAL | A | 795 | −18.311 | 14.807 | 5.199 | 1.00 | 58.23 |
| ATOM | 672 | CA | VAL | A | 795 | −18.025 | 13.493 | 4.635 | 1.00 | 57.20 |
| ATOM | 673 | C | VAL | A | 795 | −18.868 | 13.178 | 3.411 | 1.00 | 58.75 |
| ATOM | 674 | O | VAL | A | 795 | −20.096 | 13.062 | 3.490 | 1.00 | 57.69 |
| ATOM | 675 | CB | VAL | A | 795 | −18.257 | 12.371 | 5.680 | 1.00 | 55.92 |
| ATOM | 676 | CG1 | VAL | A | 795 | −17.708 | 11.054 | 5.163 | 1.00 | 55.27 |
| ATOM | 677 | CG2 | VAL | A | 795 | −17.604 | 12.740 | 7.003 | 1.00 | 54.71 |
| ATOM | 678 | N | THR | A | 796 | −18.184 | 13.025 | 2.283 | 1.00 | 60.18 |
| ATOM | 679 | CA | THR | A | 796 | −18.824 | 12.716 | 1.018 | 1.00 | 63.32 |
| ATOM | 680 | C | THR | A | 796 | −18.246 | 11.432 | 0.461 | 1.00 | 64.55 |
| ATOM | 681 | O | THR | A | 796 | −17.150 | 11.012 | 0.838 | 1.00 | 64.79 |
| ATOM | 682 | CB | THR | A | 796 | −18.555 | 13.799 | −0.050 | 1.00 | 63.87 |
| ATOM | 683 | OG1 | THR | A | 796 | −18.802 | 15.099 | 0.494 | 1.00 | 67.06 |
| ATOM | 684 | CG2 | THR | A | 796 | −19.463 | 13.586 | −1.255 | 1.00 | 63.95 |
| ATOM | 685 | N | GLN | A | 797 | −18.991 | 10.815 | −0.445 | 1.00 | 65.24 |
| ATOM | 686 | CA | GLN | A | 797 | −18.522 | 9.611 | −1.100 | 1.00 | 66.49 |
| ATOM | 687 | C | GLN | A | 797 | −17.288 | 10.093 | −1.848 | 1.00 | 66.60 |
| ATOM | 688 | O | GLN | A | 797 | −17.234 | 11.249 | −2.278 | 1.00 | 67.28 |
| ATOM | 689 | CB | GLN | A | 797 | −19.569 | 9.120 | −2.089 | 1.00 | 67.59 |
| ATOM | 690 | CG | GLN | A | 797 | −19.197 | 7.860 | −2.834 | 1.00 | 71.35 |
| ATOM | 691 | CD | GLN | A | 797 | −20.192 | 7.552 | −3.940 | 1.00 | 74.61 |
| ATOM | 692 | OE1 | GLN | A | 797 | −21.400 | 7.453 | −3.696 | 1.00 | 75.46 |
| ATOM | 693 | NE2 | GLN | A | 797 | −19.693 | 7.405 | −5.166 | 1.00 | 74.15 |
| ATOM | 694 | N | LEU | A | 798 | −16.287 | 9.234 | −1.986 | 1.00 | 65.62 |
| ATOM | 695 | CA | LEU | A | 798 | −15.082 | 9.631 | −2.697 | 1.00 | 64.21 |
| ATOM | 696 | C | LEU | A | 798 | −15.309 | 9.410 | −4.187 | 1.00 | 65.69 |
| ATOM | 697 | O | LEU | A | 798 | −16.029 | 8.487 | −4.582 | 1.00 | 66.02 |
| ATOM | 698 | CB | LEU | A | 798 | −13.888 | 8.801 | −2.228 | 1.00 | 61.04 |
| ATOM | 699 | CG | LEU | A | 798 | −12.541 | 9.140 | −2.860 | 1.00 | 60.59 |
| ATOM | 700 | CD1 | LEU | A | 798 | −12.125 | 10.543 | −2.451 | 1.00 | 60.17 |
| ATOM | 701 | CD2 | LEU | A | 798 | −11.498 | 8.133 | −2.415 | 1.00 | 59.95 |
| ATOM | 702 | N | MET | A | 799 | −14.722 | 10.272 | −5.013 | 1.00 | 65.98 |
| ATOM | 703 | CA | MET | A | 799 | −14.846 | 10.135 | −6.459 | 1.00 | 65.14 |
| ATOM | 704 | C | MET | A | 799 | −13.531 | 9.518 | −6.915 | 1.00 | 64.98 |
| ATOM | 705 | O | MET | A | 799 | −12.497 | 10.186 | −6.964 | 1.00 | 65.11 |
| ATOM | 706 | CB | MET | A | 799 | −15.076 | 11.499 | −7.123 | 1.00 | 64.55 |
| ATOM | 707 | CG | MET | A | 799 | −16.418 | 12.140 | −6.791 | 1.00 | 62.41 |
| ATOM | 708 | SD | MET | A | 799 | −17.821 | 11.182 | −7.391 | 1.00 | 63.79 |
| ATOM | 709 | CE | MET | A | 799 | −18.278 | 10.242 | −5.953 | 1.00 | 63.53 |
| ATOM | 710 | N | PRO | A | 800 | −13.559 | 8.221 | −7.249 | 1.00 | 65.17 |
| ATOM | 711 | CA | PRO | A | 800 | −12.386 | 7.464 | −7.697 | 1.00 | 65.94 |
| ATOM | 712 | C | PRO | A | 800 | −11.452 | 8.149 | −8.692 | 1.00 | 65.86 |
| ATOM | 713 | O | PRO | A | 800 | −10.254 | 8.255 | −8.443 | 1.00 | 64.64 |
| ATOM | 714 | CB | PRO | A | 800 | −12.999 | 6.173 | −8.257 | 1.00 | 66.48 |
| ATOM | 715 | CG | PRO | A | 800 | −14.391 | 6.583 | −8.651 | 1.00 | 65.65 |
| ATOM | 716 | CD | PRO | A | 800 | −14.792 | 7.454 | −7.496 | 1.00 | 65.28 |
| ATOM | 717 | N | HIS | A | 801 | −11.998 | 8.614 | −9.811 | 1.00 | 66.24 |
| ATOM | 718 | CA | HIS | A | 801 | −11.185 | 9.251 | −10.834 | 1.00 | 66.32 |
| ATOM | 719 | C | HIS | A | 801 | −10.814 | 10.712 | −10.584 | 1.00 | 65.51 |
| ATOM | 720 | O | HIS | A | 801 | −10.474 | 11.439 | −11.512 | 1.00 | 66.97 |
| ATOM | 721 | CB | HIS | A | 801 | −11.861 | 9.082 | −12.195 | 1.00 | 68.47 |
| ATOM | 722 | CG | HIS | A | 801 | −11.882 | 7.661 | −12.679 | 1.00 | 70.66 |
| ATOM | 723 | ND1 | HIS | A | 801 | −10.742 | 6.990 | −13.071 | 1.00 | 70.46 |
| ATOM | 724 | CD2 | HIS | A | 801 | −12.900 | 6.773 | −12.798 | 1.00 | 71.35 |
| ATOM | 725 | CE1 | HIS | A | 801 | −11.057 | 5.751 | −13.407 | 1.00 | 70.98 |
| ATOM | 726 | NE2 | HIS | A | 801 | −12.359 | 5.593 | −13.250 | 1.00 | 71.18 |
| ATOM | 727 | N | GLY | A | 802 | −10.885 | 11.130 | −9.324 | 1.00 | 64.19 |
| ATOM | 728 | CA | GLY | A | 802 | −10.503 | 12.479 | −8.929 | 1.00 | 61.92 |
| ATOM | 729 | C | GLY | A | 802 | −11.137 | 13.756 | −9.473 | 1.00 | 60.42 |
| ATOM | 730 | O | GLY | A | 802 | −12.277 | 13.794 | −9.935 | 1.00 | 57.28 |
| ATOM | 731 | N | CYS | A | 803 | −10.355 | 14.825 | −9.374 | 1.00 | 59.58 |
| ATOM | 732 | CA | CYS | A | 803 | −10.755 | 16.146 | −9.812 | 1.00 | 58.64 |
| ATOM | 733 | C | CYS | A | 803 | −10.613 | 16.269 | −11.316 | 1.00 | 59.22 |
| ATOM | 734 | O | CYS | A | 803 | −9.576 | 15.912 | −11.881 | 1.00 | 59.33 |
| ATOM | 735 | CB | CYS | A | 803 | −9.891 | 17.202 | −9.123 | 1.00 | 58.78 |
| ATOM | 736 | SG | CYS | A | 803 | −10.148 | 17.335 | −7.322 | 1.00 | 60.97 |
| ATOM | 737 | N | LEU | A | 804 | −11.660 | 16.786 | −11.955 | 1.00 | 58.47 |
| ATOM | 738 | CA | LEU | A | 804 | −11.686 | 16.960 | −13.399 | 1.00 | 57.67 |
| ATOM | 739 | C | LEU | A | 804 | −10.524 | 17.792 | −13.938 | 1.00 | 57.23 |
| ATOM | 740 | O | LEU | A | 804 | −9.973 | 17.494 | −14.995 | 1.00 | 57.42 |
| ATOM | 741 | CB | LEU | A | 804 | −13.010 | 17.592 | −13.822 | 1.00 | 57.36 |
| ATOM | 742 | CG | LEU | A | 804 | −13.136 | 17.853 | −15.323 | 1.00 | 56.02 |
| ATOM | 743 | CD1 | LEU | A | 804 | −12.864 | 16.566 | −16.073 | 1.00 | 56.06 |
| ATOM | 744 | CD2 | LEU | A | 804 | −14.522 | 18.392 | −15.646 | 1.00 | 56.14 |
| ATOM | 745 | N | LEU | A | 805 | −10.151 | 18.839 | −13.219 | 1.00 | 56.60 |
| ATOM | 746 | CA | LEU | A | 805 | −9.054 | 19.674 | −13.666 | 1.00 | 57.25 |
| ATOM | 747 | C | LEU | A | 805 | −7.770 | 18.871 | −13.836 | 1.00 | 59.50 |
| ATOM | 748 | O | LEU | A | 805 | −7.068 | 19.041 | −14.826 | 1.00 | 61.05 |
| ATOM | 749 | CB | LEU | A | 805 | −8.821 | 20.814 | −12.681 | 1.00 | 54.99 |
| ATOM | 750 | CG | LEU | A | 805 | −7.600 | 21.668 | −12.995 | 1.00 | 52.96 |
| ATOM | 751 | CD1 | LEU | A | 805 | −7.614 | 22.102 | −14.453 | 1.00 | 51.15 |
| ATOM | 752 | CD2 | LEU | A | 805 | −7.596 | 22.863 | −12.072 | 1.00 | 53.82 |
| ATOM | 753 | N | GLU | A | 806 | −7.458 | 18.006 | −12.873 | 1.00 | 61.71 |
| ATOM | 754 | CA | GLU | A | 806 | −6.249 | 17.186 | −12.952 | 1.00 | 64.25 |
| ATOM | 755 | C | GLU | A | 806 | −6.435 | 16.119 | −14.027 | 1.00 | 63.97 |
| ATOM | 756 | O | GLU | A | 806 | −5.477 | 15.676 | −14.665 | 1.00 | 63.45 |
| ATOM | 757 | CB | GLU | A | 806 | −5.957 | 16.495 | −11.611 | 1.00 | 67.06 |
| ATOM | 758 | CG | GLU | A | 806 | −5.509 | 17.425 | −10.477 | 1.00 | 73.60 |
| ATOM | 759 | CD | GLU | A | 806 | −5.169 | 16.666 | −9.188 | 1.00 | 76.72 |
| ATOM | 760 | OE1 | GLU | A | 806 | −5.484 | 15.455 | −9.105 | 1.00 | 77.37 |
| ATOM | 761 | OE2 | GLU | A | 806 | −4.593 | 17.279 | −8.259 | 1.00 | 78.10 |
| ATOM | 762 | N | TYR | A | 807 | −7.684 | 15.719 | −14.228 | 1.00 | 63.27 |
| ATOM | 763 | CA | TYR | A | 807 | −8.015 | 14.694 | −15.203 | 1.00 | 63.32 |
| ATOM | 764 | C | TYR | A | 807 | −7.749 | 15.098 | −16.649 | 1.00 | 62.69 |
| ATOM | 765 | O | TYR | A | 807 | −7.050 | 14.391 | −17.364 | 1.00 | 61.57 |
| ATOM | 766 | CB | TYR | A | 807 | −9.478 | 14.294 | −15.058 | 1.00 | 63.62 |
| ATOM | 767 | CG | TYR | A | 807 | −9.822 | 13.015 | −15.769 | 1.00 | 63.28 |
| ATOM | 768 | CD1 | TYR | A | 807 | −9.545 | 11.778 | −15.189 | 1.00 | 62.76 |
| ATOM | 769 | CD2 | TYR | A | 807 | −10.436 | 13.040 | −17.017 | 1.00 | 63.66 |
| ATOM | 770 | CE1 | TYR | A | 807 | −9.879 | 10.597 | −15.834 | 1.00 | 63.41 |
| ATOM | 771 | CE2 | TYR | A | 807 | −10.771 | 11.867 | −17.671 | 1.00 | 64.64 |
| ATOM | 772 | CZ | TYR | A | 807 | −10.494 | 10.650 | −17.074 | 1.00 | 64.30 |
| ATOM | 773 | OH | TYR | A | 807 | −10.854 | 9.494 | −17.719 | 1.00 | 65.98 |
| ATOM | 774 | N | VAL | A | 808 | −8.305 | 16.223 | −17.087 | 1.00 | 62.57 |
| ATOM | 775 | CA | VAL | A | 808 | −8.087 | 16.644 | −18.466 | 1.00 | 64.21 |
| ATOM | 776 | C | VAL | A | 808 | −6.626 | 16.901 | −18.773 | 1.00 | 65.63 |
| ATOM | 777 | O | VAL | A | 808 | −6.238 | 16.927 | −19.933 | 1.00 | 66.29 |
| ATOM | 778 | CB | VAL | A | 808 | −8.865 | 17.914 | −18.831 | 1.00 | 61.95 |
| ATOM | 779 | CG1 | VAL | A | 808 | −10.337 | 17.614 | −18.891 | 1.00 | 61.31 |
| ATOM | 780 | CG2 | VAL | A | 808 | −8.568 | 19.003 | −17.832 | 1.00 | 61.93 |
| ATOM | 781 | N | HIS | A | 809 | −5.811 | 17.093 | −17.746 | 1.00 | 68.17 |
| ATOM | 782 | CA | HIS | A | 809 | −4.398 | 17.337 | −17.986 | 1.00 | 72.57 |
| ATOM | 783 | C | HIS | A | 809 | −3.690 | 16.011 | −18.246 | 1.00 | 72.86 |
| ATOM | 784 | O | HIS | A | 809 | −3.024 | 15.834 | −19.266 | 1.00 | 73.15 |
| ATOM | 785 | CB | HIS | A | 809 | −3.756 | 18.034 | −16.787 | 1.00 | 76.24 |
| ATOM | 786 | CG | HIS | A | 809 | −2.334 | 18.446 | −17.018 | 1.00 | 80.69 |
| ATOM | 787 | ND1 | HIS | A | 809 | −1.989 | 19.505 | −17.831 | 1.00 | 82.82 |
| ATOM | 788 | CD2 | HIS | A | 809 | −1.169 | 17.940 | −16.544 | 1.00 | 82.57 |
| ATOM | 789 | CE1 | HIS | A | 809 | −0.673 | 19.635 | −17.846 | 1.00 | 83.54 |
| ATOM | 790 | NE2 | HIS | A | 809 | −0.152 | 18.698 | −17.074 | 1.00 | 83.63 |
| ATOM | 791 | N | GLU | A | 810 | −3.851 | 15.077 | −17.320 | 1.00 | 73.25 |
| ATOM | 792 | CA | GLU | A | 810 | −3.220 | 13.776 | −17.441 | 1.00 | 74.27 |
| ATOM | 793 | C | GLU | A | 810 | −3.700 | 12.994 | −18.655 | 1.00 | 73.56 |
| ATOM | 794 | O | GLU | A | 810 | −2.901 | 12.341 | −19.331 | 1.00 | 73.89 |
| ATOM | 795 | CB | GLU | A | 810 | −3.466 | 12.963 | −16.172 | 1.00 | 76.41 |
| ATOM | 796 | CG | GLU | A | 810 | −2.767 | 13.517 | −14.941 | 1.00 | 80.78 |
| ATOM | 797 | CD | GLU | A | 810 | −3.185 | 12.795 | −13.670 | 1.00 | 84.23 |
| ATOM | 798 | OE1 | GLU | A | 810 | −3.195 | 11.542 | −13.672 | 1.00 | 85.68 |
| ATOM | 799 | OE2 | GLU | A | 810 | −3.498 | 13.479 | −12.668 | 1.00 | 84.74 |
| ATOM | 800 | N | HIS | A | 811 | −4.998 | 13.070 | −18.937 | 1.00 | 72.66 |
| ATOM | 801 | CA | HIS | A | 811 | −5.582 | 12.346 | −20.063 | 1.00 | 72.30 |
| ATOM | 802 | C | HIS | A | 811 | −5.823 | 13.195 | −21.303 | 1.00 | 72.06 |
| ATOM | 803 | O | HIS | A | 811 | −6.591 | 12.801 | −22.187 | 1.00 | 71.12 |
| ATOM | 804 | CB | HIS | A | 811 | −6.899 | 11.701 | −19.638 | 1.00 | 73.09 |
| ATOM | 805 | CG | HIS | A | 811 | −6.750 | 10.700 | −18.539 | 1.00 | 74.95 |
| ATOM | 806 | ND1 | HIS | A | 811 | −6.018 | 10.951 | −17.399 | 1.00 | 76.95 |
| ATOM | 807 | CD2 | HIS | A | 811 | −7.260 | 9.455 | −18.392 | 1.00 | 76.68 |
| ATOM | 808 | CE1 | HIS | A | 811 | −6.084 | 9.904 | −16.596 | 1.00 | 77.89 |
| ATOM | 809 | NE2 | HIS | A | 811 | −6.833 | 8.983 | −17.175 | 1.00 | 77.93 |
| ATOM | 810 | N | LYS | A | 812 | −5.167 | 14.351 | −21.369 | 1.00 | 72.19 |
| ATOM | 811 | CA | LYS | A | 812 | −5.321 | 15.247 | −22.510 | 1.00 | 72.68 |
| ATOM | 812 | C | LYS | A | 812 | −5.330 | 14.462 | −23.805 | 1.00 | 73.33 |
| ATOM | 813 | O | LYS | A | 812 | −6.280 | 14.518 | −24.584 | 1.00 | 73.60 |
| ATOM | 814 | CB | LYS | A | 812 | −4.180 | 16.271 | −22.570 | 1.00 | 72.21 |
| ATOM | 815 | CG | LYS | A | 812 | −4.116 | 16.982 | −23.917 | 1.00 | 71.18 |
| ATOM | 816 | CD | LYS | A | 812 | −3.146 | 18.148 | −23.951 | 1.00 | 70.28 |
| ATOM | 817 | CE | LYS | A | 812 | −3.235 | 18.834 | −25.308 | 1.00 | 68.88 |
| ATOM | 818 | NZ | LYS | A | 812 | −2.481 | 20.108 | −25.344 | 1.00 | 68.31 |
| ATOM | 819 | N | ASP | A | 813 | −4.256 | 13.717 | −24.015 | 1.00 | 75.12 |
| ATOM | 820 | CA | ASP | A | 813 | −4.092 | 12.917 | −25.213 | 1.00 | 76.11 |
| ATOM | 821 | C | ASP | A | 813 | −5.038 | 11.712 | −25.213 | 1.00 | 75.70 |
| ATOM | 822 | O | ASP | A | 813 | −4.692 | 10.641 | −25.697 | 1.00 | 75.57 |
| ATOM | 823 | CB | ASP | A | 813 | −2.627 | 12.469 | −25.312 | 1.00 | 78.16 |
| ATOM | 824 | CG | ASP | A | 813 | −1.639 | 13.629 | −25.104 | 1.00 | 79.48 |
| ATOM | 825 | OD1 | ASP | A | 813 | −1.703 | 14.618 | −25.867 | 1.00 | 78.24 |
| ATOM | 826 | OD2 | ASP | A | 813 | −0.795 | 13.554 | −24.176 | 1.00 | 81.15 |
| ATOM | 827 | N | ASN | A | 814 | −6.237 | 11.901 | −24.671 | 1.00 | 75.59 |
| ATOM | 828 | CA | ASN | A | 814 | −7.232 | 10.835 | −24.609 | 1.00 | 75.15 |
| ATOM | 829 | C | ASN | A | 814 | −8.639 | 11.421 | −24.602 | 1.00 | 73.09 |
| ATOM | 830 | O | ASN | A | 814 | −9.629 | 10.690 | −24.620 | 1.00 | 72.17 |
| ATOM | 831 | CB | ASN | A | 814 | −7.038 | 10.003 | −23.340 | 1.00 | 79.73 |
| ATOM | 832 | CG | ASN | A | 814 | −8.095 | 8.910 | −23.182 | 1.00 | 83.39 |
| ATOM | 833 | OD1 | ASN | A | 814 | −8.417 | 8.495 | −22.059 | 1.00 | 85.44 |
| ATOM | 834 | ND2 | ASN | A | 814 | −8.628 | 8.429 | −24.306 | 1.00 | 82.76 |
| ATOM | 835 | N | ILE | A | 815 | −8.725 | 12.746 | −24.575 | 1.00 | 70.07 |
| ATOM | 836 | CA | ILE | A | 815 | −10.018 | 13.414 | −24.543 | 1.00 | 66.45 |
| ATOM | 837 | C | ILE | A | 815 | −10.548 | 13.773 | −25.923 | 1.00 | 64.58 |
| ATOM | 838 | O | ILE | A | 815 | −9.910 | 14.515 | −26.670 | 1.00 | 65.16 |
| ATOM | 839 | CB | ILE | A | 815 | −9.940 | 14.686 | −23.701 | 1.00 | 66.17 |
| ATOM | 840 | CG1 | ILE | A | 815 | −9.416 | 14.337 | −22.310 | 1.00 | 64.41 |
| ATOM | 841 | CG2 | ILE | A | 815 | −11.308 | 15.341 | −23.616 | 1.00 | 65.95 |
| ATOM | 842 | CD1 | ILE | A | 815 | −10.283 | 13.377 | −21.576 | 1.00 | 64.44 |
| ATOM | 843 | N | GLY | A | 816 | −11.727 | 13.244 | −26.244 | 1.00 | 61.95 |
| ATOM | 844 | CA | GLY | A | 816 | −12.348 | 13.502 | −27.529 | 1.00 | 58.49 |
| ATOM | 845 | C | GLY | A | 816 | −13.391 | 14.602 | −27.474 | 1.00 | 57.57 |
| ATOM | 846 | O | GLY | A | 816 | −13.798 | 15.049 | −26.400 | 1.00 | 56.05 |
| ATOM | 847 | N | SER | A | 817 | −13.836 | 15.036 | −28.645 | 1.00 | 55.76 |
| ATOM | 848 | CA | SER | A | 817 | −14.814 | 16.100 | −28.723 | 1.00 | 54.42 |
| ATOM | 849 | C | SER | A | 817 | −16.087 | 15.748 | −27.976 | 1.00 | 53.51 |
| ATOM | 850 | O | SER | A | 817 | −16.653 | 16.588 | −27.277 | 1.00 | 53.70 |
| ATOM | 851 | CB | SER | A | 817 | −15.140 | 16.417 | −30.188 | 1.00 | 54.21 |
| ATOM | 852 | OG | SER | A | 817 | −15.754 | 15.315 | −30.830 | 1.00 | 53.76 |
| ATOM | 853 | N | GLN | A | 818 | −16.526 | 14.503 | −28.118 | 1.00 | 52.59 |
| ATOM | 854 | CA | GLN | A | 818 | −17.755 | 14.046 | −27.481 | 1.00 | 52.58 |
| ATOM | 855 | C | GLN | A | 818 | −17.711 | 14.105 | −25.962 | 1.00 | 52.52 |
| ATOM | 856 | O | GLN | A | 818 | −18.606 | 14.649 | −25.316 | 1.00 | 53.30 |
| ATOM | 857 | CB | GLN | A | 818 | −18.071 | 12.624 | −27.929 | 1.00 | 52.77 |
| ATOM | 858 | CG | GLN | A | 818 | −19.386 | 12.096 | −27.396 | 1.00 | 55.94 |
| ATOM | 859 | CD | GLN | A | 818 | −19.734 | 10.728 | −27.960 | 1.00 | 57.95 |
| ATOM | 860 | OE1 | GLN | A | 818 | −19.015 | 9.749 | −27.741 | 1.00 | 55.96 |
| ATOM | 861 | NE2 | GLN | A | 818 | −20.841 | 10.657 | −28.694 | 1.00 | 58.90 |
| ATOM | 862 | N | LEU | A | 819 | −16.663 | 13.535 | −25.392 | 1.00 | 52.33 |
| ATOM | 863 | CA | LEU | A | 819 | −16.487 | 13.519 | −23.951 | 1.00 | 53.05 |
| ATOM | 864 | C | LEU | A | 819 | −16.530 | 14.955 | −23.374 | 1.00 | 52.97 |
| ATOM | 865 | O | LEU | A | 819 | −17.199 | 15.211 | −22.372 | 1.00 | 51.99 |
| ATOM | 866 | CB | LEU | A | 819 | −15.147 | 12.846 | −23.641 | 1.00 | 55.20 |
| ATOM | 867 | CG | LEU | A | 819 | −14.857 | 12.305 | −22.246 | 1.00 | 58.07 |
| ATOM | 868 | CD1 | LEU | A | 819 | −15.883 | 11.248 | −21.893 | 1.00 | 59.70 |
| ATOM | 869 | CD2 | LEU | A | 819 | −13.454 | 11.722 | −22.216 | 1.00 | 57.59 |
| ATOM | 870 | N | LEU | A | 820 | −15.823 | 15.881 | −24.024 | 1.00 | 51.18 |
| ATOM | 871 | CA | LEU | A | 820 | −15.761 | 17.274 | −23.592 | 1.00 | 49.17 |
| ATOM | 872 | C | LEU | A | 820 | −17.089 | 18.021 | −23.663 | 1.00 | 48.89 |
| ATOM | 873 | O | LEU | A | 820 | −17.395 | 18.820 | −22.776 | 1.00 | 48.95 |
| ATOM | 874 | CB | LEU | A | 820 | −14.725 | 18.041 | −24.415 | 1.00 | 48.36 |
| ATOM | 875 | CG | LEU | A | 820 | −13.252 | 17.791 | −24.106 | 1.00 | 47.62 |
| ATOM | 876 | CD1 | LEU | A | 820 | −12.411 | 18.277 | −25.275 | 1.00 | 47.48 |
| ATOM | 877 | CD2 | LEU | A | 820 | −12.859 | 18.485 | −22.813 | 1.00 | 46.35 |
| ATOM | 878 | N | LEU | A | 821 | −17.867 | 17.794 | −24.719 | 1.00 | 47.18 |
| ATOM | 879 | CA | LEU | A | 821 | −19.158 | 18.470 | −24.845 | 1.00 | 45.74 |
| ATOM | 880 | C | LEU | A | 821 | −20.174 | 17.912 | −23.858 | 1.00 | 44.93 |
| ATOM | 881 | O | LEU | A | 821 | −21.035 | 18.636 | −23.366 | 1.00 | 44.43 |
| ATOM | 882 | CB | LEU | A | 821 | −19.699 | 18.350 | −26.269 | 1.00 | 43.56 |
| ATOM | 883 | CG | LEU | A | 821 | −19.043 | 19.245 | −27.324 | 1.00 | 44.32 |
| ATOM | 884 | CD1 | LEU | A | 821 | −19.595 | 18.904 | −28.686 | 1.00 | 42.72 |
| ATOM | 885 | CD2 | LEU | A | 821 | −19.299 | 20.706 | −27.015 | 1.00 | 41.61 |
| ATOM | 886 | N | ASN | A | 822 | −20.074 | 16.620 | −23.575 | 1.00 | 45.14 |
| ATOM | 887 | CA | ASN | A | 822 | −20.978 | 15.992 | −22.630 | 1.00 | 46.84 |
| ATOM | 888 | C | ASN | A | 822 | −20.710 | 16.514 | −21.216 | 1.00 | 46.44 |
| ATOM | 889 | O | ASN | A | 822 | −21.629 | 16.712 | −20.429 | 1.00 | 47.65 |
| ATOM | 890 | CB | ASN | A | 822 | −20.803 | 14.480 | −22.683 | 1.00 | 50.43 |
| ATOM | 891 | CG | ASN | A | 822 | −21.407 | 13.874 | −23.930 | 1.00 | 53.27 |
| ATOM | 892 | OD1 | ASN | A | 822 | −22.624 | 13.914 | −24.127 | 1.00 | 55.44 |
| ATOM | 893 | ND2 | ASN | A | 822 | −20.564 | 13.310 | −24.782 | 1.00 | 55.67 |
| ATOM | 894 | N | TRP | A | 823 | −19.448 | 16.736 | −20.886 | 1.00 | 44.44 |
| ATOM | 895 | CA | TRP | A | 823 | −19.126 | 17.265 | −19.571 | 1.00 | 44.31 |
| ATOM | 896 | C | TRP | A | 823 | −19.734 | 18.662 | −19.418 | 1.00 | 42.62 |
| ATOM | 897 | O | TRP | A | 823 | −20.172 | 19.051 | −18.331 | 1.00 | 41.36 |
| ATOM | 898 | CB | TRP | A | 823 | −17.609 | 17.334 | −19.376 | 1.00 | 44.91 |
| ATOM | 899 | CG | TRP | A | 823 | −16.949 | 15.987 | −19.186 | 1.00 | 46.37 |
| ATOM | 900 | CD1 | TRP | A | 823 | −17.551 | 14.826 | −18.785 | 1.00 | 45.40 |
| ATOM | 901 | CD2 | TRP | A | 823 | −15.552 | 15.689 | −19.318 | 1.00 | 46.95 |
| ATOM | 902 | NE1 | TRP | A | 823 | −16.615 | 13.828 | −18.656 | 1.00 | 46.44 |
| ATOM | 903 | CE2 | TRP | A | 823 | −15.380 | 14.328 | −18.977 | 1.00 | 46.59 |
| ATOM | 904 | CE3 | TRP | A | 823 | −14.425 | 16.441 | −19.693 | 1.00 | 49.03 |
| ATOM | 905 | CZ2 | TRP | A | 823 | −14.128 | 13.699 | −18.998 | 1.00 | 46.68 |
| ATOM | 906 | CZ3 | TRP | A | 823 | −13.169 | 15.808 | −19.715 | 1.00 | 50.33 |
| ATOM | 907 | CH2 | TRP | A | 823 | −13.038 | 14.451 | −19.368 | 1.00 | 47.90 |
| ATOM | 908 | N | CYS | A | 824 | −19.773 | 19.407 | −20.514 | 1.00 | 40.45 |
| ATOM | 909 | CA | CYS | A | 824 | −20.329 | 20.746 | −20.489 | 1.00 | 42.24 |
| ATOM | 910 | C | CYS | A | 824 | −21.799 | 20.690 | −20.146 | 1.00 | 43.66 |
| ATOM | 911 | O | CYS | A | 824 | −22.309 | 21.548 | −19.426 | 1.00 | 43.87 |
| ATOM | 912 | CB | CYS | A | 824 | −20.138 | 21.439 | −21.836 | 1.00 | 43.69 |
| ATOM | 913 | SG | CYS | A | 824 | −18.428 | 21.910 | −22.159 | 1.00 | 44.22 |
| ATOM | 914 | N | VAL | A | 825 | −22.472 | 19.668 | −20.667 | 1.00 | 45.00 |
| ATOM | 915 | CA | VAL | A | 825 | −23.896 | 19.459 | −20.425 | 1.00 | 42.03 |
| ATOM | 916 | C | VAL | A | 825 | −24.109 | 19.061 | −18.973 | 1.00 | 42.11 |
| ATOM | 917 | O | VAL | A | 825 | −24.921 | 19.653 | −18.254 | 1.00 | 40.65 |
| ATOM | 918 | CB | VAL | A | 825 | −24.441 | 18.353 | −21.337 | 1.00 | 41.91 |
| ATOM | 919 | CG1 | VAL | A | 825 | −25.865 | 17.993 | −20.940 | 1.00 | 41.30 |
| ATOM | 920 | CG2 | VAL | A | 825 | −24.392 | 18.821 | −22.788 | 1.00 | 41.84 |
| ATOM | 921 | N | GLN | A | 826 | −23.362 | 18.053 | −18.543 | 1.00 | 42.60 |
| ATOM | 922 | CA | GLN | A | 826 | −23.460 | 17.573 | −17.175 | 1.00 | 42.94 |
| ATOM | 923 | C | GLN | A | 826 | −23.232 | 18.697 | −16.182 | 1.00 | 40.98 |
| ATOM | 924 | O | GLN | A | 826 | −24.012 | 18.858 | −15.253 | 1.00 | 41.31 |
| ATOM | 925 | CB | GLN | A | 826 | −22.475 | 16.428 | −16.965 | 1.00 | 43.82 |
| ATOM | 926 | CG | GLN | A | 826 | −22.954 | 15.187 | −17.689 | 1.00 | 46.63 |
| ATOM | 927 | CD | GLN | A | 826 | −21.996 | 14.029 | −17.594 | 1.00 | 47.66 |
| ATOM | 928 | OE1 | GLN | A | 826 | −21.205 | 13.938 | −16.660 | 1.00 | 47.25 |
| ATOM | 929 | NE2 | GLN | A | 826 | −22.077 | 13.119 | −18.559 | 1.00 | 49.33 |
| ATOM | 930 | N | ILE | A | 827 | −22.180 | 19.485 | −16.381 | 1.00 | 39.59 |
| ATOM | 931 | CA | ILE | A | 827 | −21.920 | 20.598 | −15.479 | 1.00 | 38.36 |
| ATOM | 932 | C | ILE | A | 827 | −23.090 | 21.596 | −15.527 | 1.00 | 39.97 |
| ATOM | 933 | O | ILE | A | 827 | −23.643 | 21.967 | −14.485 | 1.00 | 41.14 |
| ATOM | 934 | CB | ILE | A | 827 | −20.607 | 21.329 | −15.834 | 1.00 | 36.75 |
| ATOM | 935 | CG1 | ILE | A | 827 | −19.449 | 20.320 | −15.852 | 1.00 | 36.42 |
| ATOM | 936 | CG2 | ILE | A | 827 | −20.335 | 22.436 | −14.816 | 1.00 | 32.91 |
| ATOM | 937 | CD1 | ILE | A | 827 | −18.064 | 20.930 | −15.996 | 1.00 | 33.58 |
| ATOM | 938 | N | ALA | A | 828 | −23.479 | 22.016 | −16.727 | 1.00 | 38.03 |
| ATOM | 939 | CA | ALA | A | 828 | −24.581 | 22.957 | −16.867 | 1.00 | 38.98 |
| ATOM | 940 | C | ALA | A | 828 | −25.848 | 22.407 | −16.203 | 1.00 | 41.61 |
| ATOM | 941 | O | ALA | A | 828 | −26.562 | 23.133 | −15.514 | 1.00 | 41.11 |
| ATOM | 942 | CB | ALA | A | 828 | −24.835 | 23.253 | −18.339 | 1.00 | 37.80 |
| ATOM | 943 | N | LYS | A | 829 | −26.120 | 21.118 | −16.398 | 1.00 | 43.40 |
| ATOM | 944 | CA | LYS | A | 829 | −27.302 | 20.502 | −15.803 | 1.00 | 43.58 |
| ATOM | 945 | C | LYS | A | 829 | −27.298 | 20.557 | −14.277 | 1.00 | 45.12 |
| ATOM | 946 | O | LYS | A | 829 | −28.319 | 20.849 | −13.666 | 1.00 | 45.69 |
| ATOM | 947 | CB | LYS | A | 829 | −27.444 | 19.055 | −16.272 | 1.00 | 43.15 |
| ATOM | 948 | CG | LYS | A | 829 | −28.315 | 18.892 | −17.508 | 1.00 | 43.32 |
| ATOM | 949 | CD | LYS | A | 829 | −28.403 | 17.433 | −17.912 | 1.00 | 47.16 |
| ATOM | 950 | CE | LYS | A | 829 | −29.243 | 17.228 | −19.164 | 1.00 | 49.43 |
| ATOM | 951 | NZ | LYS | A | 829 | −29.209 | 15.792 | −19.577 | 1.00 | 50.93 |
| ATOM | 952 | N | GLY | A | 830 | −26.159 | 20.268 | −13.661 | 1.00 | 46.16 |
| ATOM | 953 | CA | GLY | A | 830 | −26.090 | 20.327 | −12.214 | 1.00 | 46.02 |
| ATOM | 954 | C | GLY | A | 830 | −26.349 | 21.744 | −11.721 | 1.00 | 46.34 |
| ATOM | 955 | O | GLY | A | 830 | −27.060 | 21.948 | −10.729 | 1.00 | 46.09 |
| ATOM | 956 | N | MET | A | 831 | −25.770 | 22.731 | −12.399 | 1.00 | 45.04 |
| ATOM | 957 | CA | MET | A | 831 | −25.982 | 24.119 | −11.999 | 1.00 | 46.30 |
| ATOM | 958 | C | MET | A | 831 | −27.484 | 24.407 | −12.125 | 1.00 | 47.00 |
| ATOM | 959 | O | MET | A | 831 | −28.076 | 25.117 | −11.306 | 1.00 | 45.79 |
| ATOM | 960 | CB | MET | A | 831 | −25.173 | 25.069 | −12.896 | 1.00 | 43.99 |
| ATOM | 961 | CG | MET | A | 831 | −23.675 | 24.923 | −12.748 | 1.00 | 40.18 |
| ATOM | 962 | SD | MET | A | 831 | −23.169 | 25.141 | −11.023 | 1.00 | 41.93 |
| ATOM | 963 | CE | MET | A | 831 | −23.711 | 26.840 | −10.757 | 1.00 | 41.68 |
| ATOM | 964 | N | MET | A | 832 | −28.084 | 23.819 | −13.156 | 1.00 | 46.35 |
| ATOM | 965 | CA | MET | A | 832 | −29.503 | 23.954 | −13.444 | 1.00 | 48.31 |
| ATOM | 966 | C | MET | A | 832 | −30.330 | 23.517 | −12.236 | 1.00 | 47.54 |
| ATOM | 967 | O | MET | A | 832 | −31.274 | 24.197 | −11.828 | 1.00 | 49.03 |
| ATOM | 968 | CB | MET | A | 832 | −29.835 | 23.080 | −14.648 | 1.00 | 52.33 |
| ATOM | 969 | CG | MET | A | 832 | −31.143 | 23.377 | −15.345 | 1.00 | 57.13 |
| ATOM | 970 | SD | MET | A | 832 | −31.249 | 22.334 | −16.823 | 1.00 | 62.53 |
| ATOM | 971 | CE | MET | A | 832 | −30.102 | 23.157 | −17.832 | 1.00 | 58.95 |
| ATOM | 972 | N | TYR | A | 833 | −29.963 | 22.375 | −11.670 | 1.00 | 45.22 |
| ATOM | 973 | CA | TYR | A | 833 | −30.637 | 21.832 | −10.508 | 1.00 | 43.85 |
| ATOM | 974 | C | TYR | A | 833 | −30.446 | 22.781 | −9.332 | 1.00 | 45.57 |
| ATOM | 975 | O | TYR | A | 833 | −31.382 | 23.033 | −8.571 | 1.00 | 44.47 |
| ATOM | 976 | CB | TYR | A | 833 | −30.041 | 20.475 | −10.161 | 1.00 | 42.10 |
| ATOM | 977 | CG | TYR | A | 833 | −30.709 | 19.759 | −9.006 | 1.00 | 42.47 |
| ATOM | 978 | CD1 | TYR | A | 833 | −31.781 | 18.877 | −9.223 | 1.00 | 41.47 |
| ATOM | 979 | CD2 | TYR | A | 833 | −30.226 | 19.902 | −7.702 | 1.00 | 39.44 |
| ATOM | 980 | CE1 | TYR | A | 833 | −32.344 | 18.143 | −8.165 | 1.00 | 37.67 |
| ATOM | 981 | CE2 | TYR | A | 833 | −30.784 | 19.184 | −6.639 | 1.00 | 39.15 |
| ATOM | 982 | CZ | TYR | A | 833 | −31.837 | 18.306 | −6.877 | 1.00 | 38.87 |
| ATOM | 983 | OH | TYR | A | 833 | −32.378 | 17.600 | −5.827 | 1.00 | 38.16 |
| ATOM | 984 | N | LEU | A | 834 | −29.232 | 23.304 | −9.176 | 1.00 | 45.55 |
| ATOM | 985 | CA | LEU | A | 834 | −28.958 | 24.225 | −8.072 | 1.00 | 46.56 |
| ATOM | 986 | C | LEU | A | 834 | −29.800 | 25.496 | −8.168 | 1.00 | 45.77 |
| ATOM | 987 | O | LEU | A | 834 | −30.432 | 25.894 | −7.199 | 1.00 | 45.06 |
| ATOM | 988 | CB | LEU | A | 834 | −27.468 | 24.601 | −8.022 | 1.00 | 46.73 |
| ATOM | 989 | CG | LEU | A | 834 | −26.445 | 23.528 | −7.609 | 1.00 | 47.94 |
| ATOM | 990 | CD1 | LEU | A | 834 | −25.044 | 24.081 | −7.736 | 1.00 | 45.98 |
| ATOM | 991 | CD2 | LEU | A | 834 | −26.679 | 23.094 | −6.184 | 1.00 | 46.82 |
| ATOM | 992 | N | GLU | A | 835 | −29.803 | 26.126 | −9.337 | 1.00 | 46.46 |
| ATOM | 993 | CA | GLU | A | 835 | −30.561 | 27.359 | −9.559 | 1.00 | 48.02 |
| ATOM | 994 | C | GLU | A | 835 | −32.059 | 27.172 | −9.298 | 1.00 | 48.70 |
| ATOM | 995 | O | GLU | A | 835 | −32.749 | 28.101 | −8.872 | 1.00 | 44.97 |
| ATOM | 996 | CB | GLU | A | 835 | −30.357 | 27.835 | −10.997 | 1.00 | 48.88 |
| ATOM | 997 | CG | GLU | A | 835 | −30.994 | 29.167 | −11.320 | 1.00 | 51.38 |
| ATOM | 998 | CD | GLU | A | 835 | −30.899 | 29.497 | −12.791 | 1.00 | 55.29 |
| ATOM | 999 | OE1 | GLU | A | 835 | −31.031 | 30.682 | −13.160 | 1.00 | 61.79 |
| ATOM | 1000 | OE2 | GLU | A | 835 | −30.699 | 28.562 | −13.591 | 1.00 | 59.22 |
| ATOM | 1001 | N | GLU | A | 836 | −32.547 | 25.966 | −9.585 | 1.00 | 49.61 |
| ATOM | 1002 | CA | GLU | A | 836 | −33.941 | 25.609 | −9.393 | 1.00 | 51.33 |
| ATOM | 1003 | C | GLU | A | 836 | −34.251 | 25.644 | −7.893 | 1.00 | 52.42 |
| ATOM | 1004 | O | GLU | A | 836 | −35.376 | 25.933 | −7.476 | 1.00 | 53.59 |
| ATOM | 1005 | CB | GLU | A | 836 | −34.172 | 24.213 | −9.976 | 1.00 | 53.43 |
| ATOM | 1006 | CG | GLU | A | 836 | −35.554 | 23.632 | −9.752 | 1.00 | 61.91 |
| ATOM | 1007 | CD | GLU | A | 836 | −35.694 | 22.222 | −10.322 | 1.00 | 68.30 |
| ATOM | 1008 | OE1 | GLU | A | 836 | −34.777 | 21.393 | −10.103 | 1.00 | 70.37 |
| ATOM | 1009 | OE2 | GLU | A | 836 | −36.723 | 21.938 | −10.984 | 1.00 | 72.73 |
| ATOM | 1010 | N | ARG | A | 837 | −33.232 | 25.366 | −7.089 | 1.00 | 52.07 |
| ATOM | 1011 | CA | ARG | A | 837 | −33.342 | 25.363 | −5.632 | 1.00 | 53.28 |
| ATOM | 1012 | C | ARG | A | 837 | −32.980 | 26.731 | −5.018 | 1.00 | 53.42 |
| ATOM | 1013 | O | ARG | A | 837 | −32.909 | 26.859 | −3.800 | 1.00 | 52.59 |
| ATOM | 1014 | CB | ARG | A | 837 | −32.401 | 24.300 | −5.041 | 1.00 | 54.24 |
| ATOM | 1015 | CG | ARG | A | 837 | −32.654 | 22.870 | −5.507 | 1.00 | 54.61 |
| ATOM | 1016 | CD | ARG | A | 837 | −33.996 | 22.419 | −5.013 | 1.00 | 54.07 |
| ATOM | 1017 | NE | ARG | A | 837 | −34.338 | 21.032 | −5.316 | 1.00 | 54.63 |
| ATOM | 1018 | CZ | ARG | A | 837 | −34.342 | 20.493 | −6.529 | 1.00 | 53.35 |
| ATOM | 1019 | NH1 | ARG | A | 837 | −33.993 | 21.207 | −7.590 | 1.00 | 54.44 |
| ATOM | 1020 | NH2 | ARG | A | 837 | −34.777 | 19.250 | −6.687 | 1.00 | 52.90 |
| ATOM | 1021 | N | ARG | A | 838 | −32.739 | 27.734 | −5.862 | 1.00 | 54.60 |
| ATOM | 1022 | CA | ARG | A | 838 | −32.366 | 29.084 | −5.417 | 1.00 | 56.39 |
| ATOM | 1023 | C | ARG | A | 838 | −30.963 | 29.097 | −4.789 | 1.00 | 55.44 |
| ATOM | 1024 | O | ARG | A | 838 | −30.669 | 29.912 | −3.911 | 1.00 | 55.30 |
| ATOM | 1025 | CB | ARG | A | 838 | −33.378 | 29.619 | −4.393 | 1.00 | 60.89 |
| ATOM | 1026 | CG | ARG | A | 838 | −34.852 | 29.577 | −4.822 | 1.00 | 68.36 |
| ATOM | 1027 | CD | ARG | A | 838 | −35.206 | 30.654 | −5.846 | 1.00 | 73.13 |
| ATOM | 1028 | NE | ARG | A | 838 | −36.650 | 30.723 | −6.105 | 1.00 | 77.28 |
| ATOM | 1029 | CZ | ARG | A | 838 | −37.320 | 29.908 | −6.920 | 1.00 | 78.68 |
| ATOM | 1030 | NH1 | ARG | A | 838 | −38.629 | 30.061 | −7.074 | 1.00 | 78.70 |
| ATOM | 1031 | NH2 | ARG | A | 838 | −36.686 | 28.951 | −7.592 | 1.00 | 78.88 |
| ATOM | 1032 | N | LEU | A | 839 | −30.091 | 28.207 | −5.253 | 1.00 | 52.78 |
| ATOM | 1033 | CA | LEU | A | 839 | −28.744 | 28.115 | −4.703 | 1.00 | 50.80 |
| ATOM | 1034 | C | LEU | A | 839 | −27.635 | 28.587 | −5.644 | 1.00 | 49.61 |
| ATOM | 1035 | O | LEU | A | 839 | −27.558 | 28.180 | −6.801 | 1.00 | 51.46 |
| ATOM | 1036 | CB | LEU | A | 839 | −28.472 | 26.674 | −4.275 | 1.00 | 51.33 |
| ATOM | 1037 | CG | LEU | A | 839 | −29.535 | 26.036 | −3.370 | 1.00 | 52.40 |
| ATOM | 1038 | CD1 | LEU | A | 839 | −29.216 | 24.548 | −3.176 | 1.00 | 51.01 |
| ATOM | 1039 | CD2 | LEU | A | 839 | −29.593 | 26.778 | −2.026 | 1.00 | 50.05 |
| ATOM | 1040 | N | VAL | A | 840 | −26.773 | 29.455 | −5.138 | 1.00 | 46.94 |
| ATOM | 1041 | CA | VAL | A | 840 | −25.671 | 29.958 | −5.933 | 1.00 | 45.16 |
| ATOM | 1042 | C | VAL | A | 840 | −24.388 | 29.267 | −5.497 | 1.00 | 45.42 |
| ATOM | 1043 | O | VAL | A | 840 | −24.124 | 29.147 | −4.309 | 1.00 | 45.89 |
| ATOM | 1044 | CB | VAL | A | 840 | −25.515 | 31.472 | −5.750 | 1.00 | 44.00 |
| ATOM | 1045 | CG1 | VAL | A | 840 | −24.412 | 32.005 | −6.673 | 1.00 | 39.24 |
| ATOM | 1046 | CG2 | VAL | A | 840 | −26.846 | 32.150 | −6.036 | 1.00 | 42.54 |
| ATOM | 1047 | N | HIS | A | 841 | −23.588 | 28.805 | −6.450 | 1.00 | 45.68 |
| ATOM | 1048 | CA | HIS | A | 841 | −22.343 | 28.137 | −6.097 | 1.00 | 45.32 |
| ATOM | 1049 | C | HIS | A | 841 | −21.307 | 29.133 | −5.578 | 1.00 | 45.51 |
| ATOM | 1050 | O | HIS | A | 841 | −20.752 | 28.958 | −4.496 | 1.00 | 46.26 |
| ATOM | 1051 | CB | HIS | A | 841 | −21.778 | 27.396 | −7.305 | 1.00 | 44.31 |
| ATOM | 1052 | CG | HIS | A | 841 | −20.707 | 26.410 | −6.956 | 1.00 | 43.65 |
| ATOM | 1053 | ND1 | HIS | A | 841 | −19.583 | 26.755 | −6.240 | 1.00 | 41.64 |
| ATOM | 1054 | CD2 | HIS | A | 841 | −20.592 | 25.088 | −7.222 | 1.00 | 43.78 |
| ATOM | 1055 | CE1 | HIS | A | 841 | −18.821 | 25.689 | −6.080 | 1.00 | 42.85 |
| ATOM | 1056 | NE2 | HIS | A | 841 | −19.410 | 24.664 | −6.667 | 1.00 | 43.25 |
| ATOM | 1057 | N | ARG | A | 842 | −21.050 | 30.173 | −6.363 | 1.00 | 46.38 |
| ATOM | 1058 | CA | ARG | A | 842 | −20.086 | 31.213 | −6.009 | 1.00 | 48.44 |
| ATOM | 1059 | C | ARG | A | 842 | −18.643 | 30.854 | −6.304 | 1.00 | 47.29 |
| ATOM | 1060 | O | ARG | A | 842 | −17.800 | 31.737 | −6.407 | 1.00 | 47.23 |
| ATOM | 1061 | CB | ARG | A | 842 | −20.199 | 31.600 | −4.533 | 1.00 | 50.08 |
| ATOM | 1062 | CG | ARG | A | 842 | −21.388 | 32.452 | −4.221 | 1.00 | 55.04 |
| ATOM | 1063 | CD | ARG | A | 842 | −21.436 | 32.802 | −2.759 | 1.00 | 59.44 |
| ATOM | 1064 | NE | ARG | A | 842 | −22.511 | 33.746 | −2.470 | 1.00 | 62.17 |
| ATOM | 1065 | CZ | ARG | A | 842 | −22.698 | 34.317 | −1.286 | 1.00 | 63.69 |
| ATOM | 1066 | NH1 | ARG | A | 842 | −21.881 | 34.037 | −0.282 | 1.00 | 65.35 |
| ATOM | 1067 | NH2 | ARG | A | 842 | −23.699 | 35.166 | −1.106 | 1.00 | 64.62 |
| ATOM | 1068 | N | ASP | A | 843 | −18.349 | 29.568 | −6.440 | 1.00 | 46.08 |
| ATOM | 1069 | CA | ASP | A | 843 | −16.978 | 29.160 | −6.714 | 1.00 | 44.50 |
| ATOM | 1070 | C | ASP | A | 843 | −16.877 | 27.969 | −7.671 | 1.00 | 42.28 |
| ATOM | 1071 | O | ASP | A | 843 | −16.163 | 26.997 | −7.412 | 1.00 | 39.78 |
| ATOM | 1072 | CB | ASP | A | 843 | −16.260 | 28.857 | −5.393 | 1.00 | 44.95 |
| ATOM | 1073 | CG | ASP | A | 843 | −14.794 | 28.536 | −5.588 | 1.00 | 45.84 |
| ATOM | 1074 | OD1 | ASP | A | 843 | −14.145 | 29.230 | −6.403 | 1.00 | 47.66 |
| ATOM | 1075 | OD2 | ASP | A | 843 | −14.293 | 27.600 | −4.925 | 1.00 | 44.46 |
| ATOM | 1076 | N | LEU | A | 844 | −17.601 | 28.052 | −8.781 | 1.00 | 39.91 |
| ATOM | 1077 | CA | LEU | A | 844 | −17.569 | 26.997 | −9.774 | 1.00 | 38.68 |
| ATOM | 1078 | C | LEU | A | 844 | −16.267 | 27.143 | −10.579 | 1.00 | 39.36 |
| ATOM | 1079 | O | LEU | A | 844 | −15.909 | 28.244 | −11.012 | 1.00 | 36.39 |
| ATOM | 1080 | CB | LEU | A | 844 | −18.787 | 27.110 | −10.690 | 1.00 | 37.44 |
| ATOM | 1081 | CG | LEU | A | 844 | −18.818 | 26.190 | −11.918 | 1.00 | 35.03 |
| ATOM | 1082 | CD1 | LEU | A | 844 | −18.793 | 24.735 | −11.502 | 1.00 | 33.46 |
| ATOM | 1083 | CD2 | LEU | A | 844 | −20.061 | 26.486 | −12.710 | 1.00 | 33.77 |
| ATOM | 1084 | N | ALA | A | 845 | −15.554 | 26.035 | −10.749 | 1.00 | 38.08 |
| ATOM | 1085 | CA | ALA | A | 845 | −14.294 | 26.041 | −11.481 | 1.00 | 41.74 |
| ATOM | 1086 | C | ALA | A | 845 | −13.890 | 24.602 | −11.766 | 1.00 | 43.66 |
| ATOM | 1087 | O | ALA | A | 845 | −14.272 | 23.692 | −11.029 | 1.00 | 45.06 |
| ATOM | 1088 | CB | ALA | A | 845 | −13.209 | 26.740 | −10.653 | 1.00 | 41.01 |
| ATOM | 1089 | N | ALA | A | 846 | −13.128 | 24.388 | −12.832 | 1.00 | 45.09 |
| ATOM | 1090 | CA | ALA | A | 846 | −12.706 | 23.034 | −13.183 | 1.00 | 46.69 |
| ATOM | 1091 | C | ALA | A | 846 | −12.244 | 22.297 | −11.933 | 1.00 | 47.45 |
| ATOM | 1092 | O | ALA | A | 846 | −12.454 | 21.094 | −11.789 | 1.00 | 49.65 |
| ATOM | 1093 | CB | ALA | A | 846 | −11.591 | 23.083 | −14.208 | 1.00 | 45.74 |
| ATOM | 1094 | N | ARG | A | 847 | −11.632 | 23.047 | −11.027 | 1.00 | 49.09 |
| ATOM | 1095 | CA | ARG | A | 847 | −11.117 | 22.527 | −9.759 | 1.00 | 50.53 |
| ATOM | 1096 | C | ARG | A | 847 | −12.175 | 21.893 | −8.858 | 1.00 | 50.50 |
| ATOM | 1097 | O | ARG | A | 847 | −11.872 | 20.978 | −8.100 | 1.00 | 51.14 |
| ATOM | 1098 | CB | ARG | A | 847 | −10.421 | 23.664 | −9.015 | 1.00 | 52.11 |
| ATOM | 1099 | CG | ARG | A | 847 | −10.116 | 23.414 | −7.567 | 1.00 | 55.22 |
| ATOM | 1100 | CD | ARG | A | 847 | −9.438 | 24.635 | −7.002 | 1.00 | 54.87 |
| ATOM | 1101 | NE | ARG | A | 847 | −10.266 | 25.814 | −7.214 | 1.00 | 59.60 |
| ATOM | 1102 | CZ | ARG | A | 847 | −11.222 | 26.221 | −6.381 | 1.00 | 62.29 |
| ATOM | 1103 | NH1 | ARG | A | 847 | −11.468 | 25.543 | −5.262 | 1.00 | 63.16 |
| ATOM | 1104 | NH2 | ARG | A | 847 | −11.942 | 27.298 | −6.676 | 1.00 | 61.26 |
| ATOM | 1105 | N | ASN | A | 848 | −13.412 | 22.374 | −8.931 | 1.00 | 49.43 |
| ATOM | 1106 | CA | ASN | A | 848 | −14.469 | 21.819 | −8.099 | 1.00 | 47.44 |
| ATOM | 1107 | C | ASN | A | 848 | −15.438 | 20.903 | −8.840 | 1.00 | 48.04 |
| ATOM | 1108 | O | ASN | A | 848 | −16.612 | 20.784 | −8.472 | 1.00 | 48.54 |
| ATOM | 1109 | CB | ASN | A | 848 | −15.236 | 22.934 | −7.384 | 1.00 | 45.48 |
| ATOM | 1110 | CG | ASN | A | 848 | −14.397 | 23.628 | −6.337 | 1.00 | 45.91 |
| ATOM | 1111 | OD1 | ASN | A | 848 | −13.754 | 22.979 | −5.521 | 1.00 | 50.11 |
| ATOM | 1112 | ND2 | ASN | A | 848 | −14.406 | 24.950 | −6.347 | 1.00 | 43.85 |
| ATOM | 1113 | N | VAL | A | 849 | −14.951 | 20.265 | −9.897 | 1.00 | 46.96 |
| ATOM | 1114 | CA | VAL | A | 849 | −15.771 | 19.318 | −10.637 | 1.00 | 47.48 |
| ATOM | 1115 | C | VAL | A | 849 | −15.026 | 17.994 | −10.477 | 1.00 | 48.51 |
| ATOM | 1116 | O | VAL | A | 849 | −13.826 | 17.913 | −10.729 | 1.00 | 49.29 |
| ATOM | 1117 | CB | VAL | A | 849 | −15.896 | 19.688 | −12.145 | 1.00 | 47.07 |
| ATOM | 1118 | CG1 | VAL | A | 849 | −16.754 | 18.653 | −12.871 | 1.00 | 47.56 |
| ATOM | 1119 | CG2 | VAL | A | 849 | −16.517 | 21.065 | −12.298 | 1.00 | 45.99 |
| ATOM | 1120 | N | LEU | A | 850 | −15.730 | 16.965 | −10.026 | 1.00 | 48.67 |
| ATOM | 1121 | CA | LEU | A | 850 | −15.109 | 15.667 | −9.813 | 1.00 | 47.31 |
| ATOM | 1122 | C | LEU | A | 850 | −15.554 | 14.634 | −10.855 | 1.00 | 48.46 |
| ATOM | 1123 | O | LEU | A | 850 | −16.641 | 14.733 | −11.430 | 1.00 | 47.00 |
| ATOM | 1124 | CB | LEU | A | 850 | −15.437 | 15.165 | −8.402 | 1.00 | 43.85 |
| ATOM | 1125 | CG | LEU | A | 850 | −15.177 | 16.144 | −7.255 | 1.00 | 42.97 |
| ATOM | 1126 | CD1 | LEU | A | 850 | −15.709 | 15.561 | −5.970 | 1.00 | 40.25 |
| ATOM | 1127 | CD2 | LEU | A | 850 | −13.694 | 16.439 | −7.137 | 1.00 | 42.26 |
| ATOM | 1128 | N | VAL | A | 851 | −14.701 | 13.638 | −11.082 | 1.00 | 50.16 |
| ATOM | 1129 | CA | VAL | A | 851 | −14.985 | 12.588 | −12.045 | 1.00 | 51.71 |
| ATOM | 1130 | C | VAL | A | 851 | −15.343 | 11.257 | −11.371 | 1.00 | 54.08 |
| ATOM | 1131 | O | VAL | A | 851 | −14.485 | 10.615 | −10.757 | 1.00 | 53.24 |
| ATOM | 1132 | CB | VAL | A | 851 | −13.772 | 12.355 | −12.963 | 1.00 | 51.18 |
| ATOM | 1133 | CG1 | VAL | A | 851 | −14.208 | 11.572 | −14.195 | 1.00 | 53.03 |
| ATOM | 1134 | CG2 | VAL | A | 851 | −13.136 | 13.683 | −13.351 | 1.00 | 48.08 |
| ATOM | 1135 | N | LYS | A | 852 | −16.610 | 10.854 | −11.473 | 1.00 | 56.51 |
| ATOM | 1136 | CA | LYS | A | 852 | −17.060 | 9.581 | −10.897 | 1.00 | 60.15 |
| ATOM | 1137 | C | LYS | A | 852 | −16.552 | 8.520 | −11.863 | 1.00 | 62.24 |
| ATOM | 1138 | O | LYS | A | 852 | −16.067 | 7.461 | −11.464 | 1.00 | 64.74 |
| ATOM | 1139 | CB | LYS | A | 852 | −18.584 | 9.524 | −10.829 | 1.00 | 60.47 |
| ATOM | 1140 | CG | LYS | A | 852 | −19.156 | 8.246 | −10.229 | 1.00 | 61.94 |
| ATOM | 1141 | CD | LYS | A | 852 | −20.672 | 8.239 | −10.404 | 1.00 | 66.23 |
| ATOM | 1142 | CE | LYS | A | 852 | −21.375 | 7.079 | −9.703 | 1.00 | 66.46 |
| ATOM | 1143 | NZ | LYS | A | 852 | −22.871 | 7.181 | −9.873 | 1.00 | 66.94 |
| ATOM | 1144 | N | SER | A | 853 | −16.671 | 8.837 | −13.145 | 1.00 | 62.50 |
| ATOM | 1145 | CA | SER | A | 853 | −16.212 | 7.989 | −14.233 | 1.00 | 62.66 |
| ATOM | 1146 | C | SER | A | 853 | −16.012 | 8.924 | −15.431 | 1.00 | 61.67 |
| ATOM | 1147 | O | SER | A | 853 | −16.624 | 9.988 | −15.499 | 1.00 | 60.83 |
| ATOM | 1148 | CB | SER | A | 853 | −17.233 | 6.882 | −14.536 | 1.00 | 63.56 |
| ATOM | 1149 | OG | SER | A | 853 | −18.560 | 7.374 | −14.594 | 1.00 | 66.67 |
| ATOM | 1150 | N | PRO | A | 854 | −15.134 | 8.551 | −16.373 | 1.00 | 60.36 |
| ATOM | 1151 | CA | PRO | A | 854 | −14.849 | 9.364 | −17.558 | 1.00 | 59.12 |
| ATOM | 1152 | C | PRO | A | 854 | −16.065 | 10.032 | −18.179 | 1.00 | 57.95 |
| ATOM | 1153 | O | PRO | A | 854 | −16.003 | 11.182 | −18.610 | 1.00 | 56.93 |
| ATOM | 1154 | CB | PRO | A | 854 | −14.188 | 8.365 | −18.500 | 1.00 | 60.62 |
| ATOM | 1155 | CG | PRO | A | 854 | −13.413 | 7.508 | −17.550 | 1.00 | 60.96 |
| ATOM | 1156 | CD | PRO | A | 854 | −14.418 | 7.263 | −16.443 | 1.00 | 61.47 |
| ATOM | 1157 | N | ALA | A | 855 | −17.175 | 9.313 | −18.210 | 1.00 | 56.59 |
| ATOM | 1158 | CA | ALA | A | 855 | −18.391 | 9.842 | −18.801 | 1.00 | 56.60 |
| ATOM | 1159 | C | ALA | A | 855 | −19.414 | 10.316 | −17.762 | 1.00 | 55.94 |
| ATOM | 1160 | O | ALA | A | 855 | −20.605 | 10.376 | −18.046 | 1.00 | 55.12 |
| ATOM | 1161 | CB | ALA | A | 855 | −19.009 | 8.779 | −19.699 | 1.00 | 57.53 |
| ATOM | 1162 | N | HIS | A | 856 | −18.954 | 10.674 | −16.568 | 1.00 | 55.98 |
| ATOM | 1163 | CA | HIS | A | 856 | −19.869 | 11.100 | −15.513 | 1.00 | 53.27 |
| ATOM | 1164 | C | HIS | A | 856 | −19.169 | 11.954 | −14.460 | 1.00 | 51.77 |
| ATOM | 1165 | O | HIS | A | 856 | −18.433 | 11.446 | −13.618 | 1.00 | 53.22 |
| ATOM | 1166 | CB | HIS | A | 856 | −20.477 | 9.858 | −14.858 | 1.00 | 54.21 |
| ATOM | 1167 | CG | HIS | A | 856 | −21.564 | 10.146 | −13.868 | 1.00 | 56.69 |
| ATOM | 1168 | ND1 | HIS | A | 856 | −22.263 | 9.145 | −13.227 | 1.00 | 56.79 |
| ATOM | 1169 | CD2 | HIS | A | 856 | −22.080 | 11.312 | −13.416 | 1.00 | 55.96 |
| ATOM | 1170 | CE1 | HIS | A | 856 | −23.163 | 9.681 | −12.425 | 1.00 | 57.20 |
| ATOM | 1171 | NE2 | HIS | A | 856 | −23.073 | 10.995 | −12.521 | 1.00 | 57.31 |
| ATOM | 1172 | N | VAL | A | 857 | −19.397 | 13.259 | −14.514 | 1.00 | 49.26 |
| ATOM | 1173 | CA | VAL | A | 857 | −18.794 | 14.161 | −13.550 | 1.00 | 46.76 |
| ATOM | 1174 | C | VAL | A | 857 | −19.864 | 14.686 | −12.589 | 1.00 | 46.86 |
| ATOM | 1175 | O | VAL | A | 857 | −21.065 | 14.579 | −12.861 | 1.00 | 46.96 |
| ATOM | 1176 | CB | VAL | A | 857 | −18.058 | 15.343 | −14.260 | 1.00 | 46.02 |
| ATOM | 1177 | CG1 | VAL | A | 857 | −16.777 | 14.830 | −14.928 | 1.00 | 41.78 |
| ATOM | 1178 | CG2 | VAL | A | 857 | −18.961 | 15.992 | −15.285 | 1.00 | 38.84 |
| ATOM | 1179 | N | LYS | A | 858 | −19.422 | 15.226 | −11.458 | 1.00 | 45.57 |
| ATOM | 1180 | CA | LYS | A | 858 | −20.322 | 15.767 | −10.437 | 1.00 | 45.68 |
| ATOM | 1181 | C | LYS | A | 858 | −19.765 | 17.090 | −9.923 | 1.00 | 45.10 |
| ATOM | 1182 | O | LYS | A | 858 | −18.552 | 17.220 | −9.727 | 1.00 | 43.10 |
| ATOM | 1183 | CB | LYS | A | 858 | −20.442 | 14.804 | −9.242 | 1.00 | 46.28 |
| ATOM | 1184 | CG | LYS | A | 858 | −21.257 | 13.521 | −9.451 | 1.00 | 45.72 |
| ATOM | 1185 | CD | LYS | A | 858 | −21.160 | 12.646 | −8.184 | 1.00 | 44.10 |
| ATOM | 1186 | CE | LYS | A | 858 | −21.914 | 11.324 | −8.292 | 1.00 | 45.25 |
| ATOM | 1187 | NZ | LYS | A | 858 | −23.401 | 11.471 | −8.255 | 1.00 | 47.27 |
| ATOM | 1188 | N | ILE | A | 859 | −20.643 | 18.068 | −9.703 | 1.00 | 45.57 |
| ATOM | 1189 | CA | ILE | A | 859 | −20.203 | 19.363 | −9.185 | 1.00 | 45.70 |
| ATOM | 1190 | C | ILE | A | 859 | −20.098 | 19.256 | −7.669 | 1.00 | 47.41 |
| ATOM | 1191 | O | ILE | A | 859 | −20.981 | 18.687 | −7.023 | 1.00 | 48.32 |
| ATOM | 1192 | CB | ILE | A | 859 | −21.185 | 20.482 | −9.547 | 1.00 | 44.76 |
| ATOM | 1193 | CG1 | ILE | A | 859 | −21.259 | 20.622 | −11.074 | 1.00 | 44.37 |
| ATOM | 1194 | CG2 | ILE | A | 859 | −20.724 | 21.793 | −8.916 | 1.00 | 44.21 |
| ATOM | 1195 | CD1 | ILE | A | 859 | −22.156 | 21.719 | −11.561 | 1.00 | 40.72 |
| ATOM | 1196 | N | THR | A | 860 | −19.018 | 19.787 | −7.104 | 1.00 | 46.98 |
| ATOM | 1197 | CA | THR | A | 860 | −18.811 | 19.711 | −5.662 | 1.00 | 47.63 |
| ATOM | 1198 | C | THR | A | 860 | −18.467 | 21.050 | −5.024 | 1.00 | 48.69 |
| ATOM | 1199 | O | THR | A | 860 | −18.155 | 22.019 | −5.711 | 1.00 | 49.80 |
| ATOM | 1200 | CB | THR | A | 860 | −17.670 | 18.714 | −5.308 | 1.00 | 46.89 |
| ATOM | 1201 | OG1 | THR | A | 860 | −17.661 | 18.468 | −3.897 | 1.00 | 45.80 |
| ATOM | 1202 | CG2 | THR | A | 860 | −16.322 | 19.295 | −5.686 | 1.00 | 44.93 |
| ATOM | 1203 | N | ASP | A | 861 | −18.526 | 21.078 | −3.696 | 1.00 | 49.00 |
| ATOM | 1204 | CA | ASP | A | 861 | −18.195 | 22.253 | −2.897 | 1.00 | 51.12 |
| ATOM | 1205 | C | ASP | A | 861 | −19.116 | 23.454 | −2.982 | 1.00 | 52.18 |
| ATOM | 1206 | O | ASP | A | 861 | −18.722 | 24.560 | −2.630 | 1.00 | 53.11 |
| ATOM | 1207 | CB | ASP | A | 861 | −16.767 | 22.716 | −3.177 | 1.00 | 49.84 |
| ATOM | 1208 | CG | ASP | A | 861 | −16.057 | 23.146 | −1.914 | 1.00 | 52.95 |
| ATOM | 1209 | OD1 | ASP | A | 861 | −16.108 | 22.375 | −0.941 | 1.00 | 55.50 |
| ATOM | 1210 | OD2 | ASP | A | 861 | −15.451 | 24.233 | −1.871 | 1.00 | 55.17 |
| ATOM | 1211 | N | PHE | A | 862 | −20.340 | 23.254 | −3.440 | 1.00 | 54.22 |
| ATOM | 1212 | CA | PHE | A | 862 | −21.269 | 24.367 | −3.500 | 1.00 | 57.56 |
| ATOM | 1213 | C | PHE | A | 862 | −21.792 | 24.637 | −2.080 | 1.00 | 61.09 |
| ATOM | 1214 | O | PHE | A | 862 | −22.294 | 23.734 | −1.410 | 1.00 | 62.23 |
| ATOM | 1215 | CB | PHE | A | 862 | −22.429 | 24.032 | −4.437 | 1.00 | 55.96 |
| ATOM | 1216 | CG | PHE | A | 862 | −23.035 | 22.671 | −4.197 | 1.00 | 55.99 |
| ATOM | 1217 | CD1 | PHE | A | 862 | −22.751 | 21.609 | −5.051 | 1.00 | 56.15 |
| ATOM | 1218 | CD2 | PHE | A | 862 | −23.883 | 22.448 | −3.115 | 1.00 | 53.52 |
| ATOM | 1219 | CE1 | PHE | A | 862 | −23.302 | 20.348 | −4.829 | 1.00 | 56.04 |
| ATOM | 1220 | CE2 | PHE | A | 862 | −24.435 | 21.195 | −2.886 | 1.00 | 52.92 |
| ATOM | 1221 | CZ | PHE | A | 862 | −24.144 | 20.141 | −3.746 | 1.00 | 54.63 |
| ATOM | 1222 | N | GLY | A | 863 | −21.651 | 25.866 | −1.600 | 1.00 | 64.10 |
| ATOM | 1223 | CA | GLY | A | 863 | −22.153 | 26.162 | −0.268 | 1.00 | 67.86 |
| ATOM | 1224 | C | GLY | A | 863 | −21.176 | 26.776 | 0.716 | 1.00 | 70.52 |
| ATOM | 1225 | O | GLY | A | 863 | −21.529 | 27.720 | 1.427 | 1.00 | 70.56 |
| ATOM | 1226 | N | LEU | A | 864 | −19.959 | 26.240 | 0.780 | 1.00 | 72.06 |
| ATOM | 1227 | CA | LEU | A | 864 | −18.953 | 26.772 | 1.691 | 1.00 | 73.84 |
| ATOM | 1228 | C | LEU | A | 864 | −18.657 | 28.229 | 1.337 | 1.00 | 75.19 |
| ATOM | 1229 | O | LEU | A | 864 | −18.297 | 29.027 | 2.199 | 1.00 | 77.44 |
| ATOM | 1230 | CB | LEU | A | 864 | −17.660 | 25.955 | 1.610 | 1.00 | 73.25 |
| ATOM | 1231 | CG | LEU | A | 864 | −17.786 | 24.442 | 1.773 | 1.00 | 72.21 |
| ATOM | 1232 | CD1 | LEU | A | 864 | −16.405 | 23.833 | 1.808 | 1.00 | 71.56 |
| ATOM | 1233 | CD2 | LEU | A | 864 | −18.543 | 24.115 | 3.037 | 1.00 | 71.95 |
| ATOM | 1234 | N | ALA | A | 865 | −18.812 | 28.569 | 0.064 | 1.00 | 75.54 |
| ATOM | 1235 | CA | ALA | A | 865 | −18.564 | 29.924 | −0.403 | 1.00 | 75.51 |
| ATOM | 1236 | C | ALA | A | 865 | −19.274 | 30.937 | 0.485 | 1.00 | 76.03 |
| ATOM | 1237 | O | ALA | A | 865 | −18.654 | 31.859 | 1.020 | 1.00 | 77.18 |
| ATOM | 1238 | CB | ALA | A | 865 | −19.048 | 30.067 | −1.834 | 1.00 | 76.59 |
| ATOM | 1239 | N | ARG | A | 866 | −20.579 | 30.757 | 0.641 | 1.00 | 75.54 |
| ATOM | 1240 | CA | ARG | A | 866 | −21.389 | 31.661 | 1.448 | 1.00 | 74.65 |
| ATOM | 1241 | C | ARG | A | 866 | −21.168 | 31.435 | 2.940 | 1.00 | 73.26 |
| ATOM | 1242 | O | ARG | A | 866 | −21.048 | 32.382 | 3.716 | 1.00 | 72.57 |
| ATOM | 1243 | CB | ARG | A | 866 | −22.869 | 31.456 | 1.116 | 1.00 | 75.86 |
| ATOM | 1244 | CG | ARG | A | 866 | −23.797 | 32.543 | 1.621 | 1.00 | 78.29 |
| ATOM | 1245 | CD | ARG | A | 866 | −25.223 | 32.212 | 1.231 | 1.00 | 81.93 |
| ATOM | 1246 | NE | ARG | A | 866 | −26.098 | 33.379 | 1.227 | 1.00 | 85.05 |
| ATOM | 1247 | CZ | ARG | A | 866 | −27.359 | 33.362 | 0.800 | 1.00 | 87.00 |
| ATOM | 1248 | NH1 | ARG | A | 866 | −27.893 | 32.233 | 0.343 | 1.00 | 86.94 |
| ATOM | 1249 | NH2 | ARG | A | 866 | −28.082 | 34.476 | 0.816 | 1.00 | 88.11 |
| ATOM | 1250 | N | LEU | A | 867 | −21.104 | 30.172 | 3.333 | 1.00 | 71.68 |
| ATOM | 1251 | CA | LEU | A | 867 | −20.929 | 29.817 | 4.732 | 1.00 | 71.24 |
| ATOM | 1252 | C | LEU | A | 867 | −19.657 | 30.365 | 5.369 | 1.00 | 70.68 |
| ATOM | 1253 | O | LEU | A | 867 | −19.642 | 30.649 | 6.566 | 1.00 | 70.39 |
| ATOM | 1254 | CB | LEU | A | 867 | −20.963 | 28.296 | 4.885 | 1.00 | 70.78 |
| ATOM | 1255 | CG | LEU | A | 867 | −21.160 | 27.766 | 6.300 | 1.00 | 69.00 |
| ATOM | 1256 | CD1 | LEU | A | 867 | −22.544 | 28.150 | 6.800 | 1.00 | 70.14 |
| ATOM | 1257 | CD2 | LEU | A | 867 | −21.006 | 26.262 | 6.299 | 1.00 | 69.35 |
| ATOM | 1258 | N | LEU | A | 868 | −18.597 | 30.519 | 4.579 | 1.00 | 70.51 |
| ATOM | 1259 | CA | LEU | A | 868 | −17.333 | 31.021 | 5.108 | 1.00 | 70.64 |
| ATOM | 1260 | C | LEU | A | 868 | −17.202 | 32.537 | 5.032 | 1.00 | 71.18 |
| ATOM | 1261 | O | LEU | A | 868 | −16.786 | 33.179 | 5.996 | 1.00 | 71.59 |
| ATOM | 1262 | CB | LEU | A | 868 | −16.152 | 30.362 | 4.390 | 1.00 | 70.84 |
| ATOM | 1263 | CG | LEU | A | 868 | −16.175 | 28.826 | 4.286 | 1.00 | 72.40 |
| ATOM | 1264 | CD1 | LEU | A | 868 | −14.780 | 28.326 | 3.889 | 1.00 | 70.82 |
| ATOM | 1265 | CD2 | LEU | A | 868 | −16.614 | 28.201 | 5.610 | 1.00 | 70.35 |
| ATOM | 1266 | N | GLU | A | 869 | −17.549 | 33.121 | 3.894 | 1.00 | 72.14 |
| ATOM | 1267 | CA | GLU | A | 869 | −17.456 | 34.567 | 3.771 | 1.00 | 72.99 |
| ATOM | 1268 | C | GLU | A | 869 | −18.658 | 35.262 | 4.389 | 1.00 | 72.22 |
| ATOM | 1269 | O | GLU | A | 869 | −18.633 | 35.645 | 5.558 | 1.00 | 72.77 |
| ATOM | 1270 | CB | GLU | A | 869 | −17.316 | 34.968 | 2.311 | 1.00 | 74.69 |
| ATOM | 1271 | CG | GLU | A | 869 | −15.925 | 34.742 | 1.782 | 1.00 | 78.22 |
| ATOM | 1272 | CD | GLU | A | 869 | −15.688 | 35.427 | 0.451 | 1.00 | 81.09 |
| ATOM | 1273 | OE1 | GLU | A | 869 | −14.505 | 35.593 | 0.097 | 1.00 | 81.80 |
| ATOM | 1274 | OE2 | GLU | A | 869 | −16.671 | 35.793 | −0.241 | 1.00 | 81.14 |
| ATOM | 1275 | N | GLY | A | 870 | −19.713 | 35.415 | 3.602 | 1.00 | 71.14 |
| ATOM | 1276 | CA | GLY | A | 870 | −20.910 | 36.064 | 4.093 | 1.00 | 69.73 |
| ATOM | 1277 | C | GLY | A | 870 | −21.585 | 36.758 | 2.941 | 1.00 | 69.34 |
| ATOM | 1278 | O | GLY | A | 870 | −21.266 | 36.488 | 1.788 | 1.00 | 67.87 |
| ATOM | 1279 | N | ASP | A | 871 | −22.514 | 37.654 | 3.245 | 1.00 | 70.63 |
| ATOM | 1280 | CA | ASP | A | 871 | −23.221 | 38.388 | 2.206 | 1.00 | 71.41 |
| ATOM | 1281 | C | ASP | A | 871 | −22.979 | 39.888 | 2.287 | 1.00 | 69.67 |
| ATOM | 1282 | O | ASP | A | 871 | −23.710 | 40.673 | 1.693 | 1.00 | 70.04 |
| ATOM | 1283 | CB | ASP | A | 871 | −24.720 | 38.092 | 2.291 | 1.00 | 75.78 |
| ATOM | 1284 | CG | ASP | A | 871 | −25.081 | 36.742 | 1.692 | 1.00 | 80.13 |
| ATOM | 1285 | OD1 | ASP | A | 871 | −25.017 | 36.611 | 0.448 | 1.00 | 82.71 |
| ATOM | 1286 | OD2 | ASP | A | 871 | −25.419 | 35.811 | 2.458 | 1.00 | 81.77 |
| ATOM | 1287 | N | GLU | A | 872 | −21.945 | 40.288 | 3.018 | 1.00 | 69.40 |
| ATOM | 1288 | CA | GLU | A | 872 | −21.638 | 41.704 | 3.156 | 1.00 | 69.92 |
| ATOM | 1289 | C | GLU | A | 872 | −21.292 | 42.261 | 1.783 | 1.00 | 67.97 |
| ATOM | 1290 | O | GLU | A | 872 | −20.261 | 41.926 | 1.204 | 1.00 | 67.08 |
| ATOM | 1291 | CB | GLU | A | 872 | −20.469 | 41.902 | 4.125 | 1.00 | 72.87 |
| ATOM | 1292 | CG | GLU | A | 872 | −20.340 | 43.319 | 4.675 | 1.00 | 78.70 |
| ATOM | 1293 | CD | GLU | A | 872 | −19.204 | 43.458 | 5.689 | 1.00 | 83.05 |
| ATOM | 1294 | OE1 | GLU | A | 872 | −19.079 | 44.535 | 6.318 | 1.00 | 83.94 |
| ATOM | 1295 | OE2 | GLU | A | 872 | −18.429 | 42.490 | 5.857 | 1.00 | 86.32 |
| ATOM | 1296 | N | LYS | A | 873 | −22.169 | 43.109 | 1.264 | 1.00 | 66.05 |
| ATOM | 1297 | CA | LYS | A | 873 | −21.976 | 43.703 | −0.046 | 1.00 | 65.17 |
| ATOM | 1298 | C | LYS | A | 873 | −20.551 | 44.188 | −0.314 | 1.00 | 64.82 |
| ATOM | 1299 | O | LYS | A | 873 | −19.998 | 43.941 | −1.380 | 1.00 | 64.23 |
| ATOM | 1300 | CB | LYS | A | 873 | −22.956 | 44.864 | −0.234 | 1.00 | 65.59 |
| ATOM | 1301 | CG | LYS | A | 873 | −22.803 | 45.611 | −1.561 | 1.00 | 68.09 |
| ATOM | 1302 | CD | LYS | A | 873 | −23.875 | 46.686 | −1.729 | 1.00 | 69.73 |
| ATOM | 1303 | CE | LYS | A | 873 | −23.672 | 47.499 | −3.007 | 1.00 | 71.60 |
| ATOM | 1304 | NZ | LYS | A | 873 | −24.749 | 48.517 | −3.211 | 1.00 | 71.19 |
| ATOM | 1305 | N | GLU | A | 874 | −19.949 | 44.873 | 0.652 | 1.00 | 65.02 |
| ATOM | 1306 | CA | GLU | A | 874 | −18.603 | 45.397 | 0.454 | 1.00 | 63.95 |
| ATOM | 1307 | C | GLU | A | 874 | −17.487 | 44.565 | 1.065 | 1.00 | 62.00 |
| ATOM | 1308 | O | GLU | A | 874 | −17.587 | 44.080 | 2.194 | 1.00 | 62.31 |
| ATOM | 1309 | CB | GLU | A | 874 | −18.518 | 46.840 | 0.964 | 1.00 | 65.71 |
| ATOM | 1310 | CG | GLU | A | 874 | −19.059 | 47.045 | 2.363 | 1.00 | 69.44 |
| ATOM | 1311 | CD | GLU | A | 874 | −20.576 | 46.946 | 2.437 | 1.00 | 71.62 |
| ATOM | 1312 | OE1 | GLU | A | 874 | −21.259 | 47.887 | 1.973 | 1.00 | 71.73 |
| ATOM | 1313 | OE2 | GLU | A | 874 | −21.083 | 45.923 | 2.955 | 1.00 | 72.76 |
| ATOM | 1314 | N | TYR | A | 875 | −16.422 | 44.399 | 0.290 | 1.00 | 59.06 |
| ATOM | 1315 | CA | TYR | A | 875 | −15.259 | 43.641 | 0.720 | 1.00 | 57.62 |
| ATOM | 1316 | C | TYR | A | 875 | −14.381 | 44.522 | 1.587 | 1.00 | 57.53 |
| ATOM | 1317 | O | TYR | A | 875 | −14.266 | 45.724 | 1.349 | 1.00 | 56.84 |
| ATOM | 1318 | CB | TYR | A | 875 | −14.414 | 43.225 | −0.480 | 1.00 | 56.65 |
| ATOM | 1319 | CG | TYR | A | 875 | −15.098 | 42.342 | −1.484 | 1.00 | 56.70 |
| ATOM | 1320 | CD1 | TYR | A | 875 | −15.421 | 41.027 | −1.173 | 1.00 | 56.15 |
| ATOM | 1321 | CD2 | TYR | A | 875 | −15.402 | 42.817 | −2.759 | 1.00 | 55.46 |
| ATOM | 1322 | CE1 | TYR | A | 875 | −16.027 | 40.205 | −2.103 | 1.00 | 56.63 |
| ATOM | 1323 | CE2 | TYR | A | 875 | −16.007 | 42.005 | −3.697 | 1.00 | 55.93 |
| ATOM | 1324 | CZ | TYR | A | 875 | −16.319 | 40.697 | −3.362 | 1.00 | 56.44 |
| ATOM | 1325 | OH | TYR | A | 875 | −16.933 | 39.879 | −4.275 | 1.00 | 55.10 |
| ATOM | 1326 | N | ASN | A | 876 | −13.760 | 43.933 | 2.597 | 1.00 | 57.67 |
| ATOM | 1327 | CA | ASN | A | 876 | −12.852 | 44.705 | 3.409 | 1.00 | 58.46 |
| ATOM | 1328 | C | ASN | A | 876 | −11.471 | 44.340 | 2.859 | 1.00 | 59.52 |
| ATOM | 1329 | O | ASN | A | 876 | −11.382 | 43.709 | 1.806 | 1.00 | 58.53 |
| ATOM | 1330 | CB | ASN | A | 876 | −13.016 | 44.393 | 4.908 | 1.00 | 59.97 |
| ATOM | 1331 | CG | ASN | A | 876 | −12.785 | 42.934 | 5.255 | 1.00 | 63.94 |
| ATOM | 1332 | OD1 | ASN | A | 876 | −11.833 | 42.311 | 4.775 | 1.00 | 67.34 |
| ATOM | 1333 | ND2 | ASN | A | 876 | −13.642 | 42.387 | 6.125 | 1.00 | 61.21 |
| ATOM | 1334 | N | ALA | A | 877 | −10.405 | 44.736 | 3.546 | 1.00 | 60.79 |
| ATOM | 1335 | CA | ALA | A | 877 | −9.041 | 44.483 | 3.081 | 1.00 | 61.10 |
| ATOM | 1336 | C | ALA | A | 877 | −8.689 | 43.019 | 2.829 | 1.00 | 62.40 |
| ATOM | 1337 | O | ALA | A | 877 | −7.782 | 42.714 | 2.046 | 1.00 | 61.52 |
| ATOM | 1338 | CB | ALA | A | 877 | −8.058 | 45.080 | 4.066 | 1.00 | 60.97 |
| ATOM | 1339 | N | ASP | A | 878 | −9.409 | 42.122 | 3.494 | 1.00 | 63.27 |
| ATOM | 1340 | CA | ASP | A | 878 | −9.172 | 40.690 | 3.370 | 1.00 | 63.19 |
| ATOM | 1341 | C | ASP | A | 878 | −9.636 | 40.145 | 2.022 | 1.00 | 63.64 |
| ATOM | 1342 | O | ASP | A | 878 | −9.158 | 39.107 | 1.569 | 1.00 | 63.47 |
| ATOM | 1343 | CB | ASP | A | 878 | −9.879 | 39.958 | 4.509 | 1.00 | 62.08 |
| ATOM | 1344 | CG | ASP | A | 878 | −9.278 | 38.604 | 4.787 | 1.00 | 64.16 |
| ATOM | 1345 | OD1 | ASP | A | 878 | −8.034 | 38.520 | 4.917 | 1.00 | 63.46 |
| ATOM | 1346 | OD2 | ASP | A | 878 | −10.047 | 37.624 | 4.885 | 1.00 | 65.45 |
| ATOM | 1347 | N | GLY | A | 879 | −10.571 | 40.848 | 1.387 | 1.00 | 64.96 |
| ATOM | 1348 | CA | GLY | A | 879 | −11.081 | 40.422 | 0.092 | 1.00 | 66.39 |
| ATOM | 1349 | C | GLY | A | 879 | −11.894 | 39.139 | 0.105 | 1.00 | 66.47 |
| ATOM | 1350 | O | GLY | A | 879 | −12.483 | 38.779 | 1.124 | 1.00 | 67.04 |
| ATOM | 1351 | N | GLY | A | 880 | −11.920 | 38.443 | −1.031 | 1.00 | 66.80 |
| ATOM | 1352 | CA | GLY | A | 880 | −12.676 | 37.204 | −1.127 | 1.00 | 66.89 |
| ATOM | 1353 | C | GLY | A | 880 | −11.850 | 35.931 | −1.217 | 1.00 | 66.50 |
| ATOM | 1354 | O | GLY | A | 880 | −10.679 | 35.976 | −1.586 | 1.00 | 67.73 |
| ATOM | 1355 | N | LYS | A | 881 | −12.461 | 34.793 | −0.892 | 1.00 | 66.57 |
| ATOM | 1356 | CA | LYS | A | 881 | −11.773 | 33.503 | −0.935 | 1.00 | 67.51 |
| ATOM | 1357 | C | LYS | A | 881 | −11.804 | 32.841 | −2.316 | 1.00 | 66.54 |
| ATOM | 1358 | O | LYS | A | 881 | −10.988 | 31.958 | −2.597 | 1.00 | 66.96 |
| ATOM | 1359 | CB | LYS | A | 881 | −12.353 | 32.538 | 0.116 | 1.00 | 69.38 |
| ATOM | 1360 | CG | LYS | A | 881 | −12.196 | 33.032 | 1.552 | 1.00 | 73.80 |
| ATOM | 1361 | CD | LYS | A | 881 | −12.514 | 31.966 | 2.608 | 1.00 | 76.95 |
| ATOM | 1362 | CE | LYS | A | 881 | −12.271 | 32.525 | 4.032 | 1.00 | 79.84 |
| ATOM | 1363 | NZ | LYS | A | 881 | −12.387 | 31.531 | 5.153 | 1.00 | 77.75 |
| ATOM | 1364 | N | MET | A | 882 | −12.738 | 33.255 | −3.172 | 1.00 | 63.86 |
| ATOM | 1365 | CA | MET | A | 882 | −12.822 | 32.682 | −4.512 | 1.00 | 62.87 |
| ATOM | 1366 | C | MET | A | 882 | −11.752 | 33.268 | −5.420 | 1.00 | 59.24 |
| ATOM | 1367 | O | MET | A | 882 | −11.448 | 34.453 | −5.344 | 1.00 | 59.24 |
| ATOM | 1368 | CB | MET | A | 882 | −14.182 | 32.957 | −5.152 | 1.00 | 67.37 |
| ATOM | 1369 | CG | MET | A | 882 | −15.308 | 32.094 | −4.663 | 1.00 | 73.66 |
| ATOM | 1370 | SD | MET | A | 882 | −15.853 | 32.567 | −3.040 | 1.00 | 80.15 |
| ATOM | 1371 | CE | MET | A | 882 | −15.822 | 30.977 | −2.239 | 1.00 | 79.51 |
| ATOM | 1372 | N | PRO | A | 883 | −11.175 | 32.447 | −6.303 | 1.00 | 55.61 |
| ATOM | 1373 | CA | PRO | A | 883 | −10.150 | 33.005 | −7.180 | 1.00 | 54.96 |
| ATOM | 1374 | C | PRO | A | 883 | −10.749 | 34.048 | −8.127 | 1.00 | 54.82 |
| ATOM | 1375 | O | PRO | A | 883 | −11.750 | 33.794 | −8.788 | 1.00 | 54.42 |
| ATOM | 1376 | CB | PRO | A | 883 | −9.598 | 31.769 | −7.892 | 1.00 | 54.93 |
| ATOM | 1377 | CG | PRO | A | 883 | −10.765 | 30.818 | −7.897 | 1.00 | 55.39 |
| ATOM | 1378 | CD | PRO | A | 883 | −11.367 | 31.006 | −6.539 | 1.00 | 54.78 |
| ATOM | 1379 | N | ILE | A | 884 | −10.122 | 35.221 | −8.166 | 1.00 | 55.44 |
| ATOM | 1380 | CA | ILE | A | 884 | −10.536 | 36.360 | −8.986 | 1.00 | 55.16 |
| ATOM | 1381 | C | ILE | A | 884 | −10.812 | 36.062 | −10.462 | 1.00 | 54.77 |
| ATOM | 1382 | O | ILE | A | 884 | −11.743 | 36.609 | −11.052 | 1.00 | 54.35 |
| ATOM | 1383 | CB | ILE | A | 884 | −9.457 | 37.490 | −8.930 | 1.00 | 57.82 |
| ATOM | 1384 | CG1 | ILE | A | 884 | −9.280 | 37.996 | −7.492 | 1.00 | 60.91 |
| ATOM | 1385 | CG2 | ILE | A | 884 | −9.863 | 38.658 | −9.812 | 1.00 | 57.84 |
| ATOM | 1386 | CD1 | ILE | A | 884 | −10.456 | 38.817 | −6.963 | 1.00 | 61.63 |
| ATOM | 1387 | N | LYS | A | 885 | −10.003 | 35.196 | −11.057 | 1.00 | 54.04 |
| ATOM | 1388 | CA | LYS | A | 885 | −10.126 | 34.878 | −12.476 | 1.00 | 53.74 |
| ATOM | 1389 | C | LYS | A | 885 | −11.342 | 34.054 | −12.922 | 1.00 | 52.87 |
| ATOM | 1390 | O | LYS | A | 885 | −11.586 | 33.890 | −14.117 | 1.00 | 53.63 |
| ATOM | 1391 | CB | LYS | A | 885 | −8.823 | 34.222 | −12.937 | 1.00 | 55.38 |
| ATOM | 1392 | CG | LYS | A | 885 | −7.609 | 35.112 | −12.690 | 1.00 | 57.08 |
| ATOM | 1393 | CD | LYS | A | 885 | −6.308 | 34.437 | −13.069 | 1.00 | 59.92 |
| ATOM | 1394 | CE | LYS | A | 885 | −5.131 | 35.372 | −12.845 | 1.00 | 62.00 |
| ATOM | 1395 | NZ | LYS | A | 885 | −3.829 | 34.700 | −13.114 | 1.00 | 65.60 |
| ATOM | 1396 | N | TRP | A | 886 | −12.108 | 33.546 | −11.967 | 1.00 | 50.16 |
| ATOM | 1397 | CA | TRP | A | 886 | −13.304 | 32.766 | −12.271 | 1.00 | 47.41 |
| ATOM | 1398 | C | TRP | A | 886 | −14.526 | 33.578 | −11.836 | 1.00 | 47.34 |
| ATOM | 1399 | O | TRP | A | 886 | −15.664 | 33.109 | −11.882 | 1.00 | 47.47 |
| ATOM | 1400 | CB | TRP | A | 886 | −13.268 | 31.461 | −11.483 | 1.00 | 45.53 |
| ATOM | 1401 | CG | TRP | A | 886 | −12.483 | 30.373 | −12.121 | 1.00 | 45.12 |
| ATOM | 1402 | CD1 | TRP | A | 886 | −12.967 | 29.373 | −12.911 | 1.00 | 42.75 |
| ATOM | 1403 | CD2 | TRP | A | 886 | −11.077 | 30.149 | −12.004 | 1.00 | 44.61 |
| ATOM | 1404 | NE1 | TRP | A | 886 | −11.952 | 28.536 | −13.288 | 1.00 | 42.27 |
| ATOM | 1405 | CE2 | TRP | A | 886 | −10.778 | 28.989 | −12.747 | 1.00 | 43.80 |
| ATOM | 1406 | CE3 | TRP | A | 886 | −10.038 | 30.814 | −11.344 | 1.00 | 45.37 |
| ATOM | 1407 | CZ2 | TRP | A | 886 | −9.477 | 28.476 | −12.850 | 1.00 | 41.96 |
| ATOM | 1408 | CZ3 | TRP | A | 886 | −8.743 | 30.302 | −11.448 | 1.00 | 45.18 |
| ATOM | 1409 | CH2 | TRP | A | 886 | −8.478 | 29.144 | −12.197 | 1.00 | 40.59 |
| ATOM | 1410 | N | MET | A | 887 | −14.266 | 34.809 | −11.428 | 1.00 | 45.49 |
| ATOM | 1411 | CA | MET | A | 887 | −15.292 | 35.689 | −10.903 | 1.00 | 47.92 |
| ATOM | 1412 | C | MET | A | 887 | −15.945 | 36.606 | −11.924 | 1.00 | 47.15 |
| ATOM | 1413 | O | MET | A | 887 | −15.286 | 37.112 | −12.826 | 1.00 | 46.97 |
| ATOM | 1414 | CB | MET | A | 887 | −14.655 | 36.521 | −9.781 | 1.00 | 50.44 |
| ATOM | 1415 | CG | MET | A | 887 | −15.580 | 37.050 | −8.724 | 1.00 | 51.18 |
| ATOM | 1416 | SD | MET | A | 887 | −14.641 | 37.374 | −7.213 | 1.00 | 53.31 |
| ATOM | 1417 | CE | MET | A | 887 | −14.838 | 35.821 | −6.359 | 1.00 | 55.22 |
| ATOM | 1418 | N | ALA | A | 888 | −17.248 | 36.814 | −11.771 | 1.00 | 46.65 |
| ATOM | 1419 | CA | ALA | A | 888 | −17.992 | 37.707 | −12.653 | 1.00 | 47.77 |
| ATOM | 1420 | C | ALA | A | 888 | −17.620 | 39.138 | −12.281 | 1.00 | 48.61 |
| ATOM | 1421 | O | ALA | A | 888 | −17.515 | 39.483 | −11.104 | 1.00 | 48.25 |
| ATOM | 1422 | CB | ALA | A | 888 | −19.491 | 37.497 | −12.486 | 1.00 | 45.93 |
| ATOM | 1423 | N | LEU | A | 889 | −17.418 | 39.965 | −13.294 | 1.00 | 50.52 |
| ATOM | 1424 | CA | LEU | A | 889 | −17.030 | 41.350 | −13.098 | 1.00 | 51.06 |
| ATOM | 1425 | C | LEU | A | 889 | −17.751 | 42.039 | −11.953 | 1.00 | 52.52 |
| ATOM | 1426 | O | LEU | A | 889 | −17.122 | 42.682 | −11.104 | 1.00 | 52.90 |
| ATOM | 1427 | CB | LEU | A | 889 | −17.270 | 42.121 | −14.387 | 1.00 | 52.30 |
| ATOM | 1428 | CG | LEU | A | 889 | −16.926 | 43.607 | −14.399 | 1.00 | 53.28 |
| ATOM | 1429 | CD1 | LEU | A | 889 | −15.502 | 43.802 | −13.915 | 1.00 | 53.95 |
| ATOM | 1430 | CD2 | LEU | A | 889 | −17.114 | 44.154 | −15.816 | 1.00 | 49.72 |
| ATOM | 1431 | N | GLU | A | 890 | −19.072 | 41.894 | −11.919 | 1.00 | 52.25 |
| ATOM | 1432 | CA | GLU | A | 890 | −19.864 | 42.538 | −10.881 | 1.00 | 52.51 |
| ATOM | 1433 | C | GLU | A | 890 | −19.517 | 42.070 | −9.482 | 1.00 | 54.25 |
| ATOM | 1434 | O | GLU | A | 890 | −19.668 | 42.821 | −8.520 | 1.00 | 55.68 |
| ATOM | 1435 | CB | GLU | A | 890 | −21.361 | 42.322 | −11.116 | 1.00 | 51.54 |
| ATOM | 1436 | CG | GLU | A | 890 | −21.799 | 40.871 | −11.075 | 1.00 | 49.82 |
| ATOM | 1437 | CD | GLU | A | 890 | −21.820 | 40.236 | −12.448 | 1.00 | 47.38 |
| ATOM | 1438 | OE1 | GLU | A | 890 | −20.989 | 40.647 | −13.299 | 1.00 | 40.49 |
| ATOM | 1439 | OE2 | GLU | A | 890 | −22.664 | 39.326 | −12.650 | 1.00 | 42.65 |
| ATOM | 1440 | N | CYS | A | 891 | −19.059 | 40.832 | −9.361 | 1.00 | 55.67 |
| ATOM | 1441 | CA | CYS | A | 891 | −18.720 | 40.296 | −8.052 | 1.00 | 56.74 |
| ATOM | 1442 | C | CYS | A | 891 | −17.300 | 40.649 | −7.605 | 1.00 | 57.48 |
| ATOM | 1443 | O | CYS | A | 891 | −16.868 | 40.297 | −6.508 | 1.00 | 55.50 |
| ATOM | 1444 | CB | CYS | A | 891 | −18.956 | 38.781 | −8.036 | 1.00 | 57.62 |
| ATOM | 1445 | SG | CYS | A | 891 | −20.729 | 38.333 | −7.970 | 1.00 | 57.17 |
| ATOM | 1446 | N | ILE | A | 892 | −16.576 | 41.362 | −8.458 | 1.00 | 58.80 |
| ATOM | 1447 | CA | ILE | A | 892 | −15.224 | 41.780 | −8.112 | 1.00 | 59.01 |
| ATOM | 1448 | C | ILE | A | 892 | −15.285 | 43.087 | −7.319 | 1.00 | 59.18 |
| ATOM | 1449 | O | ILE | A | 892 | −14.529 | 43.274 | −6.374 | 1.00 | 60.68 |
| ATOM | 1450 | CB | ILE | A | 892 | −14.357 | 42.018 | −9.369 | 1.00 | 57.56 |
| ATOM | 1451 | CG1 | ILE | A | 892 | −14.003 | 40.684 | −10.024 | 1.00 | 56.20 |
| ATOM | 1452 | CG2 | ILE | A | 892 | −13.104 | 42.792 | −8.996 | 1.00 | 54.39 |
| ATOM | 1453 | CD1 | ILE | A | 892 | −13.252 | 40.841 | −11.323 | 1.00 | 56.41 |
| ATOM | 1454 | N | HIS | A | 893 | −16.193 | 43.981 | −7.707 | 1.00 | 58.73 |
| ATOM | 1455 | CA | HIS | A | 893 | −16.333 | 45.274 | −7.046 | 1.00 | 57.13 |
| ATOM | 1456 | C | HIS | A | 893 | −17.139 | 45.236 | −5.757 | 1.00 | 55.23 |
| ATOM | 1457 | O | HIS | A | 893 | −16.951 | 46.075 | −4.879 | 1.00 | 55.68 |
| ATOM | 1458 | CB | HIS | A | 893 | −16.947 | 46.276 | −8.015 | 1.00 | 58.88 |
| ATOM | 1459 | CG | HIS | A | 893 | −16.103 | 46.532 | −9.225 | 1.00 | 62.09 |
| ATOM | 1460 | ND1 | HIS | A | 893 | −16.555 | 46.318 | −10.510 | 1.00 | 62.92 |
| ATOM | 1461 | CD2 | HIS | A | 893 | −14.825 | 46.969 | −9.344 | 1.00 | 63.01 |
| ATOM | 1462 | CE1 | HIS | A | 893 | −15.593 | 46.609 | −11.368 | 1.00 | 63.20 |
| ATOM | 1463 | NE2 | HIS | A | 893 | −14.532 | 47.006 | −10.687 | 1.00 | 63.80 |
| ATOM | 1464 | N | TYR | A | 894 | −18.031 | 44.258 | −5.652 | 1.00 | 53.00 |
| ATOM | 1465 | CA | TYR | A | 894 | −18.887 | 44.072 | −4.477 | 1.00 | 51.14 |
| ATOM | 1466 | C | TYR | A | 894 | −19.364 | 42.639 | −4.549 | 1.00 | 49.31 |
| ATOM | 1467 | O | TYR | A | 894 | −19.093 | 41.949 | −5.518 | 1.00 | 50.31 |
| ATOM | 1468 | CB | TYR | A | 894 | −20.135 | 44.959 | −4.542 | 1.00 | 52.50 |
| ATOM | 1469 | CG | TYR | A | 894 | −19.895 | 46.444 | −4.526 | 1.00 | 53.58 |
| ATOM | 1470 | CD1 | TYR | A | 894 | −19.719 | 47.126 | −3.327 | 1.00 | 54.62 |
| ATOM | 1471 | CD2 | TYR | A | 894 | −19.832 | 47.167 | −5.716 | 1.00 | 52.84 |
| ATOM | 1472 | CE1 | TYR | A | 894 | −19.483 | 48.491 | −3.313 | 1.00 | 57.08 |
| ATOM | 1473 | CE2 | TYR | A | 894 | −19.594 | 48.529 | −5.718 | 1.00 | 54.76 |
| ATOM | 1474 | CZ | TYR | A | 894 | −19.417 | 49.190 | −4.515 | 1.00 | 57.74 |
| ATOM | 1475 | OH | TYR | A | 894 | −19.147 | 50.546 | −4.505 | 1.00 | 59.64 |
| ATOM | 1476 | N | ARG | A | 895 | −20.087 | 42.195 | −3.534 | 1.00 | 48.76 |
| ATOM | 1477 | CA | ARG | A | 895 | −20.624 | 40.845 | −3.535 | 1.00 | 50.39 |
| ATOM | 1478 | C | ARG | A | 895 | −22.022 | 40.867 | −4.152 | 1.00 | 50.91 |
| ATOM | 1479 | O | ARG | A | 895 | −23.002 | 41.194 | −3.486 | 1.00 | 51.94 |
| ATOM | 1480 | CB | ARG | A | 895 | −20.712 | 40.281 | −2.112 | 1.00 | 49.62 |
| ATOM | 1481 | CG | ARG | A | 895 | −19.382 | 39.914 | −1.498 | 1.00 | 49.55 |
| ATOM | 1482 | CD | ARG | A | 895 | −19.526 | 38.808 | −0.460 | 1.00 | 49.12 |
| ATOM | 1483 | NE | ARG | A | 895 | −18.216 | 38.395 | 0.034 | 1.00 | 53.76 |
| ATOM | 1484 | CZ | ARG | A | 895 | −17.475 | 39.124 | 0.866 | 1.00 | 56.50 |
| ATOM | 1485 | NH1 | ARG | A | 895 | −16.284 | 38.688 | 1.268 | 1.00 | 52.02 |
| ATOM | 1486 | NH2 | ARG | A | 895 | −17.940 | 40.288 | 1.316 | 1.00 | 57.79 |
| ATOM | 1487 | N | ALA | A | 896 | −22.109 | 40.534 | −5.432 | 1.00 | 51.10 |
| ATOM | 1488 | CA | ALA | A | 896 | −23.392 | 40.505 | −6.122 | 1.00 | 49.65 |
| ATOM | 1489 | C | ALA | A | 896 | −23.571 | 39.101 | −6.670 | 1.00 | 47.43 |
| ATOM | 1490 | O | ALA | A | 896 | −23.849 | 38.925 | −7.843 | 1.00 | 46.98 |
| ATOM | 1491 | CB | ALA | A | 896 | −23.405 | 41.530 | −7.266 | 1.00 | 49.62 |
| ATOM | 1492 | N | PHE | A | 897 | −23.417 | 38.098 | −5.816 | 1.00 | 46.40 |
| ATOM | 1493 | CA | PHE | A | 897 | −23.540 | 36.721 | −6.276 | 1.00 | 46.89 |
| ATOM | 1494 | C | PHE | A | 897 | −24.943 | 36.276 | −6.689 | 1.00 | 45.30 |
| ATOM | 1495 | O | PHE | A | 897 | −25.889 | 36.296 | −5.902 | 1.00 | 44.45 |
| ATOM | 1496 | CB | PHE | A | 897 | −22.965 | 35.759 | −5.231 | 1.00 | 47.33 |
| ATOM | 1497 | CG | PHE | A | 897 | −21.487 | 35.887 | −5.058 | 1.00 | 48.54 |
| ATOM | 1498 | CD1 | PHE | A | 897 | −20.952 | 36.469 | −3.909 | 1.00 | 51.22 |
| ATOM | 1499 | CD2 | PHE | A | 897 | −20.622 | 35.458 | −6.061 | 1.00 | 49.59 |
| ATOM | 1500 | CE1 | PHE | A | 897 | −19.567 | 36.627 | −3.764 | 1.00 | 50.13 |
| ATOM | 1501 | CE2 | PHE | A | 897 | −19.239 | 35.607 | −5.932 | 1.00 | 49.10 |
| ATOM | 1502 | CZ | PHE | A | 897 | −18.709 | 36.194 | −4.780 | 1.00 | 50.78 |
| ATOM | 1503 | N | THR | A | 898 | −25.055 | 35.867 | −7.946 | 1.00 | 44.73 |
| ATOM | 1504 | CA | THR | A | 898 | −26.324 | 35.413 | −8.499 | 1.00 | 44.26 |
| ATOM | 1505 | C | THR | A | 898 | −26.085 | 34.245 | −9.422 | 1.00 | 43.33 |
| ATOM | 1506 | O | THR | A | 898 | −24.942 | 33.943 | −9.774 | 1.00 | 42.13 |
| ATOM | 1507 | CB | THR | A | 898 | −27.000 | 36.497 | −9.356 | 1.00 | 44.01 |
| ATOM | 1508 | OG1 | THR | A | 898 | −26.211 | 36.730 | −10.529 | 1.00 | 43.71 |
| ATOM | 1509 | CG2 | THR | A | 898 | −27.148 | 37.786 | −8.571 | 1.00 | 44.17 |
| ATOM | 1510 | N | HIS | A | 899 | −27.171 | 33.595 | −9.821 | 1.00 | 41.98 |
| ATOM | 1511 | CA | HIS | A | 899 | −27.065 | 32.487 | −10.748 | 1.00 | 42.31 |
| ATOM | 1512 | C | HIS | A | 899 | −26.317 | 32.987 | −11.982 | 1.00 | 41.21 |
| ATOM | 1513 | O | HIS | A | 899 | −25.520 | 32.255 | −12.567 | 1.00 | 42.33 |
| ATOM | 1514 | CB | HIS | A | 899 | −28.457 | 31.985 | −11.113 | 1.00 | 43.29 |
| ATOM | 1515 | CG | HIS | A | 899 | −29.246 | 31.542 | −9.927 | 1.00 | 45.33 |
| ATOM | 1516 | ND1 | HIS | A | 899 | −28.795 | 30.571 | −9.057 | 1.00 | 47.28 |
| ATOM | 1517 | CD2 | HIS | A | 899 | −30.433 | 31.968 | −9.435 | 1.00 | 46.03 |
| ATOM | 1518 | CE1 | HIS | A | 899 | −29.667 | 30.418 | −8.078 | 1.00 | 46.75 |
| ATOM | 1519 | NE2 | HIS | A | 899 | −30.670 | 31.255 | −8.283 | 1.00 | 49.17 |
| ATOM | 1520 | N | GLN | A | 900 | −26.540 | 34.243 | −12.358 | 1.00 | 39.59 |
| ATOM | 1521 | CA | GLN | A | 900 | −25.842 | 34.776 | −13.521 | 1.00 | 40.74 |
| ATOM | 1522 | C | GLN | A | 900 | −24.325 | 34.960 | −13.315 | 1.00 | 39.29 |
| ATOM | 1523 | O | GLN | A | 900 | −23.565 | 34.901 | −14.276 | 1.00 | 37.94 |
| ATOM | 1524 | CB | GLN | A | 900 | −26.497 | 36.078 | −14.002 | 1.00 | 40.33 |
| ATOM | 1525 | CG | GLN | A | 900 | −27.937 | 35.893 | −14.495 | 1.00 | 44.02 |
| ATOM | 1526 | CD | GLN | A | 900 | −28.125 | 34.695 | −15.443 | 1.00 | 47.54 |
| ATOM | 1527 | OE1 | GLN | A | 900 | −28.722 | 33.677 | −15.084 | 1.00 | 51.69 |
| ATOM | 1528 | NE2 | GLN | A | 900 | −27.610 | 34.822 | −16.660 | 1.00 | 48.77 |
| ATOM | 1529 | N | SER | A | 901 | −23.869 | 35.171 | −12.084 | 1.00 | 38.33 |
| ATOM | 1530 | CA | SER | A | 901 | −22.428 | 35.303 | −11.870 | 1.00 | 37.53 |
| ATOM | 1531 | C | SER | A | 901 | −21.856 | 33.895 | −12.004 | 1.00 | 40.00 |
| ATOM | 1532 | O | SER | A | 901 | −20.757 | 33.702 | −12.542 | 1.00 | 39.46 |
| ATOM | 1533 | CB | SER | A | 901 | −22.116 | 35.891 | −10.492 | 1.00 | 36.74 |
| ATOM | 1534 | OG | SER | A | 901 | −22.775 | 35.195 | −9.451 | 1.00 | 40.58 |
| ATOM | 1535 | N | ASP | A | 902 | −22.626 | 32.913 | −11.527 | 1.00 | 39.02 |
| ATOM | 1536 | CA | ASP | A | 902 | −22.247 | 31.511 | −11.623 | 1.00 | 37.25 |
| ATOM | 1537 | C | ASP | A | 902 | −22.061 | 31.163 | −13.110 | 1.00 | 37.82 |
| ATOM | 1538 | O | ASP | A | 902 | −21.139 | 30.436 | −13.480 | 1.00 | 37.20 |
| ATOM | 1539 | CB | ASP | A | 902 | −23.337 | 30.624 | −11.005 | 1.00 | 38.66 |
| ATOM | 1540 | CG | ASP | A | 902 | −23.065 | 30.279 | −9.533 | 1.00 | 40.83 |
| ATOM | 1541 | OD1 | ASP | A | 902 | −22.241 | 30.958 | −8.896 | 1.00 | 41.45 |
| ATOM | 1542 | OD2 | ASP | A | 902 | −23.682 | 29.326 | −9.004 | 1.00 | 42.94 |
| ATOM | 1543 | N | VAL | A | 903 | −22.929 | 31.694 | −13.966 | 1.00 | 36.71 |
| ATOM | 1544 | CA | VAL | A | 903 | −22.819 | 31.414 | −15.390 | 1.00 | 37.25 |
| ATOM | 1545 | C | VAL | A | 903 | −21.473 | 31.890 | −15.932 | 1.00 | 37.89 |
| ATOM | 1546 | O | VAL | A | 903 | −20.866 | 31.234 | −16.788 | 1.00 | 35.97 |
| ATOM | 1547 | CB | VAL | A | 903 | −23.960 | 32.075 | −16.191 | 1.00 | 37.16 |
| ATOM | 1548 | CG1 | VAL | A | 903 | −23.578 | 32.159 | −17.657 | 1.00 | 35.90 |
| ATOM | 1549 | CG2 | VAL | A | 903 | −25.232 | 31.264 | −16.043 | 1.00 | 34.45 |
| ATOM | 1550 | N | TRP | A | 904 | −21.013 | 33.035 | −15.435 | 1.00 | 38.74 |
| ATOM | 1551 | CA | TRP | A | 904 | −19.722 | 33.574 | −15.845 | 1.00 | 37.48 |
| ATOM | 1552 | C | TRP | A | 904 | −18.660 | 32.523 | −15.494 | 1.00 | 35.96 |
| ATOM | 1553 | O | TRP | A | 904 | −17.845 | 32.134 | −16.323 | 1.00 | 33.79 |
| ATOM | 1554 | CB | TRP | A | 904 | −19.426 | 34.875 | −15.084 | 1.00 | 39.00 |
| ATOM | 1555 | CG | TRP | A | 904 | −18.094 | 35.469 | −15.435 | 1.00 | 41.83 |
| ATOM | 1556 | CD1 | TRP | A | 904 | −16.870 | 34.876 | −15.304 | 1.00 | 40.23 |
| ATOM | 1557 | CD2 | TRP | A | 904 | −17.858 | 36.746 | −16.041 | 1.00 | 43.43 |
| ATOM | 1558 | NE1 | TRP | A | 904 | −15.891 | 35.695 | −15.799 | 1.00 | 41.66 |
| ATOM | 1559 | CE2 | TRP | A | 904 | −16.464 | 36.853 | −16.255 | 1.00 | 44.01 |
| ATOM | 1560 | CE3 | TRP | A | 904 | −18.688 | 37.810 | −16.425 | 1.00 | 44.89 |
| ATOM | 1561 | CZ2 | TRP | A | 904 | −15.876 | 37.989 | −16.840 | 1.00 | 45.74 |
| ATOM | 1562 | CZ3 | TRP | A | 904 | −18.105 | 38.943 | −17.010 | 1.00 | 46.53 |
| ATOM | 1563 | CH2 | TRP | A | 904 | −16.710 | 39.020 | −17.210 | 1.00 | 46.91 |
| ATOM | 1564 | N | SER | A | 905 | −18.681 | 32.070 | −14.249 | 1.00 | 34.90 |
| ATOM | 1565 | CA | SER | A | 905 | −17.731 | 31.078 | −13.794 | 1.00 | 38.24 |
| ATOM | 1566 | C | SER | A | 905 | −17.793 | 29.865 | −14.726 | 1.00 | 38.83 |
| ATOM | 1567 | O | SER | A | 905 | −16.773 | 29.409 | −15.250 | 1.00 | 41.08 |
| ATOM | 1568 | CB | SER | A | 905 | −18.059 | 30.690 | −12.354 | 1.00 | 39.02 |
| ATOM | 1569 | OG | SER | A | 905 | −18.186 | 31.852 | −11.545 | 1.00 | 39.18 |
| ATOM | 1570 | N | TYR | A | 906 | −19.000 | 29.361 | −14.935 | 1.00 | 38.14 |
| ATOM | 1571 | CA | TYR | A | 906 | −19.231 | 28.233 | −15.825 | 1.00 | 36.99 |
| ATOM | 1572 | C | TYR | A | 906 | −18.456 | 28.460 | −17.123 | 1.00 | 37.89 |
| ATOM | 1573 | O | TYR | A | 906 | −17.770 | 27.574 | −17.620 | 1.00 | 37.22 |
| ATOM | 1574 | CB | TYR | A | 906 | −20.727 | 28.127 | −16.130 | 1.00 | 37.09 |
| ATOM | 1575 | CG | TYR | A | 906 | −21.060 | 27.090 | −17.169 | 1.00 | 36.26 |
| ATOM | 1576 | CD1 | TYR | A | 906 | −21.299 | 25.767 | −16.809 | 1.00 | 33.65 |
| ATOM | 1577 | CD2 | TYR | A | 906 | −21.056 | 27.421 | −18.522 | 1.00 | 35.93 |
| ATOM | 1578 | CE1 | TYR | A | 906 | −21.513 | 24.800 | −17.765 | 1.00 | 35.24 |
| ATOM | 1579 | CE2 | TYR | A | 906 | −21.269 | 26.461 | −19.488 | 1.00 | 36.62 |
| ATOM | 1580 | CZ | TYR | A | 906 | −21.494 | 25.147 | −19.108 | 1.00 | 35.06 |
| ATOM | 1581 | OH | TYR | A | 906 | −21.659 | 24.187 | −20.077 | 1.00 | 32.16 |
| ATOM | 1582 | N | GLY | A | 907 | −18.585 | 29.658 | −17.677 | 1.00 | 38.18 |
| ATOM | 1583 | CA | GLY | A | 907 | −17.866 | 29.974 | −18.890 | 1.00 | 37.34 |
| ATOM | 1584 | C | GLY | A | 907 | −16.368 | 29.758 | −18.734 | 1.00 | 39.60 |
| ATOM | 1585 | O | GLY | A | 907 | −15.749 | 29.097 | −19.583 | 1.00 | 39.30 |
| ATOM | 1586 | N | VAL | A | 908 | −15.775 | 30.294 | −17.662 | 1.00 | 38.75 |
| ATOM | 1587 | CA | VAL | A | 908 | −14.329 | 30.144 | −17.460 | 1.00 | 37.84 |
| ATOM | 1588 | C | VAL | A | 908 | −14.006 | 28.678 | −17.254 | 1.00 | 38.99 |
| ATOM | 1589 | O | VAL | A | 908 | −12.948 | 28.189 | −17.669 | 1.00 | 38.75 |
| ATOM | 1590 | CB | VAL | A | 908 | −13.810 | 30.938 | −16.233 | 1.00 | 38.62 |
| ATOM | 1591 | CG1 | VAL | A | 908 | −12.282 | 30.912 | −16.209 | 1.00 | 35.04 |
| ATOM | 1592 | CG2 | VAL | A | 908 | −14.315 | 32.372 | −16.274 | 1.00 | 33.97 |
| ATOM | 1593 | N | THR | A | 909 | −14.939 | 27.974 | −16.627 | 1.00 | 38.38 |
| ATOM | 1594 | CA | THR | A | 909 | −14.764 | 26.552 | −16.375 | 1.00 | 37.41 |
| ATOM | 1595 | C | THR | A | 909 | −14.710 | 25.688 | −17.629 | 1.00 | 37.98 |
| ATOM | 1596 | O | THR | A | 909 | −13.837 | 24.837 | −17.747 | 1.00 | 39.71 |
| ATOM | 1597 | CB | THR | A | 909 | −15.861 | 26.011 | −15.472 | 1.00 | 34.91 |
| ATOM | 1598 | OG1 | THR | A | 909 | −15.866 | 26.753 | −14.253 | 1.00 | 33.91 |
| ATOM | 1599 | CG2 | THR | A | 909 | −15.610 | 24.541 | −15.161 | 1.00 | 34.30 |
| ATOM | 1600 | N | ILE | A | 910 | −15.629 | 25.877 | −18.569 | 1.00 | 38.77 |
| ATOM | 1601 | CA | ILE | A | 910 | −15.566 | 25.043 | −19.765 | 1.00 | 41.06 |
| ATOM | 1602 | C | ILE | A | 910 | −14.357 | 25.445 | −20.598 | 1.00 | 42.01 |
| ATOM | 1603 | O | ILE | A | 910 | −13.887 | 24.676 | −21.444 | 1.00 | 39.07 |
| ATOM | 1604 | CB | ILE | A | 910 | −16.841 | 25.128 | −20.628 | 1.00 | 40.40 |
| ATOM | 1605 | CG1 | ILE | A | 910 | −17.064 | 26.549 | −21.124 | 1.00 | 42.79 |
| ATOM | 1606 | CG2 | ILE | A | 910 | −18.022 | 24.642 | −19.822 | 1.00 | 43.12 |
| ATOM | 1607 | CD1 | ILE | A | 910 | −18.166 | 26.673 | −22.162 | 1.00 | 43.99 |
| ATOM | 1608 | N | TRP | A | 911 | −13.857 | 26.656 | −20.347 | 1.00 | 42.81 |
| ATOM | 1609 | CA | TRP | A | 911 | −12.689 | 27.146 | −21.052 | 1.00 | 43.02 |
| ATOM | 1610 | C | TRP | A | 911 | −11.482 | 26.324 | −20.573 | 1.00 | 44.92 |
| ATOM | 1611 | O | TRP | A | 911 | −10.651 | 25.916 | −21.384 | 1.00 | 45.96 |
| ATOM | 1612 | CB | TRP | A | 911 | −12.481 | 28.625 | −20.759 | 1.00 | 45.39 |
| ATOM | 1613 | CG | TRP | A | 911 | −11.281 | 29.215 | −21.435 | 1.00 | 49.15 |
| ATOM | 1614 | CD1 | TRP | A | 911 | −11.238 | 29.822 | −22.660 | 1.00 | 50.73 |
| ATOM | 1615 | CD2 | TRP | A | 911 | −9.940 | 29.249 | −20.924 | 1.00 | 50.38 |
| ATOM | 1616 | NE1 | TRP | A | 911 | −9.954 | 30.237 | −22.942 | 1.00 | 49.91 |
| ATOM | 1617 | CE2 | TRP | A | 911 | −9.138 | 29.897 | −21.892 | 1.00 | 51.44 |
| ATOM | 1618 | CE3 | TRP | A | 911 | −9.339 | 28.794 | −19.742 | 1.00 | 49.30 |
| ATOM | 1619 | CZ2 | TRP | A | 911 | −7.763 | 30.101 | −21.712 | 1.00 | 48.85 |
| ATOM | 1620 | CZ3 | TRP | A | 911 | −7.973 | 28.997 | −19.565 | 1.00 | 49.14 |
| ATOM | 1621 | CH2 | TRP | A | 911 | −7.203 | 29.646 | −20.548 | 1.00 | 48.84 |
| ATOM | 1622 | N | GLU | A | 912 | −11.388 | 26.066 | −19.268 | 1.00 | 44.35 |
| ATOM | 1623 | CA | GLU | A | 912 | −10.277 | 25.273 | −18.755 | 1.00 | 45.12 |
| ATOM | 1624 | C | GLU | A | 912 | −10.299 | 23.922 | −19.468 | 1.00 | 46.73 |
| ATOM | 1625 | O | GLU | A | 912 | −9.254 | 23.400 | −19.879 | 1.00 | 46.06 |
| ATOM | 1626 | CB | GLU | A | 912 | −10.404 | 25.030 | −17.247 | 1.00 | 44.17 |
| ATOM | 1627 | CG | GLU | A | 912 | −10.530 | 26.265 | −16.378 | 1.00 | 45.56 |
| ATOM | 1628 | CD | GLU | A | 912 | −10.733 | 25.919 | −14.899 | 1.00 | 47.51 |
| ATOM | 1629 | OE1 | GLU | A | 912 | −9.766 | 25.474 | −14.243 | 1.00 | 48.20 |
| ATOM | 1630 | OE2 | GLU | A | 912 | −11.866 | 26.080 | −14.389 | 1.00 | 46.43 |
| ATOM | 1631 | N | LEU | A | 913 | −11.502 | 23.371 | −19.622 | 1.00 | 46.87 |
| ATOM | 1632 | CA | LEU | A | 913 | −11.691 | 22.072 | −20.270 | 1.00 | 45.49 |
| ATOM | 1633 | C | LEU | A | 913 | −11.261 | 22.067 | −21.724 | 1.00 | 46.82 |
| ATOM | 1634 | O | LEU | A | 913 | −10.522 | 21.179 | −22.150 | 1.00 | 48.03 |
| ATOM | 1635 | CB | LEU | A | 913 | −13.153 | 21.643 | −20.170 | 1.00 | 43.18 |
| ATOM | 1636 | CG | LEU | A | 913 | −13.750 | 21.713 | −18.764 | 1.00 | 43.01 |
| ATOM | 1637 | CD1 | LEU | A | 913 | −15.174 | 21.210 | −18.804 | 1.00 | 43.13 |
| ATOM | 1638 | CD2 | LEU | A | 913 | −12.916 | 20.880 | −17.790 | 1.00 | 40.71 |
| ATOM | 1639 | N | MET | A | 914 | −11.726 | 23.052 | −22.487 | 1.00 | 48.63 |
| ATOM | 1640 | CA | MET | A | 914 | −11.374 | 23.148 | −23.903 | 1.00 | 50.01 |
| ATOM | 1641 | C | MET | A | 914 | −9.874 | 23.324 | −24.112 | 1.00 | 50.22 |
| ATOM | 1642 | O | MET | A | 914 | −9.363 | 23.007 | −25.182 | 1.00 | 50.31 |
| ATOM | 1643 | CB | MET | A | 914 | −12.108 | 24.309 | −24.576 | 1.00 | 51.66 |
| ATOM | 1644 | CG | MET | A | 914 | −13.611 | 24.142 | −24.660 | 1.00 | 54.12 |
| ATOM | 1645 | SD | MET | A | 914 | −14.103 | 22.502 | −25.184 | 1.00 | 55.78 |
| ATOM | 1646 | CE | MET | A | 914 | −15.162 | 22.056 | −23.847 | 1.00 | 54.82 |
| ATOM | 1647 | N | THR | A | 915 | −9.171 | 23.846 | −23.108 | 1.00 | 49.71 |
| ATOM | 1648 | CA | THR | A | 915 | −7.727 | 24.013 | −23.230 | 1.00 | 49.12 |
| ATOM | 1649 | C | THR | A | 915 | −7.024 | 22.881 | −22.487 | 1.00 | 50.59 |
| ATOM | 1650 | O | THR | A | 915 | −5.851 | 22.985 | −22.133 | 1.00 | 50.68 |
| ATOM | 1651 | CB | THR | A | 915 | −7.240 | 25.361 | −22.663 | 1.00 | 47.98 |
| ATOM | 1652 | OG1 | THR | A | 915 | −7.532 | 25.426 | −21.260 | 1.00 | 49.13 |
| ATOM | 1653 | CG2 | THR | A | 915 | −7.912 | 26.526 | −23.392 | 1.00 | 45.99 |
| ATOM | 1654 | N | PHE | A | 916 | −7.756 | 21.797 | −22.250 | 1.00 | 51.37 |
| ATOM | 1655 | CA | PHE | A | 916 | −7.208 | 20.632 | −21.571 | 1.00 | 51.95 |
| ATOM | 1656 | C | PHE | A | 916 | −6.429 | 21.033 | −20.317 | 1.00 | 53.29 |
| ATOM | 1657 | O | PHE | A | 916 | −5.255 | 20.692 | −20.160 | 1.00 | 54.44 |
| ATOM | 1658 | CB | PHE | A | 916 | −6.299 | 19.861 | −22.538 | 1.00 | 51.51 |
| ATOM | 1659 | CG | PHE | A | 916 | −7.028 | 19.251 | −23.718 | 1.00 | 50.02 |
| ATOM | 1660 | CD1 | PHE | A | 916 | −7.835 | 18.124 | −23.555 | 1.00 | 50.49 |
| ATOM | 1661 | CD2 | PHE | A | 916 | −6.896 | 19.792 | −24.988 | 1.00 | 47.97 |
| ATOM | 1662 | CE1 | PHE | A | 916 | −8.493 | 17.548 | −24.641 | 1.00 | 49.30 |
| ATOM | 1663 | CE2 | PHE | A | 916 | −7.548 | 19.225 | −26.073 | 1.00 | 49.17 |
| ATOM | 1664 | CZ | PHE | A | 916 | −8.347 | 18.102 | −25.902 | 1.00 | 49.74 |
| ATOM | 1665 | N | GLY | A | 917 | −7.090 | 21.774 | −19.433 | 1.00 | 54.12 |
| ATOM | 1666 | CA | GLY | A | 917 | −6.455 | 22.207 | −18.204 | 1.00 | 53.22 |
| ATOM | 1667 | C | GLY | A | 917 | −5.687 | 23.516 | −18.292 | 1.00 | 53.97 |
| ATOM | 1668 | O | GLY | A | 917 | −4.769 | 23.745 | −17.503 | 1.00 | 56.50 |
| ATOM | 1669 | N | GLY | A | 918 | −6.046 | 24.385 | −19.229 | 1.00 | 51.72 |
| ATOM | 1670 | CA | GLY | A | 918 | −5.339 | 25.647 | −19.336 | 1.00 | 52.43 |
| ATOM | 1671 | C | GLY | A | 918 | −5.579 | 26.544 | −18.135 | 1.00 | 54.51 |
| ATOM | 1672 | O | GLY | A | 918 | −6.514 | 26.323 | −17.366 | 1.00 | 55.43 |
| ATOM | 1673 | N | LYS | A | 919 | −4.742 | 27.561 | −17.964 | 1.00 | 55.17 |
| ATOM | 1674 | CA | LYS | A | 919 | −4.902 | 28.472 | −16.838 | 1.00 | 55.34 |
| ATOM | 1675 | C | LYS | A | 919 | −5.396 | 29.852 | −17.268 | 1.00 | 55.34 |
| ATOM | 1676 | O | LYS | A | 919 | −4.765 | 30.528 | −18.082 | 1.00 | 54.75 |
| ATOM | 1677 | CB | LYS | A | 919 | −3.584 | 28.608 | −16.082 | 1.00 | 57.86 |
| ATOM | 1678 | CG | LYS | A | 919 | −3.045 | 27.296 | −15.545 | 1.00 | 61.37 |
| ATOM | 1679 | CD | LYS | A | 919 | −1.874 | 27.539 | −14.608 | 1.00 | 65.00 |
| ATOM | 1680 | CE | LYS | A | 919 | −2.319 | 28.318 | −13.372 | 1.00 | 68.24 |
| ATOM | 1681 | NZ | LYS | A | 919 | −1.174 | 28.718 | −12.493 | 1.00 | 70.07 |
| ATOM | 1682 | N | PRO | A | 920 | −6.539 | 30.287 | −16.714 | 1.00 | 54.70 |
| ATOM | 1683 | CA | PRO | A | 920 | −7.199 | 31.571 | −16.977 | 1.00 | 54.08 |
| ATOM | 1684 | C | PRO | A | 920 | −6.248 | 32.746 | −16.833 | 1.00 | 53.70 |
| ATOM | 1685 | O | PRO | A | 920 | −5.558 | 32.860 | −15.828 | 1.00 | 54.60 |
| ATOM | 1686 | CB | PRO | A | 920 | −8.292 | 31.617 | −15.920 | 1.00 | 53.99 |
| ATOM | 1687 | CG | PRO | A | 920 | −8.618 | 30.190 | −15.718 | 1.00 | 56.24 |
| ATOM | 1688 | CD | PRO | A | 920 | −7.269 | 29.535 | −15.683 | 1.00 | 54.76 |
| ATOM | 1689 | N | TYR | A | 921 | −6.232 | 33.624 | −17.828 | 1.00 | 53.96 |
| ATOM | 1690 | CA | TYR | A | 921 | −5.362 | 34.794 | −17.810 | 1.00 | 56.43 |
| ATOM | 1691 | C | TYR | A | 921 | −3.992 | 34.423 | −17.242 | 1.00 | 59.51 |
| ATOM | 1692 | O | TYR | A | 921 | −3.519 | 35.029 | −16.276 | 1.00 | 59.77 |
| ATOM | 1693 | CB | TYR | A | 921 | −5.996 | 35.904 | −16.972 | 1.00 | 53.93 |
| ATOM | 1694 | CG | TYR | A | 921 | −7.477 | 36.069 | −17.217 | 1.00 | 50.28 |
| ATOM | 1695 | CD1 | TYR | A | 921 | −8.406 | 35.456 | −16.386 | 1.00 | 48.21 |
| ATOM | 1696 | CD2 | TYR | A | 921 | −7.947 | 36.839 | −18.281 | 1.00 | 47.29 |
| ATOM | 1697 | CE1 | TYR | A | 921 | −9.760 | 35.604 | −16.597 | 1.00 | 47.95 |
| ATOM | 1698 | CE2 | TYR | A | 921 | −9.301 | 36.993 | −18.503 | 1.00 | 46.64 |
| ATOM | 1699 | CZ | TYR | A | 921 | −10.204 | 36.373 | −17.654 | 1.00 | 48.17 |
| ATOM | 1700 | OH | TYR | A | 921 | −11.552 | 36.534 | −17.841 | 1.00 | 46.47 |
| ATOM | 1701 | N | ASP | A | 922 | −3.369 | 33.424 | −17.859 | 1.00 | 62.28 |
| ATOM | 1702 | CA | ASP | A | 922 | −2.070 | 32.918 | −17.437 | 1.00 | 65.68 |
| ATOM | 1703 | C | ASP | A | 922 | −0.996 | 33.989 | −17.291 | 1.00 | 67.52 |
| ATOM | 1704 | O | ASP | A | 922 | −0.560 | 34.580 | −18.281 | 1.00 | 68.07 |
| ATOM | 1705 | CB | ASP | A | 922 | −1.573 | 31.869 | −18.427 | 1.00 | 66.85 |
| ATOM | 1706 | CG | ASP | A | 922 | −0.510 | 30.977 | −17.830 | 1.00 | 69.00 |
| ATOM | 1707 | OD1 | ASP | A | 922 | 0.262 | 31.468 | −16.972 | 1.00 | 68.93 |
| ATOM | 1708 | OD2 | ASP | A | 922 | −0.445 | 29.790 | −18.223 | 1.00 | 69.13 |
| ATOM | 1709 | N | GLY | A | 923 | −0.564 | 34.221 | −16.055 | 1.00 | 69.33 |
| ATOM | 1710 | CA | GLY | A | 923 | 0.468 | 35.212 | −15.800 | 1.00 | 70.77 |
| ATOM | 1711 | C | GLY | A | 923 | −0.022 | 36.553 | −15.281 | 1.00 | 71.67 |
| ATOM | 1712 | O | GLY | A | 923 | 0.477 | 37.054 | −14.270 | 1.00 | 72.97 |
| ATOM | 1713 | N | ILE | A | 924 | −0.995 | 37.135 | −15.973 | 1.00 | 71.05 |
| ATOM | 1714 | CA | ILE | A | 924 | −1.548 | 38.428 | −15.592 | 1.00 | 70.63 |
| ATOM | 1715 | C | ILE | A | 924 | −1.781 | 38.545 | −14.082 | 1.00 | 70.88 |
| ATOM | 1716 | O | ILE | A | 924 | −2.336 | 37.640 | −13.457 | 1.00 | 71.74 |
| ATOM | 1717 | CB | ILE | A | 924 | −2.868 | 38.690 | −16.349 | 1.00 | 70.59 |
| ATOM | 1718 | CG1 | ILE | A | 924 | −2.652 | 38.433 | −17.848 | 1.00 | 70.80 |
| ATOM | 1719 | CG2 | ILE | A | 924 | −3.328 | 40.127 | −16.124 | 1.00 | 68.25 |
| ATOM | 1720 | CD1 | ILE | A | 924 | −3.930 | 38.442 | −18.696 | 1.00 | 72.84 |
| ATOM | 1721 | N | PRO | A | 925 | −1.340 | 39.668 | −13.480 | 1.00 | 70.93 |
| ATOM | 1722 | CA | PRO | A | 925 | −1.446 | 40.010 | −12.055 | 1.00 | 70.60 |
| ATOM | 1723 | C | PRO | A | 925 | −2.884 | 40.065 | −11.539 | 1.00 | 69.81 |
| ATOM | 1724 | O | PRO | A | 925 | −3.655 | 40.937 | −11.933 | 1.00 | 67.59 |
| ATOM | 1725 | CB | PRO | A | 925 | −0.779 | 41.384 | −11.980 | 1.00 | 71.26 |
| ATOM | 1726 | CG | PRO | A | 925 | 0.207 | 41.347 | −13.091 | 1.00 | 70.82 |
| ATOM | 1727 | CD | PRO | A | 925 | −0.599 | 40.721 | −14.196 | 1.00 | 70.92 |
| ATOM | 1728 | N | THR | A | 926 | −3.221 | 39.149 | −10.638 | 1.00 | 69.20 |
| ATOM | 1729 | CA | THR | A | 926 | −4.559 | 39.071 | −10.061 | 1.00 | 70.01 |
| ATOM | 1730 | C | THR | A | 926 | −5.184 | 40.429 | −9.730 | 1.00 | 70.43 |
| ATOM | 1731 | O | THR | A | 926 | −6.407 | 40.578 | −9.735 | 1.00 | 69.78 |
| ATOM | 1732 | CB | THR | A | 926 | −4.553 | 38.213 | −8.770 | 1.00 | 70.42 |
| ATOM | 1733 | OG1 | THR | A | 926 | −5.899 | 37.915 | −8.390 | 1.00 | 71.23 |
| ATOM | 1734 | CG2 | THR | A | 926 | −3.870 | 38.962 | −7.623 | 1.00 | 71.38 |
| ATOM | 1735 | N | ALA | A | 927 | −4.344 | 41.417 | −9.441 | 1.00 | 71.00 |
| ATOM | 1736 | CA | ALA | A | 927 | −4.830 | 42.746 | −9.095 | 1.00 | 70.88 |
| ATOM | 1737 | C | ALA | A | 927 | −5.223 | 43.542 | −10.336 | 1.00 | 71.19 |
| ATOM | 1738 | O | ALA | A | 927 | −5.820 | 44.615 | −10.237 | 1.00 | 72.20 |
| ATOM | 1739 | CB | ALA | A | 927 | −3.759 | 43.498 | −8.305 | 1.00 | 69.23 |
| ATOM | 1740 | N | GLU | A | 928 | −4.908 | 43.008 | −11.507 | 1.00 | 71.41 |
| ATOM | 1741 | CA | GLU | A | 928 | −5.209 | 43.709 | −12.747 | 1.00 | 71.50 |
| ATOM | 1742 | C | GLU | A | 928 | −6.429 | 43.196 | −13.494 | 1.00 | 68.69 |
| ATOM | 1743 | O | GLU | A | 928 | −6.968 | 43.892 | −14.355 | 1.00 | 68.39 |
| ATOM | 1744 | CB | GLU | A | 928 | −3.983 | 43.669 | −13.659 | 1.00 | 73.75 |
| ATOM | 1745 | CG | GLU | A | 928 | −2.754 | 44.252 | −12.996 | 1.00 | 78.75 |
| ATOM | 1746 | CD | GLU | A | 928 | −1.535 | 44.234 | −13.891 | 1.00 | 82.92 |
| ATOM | 1747 | OE1 | GLU | A | 928 | −0.439 | 44.579 | −13.390 | 1.00 | 85.58 |
| ATOM | 1748 | OE2 | GLU | A | 928 | −1.672 | 43.880 | −15.087 | 1.00 | 83.54 |
| ATOM | 1749 | N | ILE | A | 929 | −6.868 | 41.988 | −13.167 | 1.00 | 65.54 |
| ATOM | 1750 | CA | ILE | A | 929 | −8.027 | 41.414 | −13.831 | 1.00 | 63.43 |
| ATOM | 1751 | C | ILE | A | 929 | −9.198 | 42.407 | −13.900 | 1.00 | 62.00 |
| ATOM | 1752 | O | ILE | A | 929 | −9.742 | 42.666 | −14.973 | 1.00 | 58.78 |
| ATOM | 1753 | CB | ILE | A | 929 | −8.482 | 40.126 | −13.119 | 1.00 | 61.81 |
| ATOM | 1754 | CG1 | ILE | A | 929 | −7.336 | 39.111 | −13.105 | 1.00 | 61.74 |
| ATOM | 1755 | CG2 | ILE | A | 929 | −9.689 | 39.538 | −13.822 | 1.00 | 62.66 |
| ATOM | 1756 | CD1 | ILE | A | 929 | −6.847 | 38.691 | −14.478 | 1.00 | 59.86 |
| ATOM | 1757 | N | PRO | A | 930 | −9.584 | 42.991 | −12.755 | 1.00 | 61.97 |
| ATOM | 1758 | CA | PRO | A | 930 | −10.694 | 43.948 | −12.732 | 1.00 | 62.93 |
| ATOM | 1759 | C | PRO | A | 930 | −10.643 | 44.976 | −13.856 | 1.00 | 64.06 |
| ATOM | 1760 | O | PRO | A | 930 | −11.641 | 45.216 | −14.538 | 1.00 | 62.58 |
| ATOM | 1761 | CB | PRO | A | 930 | −10.560 | 44.593 | −11.358 | 1.00 | 61.80 |
| ATOM | 1762 | CG | PRO | A | 930 | −10.047 | 43.479 | −10.536 | 1.00 | 61.59 |
| ATOM | 1763 | CD | PRO | A | 930 | −8.984 | 42.865 | −11.417 | 1.00 | 60.70 |
| ATOM | 1764 | N | ASP | A | 931 | −9.476 | 45.580 | −14.046 | 1.00 | 66.03 |
| ATOM | 1765 | CA | ASP | A | 931 | −9.311 | 46.589 | −15.081 | 1.00 | 68.58 |
| ATOM | 1766 | C | ASP | A | 931 | −9.354 | 46.006 | −16.482 | 1.00 | 67.95 |
| ATOM | 1767 | O | ASP | A | 931 | −10.012 | 46.563 | −17.360 | 1.00 | 67.90 |
| ATOM | 1768 | CB | ASP | A | 931 | −8.007 | 47.353 | −14.866 | 1.00 | 73.60 |
| ATOM | 1769 | CG | ASP | A | 931 | −8.015 | 48.146 | −13.572 | 1.00 | 78.41 |
| ATOM | 1770 | OD1 | ASP | A | 931 | −8.154 | 47.523 | −12.485 | 1.00 | 79.78 |
| ATOM | 1771 | OD2 | ASP | A | 931 | −7.890 | 49.391 | −13.645 | 1.00 | 80.42 |
| ATOM | 1772 | N | LEU | A | 932 | −8.654 | 44.896 | −16.698 | 1.00 | 67.05 |
| ATOM | 1773 | CA | LEU | A | 932 | −8.660 | 44.252 | −18.007 | 1.00 | 66.68 |
| ATOM | 1774 | C | LEU | A | 932 | −10.110 | 44.019 | −18.434 | 1.00 | 67.26 |
| ATOM | 1775 | O | LEU | A | 932 | −10.551 | 44.492 | −19.484 | 1.00 | 67.71 |
| ATOM | 1776 | CB | LEU | A | 932 | −7.932 | 42.909 | −17.946 | 1.00 | 66.91 |
| ATOM | 1777 | CG | LEU | A | 932 | −6.415 | 42.931 | −17.754 | 1.00 | 68.66 |
| ATOM | 1778 | CD1 | LEU | A | 932 | −5.905 | 41.504 | −17.553 | 1.00 | 67.81 |
| ATOM | 1779 | CD2 | LEU | A | 932 | −5.753 | 43.581 | −18.977 | 1.00 | 68.04 |
| ATOM | 1780 | N | LEU | A | 933 | −10.849 | 43.297 | −17.599 | 1.00 | 66.10 |
| ATOM | 1781 | CA | LEU | A | 933 | −12.240 | 42.982 | −17.874 | 1.00 | 65.55 |
| ATOM | 1782 | C | LEU | A | 933 | −13.049 | 44.190 | −18.319 | 1.00 | 65.37 |
| ATOM | 1783 | O | LEU | A | 933 | −13.751 | 44.135 | −19.331 | 1.00 | 65.79 |
| ATOM | 1784 | CB | LEU | A | 933 | −12.878 | 42.355 | −16.638 | 1.00 | 64.86 |
| ATOM | 1785 | CG | LEU | A | 933 | −12.233 | 41.030 | −16.223 | 1.00 | 65.13 |
| ATOM | 1786 | CD1 | LEU | A | 933 | −12.696 | 40.652 | −14.828 | 1.00 | 65.75 |
| ATOM | 1787 | CD2 | LEU | A | 933 | −12.579 | 39.945 | −17.229 | 1.00 | 63.48 |
| ATOM | 1788 | N | GLU | A | 934 | −12.947 | 45.280 | −17.567 | 1.00 | 65.40 |
| ATOM | 1789 | CA | GLU | A | 934 | −13.687 | 46.497 | −17.885 | 1.00 | 65.09 |
| ATOM | 1790 | C | GLU | A | 934 | −13.162 | 47.174 | −19.132 | 1.00 | 63.61 |
| ATOM | 1791 | O | GLU | A | 934 | −13.890 | 47.907 | −19.797 | 1.00 | 61.44 |
| ATOM | 1792 | CB | GLU | A | 934 | −13.657 | 47.451 | −16.693 | 1.00 | 65.83 |
| ATOM | 1793 | CG | GLU | A | 934 | −14.514 | 46.926 | −15.558 | 1.00 | 71.06 |
| ATOM | 1794 | CD | GLU | A | 934 | −14.315 | 47.656 | −14.251 | 1.00 | 73.66 |
| ATOM | 1795 | OE1 | GLU | A | 934 | −13.217 | 47.534 | −13.659 | 1.00 | 75.01 |
| ATOM | 1796 | OE2 | GLU | A | 934 | −15.267 | 48.344 | −13.815 | 1.00 | 75.23 |
| ATOM | 1797 | N | LYS | A | 935 | −11.896 | 46.926 | −19.446 | 1.00 | 63.09 |
| ATOM | 1798 | CA | LYS | A | 935 | −11.305 | 47.492 | −20.644 | 1.00 | 64.10 |
| ATOM | 1799 | C | LYS | A | 935 | −11.907 | 46.696 | −21.792 | 1.00 | 64.34 |
| ATOM | 1800 | O | LYS | A | 935 | −12.094 | 47.221 | −22.884 | 1.00 | 65.68 |
| ATOM | 1801 | CB | LYS | A | 935 | −9.787 | 47.316 | −20.642 | 1.00 | 66.02 |
| ATOM | 1802 | CG | LYS | A | 935 | −9.036 | 48.128 | −19.598 | 1.00 | 68.95 |
| ATOM | 1803 | CD | LYS | A | 935 | −8.968 | 49.594 | −19.972 | 1.00 | 72.74 |
| ATOM | 1804 | CE | LYS | A | 935 | −7.957 | 50.327 | −19.106 | 1.00 | 74.63 |
| ATOM | 1805 | NZ | LYS | A | 935 | −7.835 | 51.751 | −19.525 | 1.00 | 76.76 |
| ATOM | 1806 | N | GLY | A | 936 | −12.204 | 45.422 | −21.534 | 1.00 | 62.92 |
| ATOM | 1807 | CA | GLY | A | 936 | −12.800 | 44.578 | −22.552 | 1.00 | 61.68 |
| ATOM | 1808 | C | GLY | A | 936 | −12.202 | 43.192 | −22.683 | 1.00 | 61.72 |
| ATOM | 1809 | O | GLY | A | 936 | −12.830 | 42.298 | −23.250 | 1.00 | 61.81 |
| ATOM | 1810 | N | GLU | A | 937 | −10.991 | 43.013 | −22.164 | 1.00 | 60.83 |
| ATOM | 1811 | CA | GLU | A | 937 | −10.276 | 41.734 | −22.217 | 1.00 | 59.06 |
| ATOM | 1812 | C | GLU | A | 937 | −11.148 | 40.509 | −21.889 | 1.00 | 57.16 |
| ATOM | 1813 | O | GLU | A | 937 | −12.066 | 40.588 | −21.073 | 1.00 | 56.93 |
| ATOM | 1814 | CB | GLU | A | 937 | −9.086 | 41.774 | −21.245 | 1.00 | 59.84 |
| ATOM | 1815 | CG | GLU | A | 937 | −8.222 | 40.530 | −21.268 | 1.00 | 62.61 |
| ATOM | 1816 | CD | GLU | A | 937 | −6.844 | 40.793 | −21.823 | 1.00 | 64.42 |
| ATOM | 1817 | OE1 | GLU | A | 937 | −6.690 | 41.760 | −22.595 | 1.00 | 64.76 |
| ATOM | 1818 | OE2 | GLU | A | 937 | −5.916 | 40.024 | −21.495 | 1.00 | 66.97 |
| ATOM | 1819 | N | ARG | A | 938 | −10.842 | 39.382 | −22.528 | 1.00 | 54.12 |
| ATOM | 1820 | CA | ARG | A | 938 | −11.561 | 38.127 | −22.311 | 1.00 | 51.97 |
| ATOM | 1821 | C | ARG | A | 938 | −10.653 | 36.961 | −22.658 | 1.00 | 51.14 |
| ATOM | 1822 | O | ARG | A | 938 | −9.759 | 37.104 | −23.486 | 1.00 | 54.50 |
| ATOM | 1823 | CB | ARG | A | 938 | −12.810 | 38.053 | −23.190 | 1.00 | 50.05 |
| ATOM | 1824 | CG | ARG | A | 938 | −13.914 | 38.970 | −22.756 | 1.00 | 48.91 |
| ATOM | 1825 | CD | ARG | A | 938 | −14.329 | 38.646 | −21.338 | 1.00 | 49.76 |
| ATOM | 1826 | NE | ARG | A | 938 | −14.723 | 39.860 | −20.636 | 1.00 | 54.12 |
| ATOM | 1827 | CZ | ARG | A | 938 | −15.817 | 40.555 | −20.911 | 1.00 | 53.77 |
| ATOM | 1828 | NH1 | ARG | A | 938 | −16.624 | 40.141 | −21.874 | 1.00 | 60.00 |
| ATOM | 1829 | NH2 | ARG | A | 938 | −16.104 | 41.654 | −20.235 | 1.00 | 48.85 |
| ATOM | 1830 | N | LEU | A | 939 | −10.876 | 35.810 | −22.035 | 1.00 | 47.64 |
| ATOM | 1831 | CA | LEU | A | 939 | −10.059 | 34.647 | −22.338 | 1.00 | 47.01 |
| ATOM | 1832 | C | LEU | A | 939 | −10.058 | 34.409 | −23.854 | 1.00 | 46.67 |
| ATOM | 1833 | O | LEU | A | 939 | −11.041 | 34.691 | −24.539 | 1.00 | 45.61 |
| ATOM | 1834 | CB | LEU | A | 939 | −10.591 | 33.422 | −21.587 | 1.00 | 46.49 |
| ATOM | 1835 | CG | LEU | A | 939 | −10.427 | 33.537 | −20.065 | 1.00 | 45.25 |
| ATOM | 1836 | CD1 | LEU | A | 939 | −11.279 | 32.505 | −19.343 | 1.00 | 43.35 |
| ATOM | 1837 | CD2 | LEU | A | 939 | −8.968 | 33.376 | −19.716 | 1.00 | 42.97 |
| ATOM | 1838 | N | PRO | A | 940 | −8.943 | 33.896 | −24.397 | 1.00 | 46.22 |
| ATOM | 1839 | CA | PRO | A | 940 | −8.749 | 33.604 | −25.826 | 1.00 | 44.26 |
| ATOM | 1840 | C | PRO | A | 940 | −9.532 | 32.402 | −26.327 | 1.00 | 45.20 |
| ATOM | 1841 | O | PRO | A | 940 | −9.911 | 31.533 | −25.555 | 1.00 | 46.46 |
| ATOM | 1842 | CB | PRO | A | 940 | −7.252 | 33.336 | −25.936 | 1.00 | 42.34 |
| ATOM | 1843 | CG | PRO | A | 940 | −6.672 | 33.871 | −24.654 | 1.00 | 46.14 |
| ATOM | 1844 | CD | PRO | A | 940 | −7.726 | 33.590 | −23.637 | 1.00 | 43.98 |
| ATOM | 1845 | N | GLN | A | 941 | −9.737 | 32.346 | −27.637 | 1.00 | 46.70 |
| ATOM | 1846 | CA | GLN | A | 941 | −10.448 | 31.242 | −28.263 | 1.00 | 46.31 |
| ATOM | 1847 | C | GLN | A | 941 | −9.558 | 30.012 | −28.242 | 1.00 | 46.69 |
| ATOM | 1848 | O | GLN | A | 941 | −8.479 | 30.009 | −28.824 | 1.00 | 46.60 |
| ATOM | 1849 | CB | GLN | A | 941 | −10.810 | 31.605 | −29.704 | 1.00 | 45.70 |
| ATOM | 1850 | CG | GLN | A | 941 | −11.506 | 30.500 | −30.467 | 1.00 | 46.44 |
| ATOM | 1851 | CD | GLN | A | 941 | −12.083 | 30.960 | −31.808 | 1.00 | 48.60 |
| ATOM | 1852 | OE1 | GLN | A | 941 | −12.721 | 30.178 | −32.521 | 1.00 | 47.90 |
| ATOM | 1853 | NE2 | GLN | A | 941 | −11.861 | 32.226 | −32.154 | 1.00 | 50.29 |
| ATOM | 1854 | N | PRO | A | 942 | −9.992 | 28.950 | −27.553 | 1.00 | 48.40 |
| ATOM | 1855 | CA | PRO | A | 942 | −9.184 | 27.728 | −27.490 | 1.00 | 49.09 |
| ATOM | 1856 | C | PRO | A | 942 | −8.955 | 27.174 | −28.894 | 1.00 | 50.75 |
| ATOM | 1857 | O | PRO | A | 942 | −9.822 | 27.268 | −29.755 | 1.00 | 52.32 |
| ATOM | 1858 | CB | PRO | A | 942 | −10.036 | 26.793 | −26.635 | 1.00 | 47.59 |
| ATOM | 1859 | CG | PRO | A | 942 | −10.810 | 27.745 | −25.767 | 1.00 | 49.17 |
| ATOM | 1860 | CD | PRO | A | 942 | −11.210 | 28.822 | −26.739 | 1.00 | 46.31 |
| ATOM | 1861 | N | PRO | A | 943 | −7.781 | 26.584 | −29.141 | 1.00 | 52.29 |
| ATOM | 1862 | CA | PRO | A | 943 | −7.452 | 26.017 | −30.450 | 1.00 | 51.87 |
| ATOM | 1863 | C | PRO | A | 943 | −8.535 | 25.089 | −31.025 | 1.00 | 52.45 |
| ATOM | 1864 | O | PRO | A | 943 | −9.006 | 25.300 | −32.147 | 1.00 | 53.80 |
| ATOM | 1865 | CB | PRO | A | 943 | −6.149 | 25.270 | −30.175 | 1.00 | 53.02 |
| ATOM | 1866 | CG | PRO | A | 943 | −5.511 | 26.097 | −29.120 | 1.00 | 52.76 |
| ATOM | 1867 | CD | PRO | A | 943 | −6.670 | 26.391 | −28.195 | 1.00 | 53.36 |
| ATOM | 1868 | N | ILE | A | 944 | −8.927 | 24.073 | −30.256 | 1.00 | 50.16 |
| ATOM | 1869 | CA | ILE | A | 944 | −9.928 | 23.100 | −30.702 | 1.00 | 48.57 |
| ATOM | 1870 | C | ILE | A | 944 | −11.349 | 23.635 | −30.776 | 1.00 | 49.82 |
| ATOM | 1871 | O | ILE | A | 944 | −12.259 | 22.953 | −31.268 | 1.00 | 47.90 |
| ATOM | 1872 | CB | ILE | A | 944 | −9.973 | 21.876 | −29.778 | 1.00 | 47.78 |
| ATOM | 1873 | CG1 | ILE | A | 944 | −10.608 | 22.255 | −28.430 | 1.00 | 43.48 |
| ATOM | 1874 | CG2 | ILE | A | 944 | −8.579 | 21.321 | −29.607 | 1.00 | 46.76 |
| ATOM | 1875 | CD1 | ILE | A | 944 | −10.784 | 21.080 | −27.499 | 1.00 | 42.87 |
| ATOM | 1876 | N | CYS | A | 945 | −11.542 | 24.847 | −30.275 | 1.00 | 51.05 |
| ATOM | 1877 | CA | CYS | A | 945 | −12.860 | 25.449 | −30.270 | 1.00 | 51.69 |
| ATOM | 1878 | C | CYS | A | 945 | −13.254 | 26.015 | −31.604 | 1.00 | 52.55 |
| ATOM | 1879 | O | CYS | A | 945 | −12.490 | 26.739 | −32.240 | 1.00 | 55.03 |
| ATOM | 1880 | CB | CYS | A | 945 | −12.951 | 26.557 | −29.217 | 1.00 | 52.52 |
| ATOM | 1881 | SG | CYS | A | 945 | −13.270 | 25.971 | −27.546 | 1.00 | 52.50 |
| ATOM | 1882 | N | THR | A | 946 | −14.465 | 25.666 | −32.011 | 1.00 | 51.88 |
| ATOM | 1883 | CA | THR | A | 946 | −15.059 | 26.138 | −33.246 | 1.00 | 50.67 |
| ATOM | 1884 | C | THR | A | 946 | −15.703 | 27.448 | −32.793 | 1.00 | 49.33 |
| ATOM | 1885 | O | THR | A | 946 | −16.054 | 27.571 | −31.626 | 1.00 | 49.43 |
| ATOM | 1886 | CB | THR | A | 946 | −16.126 | 25.127 | −33.733 | 1.00 | 50.13 |
| ATOM | 1887 | OG1 | THR | A | 946 | −16.468 | 25.404 | −35.091 | 1.00 | 52.85 |
| ATOM | 1888 | CG2 | THR | A | 946 | −17.376 | 25.205 | −32.871 | 1.00 | 46.57 |
| ATOM | 1889 | N | ILE | A | 947 | −15.849 | 28.424 | −33.684 | 1.00 | 49.50 |
| ATOM | 1890 | CA | ILE | A | 947 | −16.436 | 29.705 | −33.289 | 1.00 | 48.83 |
| ATOM | 1891 | C | ILE | A | 947 | −17.843 | 29.569 | −32.718 | 1.00 | 49.26 |
| ATOM | 1892 | O | ILE | A | 947 | −18.415 | 30.527 | −32.195 | 1.00 | 52.14 |
| ATOM | 1893 | CB | ILE | A | 947 | −16.481 | 30.714 | −34.454 | 1.00 | 48.12 |
| ATOM | 1894 | CG1 | ILE | A | 947 | −16.732 | 32.121 | −33.888 | 1.00 | 49.16 |
| ATOM | 1895 | CG2 | ILE | A | 947 | −17.570 | 30.321 | −35.452 | 1.00 | 46.40 |
| ATOM | 1896 | CD1 | ILE | A | 947 | −16.721 | 33.247 | −34.909 | 1.00 | 50.09 |
| ATOM | 1897 | N | ASP | A | 948 | −18.407 | 28.379 | −32.819 | 1.00 | 48.28 |
| ATOM | 1898 | CA | ASP | A | 948 | −19.734 | 28.137 | −32.279 | 1.00 | 47.63 |
| ATOM | 1899 | C | ASP | A | 948 | −19.621 | 27.996 | −30.761 | 1.00 | 46.50 |
| ATOM | 1900 | O | ASP | A | 948 | −20.413 | 28.583 | −30.012 | 1.00 | 46.41 |
| ATOM | 1901 | CB | ASP | A | 948 | −20.311 | 26.857 | −32.888 | 1.00 | 49.31 |
| ATOM | 1902 | CG | ASP | A | 948 | −20.568 | 26.988 | −34.377 | 1.00 | 49.35 |
| ATOM | 1903 | OD1 | ASP | A | 948 | −21.612 | 27.553 | −34.737 | 1.00 | 52.88 |
| ATOM | 1904 | OD2 | ASP | A | 948 | −19.729 | 26.543 | −35.186 | 1.00 | 50.17 |
| ATOM | 1905 | N | VAL | A | 949 | −18.626 | 27.226 | −30.317 | 1.00 | 43.56 |
| ATOM | 1906 | CA | VAL | A | 949 | −18.405 | 26.998 | −28.896 | 1.00 | 43.65 |
| ATOM | 1907 | C | VAL | A | 949 | −17.894 | 28.262 | −28.222 | 1.00 | 43.64 |
| ATOM | 1908 | O | VAL | A | 949 | −18.333 | 28.619 | −27.127 | 1.00 | 44.08 |
| ATOM | 1909 | CB | VAL | A | 949 | −17.381 | 25.858 | −28.654 | 1.00 | 43.97 |
| ATOM | 1910 | CG1 | VAL | A | 949 | −17.132 | 25.689 | −27.158 | 1.00 | 41.83 |
| ATOM | 1911 | CG2 | VAL | A | 949 | −17.895 | 24.551 | −29.246 | 1.00 | 41.36 |
| ATOM | 1912 | N | TYR | A | 950 | −16.965 | 28.939 | −28.885 | 1.00 | 43.36 |
| ATOM | 1913 | CA | TYR | A | 950 | −16.395 | 30.160 | −28.343 | 1.00 | 42.30 |
| ATOM | 1914 | C | TYR | A | 950 | −17.478 | 31.180 | −28.036 | 1.00 | 41.41 |
| ATOM | 1915 | O | TYR | A | 950 | −17.489 | 31.774 | −26.961 | 1.00 | 40.32 |
| ATOM | 1916 | CB | TYR | A | 950 | −15.386 | 30.760 | −29.322 | 1.00 | 43.03 |
| ATOM | 1917 | CG | TYR | A | 950 | −14.489 | 31.806 | −28.686 | 1.00 | 45.66 |
| ATOM | 1918 | CD1 | TYR | A | 950 | −13.837 | 31.549 | −27.473 | 1.00 | 45.82 |
| ATOM | 1919 | CD2 | TYR | A | 950 | −14.283 | 33.044 | −29.290 | 1.00 | 46.41 |
| ATOM | 1920 | CE1 | TYR | A | 950 | −13.004 | 32.496 | −26.879 | 1.00 | 44.71 |
| ATOM | 1921 | CE2 | TYR | A | 950 | −13.446 | 34.002 | −28.701 | 1.00 | 47.81 |
| ATOM | 1922 | CZ | TYR | A | 950 | −12.811 | 33.715 | −27.498 | 1.00 | 46.62 |
| ATOM | 1923 | OH | TYR | A | 950 | −11.967 | 34.638 | −26.931 | 1.00 | 46.10 |
| ATOM | 1924 | N | MET | A | 951 | −18.390 | 31.386 | −28.978 | 1.00 | 41.91 |
| ATOM | 1925 | CA | MET | A | 951 | −19.459 | 32.343 | −28.761 | 1.00 | 43.90 |
| ATOM | 1926 | C | MET | A | 951 | −20.267 | 31.988 | −27.532 | 1.00 | 44.53 |
| ATOM | 1927 | O | MET | A | 951 | −20.776 | 32.877 | −26.849 | 1.00 | 47.60 |
| ATOM | 1928 | CB | MET | A | 951 | −20.366 | 32.447 | −29.989 | 1.00 | 47.27 |
| ATOM | 1929 | CG | MET | A | 951 | −19.674 | 33.105 | −31.166 | 1.00 | 53.88 |
| ATOM | 1930 | SD | MET | A | 951 | −18.776 | 34.589 | −30.618 | 1.00 | 62.31 |
| ATOM | 1931 | CE | MET | A | 951 | −20.155 | 35.487 | −29.841 | 1.00 | 55.74 |
| ATOM | 1932 | N | VAL | A | 952 | −20.397 | 30.700 | −27.232 | 1.00 | 42.77 |
| ATOM | 1933 | CA | VAL | A | 952 | −21.131 | 30.334 | −26.031 | 1.00 | 42.94 |
| ATOM | 1934 | C | VAL | A | 952 | −20.352 | 30.869 | −24.819 | 1.00 | 44.09 |
| ATOM | 1935 | O | VAL | A | 952 | −20.928 | 31.493 | −23.923 | 1.00 | 44.56 |
| ATOM | 1936 | CB | VAL | A | 952 | −21.341 | 28.795 | −25.924 | 1.00 | 42.99 |
| ATOM | 1937 | CG1 | VAL | A | 952 | −21.891 | 28.430 | −24.551 | 1.00 | 39.35 |
| ATOM | 1938 | CG2 | VAL | A | 952 | −22.332 | 28.339 | −26.999 | 1.00 | 39.89 |
| ATOM | 1939 | N | MET | A | 953 | −19.039 | 30.654 | −24.807 | 1.00 | 44.23 |
| ATOM | 1940 | CA | MET | A | 953 | −18.202 | 31.142 | −23.711 | 1.00 | 45.49 |
| ATOM | 1941 | C | MET | A | 953 | −18.231 | 32.670 | −23.603 | 1.00 | 45.43 |
| ATOM | 1942 | O | MET | A | 953 | −18.393 | 33.225 | −22.510 | 1.00 | 45.00 |
| ATOM | 1943 | CB | MET | A | 953 | −16.759 | 30.683 | −23.900 | 1.00 | 44.82 |
| ATOM | 1944 | CG | MET | A | 953 | −16.587 | 29.199 | −23.816 | 1.00 | 48.13 |
| ATOM | 1945 | SD | MET | A | 953 | −14.944 | 28.724 | −24.267 | 1.00 | 52.76 |
| ATOM | 1946 | CE | MET | A | 953 | −15.146 | 26.965 | −24.437 | 1.00 | 52.38 |
| ATOM | 1947 | N | VAL | A | 954 | −18.081 | 33.353 | −24.734 | 1.00 | 42.69 |
| ATOM | 1948 | CA | VAL | A | 954 | −18.086 | 34.804 | −24.705 | 1.00 | 42.88 |
| ATOM | 1949 | C | VAL | A | 954 | −19.408 | 35.363 | −24.175 | 1.00 | 43.39 |
| ATOM | 1950 | O | VAL | A | 954 | −19.402 | 36.290 | −23.381 | 1.00 | 44.32 |
| ATOM | 1951 | CB | VAL | A | 954 | −17.792 | 35.394 | −26.090 | 1.00 | 42.98 |
| ATOM | 1952 | CG1 | VAL | A | 954 | −17.863 | 36.890 | −26.020 | 1.00 | 44.13 |
| ATOM | 1953 | CG2 | VAL | A | 954 | −16.403 | 34.961 | −26.565 | 1.00 | 43.05 |
| ATOM | 1954 | N | LYS | A | 955 | −20.545 | 34.812 | −24.589 | 1.00 | 43.09 |
| ATOM | 1955 | CA | LYS | A | 955 | −21.814 | 35.327 | −24.070 | 1.00 | 44.12 |
| ATOM | 1956 | C | LYS | A | 955 | −21.844 | 35.189 | −22.552 | 1.00 | 43.87 |
| ATOM | 1957 | O | LYS | A | 955 | −22.417 | 36.024 | −21.855 | 1.00 | 44.04 |
| ATOM | 1958 | CB | LYS | A | 955 | −23.012 | 34.573 | −24.656 | 1.00 | 45.68 |
| ATOM | 1959 | CG | LYS | A | 955 | −23.304 | 34.831 | −26.123 | 1.00 | 46.27 |
| ATOM | 1960 | CD | LYS | A | 955 | −24.430 | 33.924 | −26.563 | 1.00 | 46.94 |
| ATOM | 1961 | CE | LYS | A | 955 | −24.660 | 33.965 | −28.048 | 1.00 | 50.31 |
| ATOM | 1962 | NZ | LYS | A | 955 | −25.433 | 32.759 | −28.442 | 1.00 | 51.04 |
| ATOM | 1963 | N | CYS | A | 956 | −21.238 | 34.123 | −22.042 | 1.00 | 43.06 |
| ATOM | 1964 | CA | CYS | A | 956 | −21.199 | 33.896 | −20.602 | 1.00 | 43.04 |
| ATOM | 1965 | C | CYS | A | 956 | −20.505 | 35.042 | −19.861 | 1.00 | 44.88 |
| ATOM | 1966 | O | CYS | A | 956 | −20.712 | 35.235 | −18.652 | 1.00 | 42.74 |
| ATOM | 1967 | CB | CYS | A | 956 | −20.453 | 32.606 | −20.296 | 1.00 | 40.76 |
| ATOM | 1968 | SG | CYS | A | 956 | −21.406 | 31.114 | −20.517 | 1.00 | 40.75 |
| ATOM | 1969 | N | TRP | A | 957 | −19.684 | 35.796 | −20.593 | 1.00 | 46.16 |
| ATOM | 1970 | CA | TRP | A | 957 | −18.929 | 36.888 | −20.006 | 1.00 | 47.28 |
| ATOM | 1971 | C | TRP | A | 957 | −19.409 | 38.283 | −20.373 | 1.00 | 49.59 |
| ATOM | 1972 | O | TRP | A | 957 | −18.629 | 39.231 | −20.342 | 1.00 | 50.87 |
| ATOM | 1973 | CB | TRP | A | 957 | −17.442 | 36.749 | −20.365 | 1.00 | 44.94 |
| ATOM | 1974 | CG | TRP | A | 957 | −16.881 | 35.389 | −20.062 | 1.00 | 41.87 |
| ATOM | 1975 | CD1 | TRP | A | 957 | −17.113 | 34.636 | −18.949 | 1.00 | 40.97 |
| ATOM | 1976 | CD2 | TRP | A | 957 | −15.987 | 34.628 | −20.880 | 1.00 | 40.66 |
| ATOM | 1977 | NE1 | TRP | A | 957 | −16.423 | 33.450 | −19.022 | 1.00 | 41.10 |
| ATOM | 1978 | CE2 | TRP | A | 957 | −15.720 | 33.419 | −20.196 | 1.00 | 39.58 |
| ATOM | 1979 | CE3 | TRP | A | 957 | −15.383 | 34.850 | −22.125 | 1.00 | 39.57 |
| ATOM | 1980 | CZ2 | TRP | A | 957 | −14.877 | 32.435 | −20.712 | 1.00 | 39.64 |
| ATOM | 1981 | CZ3 | TRP | A | 957 | −14.545 | 33.871 | −22.639 | 1.00 | 40.43 |
| ATOM | 1982 | CH2 | TRP | A | 957 | −14.298 | 32.676 | −21.930 | 1.00 | 40.43 |
| ATOM | 1983 | N | MET | A | 958 | −20.682 | 38.425 | −20.720 | 1.00 | 50.92 |
| ATOM | 1984 | CA | MET | A | 958 | −21.186 | 39.752 | −21.048 | 1.00 | 52.62 |
| ATOM | 1985 | C | MET | A | 958 | −21.165 | 40.609 | −19.789 | 1.00 | 50.99 |
| ATOM | 1986 | O | MET | A | 958 | −21.272 | 40.098 | −18.676 | 1.00 | 49.32 |
| ATOM | 1987 | CB | MET | A | 958 | −22.609 | 39.682 | −21.598 | 1.00 | 54.02 |
| ATOM | 1988 | CG | MET | A | 958 | −22.708 | 38.991 | −22.941 | 1.00 | 57.72 |
| ATOM | 1989 | SD | MET | A | 958 | −21.589 | 39.707 | −24.136 | 1.00 | 61.72 |
| ATOM | 1990 | CE | MET | A | 958 | −22.626 | 41.096 | −24.754 | 1.00 | 62.55 |
| ATOM | 1991 | N | ILE | A | 959 | −21.009 | 41.913 | −19.968 | 1.00 | 51.63 |
| ATOM | 1992 | CA | ILE | A | 959 | −20.979 | 42.819 | −18.829 | 1.00 | 53.09 |
| ATOM | 1993 | C | ILE | A | 959 | −22.342 | 42.742 | −18.161 | 1.00 | 52.72 |
| ATOM | 1994 | O | ILE | A | 959 | −22.461 | 42.808 | −16.944 | 1.00 | 52.77 |
| ATOM | 1995 | CB | ILE | A | 959 | −20.740 | 44.273 | −19.267 | 1.00 | 53.98 |
| ATOM | 1996 | CG1 | ILE | A | 959 | −19.387 | 44.387 | −19.981 | 1.00 | 57.72 |
| ATOM | 1997 | CG2 | ILE | A | 959 | −20.785 | 45.191 | −18.050 | 1.00 | 53.54 |
| ATOM | 1998 | CD1 | ILE | A | 959 | −19.163 | 45.733 | −20.703 | 1.00 | 60.53 |
| ATOM | 1999 | N | ASP | A | 960 | −23.374 | 42.584 | −18.977 | 1.00 | 53.04 |
| ATOM | 2000 | CA | ASP | A | 960 | −24.733 | 42.505 | −18.472 | 1.00 | 54.30 |
| ATOM | 2001 | C | ASP | A | 960 | −25.075 | 41.102 | −17.961 | 1.00 | 53.33 |
| ATOM | 2002 | O | ASP | A | 960 | −25.337 | 40.191 | −18.739 | 1.00 | 50.47 |
| ATOM | 2003 | CB | ASP | A | 960 | −25.706 | 42.903 | −19.575 | 1.00 | 57.50 |
| ATOM | 2004 | CG | ASP | A | 960 | −26.971 | 43.511 | −19.035 | 1.00 | 59.92 |
| ATOM | 2005 | OD1 | ASP | A | 960 | −27.626 | 42.880 | −18.177 | 1.00 | 63.64 |
| ATOM | 2006 | OD2 | ASP | A | 960 | −27.312 | 44.626 | −19.476 | 1.00 | 62.47 |
| ATOM | 2007 | N | ALA | A | 961 | −25.077 | 40.942 | −16.646 | 1.00 | 53.51 |
| ATOM | 2008 | CA | ALA | A | 961 | −25.384 | 39.659 | −16.033 | 1.00 | 54.51 |
| ATOM | 2009 | C | ALA | A | 961 | −26.650 | 39.046 | −16.637 | 1.00 | 55.26 |
| ATOM | 2010 | O | ALA | A | 961 | −26.690 | 37.856 | −16.956 | 1.00 | 53.76 |
| ATOM | 2011 | CB | ALA | A | 961 | −25.549 | 39.839 | −14.528 | 1.00 | 52.04 |
| ATOM | 2012 | N | ASP | A | 962 | −27.677 | 39.870 | −16.804 | 1.00 | 56.69 |
| ATOM | 2013 | CA | ASP | A | 962 | −28.942 | 39.405 | −17.360 | 1.00 | 58.69 |
| ATOM | 2014 | C | ASP | A | 962 | −28.948 | 38.994 | −18.830 | 1.00 | 57.34 |
| ATOM | 2015 | O | ASP | A | 962 | −29.803 | 38.211 | −19.241 | 1.00 | 57.60 |
| ATOM | 2016 | CB | ASP | A | 962 | −30.043 | 40.439 | −17.118 | 1.00 | 61.14 |
| ATOM | 2017 | CG | ASP | A | 962 | −30.633 | 40.339 | −15.724 | 1.00 | 64.05 |
| ATOM | 2018 | OD1 | ASP | A | 962 | −30.863 | 39.199 | −15.259 | 1.00 | 62.71 |
| ATOM | 2019 | OD2 | ASP | A | 962 | −30.876 | 41.396 | −15.103 | 1.00 | 68.35 |
| ATOM | 2020 | N | SER | A | 963 | −28.022 | 39.509 | −19.631 | 1.00 | 56.01 |
| ATOM | 2021 | CA | SER | A | 963 | −27.996 | 39.111 | −21.031 | 1.00 | 54.55 |
| ATOM | 2022 | C | SER | A | 963 | −27.117 | 37.870 | −21.249 | 1.00 | 53.95 |
| ATOM | 2023 | O | SER | A | 963 | −26.940 | 37.415 | −22.378 | 1.00 | 55.77 |
| ATOM | 2024 | CB | SER | A | 963 | −27.537 | 40.275 | −21.925 | 1.00 | 55.92 |
| ATOM | 2025 | OG | SER | A | 963 | −26.176 | 40.600 | −21.734 | 1.00 | 55.38 |
| ATOM | 2026 | N | ARG | A | 964 | −26.565 | 37.323 | −20.170 | 1.00 | 51.26 |
| ATOM | 2027 | CA | ARG | A | 964 | −25.746 | 36.115 | −20.272 | 1.00 | 50.44 |
| ATOM | 2028 | C | ARG | A | 964 | −26.711 | 34.942 | −20.410 | 1.00 | 50.43 |
| ATOM | 2029 | O | ARG | A | 964 | −27.820 | 34.970 | −19.874 | 1.00 | 50.63 |
| ATOM | 2030 | CB | ARG | A | 964 | −24.899 | 35.900 | −19.010 | 1.00 | 47.22 |
| ATOM | 2031 | CG | ARG | A | 964 | −23.902 | 36.994 | −18.724 | 1.00 | 45.09 |
| ATOM | 2032 | CD | ARG | A | 964 | −23.324 | 36.875 | −17.329 | 1.00 | 42.47 |
| ATOM | 2033 | NE | ARG | A | 964 | −22.590 | 38.085 | −16.981 | 1.00 | 44.27 |
| ATOM | 2034 | CZ | ARG | A | 964 | −22.156 | 38.392 | −15.761 | 1.00 | 43.66 |
| ATOM | 2035 | NH1 | ARG | A | 964 | −22.368 | 37.577 | −14.736 | 1.00 | 38.19 |
| ATOM | 2036 | NH2 | ARG | A | 964 | −21.527 | 39.543 | −15.567 | 1.00 | 43.56 |
| ATOM | 2037 | N | PRO | A | 965 | −26.303 | 33.891 | −21.126 | 1.00 | 50.03 |
| ATOM | 2038 | CA | PRO | A | 965 | −27.230 | 32.762 | −21.256 | 1.00 | 50.55 |
| ATOM | 2039 | C | PRO | A | 965 | −27.593 | 32.164 | −19.890 | 1.00 | 50.02 |
| ATOM | 2040 | O | PRO | A | 965 | −26.851 | 32.318 | −18.920 | 1.00 | 50.28 |
| ATOM | 2041 | CB | PRO | A | 965 | −26.460 | 31.786 | −22.146 | 1.00 | 49.94 |
| ATOM | 2042 | CG | PRO | A | 965 | −25.011 | 32.123 | −21.855 | 1.00 | 51.67 |
| ATOM | 2043 | CD | PRO | A | 965 | −25.011 | 33.617 | −21.780 | 1.00 | 49.61 |
| ATOM | 2044 | N | LYS | A | 966 | −28.748 | 31.515 | −19.804 | 1.00 | 48.80 |
| ATOM | 2045 | CA | LYS | A | 966 | −29.153 | 30.889 | −18.554 | 1.00 | 48.28 |
| ATOM | 2046 | C | LYS | A | 966 | −28.753 | 29.418 | −18.572 | 1.00 | 47.56 |
| ATOM | 2047 | O | LYS | A | 966 | −28.629 | 28.808 | −19.642 | 1.00 | 45.03 |
| ATOM | 2048 | CB | LYS | A | 966 | −30.654 | 31.021 | −18.343 | 1.00 | 49.99 |
| ATOM | 2049 | CG | LYS | A | 966 | −31.096 | 32.436 | −18.067 | 1.00 | 55.88 |
| ATOM | 2050 | CD | LYS | A | 966 | −32.242 | 32.462 | −17.073 | 1.00 | 60.89 |
| ATOM | 2051 | CE | LYS | A | 966 | −32.579 | 33.893 | −16.660 | 1.00 | 64.65 |
| ATOM | 2052 | NZ | LYS | A | 966 | −33.599 | 33.951 | −15.563 | 1.00 | 65.07 |
| ATOM | 2053 | N | PHE | A | 967 | −28.546 | 28.842 | −17.392 | 1.00 | 45.93 |
| ATOM | 2054 | CA | PHE | A | 967 | −28.138 | 27.449 | −17.327 | 1.00 | 46.66 |
| ATOM | 2055 | C | PHE | A | 967 | −29.005 | 26.499 | −18.135 | 1.00 | 47.20 |
| ATOM | 2056 | O | PHE | A | 967 | −28.518 | 25.470 | −18.579 | 1.00 | 49.36 |
| ATOM | 2057 | CB | PHE | A | 967 | −28.029 | 26.988 | −15.875 | 1.00 | 46.03 |
| ATOM | 2058 | CG | PHE | A | 967 | −26.767 | 27.437 | −15.213 | 1.00 | 45.56 |
| ATOM | 2059 | CD1 | PHE | A | 967 | −26.792 | 28.392 | −14.199 | 1.00 | 45.88 |
| ATOM | 2060 | CD2 | PHE | A | 967 | −25.535 | 26.971 | −15.669 | 1.00 | 44.49 |
| ATOM | 2061 | CE1 | PHE | A | 967 | −25.599 | 28.886 | −13.650 | 1.00 | 47.81 |
| ATOM | 2062 | CE2 | PHE | A | 967 | −24.340 | 27.455 | −15.131 | 1.00 | 46.71 |
| ATOM | 2063 | CZ | PHE | A | 967 | −24.368 | 28.416 | −14.119 | 1.00 | 45.70 |
| ATOM | 2064 | N | ALA | A | 968 | −30.275 | 26.846 | −18.340 | 1.00 | 47.85 |
| ATOM | 2065 | CA | ALA | A | 968 | −31.183 | 26.014 | −19.136 | 1.00 | 48.27 |
| ATOM | 2066 | C | ALA | A | 968 | −30.746 | 25.988 | −20.615 | 1.00 | 49.39 |
| ATOM | 2067 | O | ALA | A | 968 | −30.753 | 24.943 | −21.264 | 1.00 | 49.53 |
| ATOM | 2068 | CB | ALA | A | 968 | −32.620 | 26.542 | −19.028 | 1.00 | 45.49 |
| ATOM | 2069 | N | GLU | A | 969 | −30.358 | 27.142 | −21.144 | 1.00 | 49.32 |
| ATOM | 2070 | CA | GLU | A | 969 | −29.939 | 27.226 | −22.533 | 1.00 | 48.74 |
| ATOM | 2071 | C | GLU | A | 969 | −28.574 | 26.587 | −22.760 | 1.00 | 48.05 |
| ATOM | 2072 | O | GLU | A | 969 | −28.325 | 25.986 | −23.812 | 1.00 | 47.73 |
| ATOM | 2073 | CB | GLU | A | 969 | −29.863 | 28.682 | −22.976 | 1.00 | 51.47 |
| ATOM | 2074 | CG | GLU | A | 969 | −31.120 | 29.492 | −22.779 | 1.00 | 55.25 |
| ATOM | 2075 | CD | GLU | A | 969 | −30.928 | 30.929 | −23.246 | 1.00 | 60.64 |
| ATOM | 2076 | OE1 | GLU | A | 969 | −30.233 | 31.703 | −22.539 | 1.00 | 58.91 |
| ATOM | 2077 | OE2 | GLU | A | 969 | −31.454 | 31.274 | −24.335 | 1.00 | 63.69 |
| ATOM | 2078 | N | LEU | A | 970 | −27.681 | 26.739 | −21.784 | 1.00 | 44.93 |
| ATOM | 2079 | CA | LEU | A | 970 | −26.343 | 26.186 | −21.918 | 1.00 | 42.24 |
| ATOM | 2080 | C | LEU | A | 970 | −26.430 | 24.686 | −22.019 | 1.00 | 41.43 |
| ATOM | 2081 | O | LEU | A | 970 | −25.822 | 24.081 | −22.901 | 1.00 | 40.90 |
| ATOM | 2082 | CB | LEU | A | 970 | −25.450 | 26.611 | −20.741 | 1.00 | 40.56 |
| ATOM | 2083 | CG | LEU | A | 970 | −25.011 | 28.089 | −20.768 | 1.00 | 37.75 |
| ATOM | 2084 | CD1 | LEU | A | 970 | −24.376 | 28.470 | −19.459 | 1.00 | 37.42 |
| ATOM | 2085 | CD2 | LEU | A | 970 | −24.058 | 28.329 | −21.906 | 1.00 | 32.61 |
| ATOM | 2086 | N | ALA | A | 971 | −27.207 | 24.079 | −21.134 | 1.00 | 41.36 |
| ATOM | 2087 | CA | ALA | A | 971 | −27.350 | 22.634 | −21.175 | 1.00 | 42.61 |
| ATOM | 2088 | C | ALA | A | 971 | −27.934 | 22.243 | −22.518 | 1.00 | 44.03 |
| ATOM | 2089 | O | ALA | A | 971 | −27.470 | 21.309 | −23.162 | 1.00 | 44.82 |
| ATOM | 2090 | CB | ALA | A | 971 | −28.252 | 22.168 | −20.074 | 1.00 | 42.94 |
| ATOM | 2091 | N | ALA | A | 972 | −28.948 | 22.980 | −22.941 | 1.00 | 43.93 |
| ATOM | 2092 | CA | ALA | A | 972 | −29.600 | 22.705 | −24.200 | 1.00 | 46.31 |
| ATOM | 2093 | C | ALA | A | 972 | −28.675 | 22.874 | −25.411 | 1.00 | 49.37 |
| ATOM | 2094 | O | ALA | A | 972 | −28.638 | 22.013 | −26.300 | 1.00 | 51.33 |
| ATOM | 2095 | CB | ALA | A | 972 | −30.825 | 23.601 | −24.342 | 1.00 | 43.28 |
| ATOM | 2096 | N | GLU | A | 973 | −27.934 | 23.976 | −25.454 | 1.00 | 50.72 |
| ATOM | 2097 | CA | GLU | A | 973 | −27.045 | 24.243 | −26.580 | 1.00 | 52.77 |
| ATOM | 2098 | C | GLU | A | 973 | −25.930 | 23.205 | −26.699 | 1.00 | 51.88 |
| ATOM | 2099 | O | GLU | A | 973 | −25.721 | 22.643 | −27.768 | 1.00 | 52.95 |
| ATOM | 2100 | CB | GLU | A | 973 | −26.463 | 25.662 | −26.450 | 1.00 | 57.46 |
| ATOM | 2101 | CG | GLU | A | 973 | −25.475 | 26.119 | −27.540 | 1.00 | 62.52 |
| ATOM | 2102 | CD | GLU | A | 973 | −26.081 | 26.196 | −28.944 | 1.00 | 67.05 |
| ATOM | 2103 | OE1 | GLU | A | 973 | −27.304 | 26.435 | −29.067 | 1.00 | 69.19 |
| ATOM | 2104 | OE2 | GLU | A | 973 | −25.323 | 26.032 | −29.930 | 1.00 | 68.06 |
| ATOM | 2105 | N | PHE | A | 974 | −25.219 | 22.935 | −25.609 | 1.00 | 49.75 |
| ATOM | 2106 | CA | PHE | A | 974 | −24.134 | 21.965 | −25.679 | 1.00 | 48.22 |
| ATOM | 2107 | C | PHE | A | 974 | −24.698 | 20.605 | −25.979 | 1.00 | 47.87 |
| ATOM | 2108 | O | PHE | A | 974 | −24.088 | 19.806 | −26.678 | 1.00 | 47.01 |
| ATOM | 2109 | CB | PHE | A | 974 | −23.345 | 21.938 | −24.374 | 1.00 | 46.42 |
| ATOM | 2110 | CG | PHE | A | 974 | −22.362 | 23.053 | −24.258 | 1.00 | 47.75 |
| ATOM | 2111 | CD1 | PHE | A | 974 | −21.276 | 23.129 | −25.128 | 1.00 | 48.10 |
| ATOM | 2112 | CD2 | PHE | A | 974 | −22.547 | 24.071 | −23.327 | 1.00 | 47.47 |
| ATOM | 2113 | CE1 | PHE | A | 974 | −20.390 | 24.204 | −25.077 | 1.00 | 46.79 |
| ATOM | 2114 | CE2 | PHE | A | 974 | −21.673 | 25.148 | −23.270 | 1.00 | 47.11 |
| ATOM | 2115 | CZ | PHE | A | 974 | −20.588 | 25.215 | −24.150 | 1.00 | 47.33 |
| ATOM | 2116 | N | SER | A | 975 | −25.886 | 20.360 | −25.446 | 1.00 | 47.80 |
| ATOM | 2117 | CA | SER | A | 975 | −26.581 | 19.102 | −25.644 | 1.00 | 47.24 |
| ATOM | 2118 | C | SER | A | 975 | −26.704 | 18.813 | −27.136 | 1.00 | 47.38 |
| ATOM | 2119 | O | SER | A | 975 | −26.389 | 17.708 | −27.588 | 1.00 | 47.22 |
| ATOM | 2120 | CB | SER | A | 975 | −27.976 | 19.182 | −25.022 | 1.00 | 48.16 |
| ATOM | 2121 | OG | SER | A | 975 | −28.591 | 17.914 | −24.947 | 1.00 | 49.36 |
| ATOM | 2122 | N | ARG | A | 976 | −27.158 | 19.796 | −27.910 | 1.00 | 45.99 |
| ATOM | 2123 | CA | ARG | A | 976 | −27.301 | 19.558 | −29.335 | 1.00 | 46.51 |
| ATOM | 2124 | C | ARG | A | 976 | −25.925 | 19.475 | −30.004 | 1.00 | 46.75 |
| ATOM | 2125 | O | ARG | A | 976 | −25.779 | 18.840 | −31.042 | 1.00 | 47.59 |
| ATOM | 2126 | CB | ARG | A | 976 | −28.189 | 20.633 | −29.988 | 1.00 | 44.73 |
| ATOM | 2127 | CG | ARG | A | 976 | −27.436 | 21.795 | −30.545 | 1.00 | 50.21 |
| ATOM | 2128 | CD | ARG | A | 976 | −28.301 | 22.825 | −31.270 | 1.00 | 51.39 |
| ATOM | 2129 | NE | ARG | A | 976 | −27.435 | 23.946 | −31.634 | 1.00 | 53.87 |
| ATOM | 2130 | CZ | ARG | A | 976 | −26.509 | 23.885 | −32.587 | 1.00 | 54.84 |
| ATOM | 2131 | NH1 | ARG | A | 976 | −25.740 | 24.936 | −32.849 | 1.00 | 56.13 |
| ATOM | 2132 | NH2 | ARG | A | 976 | −26.383 | 22.784 | −33.313 | 1.00 | 52.91 |
| ATOM | 2133 | N | MET | A | 977 | −24.909 | 20.090 | −29.406 | 1.00 | 48.59 |
| ATOM | 2134 | CA | MET | A | 977 | −23.562 | 20.036 | −29.979 | 1.00 | 50.45 |
| ATOM | 2135 | C | MET | A | 977 | −22.996 | 18.635 | −29.790 | 1.00 | 51.16 |
| ATOM | 2136 | O | MET | A | 977 | −22.230 | 18.140 | −30.620 | 1.00 | 51.33 |
| ATOM | 2137 | CB | MET | A | 977 | −22.637 | 21.080 | −29.324 | 1.00 | 51.44 |
| ATOM | 2138 | CG | MET | A | 977 | −22.833 | 22.507 | −29.861 | 1.00 | 54.98 |
| ATOM | 2139 | SD | MET | A | 977 | −22.111 | 23.823 | −28.853 | 1.00 | 58.42 |
| ATOM | 2140 | CE | MET | A | 977 | −20.982 | 24.513 | −29.911 | 1.00 | 56.62 |
| ATOM | 2141 | N | ALA | A | 978 | −23.399 | 17.990 | −28.703 | 1.00 | 51.59 |
| ATOM | 2142 | CA | ALA | A | 978 | −22.938 | 16.646 | −28.395 | 1.00 | 52.55 |
| ATOM | 2143 | C | ALA | A | 978 | −23.492 | 15.596 | −29.363 | 1.00 | 53.68 |
| ATOM | 2144 | O | ALA | A | 978 | −22.947 | 14.501 | −29.477 | 1.00 | 54.76 |
| ATOM | 2145 | CB | ALA | A | 978 | −23.312 | 16.292 | −26.959 | 1.00 | 50.13 |
| ATOM | 2146 | N | ARG | A | 979 | −24.573 | 15.922 | −30.058 | 1.00 | 55.30 |
| ATOM | 2147 | CA | ARG | A | 979 | −25.156 | 14.983 | −31.006 | 1.00 | 58.64 |
| ATOM | 2148 | C | ARG | A | 979 | −24.312 | 14.943 | −32.279 | 1.00 | 59.72 |
| ATOM | 2149 | O | ARG | A | 979 | −24.554 | 14.127 | −33.166 | 1.00 | 60.30 |
| ATOM | 2150 | CB | ARG | A | 979 | −26.586 | 15.397 | −31.370 | 1.00 | 60.38 |
| ATOM | 2151 | CG | ARG | A | 979 | −27.545 | 15.474 | −30.195 | 1.00 | 65.31 |
| ATOM | 2152 | CD | ARG | A | 979 | −28.864 | 16.123 | −30.604 | 1.00 | 67.03 |
| ATOM | 2153 | NE | ARG | A | 979 | −29.575 | 16.664 | −29.448 | 1.00 | 68.78 |
| ATOM | 2154 | CZ | ARG | A | 979 | −30.539 | 17.581 | −29.516 | 1.00 | 70.15 |
| ATOM | 2155 | NH1 | ARG | A | 979 | −30.922 | 18.067 | −30.694 | 1.00 | 70.75 |
| ATOM | 2156 | NH2 | ARG | A | 979 | −31.107 | 18.027 | −28.398 | 1.00 | 70.11 |
| ATOM | 2157 | N | ASP | A | 980 | −23.325 | 15.827 | −32.365 | 1.00 | 59.38 |
| ATOM | 2158 | CA | ASP | A | 980 | −22.464 | 15.903 | −33.541 | 1.00 | 59.02 |
| ATOM | 2159 | C | ASP | A | 980 | −21.190 | 16.645 | −33.122 | 1.00 | 57.66 |
| ATOM | 2160 | O | ASP | A | 980 | −20.858 | 17.709 | −33.649 | 1.00 | 56.21 |
| ATOM | 2161 | CB | ASP | A | 980 | −23.215 | 16.654 | −34.649 | 1.00 | 60.77 |
| ATOM | 2162 | CG | ASP | A | 980 | −22.502 | 16.607 | −35.990 | 1.00 | 62.49 |
| ATOM | 2163 | OD1 | ASP | A | 980 | −21.643 | 15.717 | −36.183 | 1.00 | 64.46 |
| ATOM | 2164 | OD2 | ASP | A | 980 | −22.821 | 17.456 | −36.858 | 1.00 | 61.55 |
| ATOM | 2165 | N | PRO | A | 981 | −20.453 | 16.066 | −32.162 | 1.00 | 56.22 |
| ATOM | 2166 | CA | PRO | A | 981 | −19.208 | 16.598 | −31.598 | 1.00 | 55.44 |
| ATOM | 2167 | C | PRO | A | 981 | −18.084 | 17.037 | −32.535 | 1.00 | 53.93 |
| ATOM | 2168 | O | PRO | A | 981 | −17.492 | 18.090 | −32.323 | 1.00 | 53.34 |
| ATOM | 2169 | CB | PRO | A | 981 | −18.772 | 15.496 | −30.629 | 1.00 | 55.07 |
| ATOM | 2170 | CG | PRO | A | 981 | −19.345 | 14.262 | −31.229 | 1.00 | 54.89 |
| ATOM | 2171 | CD | PRO | A | 981 | −20.718 | 14.715 | −31.637 | 1.00 | 55.91 |
| ATOM | 2172 | N | GLN | A | 982 | −17.773 | 16.245 | −33.556 | 1.00 | 54.01 |
| ATOM | 2173 | CA | GLN | A | 982 | −16.698 | 16.617 | −34.476 | 1.00 | 53.13 |
| ATOM | 2174 | C | GLN | A | 982 | −17.043 | 17.886 | −35.253 | 1.00 | 50.44 |
| ATOM | 2175 | O | GLN | A | 982 | −16.188 | 18.476 | −35.897 | 1.00 | 48.82 |
| ATOM | 2176 | CB | GLN | A | 982 | −16.377 | 15.470 | −35.445 | 1.00 | 55.75 |
| ATOM | 2177 | CG | GLN | A | 982 | −15.535 | 14.333 | −34.851 | 1.00 | 59.04 |
| ATOM | 2178 | CD | GLN | A | 982 | −16.299 | 13.465 | −33.854 | 1.00 | 63.65 |
| ATOM | 2179 | OE1 | GLN | A | 982 | −17.524 | 13.576 | −33.721 | 1.00 | 65.05 |
| ATOM | 2180 | NE2 | GLN | A | 982 | −15.578 | 12.605 | −33.140 | 1.00 | 65.14 |
| ATOM | 2181 | N | ARG | A | 983 | −18.296 | 18.311 | −35.179 | 1.00 | 49.29 |
| ATOM | 2182 | CA | ARG | A | 983 | −18.716 | 19.528 | −35.861 | 1.00 | 49.03 |
| ATOM | 2183 | C | ARG | A | 983 | −18.482 | 20.771 | −35.012 | 1.00 | 47.62 |
| ATOM | 2184 | O | ARG | A | 983 | −18.549 | 21.890 | −35.514 | 1.00 | 48.25 |
| ATOM | 2185 | CB | ARG | A | 983 | −20.196 | 19.479 | −36.200 | 1.00 | 50.98 |
| ATOM | 2186 | CG | ARG | A | 983 | −20.683 | 20.768 | −36.834 | 1.00 | 53.68 |
| ATOM | 2187 | CD | ARG | A | 983 | −22.171 | 20.801 | −36.932 | 1.00 | 55.23 |
| ATOM | 2188 | NE | ARG | A | 983 | −22.623 | 21.823 | −37.864 | 1.00 | 57.30 |
| ATOM | 2189 | CZ | ARG | A | 983 | −23.903 | 22.094 | −38.081 | 1.00 | 58.03 |
| ATOM | 2190 | NH1 | ARG | A | 983 | −24.838 | 21.416 | −37.424 | 1.00 | 59.36 |
| ATOM | 2191 | NH2 | ARG | A | 983 | −24.247 | 23.028 | −38.954 | 1.00 | 57.21 |
| ATOM | 2192 | N | TYR | A | 984 | −18.217 | 20.585 | −33.727 | 1.00 | 46.14 |
| ATOM | 2193 | CA | TYR | A | 984 | −18.005 | 21.728 | −32.860 | 1.00 | 46.62 |
| ATOM | 2194 | C | TYR | A | 984 | −16.629 | 21.830 | −32.178 | 1.00 | 45.34 |
| ATOM | 2195 | O | TYR | A | 984 | −16.165 | 22.923 | −31.866 | 1.00 | 44.15 |
| ATOM | 2196 | CB | TYR | A | 984 | −19.162 | 21.797 | −31.855 | 1.00 | 45.73 |
| ATOM | 2197 | CG | TYR | A | 984 | −20.483 | 22.095 | −32.545 | 1.00 | 47.52 |
| ATOM | 2198 | CD1 | TYR | A | 984 | −21.367 | 21.075 | −32.892 | 1.00 | 49.29 |
| ATOM | 2199 | CD2 | TYR | A | 984 | −20.816 | 23.401 | −32.920 | 1.00 | 47.84 |
| ATOM | 2200 | CE1 | TYR | A | 984 | −22.553 | 21.352 | −33.598 | 1.00 | 48.94 |
| ATOM | 2201 | CE2 | TYR | A | 984 | −21.987 | 23.683 | −33.622 | 1.00 | 47.24 |
| ATOM | 2202 | CZ | TYR | A | 984 | −22.847 | 22.658 | −33.957 | 1.00 | 48.48 |
| ATOM | 2203 | OH | TYR | A | 984 | −23.996 | 22.944 | −34.655 | 1.00 | 50.81 |
| ATOM | 2204 | N | LEU | A | 985 | −15.976 | 20.698 | −31.957 | 1.00 | 44.95 |
| ATOM | 2205 | CA | LEU | A | 985 | −14.648 | 20.689 | −31.353 | 1.00 | 45.72 |
| ATOM | 2206 | C | LEU | A | 985 | −13.746 | 19.932 | −32.320 | 1.00 | 47.79 |
| ATOM | 2207 | O | LEU | A | 985 | −14.094 | 18.836 | −32.770 | 1.00 | 47.32 |
| ATOM | 2208 | CB | LEU | A | 985 | −14.672 | 19.980 | −29.997 | 1.00 | 44.68 |
| ATOM | 2209 | CG | LEU | A | 985 | −15.410 | 20.665 | −28.849 | 1.00 | 44.43 |
| ATOM | 2210 | CD1 | LEU | A | 985 | −15.243 | 19.853 | −27.574 | 1.00 | 43.38 |
| ATOM | 2211 | CD2 | LEU | A | 985 | −14.860 | 22.079 | −28.660 | 1.00 | 44.96 |
| ATOM | 2212 | N | VAL | A | 986 | −12.596 | 20.510 | −32.647 | 1.00 | 48.85 |
| ATOM | 2213 | CA | VAL | A | 986 | −11.679 | 19.871 | −33.582 | 1.00 | 49.84 |
| ATOM | 2214 | C | VAL | A | 986 | −10.398 | 19.397 | −32.911 | 1.00 | 51.24 |
| ATOM | 2215 | O | VAL | A | 986 | −9.426 | 20.136 | −32.805 | 1.00 | 51.87 |
| ATOM | 2216 | CB | VAL | A | 986 | −11.312 | 20.828 | −34.716 | 1.00 | 51.14 |
| ATOM | 2217 | CG1 | VAL | A | 986 | −10.392 | 20.130 | −35.709 | 1.00 | 50.04 |
| ATOM | 2218 | CG2 | VAL | A | 986 | −12.573 | 21.325 | −35.394 | 1.00 | 49.84 |
| ATOM | 2219 | N | ILE | A | 987 | −10.406 | 18.146 | −32.473 | 1.00 | 53.09 |
| ATOM | 2220 | CA | ILE | A | 987 | −9.267 | 17.541 | −31.797 | 1.00 | 52.98 |
| ATOM | 2221 | C | ILE | A | 987 | −8.535 | 16.595 | −32.736 | 1.00 | 55.59 |
| ATOM | 2222 | O | ILE | A | 987 | −9.155 | 15.754 | −33.392 | 1.00 | 53.71 |
| ATOM | 2223 | CB | ILE | A | 987 | −9.735 | 16.727 | −30.591 | 1.00 | 50.88 |
| ATOM | 2224 | CG1 | ILE | A | 987 | −10.571 | 17.611 | −29.665 | 1.00 | 52.22 |
| ATOM | 2225 | CG2 | ILE | A | 987 | −8.549 | 16.124 | −29.884 | 1.00 | 49.91 |
| ATOM | 2226 | CD1 | ILE | A | 987 | −11.307 | 16.840 | −28.586 | 1.00 | 49.45 |
| ATOM | 2227 | N | GLN | A | 988 | −7.214 | 16.723 | −32.792 | 1.00 | 59.02 |
| ATOM | 2228 | CA | GLN | A | 988 | −6.418 | 15.860 | −33.645 | 1.00 | 61.68 |
| ATOM | 2229 | C | GLN | A | 988 | −6.654 | 14.424 | −33.178 | 1.00 | 64.13 |
| ATOM | 2230 | O | GLN | A | 988 | −6.577 | 14.127 | −31.980 | 1.00 | 62.70 |
| ATOM | 2231 | CB | GLN | A | 988 | −4.946 | 16.236 | −33.528 | 1.00 | 63.64 |
| ATOM | 2232 | CG | GLN | A | 988 | −4.134 | 15.850 | −34.745 | 1.00 | 67.15 |
| ATOM | 2233 | CD | GLN | A | 988 | −2.726 | 16.407 | −34.726 | 1.00 | 67.61 |
| ATOM | 2234 | OE1 | GLN | A | 988 | −1.961 | 16.193 | −35.659 | 1.00 | 71.31 |
| ATOM | 2235 | NE2 | GLN | A | 988 | −2.378 | 17.124 | −33.665 | 1.00 | 70.34 |
| ATOM | 2236 | N | GLY | A | 989 | −6.947 | 13.542 | −34.131 | 1.00 | 66.36 |
| ATOM | 2237 | CA | GLY | A | 989 | −7.240 | 12.156 | −33.809 | 1.00 | 68.95 |
| ATOM | 2238 | C | GLY | A | 989 | −8.751 | 12.071 | −33.788 | 1.00 | 71.82 |
| ATOM | 2239 | O | GLY | A | 989 | −9.386 | 12.067 | −34.840 | 1.00 | 73.55 |
| ATOM | 2240 | N | ASP | A | 990 | −9.317 | 12.011 | −32.586 | 1.00 | 74.05 |
| ATOM | 2241 | CA | ASP | A | 990 | −10.766 | 11.986 | −32.362 | 1.00 | 75.83 |
| ATOM | 2242 | C | ASP | A | 990 | −11.591 | 11.562 | −33.583 | 1.00 | 77.60 |
| ATOM | 2243 | O | ASP | A | 990 | −12.564 | 12.219 | −33.951 | 1.00 | 77.72 |
| ATOM | 2244 | CB | ASP | A | 990 | −11.194 | 13.387 | −31.870 | 1.00 | 73.28 |
| ATOM | 2245 | CG | ASP | A | 990 | −12.647 | 13.457 | −31.412 | 1.00 | 70.03 |
| ATOM | 2246 | OD1 | ASP | A | 990 | −13.121 | 12.530 | −30.715 | 1.00 | 70.19 |
| ATOM | 2247 | OD2 | ASP | A | 990 | −13.308 | 14.466 | −31.732 | 1.00 | 65.64 |
| ATOM | 2248 | N | ALA | A | 991 | −11.208 | 10.456 | −34.208 | 1.00 | 80.41 |
| ATOM | 2249 | CA | ALA | A | 991 | −11.936 | 9.974 | −35.374 | 1.00 | 83.98 |
| ATOM | 2250 | C | ALA | A | 991 | −12.676 | 8.672 | −35.059 | 1.00 | 86.60 |
| ATOM | 2251 | O | ALA | A | 991 | −13.837 | 8.503 | −35.441 | 1.00 | 87.41 |
| ATOM | 2252 | CB | ALA | A | 991 | −10.972 | 9.771 | −36.550 | 1.00 | 82.75 |
| ATOM | 2253 | N | ALA | A | 992 | −12.005 | 7.767 | −34.347 | 1.00 | 88.48 |
| ATOM | 2254 | CA | ALA | A | 992 | −12.582 | 6.471 | −33.992 | 1.00 | 90.40 |
| ATOM | 2255 | C | ALA | A | 992 | −12.798 | 6.298 | −32.492 | 1.00 | 91.49 |
| ATOM | 2256 | O | ALA | A | 992 | −13.875 | 5.784 | −32.113 | 1.00 | 91.08 |
| ATOM | 2257 | CB | ALA | A | 992 | −11.694 | 5.350 | −34.516 | 1.00 | 90.58 |
| ATOM | 2258 | OXT | ALA | A | 992 | −11.879 | 6.655 | −31.721 | 1.00 | 92.46 |
| ATOM | 2259 | N | ALA | B | 694 | −21.367 | 6.079 | −64.456 | 1.00 | 78.72 |
| ATOM | 2260 | CA | ALA | B | 694 | −22.190 | 6.371 | −63.252 | 1.00 | 78.66 |
| ATOM | 2261 | C | ALA | B | 694 | −21.358 | 7.182 | −62.263 | 1.00 | 78.89 |
| ATOM | 2262 | O | ALA | B | 694 | −20.424 | 7.882 | −62.653 | 1.00 | 79.39 |
| ATOM | 2263 | CB | ALA | B | 694 | −22.652 | 5.062 | −62.614 | 1.00 | 77.87 |
| ATOM | 2264 | N | ALA | B | 695 | −21.703 | 7.085 | −60.984 | 1.00 | 78.95 |
| ATOM | 2265 | CA | ALA | B | 695 | −20.990 | 7.791 | −59.923 | 1.00 | 78.98 |
| ATOM | 2266 | C | ALA | B | 695 | −20.828 | 6.832 | −58.742 | 1.00 | 79.45 |
| ATOM | 2267 | O | ALA | B | 695 | −21.816 | 6.296 | −58.240 | 1.00 | 80.20 |
| ATOM | 2268 | CB | ALA | B | 695 | −21.775 | 9.030 | −59.498 | 1.00 | 77.61 |
| ATOM | 2269 | N | ALA | B | 696 | −19.588 | 6.618 | −58.305 | 1.00 | 79.38 |
| ATOM | 2270 | CA | ALA | B | 696 | −19.298 | 5.702 | −57.200 | 1.00 | 79.53 |
| ATOM | 2271 | C | ALA | B | 696 | −20.251 | 5.840 | −56.006 | 1.00 | 80.35 |
| ATOM | 2272 | O | ALA | B | 696 | −20.595 | 6.950 | −55.604 | 1.00 | 80.03 |
| ATOM | 2273 | CB | ALA | B | 696 | −17.849 | 5.883 | −56.742 | 1.00 | 77.04 |
| ATOM | 2274 | N | PRO | B | 697 | −20.699 | 4.701 | −55.435 | 1.00 | 81.52 |
| ATOM | 2275 | CA | PRO | B | 697 | −21.611 | 4.620 | −54.284 | 1.00 | 81.54 |
| ATOM | 2276 | C | PRO | B | 697 | −21.046 | 5.326 | −53.053 | 1.00 | 82.10 |
| ATOM | 2277 | O | PRO | B | 697 | −19.916 | 5.067 | −52.635 | 1.00 | 82.80 |
| ATOM | 2278 | CB | PRO | B | 697 | −21.752 | 3.118 | −54.058 | 1.00 | 81.54 |
| ATOM | 2279 | CG | PRO | B | 697 | −21.616 | 2.564 | −55.434 | 1.00 | 82.60 |
| ATOM | 2280 | CD | PRO | B | 697 | −20.452 | 3.355 | −55.987 | 1.00 | 82.37 |
| ATOM | 2281 | N | LEU | B | 698 | −21.845 | 6.207 | −52.467 | 1.00 | 81.85 |
| ATOM | 2282 | CA | LEU | B | 698 | −21.421 | 6.969 | −51.303 | 1.00 | 80.76 |
| ATOM | 2283 | C | LEU | B | 698 | −21.365 | 6.109 | −50.051 | 1.00 | 79.77 |
| ATOM | 2284 | O | LEU | B | 698 | −21.874 | 4.994 | −50.048 | 1.00 | 80.40 |
| ATOM | 2285 | CB | LEU | B | 698 | −22.383 | 8.136 | −51.093 | 1.00 | 80.50 |
| ATOM | 2286 | CG | LEU | B | 698 | −22.038 | 9.096 | −49.959 | 1.00 | 80.61 |
| ATOM | 2287 | CD1 | LEU | B | 698 | −20.711 | 9.781 | −50.243 | 1.00 | 80.34 |
| ATOM | 2288 | CD2 | LEU | B | 698 | −23.150 | 10.118 | −49.823 | 1.00 | 80.62 |
| ATOM | 2289 | N | THR | B | 699 | −20.732 | 6.641 | −49.006 | 1.00 | 79.28 |
| ATOM | 2290 | CA | THR | B | 699 | −20.585 | 5.992 | −47.692 | 1.00 | 79.67 |
| ATOM | 2291 | C | THR | B | 699 | −19.385 | 6.573 | −46.938 | 1.00 | 78.91 |
| ATOM | 2292 | O | THR | B | 699 | −18.317 | 5.969 | −46.894 | 1.00 | 79.37 |
| ATOM | 2293 | CB | THR | B | 699 | −20.408 | 4.444 | −47.792 | 1.00 | 80.11 |
| ATOM | 2294 | OG1 | THR | B | 699 | −19.202 | 4.135 | −48.503 | 1.00 | 80.37 |
| ATOM | 2295 | CG2 | THR | B | 699 | −21.616 | 3.801 | −48.481 | 1.00 | 78.91 |
| ATOM | 2296 | N | PRO | B | 700 | −19.555 | 7.757 | −46.324 | 1.00 | 78.42 |
| ATOM | 2297 | CA | PRO | B | 700 | −18.508 | 8.454 | −45.566 | 1.00 | 78.09 |
| ATOM | 2298 | C | PRO | B | 700 | −17.960 | 7.713 | −44.354 | 1.00 | 77.41 |
| ATOM | 2299 | O | PRO | B | 700 | −18.560 | 6.759 | −43.863 | 1.00 | 77.46 |
| ATOM | 2300 | CB | PRO | B | 700 | −19.182 | 9.764 | −45.176 | 1.00 | 77.99 |
| ATOM | 2301 | CG | PRO | B | 700 | −20.601 | 9.355 | −45.002 | 1.00 | 78.57 |
| ATOM | 2302 | CD | PRO | B | 700 | −20.833 | 8.482 | −46.218 | 1.00 | 78.31 |
| ATOM | 2303 | N | SER | B | 701 | −16.811 | 8.175 | −43.876 | 1.00 | 76.96 |
| ATOM | 2304 | CA | SER | B | 701 | −16.150 | 7.577 | −42.725 | 1.00 | 76.79 |
| ATOM | 2305 | C | SER | B | 701 | −15.878 | 8.645 | −41.676 | 1.00 | 76.45 |
| ATOM | 2306 | O | SER | B | 701 | −16.079 | 9.838 | −41.920 | 1.00 | 76.52 |
| ATOM | 2307 | CB | SER | B | 701 | −14.824 | 6.944 | −43.154 | 1.00 | 77.52 |
| ATOM | 2308 | OG | SER | B | 701 | −13.944 | 7.924 | −43.690 | 1.00 | 77.51 |
| ATOM | 2309 | N | GLY | B | 702 | −15.418 | 8.221 | −40.507 | 1.00 | 75.81 |
| ATOM | 2310 | CA | GLY | B | 702 | −15.123 | 9.188 | −39.466 | 1.00 | 75.42 |
| ATOM | 2311 | C | GLY | B | 702 | −14.011 | 10.118 | −39.912 | 1.00 | 73.55 |
| ATOM | 2312 | O | GLY | B | 702 | −14.195 | 11.334 | −40.015 | 1.00 | 73.83 |
| ATOM | 2313 | N | THR | B | 703 | −12.858 | 9.521 | −40.194 | 1.00 | 71.08 |
| ATOM | 2314 | CA | THR | B | 703 | −11.662 | 10.227 | −40.629 | 1.00 | 68.76 |
| ATOM | 2315 | C | THR | B | 703 | −11.903 | 11.547 | −41.346 | 1.00 | 66.14 |
| ATOM | 2316 | O | THR | B | 703 | −12.657 | 11.616 | −42.309 | 1.00 | 66.70 |
| ATOM | 2317 | CB | THR | B | 703 | −10.826 | 9.341 | −41.553 | 1.00 | 70.67 |
| ATOM | 2318 | OG1 | THR | B | 703 | −10.649 | 8.051 | −40.948 | 1.00 | 72.31 |
| ATOM | 2319 | CG2 | THR | B | 703 | −9.466 | 9.982 | −41.809 | 1.00 | 71.40 |
| ATOM | 2320 | N | ALA | B | 704 | −11.244 | 12.596 | −40.871 | 1.00 | 62.68 |
| ATOM | 2321 | CA | ALA | B | 704 | −11.370 | 13.915 | −41.469 | 1.00 | 59.07 |
| ATOM | 2322 | C | ALA | B | 704 | −10.277 | 14.034 | −42.529 | 1.00 | 57.68 |
| ATOM | 2323 | O | ALA | B | 704 | −9.333 | 13.236 | −42.556 | 1.00 | 56.92 |
| ATOM | 2324 | CB | ALA | B | 704 | −11.210 | 14.992 | −40.403 | 1.00 | 56.44 |
| ATOM | 2325 | N | PRO | B | 705 | −10.387 | 15.030 | −43.418 | 1.00 | 54.82 |
| ATOM | 2326 | CA | PRO | B | 705 | −9.393 | 15.215 | −44.473 | 1.00 | 53.14 |
| ATOM | 2327 | C | PRO | B | 705 | −8.001 | 15.562 | −43.982 | 1.00 | 52.08 |
| ATOM | 2328 | O | PRO | B | 705 | −7.835 | 16.297 | −43.019 | 1.00 | 52.38 |
| ATOM | 2329 | CB | PRO | B | 705 | −9.995 | 16.328 | −45.317 | 1.00 | 53.47 |
| ATOM | 2330 | CG | PRO | B | 705 | −10.713 | 17.142 | −44.308 | 1.00 | 53.49 |
| ATOM | 2331 | CD | PRO | B | 705 | −11.394 | 16.102 | −43.457 | 1.00 | 53.49 |
| ATOM | 2332 | N | ASN | B | 706 | −7.005 | 15.018 | −44.668 | 1.00 | 50.16 |
| ATOM | 2333 | CA | ASN | B | 706 | −5.614 | 15.255 | −44.352 | 1.00 | 47.68 |
| ATOM | 2334 | C | ASN | B | 706 | −5.141 | 16.419 | −45.220 | 1.00 | 48.60 |
| ATOM | 2335 | O | ASN | B | 706 | −4.817 | 16.235 | −46.394 | 1.00 | 47.17 |
| ATOM | 2336 | CB | ASN | B | 706 | −4.798 | 14.007 | −44.679 | 1.00 | 46.68 |
| ATOM | 2337 | CG | ASN | B | 706 | −3.336 | 14.152 | −44.307 | 1.00 | 47.50 |
| ATOM | 2338 | OD1 | ASN | B | 706 | −2.843 | 15.264 | −44.085 | 1.00 | 46.16 |
| ATOM | 2339 | ND2 | ASN | B | 706 | −2.624 | 13.025 | −44.252 | 1.00 | 47.14 |
| ATOM | 2340 | N | GLN | B | 707 | −5.089 | 17.616 | −44.654 | 1.00 | 48.45 |
| ATOM | 2341 | CA | GLN | B | 707 | −4.663 | 18.753 | −45.448 | 1.00 | 50.34 |
| ATOM | 2342 | C | GLN | B | 707 | −3.195 | 19.132 | −45.296 | 1.00 | 49.32 |
| ATOM | 2343 | O | GLN | B | 707 | −2.820 | 20.282 | −45.508 | 1.00 | 48.81 |
| ATOM | 2344 | CB | GLN | B | 707 | −5.565 | 19.961 | −45.172 | 1.00 | 51.33 |
| ATOM | 2345 | CG | GLN | B | 707 | −7.017 | 19.707 | −45.534 | 1.00 | 54.04 |
| ATOM | 2346 | CD | GLN | B | 707 | −7.890 | 20.942 | −45.390 | 1.00 | 57.85 |
| ATOM | 2347 | OE1 | GLN | B | 707 | −7.874 | 21.834 | −46.244 | 1.00 | 59.23 |
| ATOM | 2348 | NE2 | GLN | B | 707 | −8.651 | 21.007 | −44.296 | 1.00 | 57.51 |
| ATOM | 2349 | N | ALA | B | 708 | −2.361 | 18.160 | −44.944 | 1.00 | 49.01 |
| ATOM | 2350 | CA | ALA | B | 708 | −0.930 | 18.416 | −44.814 | 1.00 | 49.28 |
| ATOM | 2351 | C | ALA | B | 708 | −0.402 | 18.939 | −46.153 | 1.00 | 50.28 |
| ATOM | 2352 | O | ALA | B | 708 | −0.927 | 18.609 | −47.216 | 1.00 | 50.22 |
| ATOM | 2353 | CB | ALA | B | 708 | −0.201 | 17.134 | −44.434 | 1.00 | 46.73 |
| ATOM | 2354 | N | GLN | B | 709 | 0.635 | 19.761 | −46.102 | 1.00 | 52.30 |
| ATOM | 2355 | CA | GLN | B | 709 | 1.209 | 20.305 | −47.320 | 1.00 | 53.30 |
| ATOM | 2356 | C | GLN | B | 709 | 2.521 | 19.639 | −47.662 | 1.00 | 54.63 |
| ATOM | 2357 | O | GLN | B | 709 | 3.328 | 19.342 | −46.788 | 1.00 | 55.25 |
| ATOM | 2358 | CB | GLN | B | 709 | 1.438 | 21.807 | −47.173 | 1.00 | 53.77 |
| ATOM | 2359 | CG | GLN | B | 709 | 0.187 | 22.623 | −47.359 | 1.00 | 56.18 |
| ATOM | 2360 | CD | GLN | B | 709 | −0.326 | 22.555 | −48.777 | 1.00 | 58.18 |
| ATOM | 2361 | OE1 | GLN | B | 709 | −1.494 | 22.832 | −49.037 | 1.00 | 59.37 |
| ATOM | 2362 | NE2 | GLN | B | 709 | 0.553 | 22.197 | −49.710 | 1.00 | 59.96 |
| ATOM | 2363 | N | LEU | B | 710 | 2.734 | 19.394 | −48.943 | 1.00 | 56.61 |
| ATOM | 2364 | CA | LEU | B | 710 | 3.984 | 18.793 | −49.368 | 1.00 | 59.18 |
| ATOM | 2365 | C | LEU | B | 710 | 4.565 | 19.616 | −50.508 | 1.00 | 59.54 |
| ATOM | 2366 | O | LEU | B | 710 | 4.077 | 19.563 | −51.634 | 1.00 | 60.05 |
| ATOM | 2367 | CB | LEU | B | 710 | 3.788 | 17.341 | −49.826 | 1.00 | 58.84 |
| ATOM | 2368 | CG | LEU | B | 710 | 5.144 | 16.673 | −50.079 | 1.00 | 58.85 |
| ATOM | 2369 | CD1 | LEU | B | 710 | 5.941 | 16.662 | −48.780 | 1.00 | 56.82 |
| ATOM | 2370 | CD2 | LEU | B | 710 | 4.960 | 15.272 | −50.610 | 1.00 | 59.31 |
| ATOM | 2371 | N | ARG | B | 711 | 5.602 | 20.385 | −50.201 | 1.00 | 59.61 |
| ATOM | 2372 | CA | ARG | B | 711 | 6.249 | 21.218 | −51.199 | 1.00 | 59.44 |
| ATOM | 2373 | C | ARG | B | 711 | 7.209 | 20.387 | −52.020 | 1.00 | 57.96 |
| ATOM | 2374 | O | ARG | B | 711 | 8.038 | 19.665 | −51.487 | 1.00 | 56.87 |
| ATOM | 2375 | CB | ARG | B | 711 | 7.026 | 22.352 | −50.528 | 1.00 | 62.07 |
| ATOM | 2376 | CG | ARG | B | 711 | 6.173 | 23.468 | −49.947 | 1.00 | 66.77 |
| ATOM | 2377 | CD | ARG | B | 711 | 6.166 | 24.664 | −50.880 | 1.00 | 70.76 |
| ATOM | 2378 | NE | ARG | B | 711 | 5.529 | 25.830 | −50.275 | 1.00 | 73.86 |
| ATOM | 2379 | CZ | ARG | B | 711 | 5.482 | 27.030 | −50.849 | 1.00 | 73.77 |
| ATOM | 2380 | NH1 | ARG | B | 711 | 6.038 | 27.213 | −52.039 | 1.00 | 71.33 |
| ATOM | 2381 | NH2 | ARG | B | 711 | 4.872 | 28.041 | −50.241 | 1.00 | 74.12 |
| ATOM | 2382 | N | ILE | B | 712 | 7.073 | 20.472 | −53.331 | 1.00 | 58.48 |
| ATOM | 2383 | CA | ILE | B | 712 | 7.968 | 19.765 | −54.221 | 1.00 | 59.27 |
| ATOM | 2384 | C | ILE | B | 712 | 8.952 | 20.858 | −54.638 | 1.00 | 60.66 |
| ATOM | 2385 | O | ILE | B | 712 | 8.601 | 21.793 | −55.355 | 1.00 | 60.01 |
| ATOM | 2386 | CB | ILE | B | 712 | 7.177 | 19.162 | −55.390 | 1.00 | 58.30 |
| ATOM | 2387 | CG1 | ILE | B | 712 | 6.266 | 18.066 | −54.826 | 1.00 | 57.96 |
| ATOM | 2388 | CG2 | ILE | B | 712 | 8.117 | 18.601 | −56.448 | 1.00 | 56.69 |
| ATOM | 2389 | CD1 | ILE | B | 712 | 5.405 | 17.380 | −55.840 | 1.00 | 61.91 |
| ATOM | 2390 | N | LEU | B | 713 | 10.178 | 20.744 | −54.139 | 1.00 | 60.97 |
| ATOM | 2391 | CA | LEU | B | 713 | 11.201 | 21.748 | −54.374 | 1.00 | 61.57 |
| ATOM | 2392 | C | LEU | B | 713 | 12.091 | 21.583 | −55.583 | 1.00 | 62.89 |
| ATOM | 2393 | O | LEU | B | 713 | 12.517 | 20.476 | −55.921 | 1.00 | 62.63 |
| ATOM | 2394 | CB | LEU | B | 713 | 12.093 | 21.864 | −53.141 | 1.00 | 60.62 |
| ATOM | 2395 | CG | LEU | B | 713 | 11.375 | 21.861 | −51.796 | 1.00 | 60.72 |
| ATOM | 2396 | CD1 | LEU | B | 713 | 12.345 | 22.362 | −50.739 | 1.00 | 60.07 |
| ATOM | 2397 | CD2 | LEU | B | 713 | 10.134 | 22.743 | −51.848 | 1.00 | 58.84 |
| ATOM | 2398 | N | ALA | B | 714 | 12.388 | 22.713 | −56.216 | 1.00 | 63.60 |
| ATOM | 2399 | CA | ALA | B | 714 | 13.257 | 22.724 | −57.373 | 1.00 | 64.47 |
| ATOM | 2400 | C | ALA | B | 714 | 14.663 | 22.544 | −56.828 | 1.00 | 65.36 |
| ATOM | 2401 | O | ALA | B | 714 | 15.049 | 23.194 | −55.857 | 1.00 | 64.64 |
| ATOM | 2402 | CB | ALA | B | 714 | 13.136 | 24.052 | −58.115 | 1.00 | 65.35 |
| ATOM | 2403 | N | GLU | B | 715 | 15.409 | 21.642 | −57.451 | 1.00 | 66.38 |
| ATOM | 2404 | CA | GLU | B | 715 | 16.786 | 21.337 | −57.075 | 1.00 | 68.71 |
| ATOM | 2405 | C | GLU | B | 715 | 17.570 | 22.608 | −56.764 | 1.00 | 69.58 |
| ATOM | 2406 | O | GLU | B | 715 | 18.489 | 22.606 | −55.945 | 1.00 | 70.53 |
| ATOM | 2407 | CB | GLU | B | 715 | 17.451 | 20.590 | −58.233 | 1.00 | 71.46 |
| ATOM | 2408 | CG | GLU | B | 715 | 18.838 | 20.031 | −57.972 | 1.00 | 75.03 |
| ATOM | 2409 | CD | GLU | B | 715 | 19.366 | 19.255 | −59.174 | 1.00 | 78.03 |
| ATOM | 2410 | OE1 | GLU | B | 715 | 20.444 | 18.627 | −59.064 | 1.00 | 81.21 |
| ATOM | 2411 | OE2 | GLU | B | 715 | 18.699 | 19.275 | −60.234 | 1.00 | 78.12 |
| ATOM | 2412 | N | THR | B | 716 | 17.178 | 23.692 | −57.422 | 1.00 | 69.78 |
| ATOM | 2413 | CA | THR | B | 716 | 17.825 | 24.987 | −57.283 | 1.00 | 69.47 |
| ATOM | 2414 | C | THR | B | 716 | 17.451 | 25.789 | −56.041 | 1.00 | 69.52 |
| ATOM | 2415 | O | THR | B | 716 | 18.240 | 26.611 | −55.573 | 1.00 | 70.64 |
| ATOM | 2416 | CB | THR | B | 716 | 17.536 | 25.866 | −58.513 | 1.00 | 70.06 |
| ATOM | 2417 | OG1 | THR | B | 716 | 18.171 | 27.138 | −58.351 | 1.00 | 71.27 |
| ATOM | 2418 | CG2 | THR | B | 716 | 16.036 | 26.076 | −58.678 | 1.00 | 69.60 |
| ATOM | 2419 | N | GLU | B | 717 | 16.253 | 25.577 | −55.511 | 1.00 | 68.00 |
| ATOM | 2420 | CA | GLU | B | 717 | 15.845 | 26.321 | −54.330 | 1.00 | 66.43 |
| ATOM | 2421 | C | GLU | B | 717 | 16.699 | 25.898 | −53.135 | 1.00 | 66.27 |
| ATOM | 2422 | O | GLU | B | 717 | 16.735 | 26.588 | −52.117 | 1.00 | 65.77 |
| ATOM | 2423 | CB | GLU | B | 717 | 14.363 | 26.072 | −54.032 | 1.00 | 67.50 |
| ATOM | 2424 | CG | GLU | B | 717 | 13.430 | 26.254 | −55.234 | 1.00 | 67.97 |
| ATOM | 2425 | CD | GLU | B | 717 | 11.971 | 25.930 | −54.911 | 1.00 | 68.95 |
| ATOM | 2426 | OE1 | GLU | B | 717 | 11.314 | 25.253 | −55.736 | 1.00 | 69.80 |
| ATOM | 2427 | OE2 | GLU | B | 717 | 11.479 | 26.355 | −53.843 | 1.00 | 67.07 |
| ATOM | 2428 | N | LEU | B | 718 | 17.409 | 24.778 | −53.277 | 1.00 | 66.24 |
| ATOM | 2429 | CA | LEU | B | 718 | 18.240 | 24.241 | −52.197 | 1.00 | 67.00 |
| ATOM | 2430 | C | LEU | B | 718 | 19.741 | 24.416 | −52.344 | 1.00 | 68.38 |
| ATOM | 2431 | O | LEU | B | 718 | 20.308 | 24.173 | −53.410 | 1.00 | 68.22 |
| ATOM | 2432 | CB | LEU | B | 718 | 17.961 | 22.746 | −51.989 | 1.00 | 64.67 |
| ATOM | 2433 | CG | LEU | B | 718 | 16.592 | 22.299 | −51.473 | 1.00 | 64.09 |
| ATOM | 2434 | CD1 | LEU | B | 718 | 16.642 | 20.805 | −51.218 | 1.00 | 64.50 |
| ATOM | 2435 | CD2 | LEU | B | 718 | 16.221 | 23.038 | −50.196 | 1.00 | 61.97 |
| ATOM | 2436 | N | ALA | B | 719 | 20.376 | 24.807 | −51.240 | 1.00 | 69.63 |
| ATOM | 2437 | CA | ALA | B | 719 | 21.820 | 25.008 | −51.188 | 1.00 | 70.57 |
| ATOM | 2438 | C | ALA | B | 719 | 22.409 | 24.242 | −50.006 | 1.00 | 71.59 |
| ATOM | 2439 | O | ALA | B | 719 | 21.972 | 24.404 | −48.859 | 1.00 | 71.07 |
| ATOM | 2440 | CB | ALA | B | 719 | 22.152 | 26.499 | −51.062 | 1.00 | 69.58 |
| ATOM | 2441 | N | ARG | B | 720 | 23.399 | 23.404 | −50.303 | 1.00 | 72.39 |
| ATOM | 2442 | CA | ARG | B | 720 | 24.083 | 22.601 | −49.296 | 1.00 | 73.45 |
| ATOM | 2443 | C | ARG | B | 720 | 25.143 | 23.436 | −48.577 | 1.00 | 74.60 |
| ATOM | 2444 | O | ARG | B | 720 | 25.385 | 24.593 | −48.911 | 1.00 | 75.29 |
| ATOM | 2445 | CB | ARG | B | 720 | 24.786 | 21.414 | −49.952 | 1.00 | 73.03 |
| ATOM | 2446 | CG | ARG | B | 720 | 23.902 | 20.485 | −50.756 | 1.00 | 74.45 |
| ATOM | 2447 | CD | ARG | B | 720 | 23.856 | 19.106 | −50.118 | 1.00 | 75.59 |
| ATOM | 2448 | NE | ARG | B | 720 | 23.875 | 18.018 | −51.097 | 1.00 | 76.81 |
| ATOM | 2449 | CZ | ARG | B | 720 | 22.976 | 17.855 | −52.063 | 1.00 | 76.86 |
| ATOM | 2450 | NH1 | ARG | B | 720 | 21.972 | 18.714 | −52.198 | 1.00 | 76.25 |
| ATOM | 2451 | NH2 | ARG | B | 720 | 23.074 | 16.818 | −52.886 | 1.00 | 77.66 |
| ATOM | 2452 | N | VAL | B | 721 | 25.774 | 22.831 | −47.583 | 1.00 | 75.37 |
| ATOM | 2453 | CA | VAL | B | 721 | 26.835 | 23.472 | −46.824 | 1.00 | 75.73 |
| ATOM | 2454 | C | VAL | B | 721 | 27.804 | 22.356 | −46.447 | 1.00 | 76.50 |
| ATOM | 2455 | O | VAL | B | 721 | 28.600 | 21.912 | −47.275 | 1.00 | 76.71 |
| ATOM | 2456 | CB | VAL | B | 721 | 26.297 | 24.148 | −45.550 | 1.00 | 76.20 |
| ATOM | 2457 | CG1 | VAL | B | 721 | 27.451 | 24.684 | −44.722 | 1.00 | 76.08 |
| ATOM | 2458 | CG2 | VAL | B | 721 | 25.358 | 25.282 | −45.925 | 1.00 | 75.75 |
| ATOM | 2459 | N | ALA | B | 722 | 27.722 | 21.889 | −45.208 | 1.00 | 77.05 |
| ATOM | 2460 | CA | ALA | B | 722 | 28.584 | 20.809 | −44.751 | 1.00 | 76.34 |
| ATOM | 2461 | C | ALA | B | 722 | 27.735 | 19.558 | −44.559 | 1.00 | 75.78 |
| ATOM | 2462 | O | ALA | B | 722 | 26.537 | 19.548 | −44.868 | 1.00 | 75.60 |
| ATOM | 2463 | CB | ALA | B | 722 | 29.262 | 21.196 | −43.433 | 1.00 | 76.05 |
| ATOM | 2464 | N | VAL | B | 723 | 28.373 | 18.507 | −44.053 | 1.00 | 74.68 |
| ATOM | 2465 | CA | VAL | B | 723 | 27.714 | 17.239 | −43.777 | 1.00 | 71.94 |
| ATOM | 2466 | C | VAL | B | 723 | 27.423 | 17.159 | −42.283 | 1.00 | 71.54 |
| ATOM | 2467 | O | VAL | B | 723 | 28.310 | 17.399 | −41.461 | 1.00 | 71.55 |
| ATOM | 2468 | CB | VAL | B | 723 | 28.611 | 16.058 | −44.160 | 1.00 | 71.07 |
| ATOM | 2469 | CG1 | VAL | B | 723 | 28.020 | 14.767 | −43.629 | 1.00 | 70.27 |
| ATOM | 2470 | CG2 | VAL | B | 723 | 28.764 | 15.998 | −45.672 | 1.00 | 71.21 |
| ATOM | 2471 | N | LEU | B | 724 | 26.182 | 16.836 | −41.931 | 1.00 | 69.67 |
| ATOM | 2472 | CA | LEU | B | 724 | 25.806 | 16.721 | −40.529 | 1.00 | 66.81 |
| ATOM | 2473 | C | LEU | B | 724 | 26.058 | 15.300 | −40.057 | 1.00 | 66.43 |
| ATOM | 2474 | O | LEU | B | 724 | 26.094 | 15.032 | −38.859 | 1.00 | 68.21 |
| ATOM | 2475 | CB | LEU | B | 724 | 24.334 | 17.080 | −40.347 | 1.00 | 64.61 |
| ATOM | 2476 | CG | LEU | B | 724 | 23.992 | 18.555 | −40.555 | 1.00 | 64.25 |
| ATOM | 2477 | CD1 | LEU | B | 724 | 22.493 | 18.740 | −40.600 | 1.00 | 62.81 |
| ATOM | 2478 | CD2 | LEU | B | 724 | 24.593 | 19.379 | −39.431 | 1.00 | 63.59 |
| ATOM | 2479 | N | GLY | B | 725 | 26.248 | 14.403 | −41.018 | 1.00 | 65.74 |
| ATOM | 2480 | CA | GLY | B | 725 | 26.484 | 13.004 | −40.723 | 1.00 | 65.55 |
| ATOM | 2481 | C | GLY | B | 725 | 25.812 | 12.138 | −41.777 | 1.00 | 67.20 |
| ATOM | 2482 | O | GLY | B | 725 | 24.919 | 12.602 | −42.487 | 1.00 | 67.07 |
| ATOM | 2483 | N | SER | B | 726 | 26.241 | 10.884 | −41.888 | 1.00 | 68.22 |
| ATOM | 2484 | CA | SER | B | 726 | 25.668 | 9.957 | −42.860 | 1.00 | 69.48 |
| ATOM | 2485 | C | SER | B | 726 | 25.632 | 8.553 | −42.275 | 1.00 | 70.10 |
| ATOM | 2486 | O | SER | B | 726 | 26.012 | 8.344 | −41.126 | 1.00 | 71.12 |
| ATOM | 2487 | CB | SER | B | 726 | 26.504 | 9.935 | −44.139 | 1.00 | 70.71 |
| ATOM | 2488 | OG | SER | B | 726 | 27.715 | 9.214 | −43.947 | 1.00 | 71.56 |
| ATOM | 2489 | N | GLY | B | 727 | 25.186 | 7.593 | −43.079 | 1.00 | 71.04 |
| ATOM | 2490 | CA | GLY | B | 727 | 25.109 | 6.217 | −42.623 | 1.00 | 72.17 |
| ATOM | 2491 | C | GLY | B | 727 | 24.190 | 5.350 | −43.469 | 1.00 | 72.74 |
| ATOM | 2492 | O | GLY | B | 727 | 23.941 | 5.645 | −44.642 | 1.00 | 73.49 |
| ATOM | 2493 | N | ALA | B | 728 | 23.683 | 4.280 | −42.865 | 1.00 | 72.58 |
| ATOM | 2494 | CA | ALA | B | 728 | 22.794 | 3.342 | −43.539 | 1.00 | 72.96 |
| ATOM | 2495 | C | ALA | B | 728 | 21.657 | 4.049 | −44.263 | 1.00 | 74.25 |
| ATOM | 2496 | O | ALA | B | 728 | 21.285 | 3.682 | −45.381 | 1.00 | 73.50 |
| ATOM | 2497 | CB | ALA | B | 728 | 22.226 | 2.361 | −42.527 | 1.00 | 71.89 |
| ATOM | 2498 | N | PHE | B | 729 | 21.110 | 5.071 | −43.618 | 1.00 | 76.13 |
| ATOM | 2499 | CA | PHE | B | 729 | 20.003 | 5.825 | −44.186 | 1.00 | 78.00 |
| ATOM | 2500 | C | PHE | B | 729 | 20.488 | 7.116 | −44.823 | 1.00 | 78.39 |
| ATOM | 2501 | O | PHE | B | 729 | 20.531 | 8.153 | −44.167 | 1.00 | 80.61 |
| ATOM | 2502 | CB | PHE | B | 729 | 18.980 | 6.125 | −43.091 | 1.00 | 78.54 |
| ATOM | 2503 | CG | PHE | B | 729 | 18.734 | 4.958 | −42.176 | 1.00 | 80.05 |
| ATOM | 2504 | CD1 | PHE | B | 729 | 19.552 | 4.741 | −41.067 | 1.00 | 80.31 |
| ATOM | 2505 | CD2 | PHE | B | 729 | 17.731 | 4.029 | −42.464 | 1.00 | 80.12 |
| ATOM | 2506 | CE1 | PHE | B | 729 | 19.379 | 3.613 | −40.258 | 1.00 | 79.48 |
| ATOM | 2507 | CE2 | PHE | B | 729 | 17.551 | 2.902 | −41.666 | 1.00 | 79.41 |
| ATOM | 2508 | CZ | PHE | B | 729 | 18.378 | 2.694 | −40.561 | 1.00 | 80.03 |
| ATOM | 2509 | N | GLY | B | 730 | 20.867 | 7.040 | −46.098 | 1.00 | 77.21 |
| ATOM | 2510 | CA | GLY | B | 730 | 21.343 | 8.209 | −46.819 | 1.00 | 75.57 |
| ATOM | 2511 | C | GLY | B | 730 | 22.288 | 9.121 | −46.055 | 1.00 | 74.55 |
| ATOM | 2512 | O | GLY | B | 730 | 22.876 | 8.721 | −45.051 | 1.00 | 74.34 |
| ATOM | 2513 | N | THR | B | 731 | 22.447 | 10.349 | −46.540 | 1.00 | 73.71 |
| ATOM | 2514 | CA | THR | B | 731 | 23.322 | 11.316 | −45.887 | 1.00 | 72.89 |
| ATOM | 2515 | C | THR | B | 731 | 22.575 | 12.607 | −45.597 | 1.00 | 72.69 |
| ATOM | 2516 | O | THR | B | 731 | 21.765 | 13.069 | −46.406 | 1.00 | 72.79 |
| ATOM | 2517 | CB | THR | B | 731 | 24.547 | 11.650 | −46.751 | 1.00 | 73.00 |
| ATOM | 2518 | OG1 | THR | B | 731 | 25.364 | 10.484 | −46.894 | 1.00 | 72.99 |
| ATOM | 2519 | CG2 | THR | B | 731 | 25.367 | 12.753 | −46.100 | 1.00 | 72.78 |
| ATOM | 2520 | N | VAL | B | 732 | 22.854 | 13.186 | −44.434 | 1.00 | 72.09 |
| ATOM | 2521 | CA | VAL | B | 732 | 22.204 | 14.422 | −44.025 | 1.00 | 71.72 |
| ATOM | 2522 | C | VAL | B | 732 | 23.109 | 15.616 | −44.283 | 1.00 | 71.79 |
| ATOM | 2523 | O | VAL | B | 732 | 24.312 | 15.553 | −44.053 | 1.00 | 73.24 |
| ATOM | 2524 | CB | VAL | B | 732 | 21.817 | 14.385 | −42.525 | 1.00 | 71.31 |
| ATOM | 2525 | CG1 | VAL | B | 732 | 21.170 | 15.704 | −42.124 | 1.00 | 70.14 |
| ATOM | 2526 | CG2 | VAL | B | 732 | 20.858 | 13.220 | −42.256 | 1.00 | 69.55 |
| ATOM | 2527 | N | TYR | B | 733 | 22.517 | 16.703 | −44.762 | 1.00 | 71.37 |
| ATOM | 2528 | CA | TYR | B | 733 | 23.256 | 17.915 | −45.072 | 1.00 | 70.26 |
| ATOM | 2529 | C | TYR | B | 733 | 22.636 | 19.144 | −44.437 | 1.00 | 70.40 |
| ATOM | 2530 | O | TYR | B | 733 | 21.423 | 19.328 | −44.475 | 1.00 | 69.19 |
| ATOM | 2531 | CB | TYR | B | 733 | 23.280 | 18.136 | −46.581 | 1.00 | 71.02 |
| ATOM | 2532 | CG | TYR | B | 733 | 24.205 | 17.228 | −47.350 | 1.00 | 72.14 |
| ATOM | 2533 | CD1 | TYR | B | 733 | 25.552 | 17.550 | −47.516 | 1.00 | 72.40 |
| ATOM | 2534 | CD2 | TYR | B | 733 | 23.733 | 16.051 | −47.928 | 1.00 | 72.72 |
| ATOM | 2535 | CE1 | TYR | B | 733 | 26.403 | 16.722 | −48.244 | 1.00 | 72.86 |
| ATOM | 2536 | CE2 | TYR | B | 733 | 24.573 | 15.215 | −48.654 | 1.00 | 72.43 |
| ATOM | 2537 | CZ | TYR | B | 733 | 25.904 | 15.556 | −48.807 | 1.00 | 72.95 |
| ATOM | 2538 | OH | TYR | B | 733 | 26.734 | 14.728 | −49.519 | 1.00 | 73.75 |
| ATOM | 2539 | N | LYS | B | 734 | 23.468 | 19.988 | −43.845 | 1.00 | 71.52 |
| ATOM | 2540 | CA | LYS | B | 734 | 22.971 | 21.235 | −43.278 | 1.00 | 71.70 |
| ATOM | 2541 | C | LYS | B | 734 | 22.836 | 22.110 | −44.526 | 1.00 | 70.70 |
| ATOM | 2542 | O | LYS | B | 734 | 23.636 | 21.981 | −45.457 | 1.00 | 69.26 |
| ATOM | 2543 | CB | LYS | B | 734 | 24.000 | 21.843 | −42.323 | 1.00 | 72.03 |
| ATOM | 2544 | CG | LYS | B | 734 | 23.619 | 23.219 | −41.810 | 1.00 | 73.15 |
| ATOM | 2545 | CD | LYS | B | 734 | 24.811 | 23.927 | −41.173 | 1.00 | 74.86 |
| ATOM | 2546 | CE | LYS | B | 734 | 24.469 | 25.371 | −40.815 | 1.00 | 75.25 |
| ATOM | 2547 | NZ | LYS | B | 734 | 25.609 | 26.084 | −40.177 | 1.00 | 75.36 |
| ATOM | 2548 | N | GLY | B | 735 | 21.836 | 22.983 | −44.570 | 1.00 | 69.60 |
| ATOM | 2549 | CA | GLY | B | 735 | 21.694 | 23.804 | −45.757 | 1.00 | 70.34 |
| ATOM | 2550 | C | GLY | B | 735 | 20.674 | 24.921 | −45.717 | 1.00 | 70.22 |
| ATOM | 2551 | O | GLY | B | 735 | 20.003 | 25.146 | −44.709 | 1.00 | 70.41 |
| ATOM | 2552 | N | ILE | B | 736 | 20.559 | 25.626 | −46.835 | 1.00 | 70.46 |
| ATOM | 2553 | CA | ILE | B | 736 | 19.619 | 26.730 | −46.932 | 1.00 | 71.88 |
| ATOM | 2554 | C | ILE | B | 736 | 18.619 | 26.490 | −48.053 | 1.00 | 70.97 |
| ATOM | 2555 | O | ILE | B | 736 | 18.988 | 26.084 | −49.158 | 1.00 | 70.48 |
| ATOM | 2556 | CB | ILE | B | 736 | 20.344 | 28.065 | −47.216 | 1.00 | 73.37 |
| ATOM | 2557 | CG1 | ILE | B | 736 | 21.503 | 28.253 | −46.232 | 1.00 | 75.08 |
| ATOM | 2558 | CG2 | ILE | B | 736 | 19.359 | 29.225 | −47.099 | 1.00 | 72.22 |
| ATOM | 2559 | CD1 | ILE | B | 736 | 22.363 | 29.482 | −46.523 | 1.00 | 77.81 |
| ATOM | 2560 | N | TRP | B | 737 | 17.349 | 26.734 | −47.756 | 1.00 | 69.75 |
| ATOM | 2561 | CA | TRP | B | 737 | 16.301 | 26.571 | −48.746 | 1.00 | 69.68 |
| ATOM | 2562 | C | TRP | B | 737 | 15.679 | 27.924 | −49.044 | 1.00 | 71.04 |
| ATOM | 2563 | O | TRP | B | 737 | 15.167 | 28.593 | −48.142 | 1.00 | 70.73 |
| ATOM | 2564 | CB | TRP | B | 737 | 15.219 | 25.612 | −48.247 | 1.00 | 67.06 |
| ATOM | 2565 | CG | TRP | B | 737 | 13.973 | 25.642 | −49.085 | 1.00 | 63.60 |
| ATOM | 2566 | CD1 | TRP | B | 737 | 13.900 | 25.681 | −50.449 | 1.00 | 62.26 |
| ATOM | 2567 | CD2 | TRP | B | 737 | 12.623 | 25.615 | −48.614 | 1.00 | 62.62 |
| ATOM | 2568 | NE1 | TRP | B | 737 | 12.589 | 25.683 | −50.857 | 1.00 | 61.65 |
| ATOM | 2569 | CE2 | TRP | B | 737 | 11.783 | 25.641 | −49.752 | 1.00 | 61.65 |
| ATOM | 2570 | CE3 | TRP | B | 737 | 12.041 | 25.567 | −47.342 | 1.00 | 62.03 |
| ATOM | 2571 | CZ2 | TRP | B | 737 | 10.393 | 25.621 | −49.658 | 1.00 | 61.01 |
| ATOM | 2572 | CZ3 | TRP | B | 737 | 10.657 | 25.545 | −47.246 | 1.00 | 63.21 |
| ATOM | 2573 | CH2 | TRP | B | 737 | 9.847 | 25.573 | −48.402 | 1.00 | 62.84 |
| ATOM | 2574 | N | VAL | B | 738 | 15.727 | 28.320 | −50.313 | 1.00 | 71.93 |
| ATOM | 2575 | CA | VAL | B | 738 | 15.162 | 29.590 | −50.738 | 1.00 | 72.89 |
| ATOM | 2576 | C | VAL | B | 738 | 13.895 | 29.317 | −51.539 | 1.00 | 74.11 |
| ATOM | 2577 | O | VAL | B | 738 | 13.960 | 29.000 | −52.722 | 1.00 | 74.51 |
| ATOM | 2578 | CB | VAL | B | 738 | 16.155 | 30.357 | −51.614 | 1.00 | 73.24 |
| ATOM | 2579 | CG1 | VAL | B | 738 | 15.753 | 31.811 | −51.677 | 1.00 | 72.41 |
| ATOM | 2580 | CG2 | VAL | B | 738 | 17.573 | 30.189 | −51.067 | 1.00 | 71.96 |
| ATOM | 2581 | N | PRO | B | 739 | 12.723 | 29.441 | −50.898 | 1.00 | 75.71 |
| ATOM | 2582 | CA | PRO | B | 739 | 11.408 | 29.212 | −51.504 | 1.00 | 78.23 |
| ATOM | 2583 | C | PRO | B | 739 | 11.246 | 29.754 | −52.922 | 1.00 | 80.64 |
| ATOM | 2584 | O | PRO | B | 739 | 11.912 | 30.716 | −53.309 | 1.00 | 80.64 |
| ATOM | 2585 | CB | PRO | B | 739 | 10.462 | 29.882 | −50.518 | 1.00 | 77.56 |
| ATOM | 2586 | CG | PRO | B | 739 | 11.135 | 29.618 | −49.210 | 1.00 | 77.83 |
| ATOM | 2587 | CD | PRO | B | 739 | 12.574 | 29.957 | −49.527 | 1.00 | 76.20 |
| ATOM | 2588 | N | ALA | B | 740 | 10.348 | 29.128 | −53.682 | 1.00 | 83.02 |
| ATOM | 2589 | CA | ALA | B | 740 | 10.077 | 29.521 | −55.062 | 1.00 | 84.99 |
| ATOM | 2590 | C | ALA | B | 740 | 9.540 | 30.948 | −55.142 | 1.00 | 86.49 |
| ATOM | 2591 | O | ALA | B | 740 | 8.328 | 31.168 | −55.063 | 1.00 | 87.30 |
| ATOM | 2592 | CB | ALA | B | 740 | 9.080 | 28.554 | −55.695 | 1.00 | 84.35 |
| ATOM | 2593 | N | GLY | B | 741 | 10.449 | 31.912 | −55.293 | 1.00 | 87.21 |
| ATOM | 2594 | CA | GLY | B | 741 | 10.048 | 33.306 | −55.390 | 1.00 | 88.15 |
| ATOM | 2595 | C | GLY | B | 741 | 10.039 | 34.051 | −54.069 | 1.00 | 88.91 |
| ATOM | 2596 | O | GLY | B | 741 | 9.134 | 34.841 | −53.796 | 1.00 | 89.07 |
| ATOM | 2597 | N | GLU | B | 742 | 11.051 | 33.804 | −53.245 | 1.00 | 89.01 |
| ATOM | 2598 | CA | GLU | B | 742 | 11.146 | 34.460 | −51.949 | 1.00 | 89.25 |
| ATOM | 2599 | C | GLU | B | 742 | 12.605 | 34.733 | −51.597 | 1.00 | 89.18 |
| ATOM | 2600 | O | GLU | B | 742 | 13.514 | 34.121 | −52.163 | 1.00 | 87.36 |
| ATOM | 2601 | CB | GLU | B | 742 | 10.486 | 33.592 | −50.874 | 1.00 | 89.33 |
| ATOM | 2602 | CG | GLU | B | 742 | 9.030 | 33.270 | −51.166 | 1.00 | 89.86 |
| ATOM | 2603 | CD | GLU | B | 742 | 8.394 | 32.409 | −50.096 | 1.00 | 91.41 |
| ATOM | 2604 | OE1 | GLU | B | 742 | 7.217 | 32.022 | −50.259 | 1.00 | 92.46 |
| ATOM | 2605 | OE2 | GLU | B | 742 | 9.068 | 32.116 | −49.089 | 1.00 | 91.98 |
| ATOM | 2606 | N | ALA | B | 743 | 12.818 | 35.659 | −50.664 | 1.00 | 89.63 |
| ATOM | 2607 | CA | ALA | B | 743 | 14.163 | 36.037 | −50.241 | 1.00 | 89.61 |
| ATOM | 2608 | C | ALA | B | 743 | 14.626 | 35.259 | −49.016 | 1.00 | 89.33 |
| ATOM | 2609 | O | ALA | B | 743 | 15.813 | 34.951 | −48.884 | 1.00 | 89.32 |
| ATOM | 2610 | CB | ALA | B | 743 | 14.219 | 37.538 | −49.956 | 1.00 | 88.93 |
| ATOM | 2611 | N | VAL | B | 744 | 13.692 | 34.944 | −48.122 | 1.00 | 88.36 |
| ATOM | 2612 | CA | VAL | B | 744 | 14.016 | 34.203 | −46.902 | 1.00 | 86.73 |
| ATOM | 2613 | C | VAL | B | 744 | 14.987 | 33.060 | −47.185 | 1.00 | 85.49 |
| ATOM | 2614 | O | VAL | B | 744 | 14.800 | 32.293 | −48.129 | 1.00 | 86.20 |
| ATOM | 2615 | CB | VAL | B | 744 | 12.744 | 33.615 | −46.239 | 1.00 | 86.40 |
| ATOM | 2616 | CG1 | VAL | B | 744 | 11.827 | 34.737 | −45.777 | 1.00 | 86.46 |
| ATOM | 2617 | CG2 | VAL | B | 744 | 12.018 | 32.709 | −47.217 | 1.00 | 86.17 |
| ATOM | 2618 | N | ALA | B | 745 | 16.033 | 32.959 | −46.373 | 1.00 | 83.22 |
| ATOM | 2619 | CA | ALA | B | 745 | 17.020 | 31.901 | −46.537 | 1.00 | 80.32 |
| ATOM | 2620 | C | ALA | B | 745 | 16.858 | 30.887 | −45.402 | 1.00 | 78.54 |
| ATOM | 2621 | O | ALA | B | 745 | 17.722 | 30.780 | −44.537 | 1.00 | 77.78 |
| ATOM | 2622 | CB | ALA | B | 745 | 18.426 | 32.497 | −46.528 | 1.00 | 79.74 |
| ATOM | 2623 | N | ILE | B | 746 | 15.742 | 30.154 | −45.415 | 1.00 | 76.18 |
| ATOM | 2624 | CA | ILE | B | 746 | 15.427 | 29.144 | −44.394 | 1.00 | 73.00 |
| ATOM | 2625 | C | ILE | B | 746 | 16.506 | 28.085 | −44.148 | 1.00 | 70.60 |
| ATOM | 2626 | O | ILE | B | 746 | 16.926 | 27.379 | −45.068 | 1.00 | 70.94 |
| ATOM | 2627 | CB | ILE | B | 746 | 14.133 | 28.384 | −44.740 | 1.00 | 73.31 |
| ATOM | 2628 | CG1 | ILE | B | 746 | 12.939 | 29.338 | −44.735 | 1.00 | 74.65 |
| ATOM | 2629 | CG2 | ILE | B | 746 | 13.906 | 27.271 | −43.733 | 1.00 | 73.54 |
| ATOM | 2630 | CD1 | ILE | B | 746 | 11.630 | 28.690 | −45.169 | 1.00 | 74.90 |
| ATOM | 2631 | N | PRO | B | 747 | 16.963 | 27.959 | −42.890 | 1.00 | 68.46 |
| ATOM | 2632 | CA | PRO | B | 747 | 17.991 | 26.973 | −42.542 | 1.00 | 66.18 |
| ATOM | 2633 | C | PRO | B | 747 | 17.322 | 25.609 | −42.442 | 1.00 | 63.94 |
| ATOM | 2634 | O | PRO | B | 747 | 16.327 | 25.441 | −41.727 | 1.00 | 62.47 |
| ATOM | 2635 | CB | PRO | B | 747 | 18.499 | 27.475 | −41.198 | 1.00 | 65.24 |
| ATOM | 2636 | CG | PRO | B | 747 | 17.258 | 28.014 | −40.581 | 1.00 | 65.83 |
| ATOM | 2637 | CD | PRO | B | 747 | 16.625 | 28.788 | −41.718 | 1.00 | 67.44 |
| ATOM | 2638 | N | VAL | B | 748 | 17.864 | 24.638 | −43.162 | 1.00 | 61.19 |
| ATOM | 2639 | CA | VAL | B | 748 | 17.273 | 23.316 | −43.161 | 1.00 | 60.40 |
| ATOM | 2640 | C | VAL | B | 748 | 18.273 | 22.179 | −43.190 | 1.00 | 60.05 |
| ATOM | 2641 | O | VAL | B | 748 | 19.484 | 22.383 | −43.273 | 1.00 | 59.27 |
| ATOM | 2642 | CB | VAL | B | 748 | 16.346 | 23.121 | −44.382 | 1.00 | 60.82 |
| ATOM | 2643 | CG1 | VAL | B | 748 | 15.250 | 24.166 | −44.384 | 1.00 | 60.94 |
| ATOM | 2644 | CG2 | VAL | B | 748 | 17.159 | 23.192 | −45.662 | 1.00 | 58.19 |
| ATOM | 2645 | N | ALA | B | 749 | 17.726 | 20.973 | −43.110 | 1.00 | 59.07 |
| ATOM | 2646 | CA | ALA | B | 749 | 18.503 | 19.757 | −43.177 | 1.00 | 59.25 |
| ATOM | 2647 | C | ALA | B | 749 | 18.025 | 19.161 | −44.489 | 1.00 | 60.46 |
| ATOM | 2648 | O | ALA | B | 749 | 16.899 | 19.422 | −44.910 | 1.00 | 61.58 |
| ATOM | 2649 | CB | ALA | B | 749 | 18.159 | 18.837 | −42.019 | 1.00 | 57.97 |
| ATOM | 2650 | N | ILE | B | 750 | 18.881 | 18.388 | −45.144 | 1.00 | 60.55 |
| ATOM | 2651 | CA | ILE | B | 750 | 18.536 | 17.762 | −46.407 | 1.00 | 59.69 |
| ATOM | 2652 | C | ILE | B | 750 | 19.113 | 16.364 | −46.376 | 1.00 | 60.35 |
| ATOM | 2653 | O | ILE | B | 750 | 20.304 | 16.182 | −46.120 | 1.00 | 59.91 |
| ATOM | 2654 | CB | ILE | B | 750 | 19.152 | 18.519 | −47.593 | 1.00 | 60.90 |
| ATOM | 2655 | CG1 | ILE | B | 750 | 18.855 | 20.014 | −47.470 | 1.00 | 61.45 |
| ATOM | 2656 | CG2 | ILE | B | 750 | 18.593 | 17.976 | −48.898 | 1.00 | 61.29 |
| ATOM | 2657 | CD1 | ILE | B | 750 | 19.552 | 20.868 | −48.512 | 1.00 | 62.03 |
| ATOM | 2658 | N | LYS | B | 751 | 18.272 | 15.373 | −46.629 | 1.00 | 60.64 |
| ATOM | 2659 | CA | LYS | B | 751 | 18.736 | 14.003 | −46.608 | 1.00 | 61.71 |
| ATOM | 2660 | C | LYS | B | 751 | 18.568 | 13.371 | −47.970 | 1.00 | 64.10 |
| ATOM | 2661 | O | LYS | B | 751 | 17.569 | 13.593 | −48.652 | 1.00 | 63.74 |
| ATOM | 2662 | CB | LYS | B | 751 | 17.962 | 13.216 | −45.557 | 1.00 | 60.11 |
| ATOM | 2663 | CG | LYS | B | 751 | 18.422 | 11.788 | −45.379 | 1.00 | 58.61 |
| ATOM | 2664 | CD | LYS | B | 751 | 17.930 | 11.241 | −44.049 | 1.00 | 58.06 |
| ATOM | 2665 | CE | LYS | B | 751 | 18.424 | 9.840 | −43.818 | 1.00 | 56.70 |
| ATOM | 2666 | NZ | LYS | B | 751 | 18.112 | 9.366 | −42.448 | 1.00 | 57.25 |
| ATOM | 2667 | N | ILE | B | 752 | 19.562 | 12.590 | −48.367 | 1.00 | 67.30 |
| ATOM | 2668 | CA | ILE | B | 752 | 19.532 | 11.909 | −49.650 | 1.00 | 70.25 |
| ATOM | 2669 | C | ILE | B | 752 | 19.714 | 10.425 | −49.393 | 1.00 | 71.94 |
| ATOM | 2670 | O | ILE | B | 752 | 20.740 | 10.013 | −48.867 | 1.00 | 73.06 |
| ATOM | 2671 | CB | ILE | B | 752 | 20.666 | 12.407 | −50.556 | 1.00 | 70.74 |
| ATOM | 2672 | CG1 | ILE | B | 752 | 20.578 | 13.930 | −50.705 | 1.00 | 70.64 |
| ATOM | 2673 | CG2 | ILE | B | 752 | 20.578 | 11.721 | −51.912 | 1.00 | 71.95 |
| ATOM | 2674 | CD1 | ILE | B | 752 | 21.674 | 14.538 | −51.555 | 1.00 | 69.52 |
| ATOM | 2675 | N | ALA | B | 753 | 18.723 | 9.623 | −49.760 | 1.00 | 74.71 |
| ATOM | 2676 | CA | ALA | B | 753 | 18.799 | 8.185 | −49.536 | 1.00 | 78.36 |
| ATOM | 2677 | C | ALA | B | 753 | 19.570 | 7.481 | −50.639 | 1.00 | 81.19 |
| ATOM | 2678 | O | ALA | B | 753 | 19.369 | 7.757 | −51.821 | 1.00 | 81.29 |
| ATOM | 2679 | CB | ALA | B | 753 | 17.399 | 7.601 | −49.423 | 1.00 | 77.47 |
| ATOM | 2680 | N | VAL | B | 754 | 20.453 | 6.570 | −50.238 | 1.00 | 85.07 |
| ATOM | 2681 | CA | VAL | B | 754 | 21.281 | 5.798 | −51.170 | 1.00 | 88.42 |
| ATOM | 2682 | C | VAL | B | 754 | 20.468 | 5.271 | −52.358 | 1.00 | 90.41 |
| ATOM | 2683 | O | VAL | B | 754 | 19.817 | 4.229 | −52.260 | 1.00 | 91.29 |
| ATOM | 2684 | CB | VAL | B | 754 | 21.940 | 4.581 | −50.459 | 1.00 | 88.45 |
| ATOM | 2685 | CG1 | VAL | B | 754 | 23.015 | 3.969 | −51.357 | 1.00 | 86.93 |
| ATOM | 2686 | CG2 | VAL | B | 754 | 22.519 | 5.009 | −49.108 | 1.00 | 88.93 |
| ATOM | 2687 | N | ALA | B | 755 | 20.517 | 5.991 | −53.477 | 1.00 | 91.96 |
| ATOM | 2688 | CA | ALA | B | 755 | 19.789 | 5.605 | −54.684 | 1.00 | 93.14 |
| ATOM | 2689 | CB | ALA | B | 755 | 20.303 | 6.403 | −55.878 | 1.00 | 92.70 |
| ATOM | 2690 | C | ALA | B | 755 | 19.890 | 4.104 | −54.975 | 1.00 | 94.28 |
| ATOM | 2691 | OT1 | ALA | B | 755 | 18.831 | 3.482 | −55.224 | 1.00 | 94.26 |
| ATOM | 2692 | OT2 | ALA | B | 755 | 21.023 | 3.569 | −54.962 | 1.00 | 94.42 |
| ATOM | 2693 | N | ALA | B | 761 | 9.723 | 1.143 | −56.947 | 1.00 | 87.14 |
| ATOM | 2694 | CA | ALA | B | 761 | 10.011 | 1.599 | −55.557 | 1.00 | 87.01 |
| ATOM | 2695 | C | ALA | B | 761 | 9.596 | 3.053 | −55.300 | 1.00 | 87.49 |
| ATOM | 2696 | O | ALA | B | 761 | 9.028 | 3.358 | −54.254 | 1.00 | 88.29 |
| ATOM | 2697 | CB | ALA | B | 761 | 11.492 | 1.420 | −55.252 | 1.00 | 86.17 |
| ATOM | 2698 | N | ASN | B | 762 | 9.879 | 3.946 | −56.245 | 1.00 | 87.30 |
| ATOM | 2699 | CA | ASN | B | 762 | 9.532 | 5.358 | −56.091 | 1.00 | 86.68 |
| ATOM | 2700 | C | ASN | B | 762 | 8.123 | 5.587 | −55.563 | 1.00 | 85.76 |
| ATOM | 2701 | O | ASN | B | 762 | 7.796 | 6.676 | −55.095 | 1.00 | 85.55 |
| ATOM | 2702 | CB | ASN | B | 762 | 9.711 | 6.095 | −57.417 | 1.00 | 87.83 |
| ATOM | 2703 | CG | ASN | B | 762 | 11.145 | 6.515 | −57.652 | 1.00 | 88.91 |
| ATOM | 2704 | OD1 | ASN | B | 762 | 12.065 | 5.696 | −57.587 | 1.00 | 88.14 |
| ATOM | 2705 | ND2 | ASN | B | 762 | 11.347 | 7.800 | −57.923 | 1.00 | 89.08 |
| ATOM | 2706 | N | VAL | B | 763 | 7.288 | 4.559 | −55.645 | 1.00 | 84.95 |
| ATOM | 2707 | CA | VAL | B | 763 | 5.919 | 4.652 | −55.152 | 1.00 | 83.59 |
| ATOM | 2708 | C | VAL | B | 763 | 5.981 | 4.500 | −53.634 | 1.00 | 82.82 |
| ATOM | 2709 | O | VAL | B | 763 | 5.380 | 5.277 | −52.891 | 1.00 | 82.67 |
| ATOM | 2710 | CB | VAL | B | 763 | 5.039 | 3.517 | −55.713 | 1.00 | 83.22 |
| ATOM | 2711 | CG1 | VAL | B | 763 | 3.574 | 3.913 | −55.639 | 1.00 | 83.32 |
| ATOM | 2712 | CG2 | VAL | B | 763 | 5.452 | 3.190 | −57.134 | 1.00 | 83.28 |
| ATOM | 2713 | N | ALA | B | 764 | 6.723 | 3.485 | −53.192 | 1.00 | 81.06 |
| ATOM | 2714 | CA | ALA | B | 764 | 6.899 | 3.188 | −51.774 | 1.00 | 79.39 |
| ATOM | 2715 | C | ALA | B | 764 | 7.374 | 4.428 | −51.024 | 1.00 | 77.74 |
| ATOM | 2716 | O | ALA | B | 764 | 7.068 | 4.616 | −49.841 | 1.00 | 77.59 |
| ATOM | 2717 | CB | ALA | B | 764 | 7.911 | 2.048 | −51.601 | 1.00 | 78.81 |
| ATOM | 2718 | N | PHE | B | 765 | 8.124 | 5.269 | −51.727 | 1.00 | 75.13 |
| ATOM | 2719 | CA | PHE | B | 765 | 8.653 | 6.497 | −51.156 | 1.00 | 72.04 |
| ATOM | 2720 | C | PHE | B | 765 | 7.617 | 7.621 | −51.141 | 1.00 | 69.21 |
| ATOM | 2721 | O | PHE | B | 765 | 7.398 | 8.271 | −50.120 | 1.00 | 67.19 |
| ATOM | 2722 | CB | PHE | B | 765 | 9.890 | 6.937 | −51.939 | 1.00 | 72.95 |
| ATOM | 2723 | CG | PHE | B | 765 | 10.256 | 8.364 | −51.716 | 1.00 | 74.51 |
| ATOM | 2724 | CD1 | PHE | B | 765 | 9.604 | 9.378 | −52.416 | 1.00 | 76.66 |
| ATOM | 2725 | CD2 | PHE | B | 765 | 11.187 | 8.708 | −50.749 | 1.00 | 74.99 |
| ATOM | 2726 | CE1 | PHE | B | 765 | 9.868 | 10.717 | −52.152 | 1.00 | 76.83 |
| ATOM | 2727 | CE2 | PHE | B | 765 | 11.460 | 10.042 | −50.474 | 1.00 | 76.87 |
| ATOM | 2728 | CZ | PHE | B | 765 | 10.797 | 11.053 | −51.177 | 1.00 | 77.47 |
| ATOM | 2729 | N | MET | B | 766 | 6.989 | 7.858 | −52.281 | 1.00 | 67.05 |
| ATOM | 2730 | CA | MET | B | 766 | 5.990 | 8.903 | −52.362 | 1.00 | 66.91 |
| ATOM | 2731 | C | MET | B | 766 | 4.876 | 8.664 | −51.351 | 1.00 | 66.11 |
| ATOM | 2732 | O | MET | B | 766 | 4.201 | 9.598 | −50.924 | 1.00 | 66.81 |
| ATOM | 2733 | CB | MET | B | 766 | 5.412 | 8.975 | −53.778 | 1.00 | 68.18 |
| ATOM | 2734 | CG | MET | B | 766 | 6.425 | 9.399 | −54.826 | 1.00 | 68.28 |
| ATOM | 2735 | SD | MET | B | 766 | 7.326 | 10.874 | −54.318 | 1.00 | 68.93 |
| ATOM | 2736 | CE | MET | B | 766 | 6.095 | 12.100 | −54.463 | 1.00 | 67.19 |
| ATOM | 2737 | N | ASP | B | 767 | 4.678 | 7.412 | −50.966 | 1.00 | 64.47 |
| ATOM | 2738 | CA | ASP | B | 767 | 3.642 | 7.109 | −49.997 | 1.00 | 63.79 |
| ATOM | 2739 | C | ASP | B | 767 | 4.142 | 7.370 | −48.587 | 1.00 | 61.82 |
| ATOM | 2740 | O | ASP | B | 767 | 3.420 | 7.922 | −47.761 | 1.00 | 61.10 |
| ATOM | 2741 | CB | ASP | B | 767 | 3.186 | 5.660 | −50.146 | 1.00 | 67.06 |
| ATOM | 2742 | CG | ASP | B | 767 | 2.150 | 5.490 | −51.246 | 1.00 | 70.14 |
| ATOM | 2743 | OD1 | ASP | B | 767 | 1.000 | 5.939 | −51.059 | 1.00 | 71.60 |
| ATOM | 2744 | OD2 | ASP | B | 767 | 2.484 | 4.917 | −52.304 | 1.00 | 73.46 |
| ATOM | 2745 | N | GLU | B | 768 | 5.385 | 6.984 | −48.320 | 1.00 | 60.15 |
| ATOM | 2746 | CA | GLU | B | 768 | 5.978 | 7.194 | −47.008 | 1.00 | 58.81 |
| ATOM | 2747 | C | GLU | B | 768 | 6.184 | 8.689 | −46.770 | 1.00 | 57.14 |
| ATOM | 2748 | O | GLU | B | 768 | 6.001 | 9.184 | −45.654 | 1.00 | 54.76 |
| ATOM | 2749 | CB | GLU | B | 768 | 7.319 | 6.459 | −46.906 | 1.00 | 61.07 |
| ATOM | 2750 | CG | GLU | B | 768 | 7.904 | 6.444 | −45.495 | 1.00 | 64.70 |
| ATOM | 2751 | CD | GLU | B | 768 | 7.060 | 5.642 | −44.506 | 1.00 | 66.61 |
| ATOM | 2752 | OE1 | GLU | B | 768 | 7.238 | 5.825 | −43.283 | 1.00 | 68.31 |
| ATOM | 2753 | OE2 | GLU | B | 768 | 6.227 | 4.823 | −44.948 | 1.00 | 66.65 |
| ATOM | 2754 | N | ALA | B | 769 | 6.553 | 9.405 | −47.829 | 1.00 | 55.28 |
| ATOM | 2755 | CA | ALA | B | 769 | 6.773 | 10.841 | −47.736 | 1.00 | 54.43 |
| ATOM | 2756 | C | ALA | B | 769 | 5.491 | 11.555 | −47.326 | 1.00 | 54.12 |
| ATOM | 2757 | O | ALA | B | 769 | 5.535 | 12.625 | −46.717 | 1.00 | 56.14 |
| ATOM | 2758 | CB | ALA | B | 769 | 7.274 | 11.385 | −49.065 | 1.00 | 53.75 |
| ATOM | 2759 | N | LEU | B | 770 | 4.346 | 10.970 | −47.657 | 1.00 | 52.05 |
| ATOM | 2760 | CA | LEU | B | 770 | 3.077 | 11.576 | −47.287 | 1.00 | 51.69 |
| ATOM | 2761 | C | LEU | B | 770 | 2.921 | 11.494 | −45.774 | 1.00 | 51.45 |
| ATOM | 2762 | O | LEU | B | 770 | 2.470 | 12.440 | −45.125 | 1.00 | 52.01 |
| ATOM | 2763 | CB | LEU | B | 770 | 1.910 | 10.864 | −47.991 | 1.00 | 52.43 |
| ATOM | 2764 | CG | LEU | B | 770 | 0.511 | 11.393 | −47.636 | 1.00 | 53.86 |
| ATOM | 2765 | CD1 | LEU | B | 770 | −0.417 | 11.306 | −48.839 | 1.00 | 54.92 |
| ATOM | 2766 | CD2 | LEU | B | 770 | −0.041 | 10.608 | −46.450 | 1.00 | 52.30 |
| ATOM | 2767 | N | ILE | B | 771 | 3.307 | 10.357 | −45.211 | 1.00 | 50.57 |
| ATOM | 2768 | CA | ILE | B | 771 | 3.216 | 10.175 | −43.775 | 1.00 | 50.50 |
| ATOM | 2769 | C | ILE | B | 771 | 4.203 | 11.092 | −43.060 | 1.00 | 50.19 |
| ATOM | 2770 | O | ILE | B | 771 | 3.883 | 11.700 | −42.047 | 1.00 | 51.29 |
| ATOM | 2771 | CB | ILE | B | 771 | 3.511 | 8.726 | −43.377 | 1.00 | 50.14 |
| ATOM | 2772 | CG1 | ILE | B | 771 | 2.554 | 7.778 | −44.105 | 1.00 | 51.79 |
| ATOM | 2773 | CG2 | ILE | B | 771 | 3.340 | 8.569 | −41.885 | 1.00 | 48.70 |
| ATOM | 2774 | CD1 | ILE | B | 771 | 1.078 | 8.018 | −43.775 | 1.00 | 51.92 |
| ATOM | 2775 | N | MET | B | 772 | 5.409 | 11.197 | −43.587 | 1.00 | 50.96 |
| ATOM | 2776 | CA | MET | B | 772 | 6.395 | 12.056 | −42.962 | 1.00 | 52.41 |
| ATOM | 2777 | C | MET | B | 772 | 5.918 | 13.499 | −42.960 | 1.00 | 53.45 |
| ATOM | 2778 | O | MET | B | 772 | 6.222 | 14.262 | −42.053 | 1.00 | 56.84 |
| ATOM | 2779 | CB | MET | B | 772 | 7.727 | 11.960 | −43.698 | 1.00 | 51.86 |
| ATOM | 2780 | CG | MET | B | 772 | 8.323 | 10.581 | −43.694 | 1.00 | 51.45 |
| ATOM | 2781 | SD | MET | B | 772 | 9.869 | 10.566 | −44.572 | 1.00 | 54.95 |
| ATOM | 2782 | CE | MET | B | 772 | 10.510 | 8.976 | −44.043 | 1.00 | 57.12 |
| ATOM | 2783 | N | ALA | B | 773 | 5.156 | 13.875 | −43.971 | 1.00 | 53.69 |
| ATOM | 2784 | CA | ALA | B | 773 | 4.680 | 15.240 | −44.048 | 1.00 | 53.73 |
| ATOM | 2785 | C | ALA | B | 773 | 3.350 | 15.455 | −43.331 | 1.00 | 53.40 |
| ATOM | 2786 | O | ALA | B | 773 | 2.955 | 16.588 | −43.076 | 1.00 | 54.15 |
| ATOM | 2787 | CB | ALA | B | 773 | 4.567 | 15.651 | −45.512 | 1.00 | 55.36 |
| ATOM | 2788 | N | SER | B | 774 | 2.653 | 14.380 | −42.995 | 1.00 | 53.47 |
| ATOM | 2789 | CA | SER | B | 774 | 1.370 | 14.536 | −42.321 | 1.00 | 54.15 |
| ATOM | 2790 | C | SER | B | 774 | 1.499 | 14.673 | −40.805 | 1.00 | 54.10 |
| ATOM | 2791 | O | SER | B | 774 | 0.512 | 14.933 | −40.126 | 1.00 | 55.42 |
| ATOM | 2792 | CB | SER | B | 774 | 0.457 | 13.352 | −42.637 | 1.00 | 54.46 |
| ATOM | 2793 | OG | SER | B | 774 | 0.363 | 13.121 | −44.027 | 1.00 | 57.39 |
| ATOM | 2794 | N | MET | B | 775 | 2.705 | 14.509 | −40.275 | 1.00 | 53.87 |
| ATOM | 2795 | CA | MET | B | 775 | 2.924 | 14.594 | −38.830 | 1.00 | 54.89 |
| ATOM | 2796 | C | MET | B | 775 | 2.843 | 15.992 | −38.190 | 1.00 | 54.27 |
| ATOM | 2797 | O | MET | B | 775 | 3.665 | 16.858 | −38.460 | 1.00 | 54.83 |
| ATOM | 2798 | CB | MET | B | 775 | 4.265 | 13.946 | −38.496 | 1.00 | 55.60 |
| ATOM | 2799 | CG | MET | B | 775 | 4.357 | 12.524 | −39.000 | 1.00 | 57.72 |
| ATOM | 2800 | SD | MET | B | 775 | 2.881 | 11.559 | −38.576 | 1.00 | 63.74 |
| ATOM | 2801 | CE | MET | B | 775 | 3.577 | 10.256 | −37.670 | 1.00 | 57.19 |
| ATOM | 2802 | N | ASP | B | 776 | 1.853 | 16.194 | −37.323 | 1.00 | 54.06 |
| ATOM | 2803 | CA | ASP | B | 776 | 1.660 | 17.475 | −36.645 | 1.00 | 53.45 |
| ATOM | 2804 | C | ASP | B | 776 | 1.627 | 17.322 | −35.119 | 1.00 | 52.22 |
| ATOM | 2805 | O | ASP | B | 776 | 0.565 | 17.293 | −34.493 | 1.00 | 51.95 |
| ATOM | 2806 | CB | ASP | B | 776 | 0.369 | 18.136 | −37.133 | 1.00 | 55.05 |
| ATOM | 2807 | CG | ASP | B | 776 | 0.193 | 19.550 | −36.595 | 1.00 | 57.95 |
| ATOM | 2808 | OD1 | ASP | B | 776 | 1.211 | 20.168 | −36.195 | 1.00 | 56.53 |
| ATOM | 2809 | OD2 | ASP | B | 776 | −0.961 | 20.046 | −36.589 | 1.00 | 56.88 |
| ATOM | 2810 | N | HIS | B | 777 | 2.810 | 17.234 | −34.529 | 1.00 | 50.34 |
| ATOM | 2811 | CA | HIS | B | 777 | 2.948 | 17.073 | −33.094 | 1.00 | 47.37 |
| ATOM | 2812 | C | HIS | B | 777 | 4.238 | 17.764 | −32.718 | 1.00 | 47.49 |
| ATOM | 2813 | O | HIS | B | 777 | 5.264 | 17.580 | −33.374 | 1.00 | 45.96 |
| ATOM | 2814 | CB | HIS | B | 777 | 3.009 | 15.589 | −32.737 | 1.00 | 45.58 |
| ATOM | 2815 | CG | HIS | B | 777 | 3.097 | 15.319 | −31.268 | 1.00 | 45.70 |
| ATOM | 2816 | ND1 | HIS | B | 777 | 4.262 | 15.481 | −30.549 | 1.00 | 46.20 |
| ATOM | 2817 | CD2 | HIS | B | 777 | 2.168 | 14.882 | −30.386 | 1.00 | 44.55 |
| ATOM | 2818 | CE1 | HIS | B | 777 | 4.048 | 15.154 | −29.287 | 1.00 | 44.22 |
| ATOM | 2819 | NE2 | HIS | B | 777 | 2.785 | 14.786 | −29.162 | 1.00 | 46.79 |
| ATOM | 2820 | N | PRO | B | 778 | 4.204 | 18.572 | −31.649 | 1.00 | 48.64 |
| ATOM | 2821 | CA | PRO | B | 778 | 5.382 | 19.308 | −31.183 | 1.00 | 48.65 |
| ATOM | 2822 | C | PRO | B | 778 | 6.636 | 18.467 | −30.938 | 1.00 | 48.69 |
| ATOM | 2823 | O | PRO | B | 778 | 7.755 | 18.986 | −30.992 | 1.00 | 50.23 |
| ATOM | 2824 | CB | PRO | B | 778 | 4.871 | 20.020 | −29.924 | 1.00 | 48.43 |
| ATOM | 2825 | CG | PRO | B | 778 | 3.796 | 19.118 | −29.435 | 1.00 | 48.56 |
| ATOM | 2826 | CD | PRO | B | 778 | 3.089 | 18.707 | −30.694 | 1.00 | 47.48 |
| ATOM | 2827 | N | HIS | B | 779 | 6.479 | 17.169 | −30.708 | 1.00 | 47.19 |
| ATOM | 2828 | CA | HIS | B | 779 | 7.666 | 16.366 | −30.468 | 1.00 | 46.69 |
| ATOM | 2829 | C | HIS | B | 779 | 8.062 | 15.405 | −31.589 | 1.00 | 46.92 |
| ATOM | 2830 | O | HIS | B | 779 | 8.806 | 14.438 | −31.387 | 1.00 | 45.42 |
| ATOM | 2831 | CB | HIS | B | 779 | 7.543 | 15.680 | −29.108 | 1.00 | 43.27 |
| ATOM | 2832 | CG | HIS | B | 779 | 7.436 | 16.657 | −27.979 | 1.00 | 42.66 |
| ATOM | 2833 | ND1 | HIS | B | 779 | 8.325 | 17.699 | −27.821 | 1.00 | 41.28 |
| ATOM | 2834 | CD2 | HIS | B | 779 | 6.504 | 16.808 | −27.010 | 1.00 | 42.33 |
| ATOM | 2835 | CE1 | HIS | B | 779 | 7.940 | 18.453 | −26.808 | 1.00 | 42.15 |
| ATOM | 2836 | NE2 | HIS | B | 779 | 6.837 | 17.935 | −26.298 | 1.00 | 39.93 |
| ATOM | 2837 | N | LEU | B | 780 | 7.583 | 15.711 | −32.788 | 1.00 | 46.23 |
| ATOM | 2838 | CA | LEU | B | 780 | 7.920 | 14.935 | −33.968 | 1.00 | 46.87 |
| ATOM | 2839 | C | LEU | B | 780 | 8.316 | 15.921 | −35.056 | 1.00 | 47.73 |
| ATOM | 2840 | O | LEU | B | 780 | 7.772 | 17.024 | −35.145 | 1.00 | 48.06 |
| ATOM | 2841 | CB | LEU | B | 780 | 6.730 | 14.107 | −34.436 | 1.00 | 45.91 |
| ATOM | 2842 | CG | LEU | B | 780 | 6.235 | 12.985 | −33.527 | 1.00 | 47.10 |
| ATOM | 2843 | CD1 | LEU | B | 780 | 5.096 | 12.287 | −34.244 | 1.00 | 48.53 |
| ATOM | 2844 | CD2 | LEU | B | 780 | 7.349 | 11.987 | −33.216 | 1.00 | 46.79 |
| ATOM | 2845 | N | VAL | B | 781 | 9.277 | 15.540 | −35.878 | 1.00 | 48.83 |
| ATOM | 2846 | CA | VAL | B | 781 | 9.691 | 16.432 | −36.944 | 1.00 | 51.89 |
| ATOM | 2847 | C | VAL | B | 781 | 8.710 | 16.336 | −38.104 | 1.00 | 53.37 |
| ATOM | 2848 | O | VAL | B | 781 | 7.995 | 15.349 | −38.258 | 1.00 | 52.89 |
| ATOM | 2849 | CB | VAL | B | 781 | 11.109 | 16.094 | −37.444 | 1.00 | 50.59 |
| ATOM | 2850 | CG1 | VAL | B | 781 | 12.078 | 16.089 | −36.266 | 1.00 | 49.98 |
| ATOM | 2851 | CG2 | VAL | B | 781 | 11.107 | 14.757 | −38.152 | 1.00 | 48.87 |
| ATOM | 2852 | N | ARG | B | 782 | 8.675 | 17.382 | −38.914 | 1.00 | 56.80 |
| ATOM | 2853 | CA | ARG | B | 782 | 7.793 | 17.423 | −40.070 | 1.00 | 59.13 |
| ATOM | 2854 | C | ARG | B | 782 | 8.643 | 17.636 | −41.316 | 1.00 | 58.23 |
| ATOM | 2855 | O | ARG | B | 782 | 9.646 | 18.364 | −41.293 | 1.00 | 57.16 |
| ATOM | 2856 | CB | ARG | B | 782 | 6.784 | 18.562 | −39.923 | 1.00 | 61.42 |
| ATOM | 2857 | CG | ARG | B | 782 | 5.654 | 18.537 | −40.920 | 1.00 | 66.10 |
| ATOM | 2858 | CD | ARG | B | 782 | 4.579 | 19.529 | −40.496 | 1.00 | 72.64 |
| ATOM | 2859 | NE | ARG | B | 782 | 3.401 | 19.499 | −41.364 | 1.00 | 77.10 |
| ATOM | 2860 | CZ | ARG | B | 782 | 2.293 | 20.207 | −41.147 | 1.00 | 78.91 |
| ATOM | 2861 | NH1 | ARG | B | 782 | 2.205 | 21.003 | −40.085 | 1.00 | 78.69 |
| ATOM | 2862 | NH2 | ARG | B | 782 | 1.273 | 20.129 | −41.997 | 1.00 | 80.27 |
| ATOM | 2863 | N | LEU | B | 783 | 8.247 | 16.980 | −42.397 | 1.00 | 56.30 |
| ATOM | 2864 | CA | LEU | B | 783 | 8.958 | 17.102 | −43.654 | 1.00 | 55.96 |
| ATOM | 2865 | C | LEU | B | 783 | 8.383 | 18.309 | −44.374 | 1.00 | 54.57 |
| ATOM | 2866 | O | LEU | B | 783 | 7.175 | 18.392 | −44.573 | 1.00 | 56.01 |
| ATOM | 2867 | CB | LEU | B | 783 | 8.745 | 15.839 | −44.486 | 1.00 | 57.21 |
| ATOM | 2868 | CG | LEU | B | 783 | 9.516 | 15.702 | −45.795 | 1.00 | 55.26 |
| ATOM | 2869 | CD1 | LEU | B | 783 | 11.009 | 15.669 | −45.514 | 1.00 | 56.03 |
| ATOM | 2870 | CD2 | LEU | B | 783 | 9.080 | 14.436 | −46.485 | 1.00 | 55.49 |
| ATOM | 2871 | N | LEU | B | 784 | 9.242 | 19.250 | −44.747 | 1.00 | 54.51 |
| ATOM | 2872 | CA | LEU | B | 784 | 8.788 | 20.450 | −45.444 | 1.00 | 54.07 |
| ATOM | 2873 | C | LEU | B | 784 | 8.526 | 20.132 | −46.903 | 1.00 | 52.39 |
| ATOM | 2874 | O | LEU | B | 784 | 7.471 | 20.466 | −47.443 | 1.00 | 52.32 |
| ATOM | 2875 | CB | LEU | B | 784 | 9.832 | 21.564 | −45.340 | 1.00 | 54.48 |
| ATOM | 2876 | CG | LEU | B | 784 | 10.072 | 22.091 | −43.920 | 1.00 | 57.09 |
| ATOM | 2877 | CD1 | LEU | B | 784 | 11.245 | 23.059 | −43.919 | 1.00 | 57.21 |
| ATOM | 2878 | CD2 | LEU | B | 784 | 8.810 | 22.765 | −43.393 | 1.00 | 56.32 |
| ATOM | 2879 | N | GLY | B | 785 | 9.488 | 19.470 | −47.535 | 1.00 | 50.88 |
| ATOM | 2880 | CA | GLY | B | 785 | 9.325 | 19.128 | −48.931 | 1.00 | 48.30 |
| ATOM | 2881 | C | GLY | B | 785 | 10.255 | 18.057 | −49.460 | 1.00 | 48.59 |
| ATOM | 2882 | O | GLY | B | 785 | 11.104 | 17.513 | −48.755 | 1.00 | 46.94 |
| ATOM | 2883 | N | VAL | B | 786 | 10.077 | 17.750 | −50.733 | 1.00 | 48.89 |
| ATOM | 2884 | CA | VAL | B | 786 | 10.891 | 16.755 | −51.381 | 1.00 | 49.74 |
| ATOM | 2885 | C | VAL | B | 786 | 11.370 | 17.317 | −52.707 | 1.00 | 51.57 |
| ATOM | 2886 | O | VAL | B | 786 | 10.638 | 18.059 | −53.385 | 1.00 | 50.67 |
| ATOM | 2887 | CB | VAL | B | 786 | 10.083 | 15.468 | −51.640 | 1.00 | 50.26 |
| ATOM | 2888 | CG1 | VAL | B | 786 | 10.931 | 14.473 | −52.414 | 1.00 | 50.36 |
| ATOM | 2889 | CG2 | VAL | B | 786 | 9.638 | 14.858 | −50.320 | 1.00 | 49.86 |
| ATOM | 2890 | N | CYS | B | 787 | 12.609 | 16.983 | −53.058 | 1.00 | 50.94 |
| ATOM | 2891 | CA | CYS | B | 787 | 13.187 | 17.414 | −54.316 | 1.00 | 51.41 |
| ATOM | 2892 | C | CYS | B | 787 | 13.398 | 16.134 | −55.113 | 1.00 | 51.83 |
| ATOM | 2893 | O | CYS | B | 787 | 14.017 | 15.193 | −54.627 | 1.00 | 51.13 |
| ATOM | 2894 | CB | CYS | B | 787 | 14.515 | 18.135 | −54.089 | 1.00 | 52.49 |
| ATOM | 2895 | SG | CYS | B | 787 | 15.263 | 18.700 | −55.644 | 1.00 | 57.81 |
| ATOM | 2896 | N | LEU | B | 788 | 12.891 | 16.105 | −56.339 | 1.00 | 54.13 |
| ATOM | 2897 | CA | LEU | B | 788 | 12.975 | 14.915 | −57.173 | 1.00 | 57.19 |
| ATOM | 2898 | C | LEU | B | 788 | 14.242 | 14.704 | −57.992 | 1.00 | 61.05 |
| ATOM | 2899 | O | LEU | B | 788 | 14.481 | 13.599 | −58.479 | 1.00 | 61.41 |
| ATOM | 2900 | CB | LEU | B | 788 | 11.751 | 14.864 | −58.089 | 1.00 | 55.35 |
| ATOM | 2901 | CG | LEU | B | 788 | 10.426 | 14.802 | −57.311 | 1.00 | 56.39 |
| ATOM | 2902 | CD1 | LEU | B | 788 | 9.251 | 14.950 | −58.250 | 1.00 | 55.47 |
| ATOM | 2903 | CD2 | LEU | B | 788 | 10.334 | 13.487 | −56.548 | 1.00 | 55.46 |
| ATOM | 2904 | N | SER | B | 789 | 15.066 | 15.737 | −58.146 | 1.00 | 64.33 |
| ATOM | 2905 | CA | SER | B | 789 | 16.285 | 15.582 | −58.938 | 1.00 | 66.33 |
| ATOM | 2906 | C | SER | B | 789 | 17.520 | 16.071 | −58.192 | 1.00 | 66.73 |
| ATOM | 2907 | O | SER | B | 789 | 17.424 | 16.955 | −57.336 | 1.00 | 66.88 |
| ATOM | 2908 | CB | SER | B | 789 | 16.141 | 16.337 | −60.264 | 1.00 | 67.97 |
| ATOM | 2909 | OG | SER | B | 789 | 17.099 | 15.892 | −61.214 | 1.00 | 72.18 |
| ATOM | 2910 | N | PRO | B | 790 | 18.697 | 15.474 | −58.477 | 1.00 | 67.24 |
| ATOM | 2911 | CA | PRO | B | 790 | 18.941 | 14.388 | −59.439 | 1.00 | 66.95 |
| ATOM | 2912 | C | PRO | B | 790 | 18.245 | 13.123 | −58.965 | 1.00 | 67.17 |
| ATOM | 2913 | O | PRO | B | 790 | 17.782 | 12.298 | −59.757 | 1.00 | 66.12 |
| ATOM | 2914 | CB | PRO | B | 790 | 20.454 | 14.242 | −59.407 | 1.00 | 66.69 |
| ATOM | 2915 | CG | PRO | B | 790 | 20.768 | 14.555 | −57.969 | 1.00 | 66.95 |
| ATOM | 2916 | CD | PRO | B | 790 | 19.933 | 15.791 | −57.739 | 1.00 | 66.62 |
| ATOM | 2917 | N | THR | B | 791 | 18.209 | 12.987 | −57.646 | 1.00 | 66.82 |
| ATOM | 2918 | CA | THR | B | 791 | 17.565 | 11.869 | −56.986 | 1.00 | 66.98 |
| ATOM | 2919 | C | THR | B | 791 | 16.862 | 12.492 | −55.791 | 1.00 | 65.80 |
| ATOM | 2920 | O | THR | B | 791 | 17.173 | 13.617 | −55.392 | 1.00 | 64.64 |
| ATOM | 2921 | CB | THR | B | 791 | 18.583 | 10.805 | −56.503 | 1.00 | 69.05 |
| ATOM | 2922 | OG1 | THR | B | 791 | 19.435 | 11.370 | −55.496 | 1.00 | 69.07 |
| ATOM | 2923 | CG2 | THR | B | 791 | 19.431 | 10.306 | −57.679 | 1.00 | 69.29 |
| ATOM | 2924 | N | ILE | B | 792 | 15.920 | 11.748 | −55.229 | 1.00 | 64.46 |
| ATOM | 2925 | CA | ILE | B | 792 | 15.117 | 12.198 | −54.103 | 1.00 | 62.56 |
| ATOM | 2926 | C | ILE | B | 792 | 15.841 | 12.710 | −52.871 | 1.00 | 61.32 |
| ATOM | 2927 | O | ILE | B | 792 | 16.659 | 12.011 | −52.283 | 1.00 | 62.26 |
| ATOM | 2928 | CB | ILE | B | 792 | 14.152 | 11.093 | −53.677 | 1.00 | 60.97 |
| ATOM | 2929 | CG1 | ILE | B | 792 | 13.035 | 10.988 | −54.710 | 1.00 | 61.00 |
| ATOM | 2930 | CG2 | ILE | B | 792 | 13.616 | 11.383 | −52.311 | 1.00 | 58.92 |
| ATOM | 2931 | CD1 | ILE | B | 792 | 12.063 | 9.871 | −54.469 | 1.00 | 65.86 |
| ATOM | 2932 | N | GLN | B | 793 | 15.510 | 13.938 | −52.483 | 1.00 | 60.22 |
| ATOM | 2933 | CA | GLN | B | 793 | 16.103 | 14.566 | −51.310 | 1.00 | 60.54 |
| ATOM | 2934 | C | GLN | B | 793 | 14.994 | 14.972 | −50.352 | 1.00 | 59.77 |
| ATOM | 2935 | O | GLN | B | 793 | 13.964 | 15.522 | −50.766 | 1.00 | 59.02 |
| ATOM | 2936 | CB | GLN | B | 793 | 16.897 | 15.817 | −51.694 | 1.00 | 61.88 |
| ATOM | 2937 | CG | GLN | B | 793 | 17.928 | 15.613 | −52.782 | 1.00 | 64.34 |
| ATOM | 2938 | CD | GLN | B | 793 | 18.587 | 16.913 | −53.200 | 1.00 | 65.85 |
| ATOM | 2939 | OE1 | GLN | B | 793 | 19.084 | 17.033 | −54.317 | 1.00 | 67.77 |
| ATOM | 2940 | NE2 | GLN | B | 793 | 18.601 | 17.893 | −52.299 | 1.00 | 66.10 |
| ATOM | 2941 | N | LEU | B | 794 | 15.213 | 14.697 | −49.072 | 1.00 | 58.48 |
| ATOM | 2942 | CA | LEU | B | 794 | 14.250 | 15.036 | −48.042 | 1.00 | 57.14 |
| ATOM | 2943 | C | LEU | B | 794 | 14.689 | 16.346 | −47.414 | 1.00 | 57.64 |
| ATOM | 2944 | O | LEU | B | 794 | 15.859 | 16.531 | −47.093 | 1.00 | 57.64 |
| ATOM | 2945 | CB | LEU | B | 794 | 14.187 | 13.922 | −46.992 | 1.00 | 55.84 |
| ATOM | 2946 | CG | LEU | B | 794 | 13.804 | 12.529 | −47.515 | 1.00 | 54.27 |
| ATOM | 2947 | CD1 | LEU | B | 794 | 13.456 | 11.624 | −46.344 | 1.00 | 53.66 |
| ATOM | 2948 | CD2 | LEU | B | 794 | 12.616 | 12.627 | −48.449 | 1.00 | 53.08 |
| ATOM | 2949 | N | VAL | B | 795 | 13.746 | 17.264 | −47.258 | 1.00 | 59.07 |
| ATOM | 2950 | CA | VAL | B | 795 | 14.047 | 18.567 | −46.690 | 1.00 | 59.85 |
| ATOM | 2951 | C | VAL | B | 795 | 13.222 | 18.812 | −45.435 | 1.00 | 62.17 |
| ATOM | 2952 | O | VAL | B | 795 | 11.987 | 18.855 | −45.472 | 1.00 | 61.88 |
| ATOM | 2953 | CB | VAL | B | 795 | 13.774 | 19.697 | −47.714 | 1.00 | 58.39 |
| ATOM | 2954 | CG1 | VAL | B | 795 | 14.231 | 21.040 | −47.157 | 1.00 | 58.05 |
| ATOM | 2955 | CG2 | VAL | B | 795 | 14.495 | 19.394 | −49.014 | 1.00 | 58.45 |
| ATOM | 2956 | N | THR | B | 796 | 13.931 | 18.969 | −44.323 | 1.00 | 63.79 |
| ATOM | 2957 | CA | THR | B | 796 | 13.311 | 19.210 | −43.034 | 1.00 | 65.36 |
| ATOM | 2958 | C | THR | B | 796 | 13.869 | 20.476 | −42.436 | 1.00 | 66.46 |
| ATOM | 2959 | O | THR | B | 796 | 14.993 | 20.890 | −42.747 | 1.00 | 66.26 |
| ATOM | 2960 | CB | THR | B | 796 | 13.606 | 18.077 | −42.033 | 1.00 | 65.06 |
| ATOM | 2961 | OG1 | THR | B | 796 | 13.043 | 16.852 | −42.509 | 1.00 | 68.33 |
| ATOM | 2962 | CG2 | THR | B | 796 | 13.007 | 18.401 | −40.672 | 1.00 | 66.50 |
| ATOM | 2963 | N | GLN | B | 797 | 13.068 | 21.090 | −41.574 | 1.00 | 66.67 |
| ATOM | 2964 | CA | GLN | B | 797 | 13.488 | 22.284 | −40.884 | 1.00 | 66.57 |
| ATOM | 2965 | C | GLN | B | 797 | 14.690 | 21.827 | −40.085 | 1.00 | 66.07 |
| ATOM | 2966 | O | GLN | B | 797 | 14.680 | 20.741 | −39.501 | 1.00 | 65.39 |
| ATOM | 2967 | CB | GLN | B | 797 | 12.393 | 22.758 | −39.935 | 1.00 | 67.57 |
| ATOM | 2968 | CG | GLN | B | 797 | 12.673 | 24.081 | −39.263 | 1.00 | 70.20 |
| ATOM | 2969 | CD | GLN | B | 797 | 11.514 | 24.526 | −38.399 | 1.00 | 73.88 |
| ATOM | 2970 | OE1 | GLN | B | 797 | 11.403 | 24.140 | −37.230 | 1.00 | 75.03 |
| ATOM | 2971 | NE2 | GLN | B | 797 | 10.623 | 25.327 | −38.979 | 1.00 | 74.39 |
| ATOM | 2972 | N | LEU | B | 798 | 15.737 | 22.638 | −40.083 | 1.00 | 66.31 |
| ATOM | 2973 | CA | LEU | B | 798 | 16.931 | 22.303 | −39.333 | 1.00 | 65.46 |
| ATOM | 2974 | C | LEU | B | 798 | 16.672 | 22.549 | −37.851 | 1.00 | 66.20 |
| ATOM | 2975 | O | LEU | B | 798 | 15.902 | 23.441 | −37.476 | 1.00 | 65.77 |
| ATOM | 2976 | CB | LEU | B | 798 | 18.097 | 23.170 | −39.791 | 1.00 | 64.87 |
| ATOM | 2977 | CG | LEU | B | 798 | 19.410 | 22.965 | −39.045 | 1.00 | 65.11 |
| ATOM | 2978 | CD1 | LEU | B | 798 | 19.933 | 21.554 | −39.290 | 1.00 | 64.89 |
| ATOM | 2979 | CD2 | LEU | B | 798 | 20.411 | 23.999 | −39.516 | 1.00 | 64.74 |
| ATOM | 2980 | N | MET | B | 799 | 17.296 | 21.733 | −37.011 | 1.00 | 66.39 |
| ATOM | 2981 | CA | MET | B | 799 | 17.169 | 21.885 | −35.571 | 1.00 | 65.45 |
| ATOM | 2982 | C | MET | B | 799 | 18.506 | 22.465 | −35.113 | 1.00 | 65.57 |
| ATOM | 2983 | O | MET | B | 799 | 19.547 | 21.798 | −35.162 | 1.00 | 64.60 |
| ATOM | 2984 | CB | MET | B | 799 | 16.881 | 20.536 | −34.911 | 1.00 | 64.87 |
| ATOM | 2985 | CG | MET | B | 799 | 15.491 | 19.990 | −35.216 | 1.00 | 64.16 |
| ATOM | 2986 | SD | MET | B | 799 | 14.163 | 21.040 | −34.582 | 1.00 | 65.40 |
| ATOM | 2987 | CE | MET | B | 799 | 13.635 | 21.891 | −36.058 | 1.00 | 64.20 |
| ATOM | 2988 | N | PRO | B | 800 | 18.486 | 23.730 | −34.669 | 1.00 | 65.90 |
| ATOM | 2989 | CA | PRO | B | 800 | 19.652 | 24.484 | −34.196 | 1.00 | 65.62 |
| ATOM | 2990 | C | PRO | B | 800 | 20.591 | 23.765 | −33.239 | 1.00 | 65.08 |
| ATOM | 2991 | O | PRO | B | 800 | 21.805 | 23.817 | −33.412 | 1.00 | 64.72 |
| ATOM | 2992 | CB | PRO | B | 800 | 19.025 | 25.731 | −33.566 | 1.00 | 66.58 |
| ATOM | 2993 | CG | PRO | B | 800 | 17.699 | 25.239 | −33.092 | 1.00 | 66.63 |
| ATOM | 2994 | CD | PRO | B | 800 | 17.241 | 24.410 | −34.269 | 1.00 | 66.65 |
| ATOM | 2995 | N | HIS | B | 801 | 20.044 | 23.088 | −32.237 | 1.00 | 63.99 |
| ATOM | 2996 | CA | HIS | B | 801 | 20.892 | 22.408 | −31.277 | 1.00 | 62.97 |
| ATOM | 2997 | C | HIS | B | 801 | 21.310 | 20.988 | −31.636 | 1.00 | 61.86 |
| ATOM | 2998 | O | HIS | B | 801 | 21.843 | 20.257 | −30.804 | 1.00 | 63.07 |
| ATOM | 2999 | CB | HIS | B | 801 | 20.238 | 22.471 | −29.900 | 1.00 | 64.77 |
| ATOM | 3000 | CG | HIS | B | 801 | 20.298 | 23.836 | −29.283 | 1.00 | 66.40 |
| ATOM | 3001 | ND1 | HIS | B | 801 | 21.486 | 24.432 | −28.909 | 1.00 | 65.11 |
| ATOM | 3002 | CD2 | HIS | B | 801 | 19.324 | 24.740 | −29.016 | 1.00 | 66.09 |
| ATOM | 3003 | CE1 | HIS | B | 801 | 21.240 | 25.641 | −28.439 | 1.00 | 64.78 |
| ATOM | 3004 | NE2 | HIS | B | 801 | 19.936 | 25.853 | −28.492 | 1.00 | 65.44 |
| ATOM | 3005 | N | GLY | B | 802 | 21.090 | 20.607 | −32.886 | 1.00 | 60.15 |
| ATOM | 3006 | CA | GLY | B | 802 | 21.490 | 19.289 | −33.331 | 1.00 | 57.51 |
| ATOM | 3007 | C | GLY | B | 802 | 20.901 | 18.064 | −32.650 | 1.00 | 56.35 |
| ATOM | 3008 | O | GLY | B | 802 | 19.810 | 18.086 | −32.075 | 1.00 | 53.17 |
| ATOM | 3009 | N | CYS | B | 803 | 21.660 | 16.978 | −32.735 | 1.00 | 56.15 |
| ATOM | 3010 | CA | CYS | B | 803 | 21.277 | 15.688 | −32.190 | 1.00 | 55.11 |
| ATOM | 3011 | C | CYS | B | 803 | 21.445 | 15.568 | −30.682 | 1.00 | 55.33 |
| ATOM | 3012 | O | CYS | B | 803 | 22.491 | 15.911 | −30.128 | 1.00 | 54.75 |
| ATOM | 3013 | CB | CYS | B | 803 | 22.074 | 14.594 | −32.899 | 1.00 | 55.30 |
| ATOM | 3014 | SG | CYS | B | 803 | 21.743 | 14.518 | −34.697 | 1.00 | 60.15 |
| ATOM | 3015 | N | LEU | B | 804 | 20.396 | 15.066 | −30.031 | 1.00 | 54.33 |
| ATOM | 3016 | CA | LEU | B | 804 | 20.376 | 14.883 | −28.588 | 1.00 | 53.12 |
| ATOM | 3017 | C | LEU | B | 804 | 21.534 | 14.014 | −28.089 | 1.00 | 52.92 |
| ATOM | 3018 | O | LEU | B | 804 | 22.177 | 14.328 | −27.088 | 1.00 | 51.69 |
| ATOM | 3019 | CB | LEU | B | 804 | 19.043 | 14.266 | −28.168 | 1.00 | 52.44 |
| ATOM | 3020 | CG | LEU | B | 804 | 18.815 | 14.078 | −26.666 | 1.00 | 50.62 |
| ATOM | 3021 | CD1 | LEU | B | 804 | 18.967 | 15.417 | −25.974 | 1.00 | 51.38 |
| ATOM | 3022 | CD2 | LEU | B | 804 | 17.428 | 13.491 | −26.413 | 1.00 | 49.29 |
| ATOM | 3023 | N | LEU | B | 805 | 21.801 | 12.920 | −28.782 | 1.00 | 52.39 |
| ATOM | 3024 | CA | LEU | B | 805 | 22.888 | 12.053 | −28.373 | 1.00 | 54.68 |
| ATOM | 3025 | C | LEU | B | 805 | 24.161 | 12.863 | −28.138 | 1.00 | 57.25 |
| ATOM | 3026 | O | LEU | B | 805 | 24.918 | 12.604 | −27.200 | 1.00 | 56.94 |
| ATOM | 3027 | CB | LEU | B | 805 | 23.156 | 10.998 | −29.438 | 1.00 | 52.27 |
| ATOM | 3028 | CG | LEU | B | 805 | 24.380 | 10.169 | −29.080 | 1.00 | 51.83 |
| ATOM | 3029 | CD1 | LEU | B | 805 | 24.151 | 9.471 | −27.737 | 1.00 | 50.62 |
| ATOM | 3030 | CD2 | LEU | B | 805 | 24.660 | 9.179 | −30.182 | 1.00 | 53.24 |
| ATOM | 3031 | N | GLU | B | 806 | 24.387 | 13.844 | −29.006 | 1.00 | 60.22 |
| ATOM | 3032 | CA | GLU | B | 806 | 25.558 | 14.705 | −28.919 | 1.00 | 62.56 |
| ATOM | 3033 | C | GLU | B | 806 | 25.351 | 15.761 | −27.836 | 1.00 | 60.94 |
| ATOM | 3034 | O | GLU | B | 806 | 26.233 | 16.013 | −27.018 | 1.00 | 60.73 |
| ATOM | 3035 | CB | GLU | B | 806 | 25.804 | 15.395 | −30.267 | 1.00 | 67.71 |
| ATOM | 3036 | CG | GLU | B | 806 | 25.695 | 14.471 | −31.488 | 1.00 | 75.24 |
| ATOM | 3037 | CD | GLU | B | 806 | 25.922 | 15.212 | −32.804 | 1.00 | 79.21 |
| ATOM | 3038 | OE1 | GLU | B | 806 | 25.254 | 16.251 | −33.026 | 1.00 | 81.47 |
| ATOM | 3039 | OE2 | GLU | B | 806 | 26.762 | 14.758 | −33.615 | 1.00 | 79.92 |
| ATOM | 3040 | N | TYR | B | 807 | 24.173 | 16.371 | −27.832 | 1.00 | 59.29 |
| ATOM | 3041 | CA | TYR | B | 807 | 23.856 | 17.400 | −26.860 | 1.00 | 59.19 |
| ATOM | 3042 | C | TYR | B | 807 | 24.157 | 16.981 | −25.424 | 1.00 | 59.38 |
| ATOM | 3043 | O | TYR | B | 807 | 24.822 | 17.708 | −24.700 | 1.00 | 58.77 |
| ATOM | 3044 | CB | TYR | B | 807 | 22.391 | 17.784 | −26.966 | 1.00 | 59.99 |
| ATOM | 3045 | CG | TYR | B | 807 | 22.053 | 19.071 | −26.262 | 1.00 | 60.81 |
| ATOM | 3046 | CD1 | TYR | B | 807 | 22.227 | 20.300 | −26.897 | 1.00 | 61.13 |
| ATOM | 3047 | CD2 | TYR | B | 807 | 21.546 | 19.064 | −24.966 | 1.00 | 61.33 |
| ATOM | 3048 | CE1 | TYR | B | 807 | 21.899 | 21.489 | −26.265 | 1.00 | 61.63 |
| ATOM | 3049 | CE2 | TYR | B | 807 | 21.215 | 20.252 | −24.319 | 1.00 | 62.89 |
| ATOM | 3050 | CZ | TYR | B | 807 | 21.392 | 21.460 | −24.977 | 1.00 | 62.83 |
| ATOM | 3051 | OH | TYR | B | 807 | 21.035 | 22.630 | −24.354 | 1.00 | 64.52 |
| ATOM | 3052 | N | VAL | B | 808 | 23.671 | 15.819 | −25.002 | 1.00 | 60.10 |
| ATOM | 3053 | CA | VAL | B | 808 | 23.927 | 15.382 | −23.630 | 1.00 | 62.18 |
| ATOM | 3054 | C | VAL | B | 808 | 25.398 | 15.164 | −23.325 | 1.00 | 63.86 |
| ATOM | 3055 | O | VAL | B | 808 | 25.855 | 15.480 | −22.235 | 1.00 | 64.12 |
| ATOM | 3056 | CB | VAL | B | 808 | 23.176 | 14.073 | −23.257 | 1.00 | 60.51 |
| ATOM | 3057 | CG1 | VAL | B | 808 | 21.704 | 14.344 | −23.088 | 1.00 | 59.12 |
| ATOM | 3058 | CG2 | VAL | B | 808 | 23.417 | 13.015 | −24.310 | 1.00 | 60.89 |
| ATOM | 3059 | N | HIS | B | 809 | 26.147 | 14.624 | −24.273 | 1.00 | 67.06 |
| ATOM | 3060 | CA | HIS | B | 809 | 27.555 | 14.383 | −24.014 | 1.00 | 70.40 |
| ATOM | 3061 | C | HIS | B | 809 | 28.269 | 15.679 | −23.658 | 1.00 | 70.99 |
| ATOM | 3062 | O | HIS | B | 809 | 28.886 | 15.797 | −22.602 | 1.00 | 72.12 |
| ATOM | 3063 | CB | HIS | B | 809 | 28.236 | 13.746 | −25.226 | 1.00 | 73.29 |
| ATOM | 3064 | CG | HIS | B | 809 | 29.639 | 13.294 | −24.956 | 1.00 | 76.84 |
| ATOM | 3065 | ND1 | HIS | B | 809 | 29.937 | 12.293 | −24.056 | 1.00 | 77.14 |
| ATOM | 3066 | CD2 | HIS | B | 809 | 30.826 | 13.724 | −25.447 | 1.00 | 77.99 |
| ATOM | 3067 | CE1 | HIS | B | 809 | 31.247 | 12.126 | −24.005 | 1.00 | 78.08 |
| ATOM | 3068 | NE2 | HIS | B | 809 | 31.809 | 12.982 | −24.839 | 1.00 | 78.17 |
| ATOM | 3069 | N | GLU | B | 810 | 28.162 | 16.660 | −24.537 | 1.00 | 71.44 |
| ATOM | 3070 | CA | GLU | B | 810 | 28.827 | 17.928 | −24.322 | 1.00 | 72.20 |
| ATOM | 3071 | C | GLU | B | 810 | 28.320 | 18.741 | −23.141 | 1.00 | 72.35 |
| ATOM | 3072 | O | GLU | B | 810 | 29.121 | 19.284 | −22.378 | 1.00 | 72.42 |
| ATOM | 3073 | CB | GLU | B | 810 | 28.760 | 18.741 | −25.604 | 1.00 | 74.24 |
| ATOM | 3074 | CG | GLU | B | 810 | 29.503 | 18.067 | −26.746 | 1.00 | 76.99 |
| ATOM | 3075 | CD | GLU | B | 810 | 29.191 | 18.694 | −28.082 | 1.00 | 80.01 |
| ATOM | 3076 | OE1 | GLU | B | 810 | 29.408 | 19.918 | −28.231 | 1.00 | 81.26 |
| ATOM | 3077 | OE2 | GLU | B | 810 | 28.726 | 17.961 | −28.982 | 1.00 | 80.87 |
| ATOM | 3078 | N | HIS | B | 811 | 27.001 | 18.822 | −22.976 | 1.00 | 72.11 |
| ATOM | 3079 | CA | HIS | B | 811 | 26.427 | 19.588 | −21.869 | 1.00 | 71.84 |
| ATOM | 3080 | C | HIS | B | 811 | 26.168 | 18.752 | −20.624 | 1.00 | 71.80 |
| ATOM | 3081 | O | HIS | B | 811 | 25.302 | 19.096 | −19.820 | 1.00 | 70.61 |
| ATOM | 3082 | CB | HIS | B | 811 | 25.115 | 20.252 | −22.286 | 1.00 | 71.49 |
| ATOM | 3083 | CG | HIS | B | 811 | 25.243 | 21.157 | −23.468 | 1.00 | 71.98 |
| ATOM | 3084 | ND1 | HIS | B | 811 | 25.549 | 20.695 | −24.729 | 1.00 | 73.94 |
| ATOM | 3085 | CD2 | HIS | B | 811 | 25.085 | 22.496 | −23.586 | 1.00 | 72.36 |
| ATOM | 3086 | CE1 | HIS | B | 811 | 25.571 | 21.709 | −25.575 | 1.00 | 74.46 |
| ATOM | 3087 | NE2 | HIS | B | 811 | 25.293 | 22.813 | −24.907 | 1.00 | 74.62 |
| ATOM | 3088 | N | LYS | B | 812 | 26.917 | 17.665 | −20.460 | 1.00 | 73.21 |
| ATOM | 3089 | CA | LYS | B | 812 | 26.736 | 16.793 | −19.300 | 1.00 | 75.11 |
| ATOM | 3090 | C | LYS | B | 812 | 26.604 | 17.575 | −18.001 | 1.00 | 75.97 |
| ATOM | 3091 | O | LYS | B | 812 | 25.592 | 17.499 | −17.305 | 1.00 | 75.98 |
| ATOM | 3092 | CB | LYS | B | 812 | 27.901 | 15.805 | −19.157 | 1.00 | 74.11 |
| ATOM | 3093 | CG | LYS | B | 812 | 27.887 | 15.118 | −17.799 | 1.00 | 74.38 |
| ATOM | 3094 | CD | LYS | B | 812 | 28.809 | 13.917 | −17.690 | 1.00 | 73.97 |
| ATOM | 3095 | CE | LYS | B | 812 | 28.613 | 13.265 | −16.322 | 1.00 | 72.70 |
| ATOM | 3096 | NZ | LYS | B | 812 | 29.394 | 12.015 | −16.154 | 1.00 | 73.35 |
| ATOM | 3097 | N | ASP | B | 813 | 27.642 | 18.330 | −17.682 | 1.00 | 77.79 |
| ATOM | 3098 | CA | ASP | B | 813 | 27.668 | 19.119 | −16.463 | 1.00 | 79.44 |
| ATOM | 3099 | C | ASP | B | 813 | 26.644 | 20.256 | −16.500 | 1.00 | 79.12 |
| ATOM | 3100 | O | ASP | B | 813 | 26.706 | 21.185 | −15.699 | 1.00 | 79.20 |
| ATOM | 3101 | CB | ASP | B | 813 | 29.085 | 19.659 | −16.258 | 1.00 | 81.16 |
| ATOM | 3102 | CG | ASP | B | 813 | 30.154 | 18.594 | −16.504 | 1.00 | 83.14 |
| ATOM | 3103 | OD1 | ASP | B | 813 | 30.044 | 17.498 | −15.912 | 1.00 | 82.59 |
| ATOM | 3104 | OD2 | ASP | B | 813 | 31.101 | 18.845 | −17.288 | 1.00 | 84.71 |
| ATOM | 3105 | N | ASN | B | 814 | 25.688 | 20.159 | −17.421 | 1.00 | 78.87 |
| ATOM | 3106 | CA | ASN | B | 814 | 24.653 | 21.178 | −17.570 | 1.00 | 77.92 |
| ATOM | 3107 | C | ASN | B | 814 | 23.247 | 20.591 | −17.474 | 1.00 | 75.00 |
| ATOM | 3108 | O | ASN | B | 814 | 22.278 | 21.320 | −17.278 | 1.00 | 73.54 |
| ATOM | 3109 | CB | ASN | B | 814 | 24.807 | 21.875 | −18.925 | 1.00 | 83.27 |
| ATOM | 3110 | CG | ASN | B | 814 | 23.809 | 23.011 | −19.123 | 1.00 | 87.37 |
| ATOM | 3111 | OD1 | ASN | B | 814 | 23.599 | 23.487 | −20.248 | 1.00 | 89.03 |
| ATOM | 3112 | ND2 | ASN | B | 814 | 23.200 | 23.462 | −18.027 | 1.00 | 88.12 |
| ATOM | 3113 | N | ILE | B | 815 | 23.136 | 19.274 | −17.610 | 1.00 | 72.06 |
| ATOM | 3114 | CA | ILE | B | 815 | 21.834 | 18.608 | −17.567 | 1.00 | 68.21 |
| ATOM | 3115 | C | ILE | B | 815 | 21.335 | 18.306 | −16.157 | 1.00 | 65.98 |
| ATOM | 3116 | O | ILE | B | 815 | 22.010 | 17.630 | −15.374 | 1.00 | 66.13 |
| ATOM | 3117 | CB | ILE | B | 815 | 21.864 | 17.285 | −18.361 | 1.00 | 67.26 |
| ATOM | 3118 | CG1 | ILE | B | 815 | 22.485 | 17.517 | −19.738 | 1.00 | 66.10 |
| ATOM | 3119 | CG2 | ILE | B | 815 | 20.461 | 16.739 | −18.512 | 1.00 | 66.10 |
| ATOM | 3120 | CD1 | ILE | B | 815 | 21.770 | 18.532 | −20.565 | 1.00 | 65.00 |
| ATOM | 3121 | N | GLY | B | 816 | 20.139 | 18.806 | −15.853 | 1.00 | 63.12 |
| ATOM | 3122 | CA | GLY | B | 816 | 19.534 | 18.592 | −14.549 | 1.00 | 59.27 |
| ATOM | 3123 | C | GLY | B | 816 | 18.502 | 17.479 | −14.584 | 1.00 | 57.42 |
| ATOM | 3124 | O | GLY | B | 816 | 18.077 | 17.046 | −15.658 | 1.00 | 56.24 |
| ATOM | 3125 | N | SER | B | 817 | 18.081 | 17.013 | −13.413 | 1.00 | 53.84 |
| ATOM | 3126 | CA | SER | B | 817 | 17.118 | 15.936 | −13.375 | 1.00 | 51.45 |
| ATOM | 3127 | C | SER | B | 817 | 15.826 | 16.343 | −14.067 | 1.00 | 50.87 |
| ATOM | 3128 | O | SER | B | 817 | 15.205 | 15.532 | −14.746 | 1.00 | 51.59 |
| ATOM | 3129 | CB | SER | B | 817 | 16.841 | 15.493 | −11.927 | 1.00 | 50.35 |
| ATOM | 3130 | OG | SER | B | 817 | 16.050 | 16.422 | −11.214 | 1.00 | 48.83 |
| ATOM | 3131 | N | GLN | B | 818 | 15.426 | 17.597 | −13.914 | 1.00 | 48.39 |
| ATOM | 3132 | CA | GLN | B | 818 | 14.189 | 18.046 | −14.531 | 1.00 | 50.14 |
| ATOM | 3133 | C | GLN | B | 818 | 14.256 | 17.872 | −16.048 | 1.00 | 50.37 |
| ATOM | 3134 | O | GLN | B | 818 | 13.397 | 17.234 | −16.664 | 1.00 | 50.57 |
| ATOM | 3135 | CB | GLN | B | 818 | 13.931 | 19.509 | −14.172 | 1.00 | 51.39 |
| ATOM | 3136 | CG | GLN | B | 818 | 12.530 | 20.005 | −14.512 | 1.00 | 53.65 |
| ATOM | 3137 | CD | GLN | B | 818 | 12.304 | 21.465 | −14.104 | 1.00 | 54.44 |
| ATOM | 3138 | OE1 | GLN | B | 818 | 13.090 | 22.354 | −14.451 | 1.00 | 51.78 |
| ATOM | 3139 | NE2 | GLN | B | 818 | 11.223 | 21.711 | −13.374 | 1.00 | 53.12 |
| ATOM | 3140 | N | LEU | B | 819 | 15.299 | 18.439 | −16.636 | 1.00 | 49.58 |
| ATOM | 3141 | CA | LEU | B | 819 | 15.542 | 18.376 | −18.071 | 1.00 | 48.96 |
| ATOM | 3142 | C | LEU | B | 819 | 15.433 | 16.942 | −18.617 | 1.00 | 48.91 |
| ATOM | 3143 | O | LEU | B | 819 | 14.665 | 16.680 | −19.537 | 1.00 | 47.63 |
| ATOM | 3144 | CB | LEU | B | 819 | 16.937 | 18.931 | −18.342 | 1.00 | 49.33 |
| ATOM | 3145 | CG | LEU | B | 819 | 17.233 | 19.667 | −19.638 | 1.00 | 50.99 |
| ATOM | 3146 | CD1 | LEU | B | 819 | 16.180 | 20.729 | −19.883 | 1.00 | 51.26 |
| ATOM | 3147 | CD2 | LEU | B | 819 | 18.620 | 20.284 | −19.528 | 1.00 | 48.42 |
| ATOM | 3148 | N | LEU | B | 820 | 16.207 | 16.027 | −18.036 | 1.00 | 48.12 |
| ATOM | 3149 | CA | LEU | B | 820 | 16.232 | 14.627 | −18.448 | 1.00 | 47.03 |
| ATOM | 3150 | C | LEU | B | 820 | 14.908 | 13.879 | −18.355 | 1.00 | 47.74 |
| ATOM | 3151 | O | LEU | B | 820 | 14.621 | 13.036 | −19.210 | 1.00 | 46.11 |
| ATOM | 3152 | CB | LEU | B | 820 | 17.284 | 13.865 | −17.648 | 1.00 | 46.94 |
| ATOM | 3153 | CG | LEU | B | 820 | 18.733 | 14.082 | −18.069 | 1.00 | 46.17 |
| ATOM | 3154 | CD1 | LEU | B | 820 | 19.655 | 13.512 | −17.010 | 1.00 | 48.56 |
| ATOM | 3155 | CD2 | LEU | B | 820 | 18.982 | 13.429 | −19.417 | 1.00 | 47.04 |
| ATOM | 3156 | N | LEU | B | 821 | 14.119 | 14.159 | −17.315 | 1.00 | 47.54 |
| ATOM | 3157 | CA | LEU | B | 821 | 12.818 | 13.508 | −17.140 | 1.00 | 47.15 |
| ATOM | 3158 | C | LEU | B | 821 | 11.781 | 14.084 | −18.094 | 1.00 | 47.22 |
| ATOM | 3159 | O | LEU | B | 821 | 10.822 | 13.407 | −18.463 | 1.00 | 48.21 |
| ATOM | 3160 | CB | LEU | B | 821 | 12.311 | 13.667 | −15.712 | 1.00 | 46.22 |
| ATOM | 3161 | CG | LEU | B | 821 | 12.974 | 12.829 | −14.621 | 1.00 | 48.11 |
| ATOM | 3162 | CD1 | LEU | B | 821 | 12.444 | 13.272 | −13.264 | 1.00 | 46.19 |
| ATOM | 3163 | CD2 | LEU | B | 821 | 12.685 | 11.353 | −14.844 | 1.00 | 46.08 |
| ATOM | 3164 | N | ASN | B | 822 | 11.970 | 15.336 | −18.489 | 1.00 | 47.10 |
| ATOM | 3165 | CA | ASN | B | 822 | 11.048 | 15.981 | −19.415 | 1.00 | 48.36 |
| ATOM | 3166 | C | ASN | B | 822 | 11.311 | 15.539 | −20.860 | 1.00 | 46.42 |
| ATOM | 3167 | O | ASN | B | 822 | 10.398 | 15.478 | −21.678 | 1.00 | 46.12 |
| ATOM | 3168 | CB | ASN | B | 822 | 11.153 | 17.499 | −19.263 | 1.00 | 52.20 |
| ATOM | 3169 | CG | ASN | B | 822 | 10.430 | 18.003 | −18.020 | 1.00 | 56.94 |
| ATOM | 3170 | OD1 | ASN | B | 822 | 9.200 | 18.106 | −18.005 | 1.00 | 60.36 |
| ATOM | 3171 | ND2 | ASN | B | 822 | 11.184 | 18.295 | −16.966 | 1.00 | 58.16 |
| ATOM | 3172 | N | TRP | B | 823 | 12.560 | 15.220 | −21.174 | 1.00 | 45.04 |
| ATOM | 3173 | CA | TRP | B | 823 | 12.878 | 14.746 | −22.512 | 1.00 | 43.92 |
| ATOM | 3174 | C | TRP | B | 823 | 12.219 | 13.378 | −22.647 | 1.00 | 43.43 |
| ATOM | 3175 | O | TRP | B | 823 | 11.667 | 13.026 | −23.698 | 1.00 | 42.53 |
| ATOM | 3176 | CB | TRP | B | 823 | 14.390 | 14.597 | −22.700 | 1.00 | 42.86 |
| ATOM | 3177 | CG | TRP | B | 823 | 15.149 | 15.901 | −22.828 | 1.00 | 44.63 |
| ATOM | 3178 | CD1 | TRP | B | 823 | 14.654 | 17.109 | −23.246 | 1.00 | 43.05 |
| ATOM | 3179 | CD2 | TRP | B | 823 | 16.553 | 16.098 | −22.612 | 1.00 | 44.09 |
| ATOM | 3180 | NE1 | TRP | B | 823 | 15.664 | 18.036 | −23.308 | 1.00 | 43.18 |
| ATOM | 3181 | CE2 | TRP | B | 823 | 16.840 | 17.445 | −22.923 | 1.00 | 44.18 |
| ATOM | 3182 | CE3 | TRP | B | 823 | 17.599 | 15.264 | −22.184 | 1.00 | 44.59 |
| ATOM | 3183 | CZ2 | TRP | B | 823 | 18.133 | 17.985 | −22.818 | 1.00 | 44.47 |
| ATOM | 3184 | CZ3 | TRP | B | 823 | 18.886 | 15.803 | −22.080 | 1.00 | 46.01 |
| ATOM | 3185 | CH2 | TRP | B | 823 | 19.139 | 17.154 | −22.397 | 1.00 | 42.03 |
| ATOM | 3186 | N | CYS | B | 824 | 12.274 | 12.617 | −21.560 | 1.00 | 41.79 |
| ATOM | 3187 | CA | CYS | B | 824 | 11.699 | 11.290 | −21.535 | 1.00 | 42.66 |
| ATOM | 3188 | C | CYS | B | 824 | 10.243 | 11.359 | −21.920 | 1.00 | 44.42 |
| ATOM | 3189 | O | CYS | B | 824 | 9.785 | 10.604 | −22.778 | 1.00 | 45.73 |
| ATOM | 3190 | CB | CYS | B | 824 | 11.846 | 10.663 | −20.144 | 1.00 | 43.86 |
| ATOM | 3191 | SG | CYS | B | 824 | 13.522 | 10.059 | −19.758 | 1.00 | 43.47 |
| ATOM | 3192 | N | VAL | B | 825 | 9.521 | 12.278 | −21.287 | 1.00 | 45.21 |
| ATOM | 3193 | CA | VAL | B | 825 | 8.100 | 12.455 | −21.549 | 1.00 | 42.85 |
| ATOM | 3194 | C | VAL | B | 825 | 7.858 | 12.902 | −22.979 | 1.00 | 42.45 |
| ATOM | 3195 | O | VAL | B | 825 | 6.970 | 12.394 | −23.665 | 1.00 | 40.16 |
| ATOM | 3196 | CB | VAL | B | 825 | 7.486 | 13.495 | −20.585 | 1.00 | 42.45 |
| ATOM | 3197 | CG1 | VAL | B | 825 | 6.062 | 13.834 | −21.014 | 1.00 | 41.54 |
| ATOM | 3198 | CG2 | VAL | B | 825 | 7.488 | 12.948 | −19.173 | 1.00 | 40.00 |
| ATOM | 3199 | N | GLN | B | 826 | 8.650 | 13.868 | −23.423 | 1.00 | 43.25 |
| ATOM | 3200 | CA | GLN | B | 826 | 8.510 | 14.379 | −24.776 | 1.00 | 43.45 |
| ATOM | 3201 | C | GLN | B | 826 | 8.769 | 13.251 | −25.762 | 1.00 | 41.87 |
| ATOM | 3202 | O | GLN | B | 826 | 7.985 | 13.044 | −26.685 | 1.00 | 42.00 |
| ATOM | 3203 | CB | GLN | B | 826 | 9.468 | 15.555 | −25.000 | 1.00 | 42.33 |
| ATOM | 3204 | CG | GLN | B | 826 | 9.022 | 16.786 | −24.250 | 1.00 | 44.18 |
| ATOM | 3205 | CD | GLN | B | 826 | 9.988 | 17.944 | −24.348 | 1.00 | 44.47 |
| ATOM | 3206 | OE1 | GLN | B | 826 | 10.716 | 18.084 | −25.321 | 1.00 | 43.53 |
| ATOM | 3207 | NE2 | GLN | B | 826 | 9.981 | 18.799 | −23.335 | 1.00 | 47.99 |
| ATOM | 3208 | N | ILE | B | 827 | 9.845 | 12.498 | −25.565 | 1.00 | 39.85 |
| ATOM | 3209 | CA | ILE | B | 827 | 10.093 | 11.406 | −26.487 | 1.00 | 40.62 |
| ATOM | 3210 | C | ILE | B | 827 | 8.928 | 10.412 | −26.458 | 1.00 | 41.30 |
| ATOM | 3211 | O | ILE | B | 827 | 8.459 | 9.988 | −27.513 | 1.00 | 43.72 |
| ATOM | 3212 | CB | ILE | B | 827 | 11.402 | 10.662 | −26.178 | 1.00 | 39.44 |
| ATOM | 3213 | CG1 | ILE | B | 827 | 12.578 | 11.636 | −26.275 | 1.00 | 38.32 |
| ATOM | 3214 | CG2 | ILE | B | 827 | 11.583 | 9.500 | −27.171 | 1.00 | 37.20 |
| ATOM | 3215 | CD1 | ILE | B | 827 | 13.934 | 11.012 | −26.017 | 1.00 | 37.82 |
| ATOM | 3216 | N | ALA | B | 828 | 8.448 | 10.059 | −25.266 | 1.00 | 38.63 |
| ATOM | 3217 | CA | ALA | B | 828 | 7.347 | 9.113 | −25.146 | 1.00 | 40.14 |
| ATOM | 3218 | C | ALA | B | 828 | 6.043 | 9.649 | −25.728 | 1.00 | 42.58 |
| ATOM | 3219 | O | ALA | B | 828 | 5.185 | 8.881 | −26.177 | 1.00 | 43.82 |
| ATOM | 3220 | CB | ALA | B | 828 | 7.140 | 8.723 | −23.692 | 1.00 | 40.67 |
| ATOM | 3221 | N | LYS | B | 829 | 5.873 | 10.963 | −25.722 | 1.00 | 44.00 |
| ATOM | 3222 | CA | LYS | B | 829 | 4.647 | 11.525 | −26.273 | 1.00 | 43.33 |
| ATOM | 3223 | C | LYS | B | 829 | 4.656 | 11.428 | −27.795 | 1.00 | 43.62 |
| ATOM | 3224 | O | LYS | B | 829 | 3.640 | 11.094 | −28.401 | 1.00 | 44.41 |
| ATOM | 3225 | CB | LYS | B | 829 | 4.461 | 12.971 | −25.809 | 1.00 | 43.70 |
| ATOM | 3226 | CG | LYS | B | 829 | 3.810 | 13.088 | −24.433 | 1.00 | 41.95 |
| ATOM | 3227 | CD | LYS | B | 829 | 3.641 | 14.542 | −24.038 | 1.00 | 44.96 |
| ATOM | 3228 | CE | LYS | B | 829 | 2.871 | 14.679 | −22.747 | 1.00 | 47.02 |
| ATOM | 3229 | NZ | LYS | B | 829 | 2.772 | 16.101 | −22.347 | 1.00 | 49.90 |
| ATOM | 3230 | N | GLY | B | 830 | 5.803 | 11.704 | −28.410 | 1.00 | 42.29 |
| ATOM | 3231 | CA | GLY | B | 830 | 5.897 | 11.603 | −29.854 | 1.00 | 42.16 |
| ATOM | 3232 | C | GLY | B | 830 | 5.631 | 10.178 | −30.327 | 1.00 | 42.55 |
| ATOM | 3233 | O | GLY | B | 830 | 4.952 | 9.958 | −31.336 | 1.00 | 43.40 |
| ATOM | 3234 | N | MET | B | 831 | 6.178 | 9.202 | −29.611 | 1.00 | 41.44 |
| ATOM | 3235 | CA | MET | B | 831 | 5.955 | 7.811 | −29.972 | 1.00 | 43.60 |
| ATOM | 3236 | C | MET | B | 831 | 4.450 | 7.563 | −29.842 | 1.00 | 43.46 |
| ATOM | 3237 | O | MET | B | 831 | 3.840 | 6.886 | −30.670 | 1.00 | 42.80 |
| ATOM | 3238 | CB | MET | B | 831 | 6.748 | 6.867 | −29.042 | 1.00 | 43.14 |
| ATOM | 3239 | CG | MET | B | 831 | 8.267 | 6.966 | −29.199 | 1.00 | 42.68 |
| ATOM | 3240 | SD | MET | B | 831 | 8.809 | 6.840 | −30.938 | 1.00 | 43.27 |
| ATOM | 3241 | CE | MET | B | 831 | 8.511 | 5.103 | −31.247 | 1.00 | 44.19 |
| ATOM | 3242 | N | MET | B | 832 | 3.860 | 8.142 | −28.802 | 1.00 | 42.18 |
| ATOM | 3243 | CA | MET | B | 832 | 2.433 | 8.006 | −28.549 | 1.00 | 43.21 |
| ATOM | 3244 | C | MET | B | 832 | 1.641 | 8.454 | −29.780 | 1.00 | 42.04 |
| ATOM | 3245 | O | MET | B | 832 | 0.683 | 7.793 | −30.201 | 1.00 | 40.20 |
| ATOM | 3246 | CB | MET | B | 832 | 2.062 | 8.866 | −27.347 | 1.00 | 46.91 |
| ATOM | 3247 | CG | MET | B | 832 | 0.984 | 8.297 | −26.452 | 1.00 | 50.62 |
| ATOM | 3248 | SD | MET | B | 832 | 0.679 | 9.445 | −25.093 | 1.00 | 58.47 |
| ATOM | 3249 | CE | MET | B | 832 | 1.936 | 8.941 | −24.018 | 1.00 | 53.62 |
| ATOM | 3250 | N | TYR | B | 833 | 2.066 | 9.581 | −30.349 | 1.00 | 40.89 |
| ATOM | 3251 | CA | TYR | B | 833 | 1.439 | 10.157 | −31.523 | 1.00 | 39.85 |
| ATOM | 3252 | C | TYR | B | 833 | 1.616 | 9.253 | −32.734 | 1.00 | 41.61 |
| ATOM | 3253 | O | TYR | B | 833 | 0.714 | 9.143 | −33.572 | 1.00 | 39.17 |
| ATOM | 3254 | CB | TYR | B | 833 | 2.048 | 11.521 | −31.824 | 1.00 | 39.98 |
| ATOM | 3255 | CG | TYR | B | 833 | 1.369 | 12.250 | −32.970 | 1.00 | 42.77 |
| ATOM | 3256 | CD1 | TYR | B | 833 | 0.311 | 13.147 | −32.737 | 1.00 | 41.95 |
| ATOM | 3257 | CD2 | TYR | B | 833 | 1.787 | 12.053 | −34.290 | 1.00 | 38.99 |
| ATOM | 3258 | CE1 | TYR | B | 833 | −0.301 | 13.832 | −33.798 | 1.00 | 38.79 |
| ATOM | 3259 | CE2 | TYR | B | 833 | 1.184 | 12.724 | −35.345 | 1.00 | 38.68 |
| ATOM | 3260 | CZ | TYR | B | 833 | 0.145 | 13.612 | −35.097 | 1.00 | 40.21 |
| ATOM | 3261 | OH | TYR | B | 833 | −0.433 | 14.279 | −36.159 | 1.00 | 40.82 |
| ATOM | 3262 | N | LEU | B | 834 | 2.784 | 8.619 | −32.829 | 1.00 | 42.19 |
| ATOM | 3263 | CA | LEU | B | 834 | 3.080 | 7.721 | −33.944 | 1.00 | 43.73 |
| ATOM | 3264 | C | LEU | B | 834 | 2.265 | 6.423 | −33.844 | 1.00 | 44.01 |
| ATOM | 3265 | O | LEU | B | 834 | 1.736 | 5.937 | −34.845 | 1.00 | 42.17 |
| ATOM | 3266 | CB | LEU | B | 834 | 4.584 | 7.384 | −33.989 | 1.00 | 45.03 |
| ATOM | 3267 | CG | LEU | B | 834 | 5.669 | 8.440 | −34.301 | 1.00 | 47.70 |
| ATOM | 3268 | CD1 | LEU | B | 834 | 7.028 | 7.851 | −33.978 | 1.00 | 46.80 |
| ATOM | 3269 | CD2 | LEU | B | 834 | 5.649 | 8.858 | −35.759 | 1.00 | 44.71 |
| ATOM | 3270 | N | GLU | B | 835 | 2.166 | 5.866 | −32.640 | 1.00 | 44.61 |
| ATOM | 3271 | CA | GLU | B | 835 | 1.428 | 4.624 | −32.444 | 1.00 | 48.10 |
| ATOM | 3272 | C | GLU | B | 835 | −0.053 | 4.827 | −32.708 | 1.00 | 50.02 |
| ATOM | 3273 | O | GLU | B | 835 | −0.735 | 3.937 | −33.215 | 1.00 | 48.65 |
| ATOM | 3274 | CB | GLU | B | 835 | 1.622 | 4.089 | −31.027 | 1.00 | 48.14 |
| ATOM | 3275 | CG | GLU | B | 835 | 0.745 | 2.893 | −30.733 | 1.00 | 51.80 |
| ATOM | 3276 | CD | GLU | B | 835 | 0.921 | 2.380 | −29.327 | 1.00 | 55.96 |
| ATOM | 3277 | OE1 | GLU | B | 835 | 1.020 | 3.212 | −28.401 | 1.00 | 58.77 |
| ATOM | 3278 | OE2 | GLU | B | 835 | 0.949 | 1.147 | −29.139 | 1.00 | 59.79 |
| ATOM | 3279 | N | GLU | B | 836 | −0.537 | 6.008 | −32.347 | 1.00 | 51.95 |
| ATOM | 3280 | CA | GLU | B | 836 | −1.926 | 6.378 | −32.545 | 1.00 | 53.09 |
| ATOM | 3281 | C | GLU | B | 836 | −2.227 | 6.270 | −34.048 | 1.00 | 52.44 |
| ATOM | 3282 | O | GLU | B | 836 | −3.325 | 5.874 | −34.442 | 1.00 | 52.73 |
| ATOM | 3283 | CB | GLU | B | 836 | −2.121 | 7.807 | −32.029 | 1.00 | 55.57 |
| ATOM | 3284 | CG | GLU | B | 836 | −3.548 | 8.296 | −31.901 | 1.00 | 62.73 |
| ATOM | 3285 | CD | GLU | B | 836 | −3.630 | 9.684 | −31.243 | 1.00 | 69.86 |
| ATOM | 3286 | OE1 | GLU | B | 836 | −4.721 | 10.308 | −31.286 | 1.00 | 71.77 |
| ATOM | 3287 | OE2 | GLU | B | 836 | −2.606 | 10.150 | −30.677 | 1.00 | 71.84 |
| ATOM | 3288 | N | ARG | B | 837 | −1.233 | 6.597 | −34.875 | 1.00 | 50.86 |
| ATOM | 3289 | CA | ARG | B | 837 | −1.361 | 6.550 | −36.339 | 1.00 | 51.45 |
| ATOM | 3290 | C | ARG | B | 837 | −0.926 | 5.206 | −36.939 | 1.00 | 50.30 |
| ATOM | 3291 | O | ARG | B | 837 | −0.750 | 5.098 | −38.148 | 1.00 | 47.12 |
| ATOM | 3292 | CB | ARG | B | 837 | −0.501 | 7.650 | −36.987 | 1.00 | 53.02 |
| ATOM | 3293 | CG | ARG | B | 837 | −0.781 | 9.066 | −36.521 | 1.00 | 55.22 |
| ATOM | 3294 | CD | ARG | B | 837 | −2.239 | 9.377 | −36.704 | 1.00 | 58.03 |
| ATOM | 3295 | NE | ARG | B | 837 | −2.603 | 10.765 | −36.431 | 1.00 | 59.91 |
| ATOM | 3296 | CZ | ARG | B | 837 | −2.371 | 11.412 | −35.294 | 1.00 | 58.31 |
| ATOM | 3297 | NH1 | ARG | B | 837 | −1.745 | 10.822 | −34.282 | 1.00 | 57.92 |
| ATOM | 3298 | NH2 | ARG | B | 837 | −2.830 | 12.645 | −35.152 | 1.00 | 57.89 |
| ATOM | 3299 | N | ARG | B | 838 | −0.754 | 4.198 | −36.086 | 1.00 | 51.66 |
| ATOM | 3300 | CA | ARG | B | 838 | −0.295 | 2.864 | −36.489 | 1.00 | 51.85 |
| ATOM | 3301 | C | ARG | B | 838 | 1.055 | 2.920 | −37.202 | 1.00 | 49.65 |
| ATOM | 3302 | O | ARG | B | 838 | 1.290 | 2.213 | −38.186 | 1.00 | 50.29 |
| ATOM | 3303 | CB | ARG | B | 838 | −1.334 | 2.143 | −37.369 | 1.00 | 53.63 |
| ATOM | 3304 | CG | ARG | B | 838 | −2.470 | 1.492 | −36.575 | 1.00 | 59.68 |
| ATOM | 3305 | CD | ARG | B | 838 | −1.932 | 0.532 | −35.496 | 1.00 | 64.00 |
| ATOM | 3306 | NE | ARG | B | 838 | −1.885 | −0.866 | −35.933 | 1.00 | 69.82 |
| ATOM | 3307 | CZ | ARG | B | 838 | −2.913 | −1.717 | −35.876 | 1.00 | 71.87 |
| ATOM | 3308 | NH1 | ARG | B | 838 | −2.764 | −2.966 | −36.307 | 1.00 | 71.46 |
| ATOM | 3309 | NH2 | ARG | B | 838 | −4.084 | −1.332 | −35.374 | 1.00 | 72.32 |
| ATOM | 3310 | N | LEU | B | 839 | 1.945 | 3.759 | −36.692 | 1.00 | 45.08 |
| ATOM | 3311 | CA | LEU | B | 839 | 3.263 | 3.886 | −37.282 | 1.00 | 44.44 |
| ATOM | 3312 | C | LEU | B | 839 | 4.350 | 3.426 | −36.319 | 1.00 | 44.15 |
| ATOM | 3313 | O | LEU | B | 839 | 4.454 | 3.900 | −35.183 | 1.00 | 43.92 |
| ATOM | 3314 | CB | LEU | B | 839 | 3.512 | 5.331 | −37.699 | 1.00 | 45.11 |
| ATOM | 3315 | CG | LEU | B | 839 | 2.429 | 5.939 | −38.601 | 1.00 | 47.37 |
| ATOM | 3316 | CD1 | LEU | B | 839 | 2.763 | 7.407 | −38.852 | 1.00 | 45.99 |
| ATOM | 3317 | CD2 | LEU | B | 839 | 2.317 | 5.161 | −39.921 | 1.00 | 43.39 |
| ATOM | 3318 | N | VAL | B | 840 | 5.152 | 2.481 | −36.785 | 1.00 | 43.12 |
| ATOM | 3319 | CA | VAL | B | 840 | 6.246 | 1.936 | −36.000 | 1.00 | 42.97 |
| ATOM | 3320 | C | VAL | B | 840 | 7.543 | 2.589 | −36.490 | 1.00 | 43.65 |
| ATOM | 3321 | O | VAL | B | 840 | 7.796 | 2.628 | −37.690 | 1.00 | 44.79 |
| ATOM | 3322 | CB | VAL | B | 840 | 6.305 | 0.408 | −36.192 | 1.00 | 42.39 |
| ATOM | 3323 | CG1 | VAL | B | 840 | 7.382 | −0.221 | −35.307 | 1.00 | 42.09 |
| ATOM | 3324 | CG2 | VAL | B | 840 | 4.945 | −0.177 | −35.881 | 1.00 | 41.99 |
| ATOM | 3325 | N | HIS | B | 841 | 8.351 | 3.110 | −35.570 | 1.00 | 43.05 |
| ATOM | 3326 | CA | HIS | B | 841 | 9.613 | 3.756 | −35.932 | 1.00 | 43.35 |
| ATOM | 3327 | C | HIS | B | 841 | 10.680 | 2.760 | −36.412 | 1.00 | 44.72 |
| ATOM | 3328 | O | HIS | B | 841 | 11.258 | 2.927 | −37.485 | 1.00 | 45.10 |
| ATOM | 3329 | CB | HIS | B | 841 | 10.150 | 4.547 | −34.736 | 1.00 | 41.90 |
| ATOM | 3330 | CG | HIS | B | 841 | 11.221 | 5.535 | −35.090 | 1.00 | 40.88 |
| ATOM | 3331 | ND1 | HIS | B | 841 | 12.406 | 5.170 | −35.690 | 1.00 | 39.11 |
| ATOM | 3332 | CD2 | HIS | B | 841 | 11.289 | 6.876 | −34.917 | 1.00 | 41.53 |
| ATOM | 3333 | CE1 | HIS | B | 841 | 13.156 | 6.242 | −35.870 | 1.00 | 40.04 |
| ATOM | 3334 | NE2 | HIS | B | 841 | 12.502 | 7.291 | −35.409 | 1.00 | 39.97 |
| ATOM | 3335 | N | ARG | B | 842 | 10.937 | 1.731 | −35.607 | 1.00 | 46.60 |
| ATOM | 3336 | CA | ARG | B | 842 | 11.927 | 0.691 | −35.915 | 1.00 | 47.69 |
| ATOM | 3337 | C | ARG | B | 842 | 13.380 | 1.106 | −35.704 | 1.00 | 47.11 |
| ATOM | 3338 | O | ARG | B | 842 | 14.260 | 0.250 | −35.650 | 1.00 | 47.21 |
| ATOM | 3339 | CB | ARG | B | 842 | 11.794 | 0.199 | −37.356 | 1.00 | 48.94 |
| ATOM | 3340 | CG | ARG | B | 842 | 10.483 | −0.442 | −37.701 | 1.00 | 53.39 |
| ATOM | 3341 | CD | ARG | B | 842 | 10.558 | −1.004 | −39.106 | 1.00 | 56.65 |
| ATOM | 3342 | NE | ARG | B | 842 | 9.354 | −1.720 | −39.505 | 1.00 | 57.96 |
| ATOM | 3343 | CZ | ARG | B | 842 | 9.326 | −2.590 | −40.504 | 1.00 | 59.81 |
| ATOM | 3344 | NH1 | ARG | B | 842 | 10.435 | −2.834 | −41.183 | 1.00 | 61.66 |
| ATOM | 3345 | NH2 | ARG | B | 842 | 8.203 | −3.216 | −40.821 | 1.00 | 61.73 |
| ATOM | 3346 | N | ASP | B | 843 | 13.639 | 2.403 | −35.587 | 1.00 | 45.43 |
| ATOM | 3347 | CA | ASP | B | 843 | 15.009 | 2.862 | −35.402 | 1.00 | 44.93 |
| ATOM | 3348 | C | ASP | B | 843 | 15.108 | 4.035 | −34.427 | 1.00 | 43.50 |
| ATOM | 3349 | O | ASP | B | 843 | 15.803 | 5.029 | −34.679 | 1.00 | 40.29 |
| ATOM | 3350 | CB | ASP | B | 843 | 15.602 | 3.234 | −36.769 | 1.00 | 48.21 |
| ATOM | 3351 | CG | ASP | B | 843 | 17.058 | 3.636 | −36.687 | 1.00 | 49.17 |
| ATOM | 3352 | OD1 | ASP | B | 843 | 17.829 | 2.925 | −36.005 | 1.00 | 50.67 |
| ATOM | 3353 | OD2 | ASP | B | 843 | 17.424 | 4.656 | −37.309 | 1.00 | 50.03 |
| ATOM | 3354 | N | LEU | B | 844 | 14.397 | 3.914 | −33.312 | 1.00 | 41.09 |
| ATOM | 3355 | CA | LEU | B | 844 | 14.409 | 4.942 | −32.296 | 1.00 | 39.45 |
| ATOM | 3356 | C | LEU | B | 844 | 15.683 | 4.757 | −31.476 | 1.00 | 41.41 |
| ATOM | 3357 | O | LEU | B | 844 | 15.962 | 3.662 | −30.982 | 1.00 | 39.94 |
| ATOM | 3358 | CB | LEU | B | 844 | 13.192 | 4.802 | −31.394 | 1.00 | 37.57 |
| ATOM | 3359 | CG | LEU | B | 844 | 13.136 | 5.718 | −30.161 | 1.00 | 37.58 |
| ATOM | 3360 | CD1 | LEU | B | 844 | 12.897 | 7.175 | −30.572 | 1.00 | 31.85 |
| ATOM | 3361 | CD2 | LEU | B | 844 | 12.023 | 5.232 | −29.248 | 1.00 | 33.61 |
| ATOM | 3362 | N | ALA | B | 845 | 16.461 | 5.825 | −31.356 | 1.00 | 41.14 |
| ATOM | 3363 | CA | ALA | B | 845 | 17.703 | 5.793 | −30.594 | 1.00 | 43.80 |
| ATOM | 3364 | C | ALA | B | 845 | 17.998 | 7.233 | −30.239 | 1.00 | 44.77 |
| ATOM | 3365 | O | ALA | B | 845 | 17.463 | 8.139 | −30.872 | 1.00 | 46.46 |
| ATOM | 3366 | CB | ALA | B | 845 | 18.839 | 5.208 | −31.436 | 1.00 | 43.20 |
| ATOM | 3367 | N | ALA | B | 846 | 18.838 | 7.462 | −29.241 | 1.00 | 44.58 |
| ATOM | 3368 | CA | ALA | B | 846 | 19.124 | 8.833 | −28.856 | 1.00 | 45.24 |
| ATOM | 3369 | C | ALA | B | 846 | 19.609 | 9.661 | −30.030 | 1.00 | 45.63 |
| ATOM | 3370 | O | ALA | B | 846 | 19.289 | 10.850 | −30.115 | 1.00 | 47.93 |
| ATOM | 3371 | CB | ALA | B | 846 | 20.142 | 8.870 | −27.734 | 1.00 | 45.17 |
| ATOM | 3372 | N | ARG | B | 847 | 20.363 | 9.045 | −30.941 | 1.00 | 44.22 |
| ATOM | 3373 | CA | ARG | B | 847 | 20.886 | 9.786 | −32.094 | 1.00 | 46.27 |
| ATOM | 3374 | C | ARG | B | 847 | 19.800 | 10.270 | −33.054 | 1.00 | 46.18 |
| ATOM | 3375 | O | ARG | B | 847 | 20.043 | 11.161 | −33.865 | 1.00 | 46.55 |
| ATOM | 3376 | CB | ARG | B | 847 | 21.895 | 8.951 | −32.877 | 1.00 | 46.11 |
| ATOM | 3377 | CG | ARG | B | 847 | 21.254 | 7.928 | −33.760 | 1.00 | 49.88 |
| ATOM | 3378 | CD | ARG | B | 847 | 22.263 | 7.195 | −34.602 | 1.00 | 50.73 |
| ATOM | 3379 | NE | ARG | B | 847 | 21.564 | 6.353 | −35.565 | 1.00 | 57.37 |
| ATOM | 3380 | CZ | ARG | B | 847 | 20.873 | 5.264 | −35.242 | 1.00 | 58.79 |
| ATOM | 3381 | NH1 | ARG | B | 847 | 20.794 | 4.872 | −33.976 | 1.00 | 60.01 |
| ATOM | 3382 | NH2 | ARG | B | 847 | 20.243 | 4.576 | −36.182 | 1.00 | 59.64 |
| ATOM | 3383 | N | ASN | B | 848 | 18.610 | 9.683 | −32.978 | 1.00 | 45.11 |
| ATOM | 3384 | CA | ASN | B | 848 | 17.532 | 10.114 | −33.848 | 1.00 | 45.22 |
| ATOM | 3385 | C | ASN | B | 848 | 16.525 | 11.015 | −33.132 | 1.00 | 47.16 |
| ATOM | 3386 | O | ASN | B | 848 | 15.351 | 11.117 | −33.525 | 1.00 | 46.11 |
| ATOM | 3387 | CB | ASN | B | 848 | 16.840 | 8.917 | −34.500 | 1.00 | 43.37 |
| ATOM | 3388 | CG | ASN | B | 848 | 17.576 | 8.434 | −35.736 | 1.00 | 44.82 |
| ATOM | 3389 | OD1 | ASN | B | 848 | 17.953 | 9.234 | −36.596 | 1.00 | 47.01 |
| ATOM | 3390 | ND2 | ASN | B | 848 | 17.781 | 7.127 | −35.838 | 1.00 | 42.89 |
| ATOM | 3391 | N | VAL | B | 849 | 16.996 | 11.657 | −32.065 | 1.00 | 47.02 |
| ATOM | 3392 | CA | VAL | B | 849 | 16.181 | 12.606 | −31.330 | 1.00 | 48.04 |
| ATOM | 3393 | C | VAL | B | 849 | 16.951 | 13.914 | −31.472 | 1.00 | 48.97 |
| ATOM | 3394 | O | VAL | B | 849 | 18.134 | 13.987 | −31.146 | 1.00 | 49.90 |
| ATOM | 3395 | CB | VAL | B | 849 | 16.026 | 12.235 | −29.829 | 1.00 | 48.21 |
| ATOM | 3396 | CG1 | VAL | B | 849 | 15.223 | 13.322 | −29.109 | 1.00 | 48.81 |
| ATOM | 3397 | CG2 | VAL | B | 849 | 15.298 | 10.899 | −29.689 | 1.00 | 46.65 |
| ATOM | 3398 | N | LEU | B | 850 | 16.281 | 14.934 | −31.995 | 1.00 | 50.30 |
| ATOM | 3399 | CA | LEU | B | 850 | 16.900 | 16.234 | −32.216 | 1.00 | 49.70 |
| ATOM | 3400 | C | LEU | B | 850 | 16.503 | 17.241 | −31.144 | 1.00 | 51.97 |
| ATOM | 3401 | O | LEU | B | 850 | 15.449 | 17.116 | −30.514 | 1.00 | 53.21 |
| ATOM | 3402 | CB | LEU | B | 850 | 16.490 | 16.768 | −33.583 | 1.00 | 46.49 |
| ATOM | 3403 | CG | LEU | B | 850 | 16.660 | 15.825 | −34.771 | 1.00 | 45.29 |
| ATOM | 3404 | CD1 | LEU | B | 850 | 16.076 | 16.481 | −35.999 | 1.00 | 43.36 |
| ATOM | 3405 | CD2 | LEU | B | 850 | 18.120 | 15.500 | −34.986 | 1.00 | 44.40 |
| ATOM | 3406 | N | VAL | B | 851 | 17.352 | 18.249 | −30.954 | 1.00 | 53.33 |
| ATOM | 3407 | CA | VAL | B | 851 | 17.112 | 19.284 | −29.960 | 1.00 | 53.66 |
| ATOM | 3408 | C | VAL | B | 851 | 16.731 | 20.610 | −30.628 | 1.00 | 55.93 |
| ATOM | 3409 | O | VAL | B | 851 | 17.595 | 21.317 | −31.153 | 1.00 | 56.57 |
| ATOM | 3410 | CB | VAL | B | 851 | 18.375 | 19.494 | −29.094 | 1.00 | 52.04 |
| ATOM | 3411 | CG1 | VAL | B | 851 | 18.081 | 20.455 | −27.966 | 1.00 | 53.25 |
| ATOM | 3412 | CG2 | VAL | B | 851 | 18.850 | 18.171 | −28.540 | 1.00 | 50.16 |
| ATOM | 3413 | N | LYS | B | 852 | 15.440 | 20.940 | −30.624 | 1.00 | 57.66 |
| ATOM | 3414 | CA | LYS | B | 852 | 14.982 | 22.196 | −31.222 | 1.00 | 60.00 |
| ATOM | 3415 | C | LYS | B | 852 | 15.497 | 23.295 | −30.317 | 1.00 | 62.13 |
| ATOM | 3416 | O | LYS | B | 852 | 15.977 | 24.328 | −30.778 | 1.00 | 64.39 |
| ATOM | 3417 | CB | LYS | B | 852 | 13.458 | 22.268 | −31.272 | 1.00 | 58.87 |
| ATOM | 3418 | CG | LYS | B | 852 | 12.923 | 23.615 | −31.741 | 1.00 | 58.96 |
| ATOM | 3419 | CD | LYS | B | 852 | 11.398 | 23.646 | −31.691 | 1.00 | 60.41 |
| ATOM | 3420 | CE | LYS | B | 852 | 10.817 | 24.892 | −32.352 | 1.00 | 60.45 |
| ATOM | 3421 | NZ | LYS | B | 852 | 9.312 | 24.862 | −32.407 | 1.00 | 61.83 |
| ATOM | 3422 | N | SER | B | 853 | 15.374 | 23.050 | −29.017 | 1.00 | 61.87 |
| ATOM | 3423 | CA | SER | B | 853 | 15.837 | 23.960 | −27.980 | 1.00 | 61.38 |
| ATOM | 3424 | C | SER | B | 853 | 15.945 | 23.145 | −26.680 | 1.00 | 61.16 |
| ATOM | 3425 | O | SER | B | 853 | 15.236 | 22.157 | −26.489 | 1.00 | 58.97 |
| ATOM | 3426 | CB | SER | B | 853 | 14.878 | 25.147 | −27.822 | 1.00 | 59.92 |
| ATOM | 3427 | OG | SER | B | 853 | 13.582 | 24.731 | −27.445 | 1.00 | 61.79 |
| ATOM | 3428 | N | PRO | B | 854 | 16.851 | 23.546 | −25.780 | 1.00 | 61.17 |
| ATOM | 3429 | CA | PRO | B | 854 | 17.076 | 22.865 | −24.503 | 1.00 | 60.56 |
| ATOM | 3430 | C | PRO | B | 854 | 15.876 | 22.143 | −23.892 | 1.00 | 59.89 |
| ATOM | 3431 | O | PRO | B | 854 | 15.957 | 20.959 | −23.555 | 1.00 | 59.58 |
| ATOM | 3432 | CB | PRO | B | 854 | 17.599 | 23.986 | −23.625 | 1.00 | 61.16 |
| ATOM | 3433 | CG | PRO | B | 854 | 18.478 | 24.738 | −24.599 | 1.00 | 62.45 |
| ATOM | 3434 | CD | PRO | B | 854 | 17.600 | 24.818 | −25.834 | 1.00 | 62.08 |
| ATOM | 3435 | N | ALA | B | 855 | 14.764 | 22.850 | −23.762 | 1.00 | 57.36 |
| ATOM | 3436 | CA | ALA | B | 855 | 13.576 | 22.269 | −23.173 | 1.00 | 55.01 |
| ATOM | 3437 | C | ALA | B | 855 | 12.621 | 21.699 | −24.210 | 1.00 | 55.18 |
| ATOM | 3438 | O | ALA | B | 855 | 11.440 | 21.533 | −23.929 | 1.00 | 54.40 |
| ATOM | 3439 | CB | ALA | B | 855 | 12.867 | 23.323 | −22.354 | 1.00 | 54.10 |
| ATOM | 3440 | N | HIS | B | 856 | 13.116 | 21.368 | −25.399 | 1.00 | 55.23 |
| ATOM | 3441 | CA | HIS | B | 856 | 12.222 | 20.881 | −26.448 | 1.00 | 52.68 |
| ATOM | 3442 | C | HIS | B | 856 | 12.891 | 19.970 | −27.478 | 1.00 | 51.26 |
| ATOM | 3443 | O | HIS | B | 856 | 13.649 | 20.427 | −28.343 | 1.00 | 51.11 |
| ATOM | 3444 | CB | HIS | B | 856 | 11.602 | 22.096 | −27.148 | 1.00 | 53.04 |
| ATOM | 3445 | CG | HIS | B | 856 | 10.492 | 21.766 | −28.097 | 1.00 | 55.71 |
| ATOM | 3446 | ND1 | HIS | B | 856 | 9.868 | 22.714 | −28.880 | 1.00 | 55.62 |
| ATOM | 3447 | CD2 | HIS | B | 856 | 9.902 | 20.583 | −28.396 | 1.00 | 54.97 |
| ATOM | 3448 | CE1 | HIS | B | 856 | 8.943 | 22.129 | −29.619 | 1.00 | 55.10 |
| ATOM | 3449 | NE2 | HIS | B | 856 | 8.942 | 20.837 | −29.344 | 1.00 | 55.98 |
| ATOM | 3450 | N | VAL | B | 857 | 12.602 | 18.677 | −27.388 | 1.00 | 48.98 |
| ATOM | 3451 | CA | VAL | B | 857 | 13.164 | 17.717 | −28.328 | 1.00 | 47.33 |
| ATOM | 3452 | C | VAL | B | 857 | 12.076 | 17.225 | −29.291 | 1.00 | 46.32 |
| ATOM | 3453 | O | VAL | B | 857 | 10.877 | 17.362 | −29.018 | 1.00 | 45.36 |
| ATOM | 3454 | CB | VAL | B | 857 | 13.827 | 16.500 | −27.593 | 1.00 | 46.33 |
| ATOM | 3455 | CG1 | VAL | B | 857 | 15.094 | 16.944 | −26.897 | 1.00 | 44.86 |
| ATOM | 3456 | CG2 | VAL | B | 857 | 12.874 | 15.902 | −26.581 | 1.00 | 44.39 |
| ATOM | 3457 | N | LYS | B | 858 | 12.516 | 16.681 | −30.425 | 1.00 | 44.31 |
| ATOM | 3458 | CA | LYS | B | 858 | 11.636 | 16.145 | −31.459 | 1.00 | 43.59 |
| ATOM | 3459 | C | LYS | B | 858 | 12.222 | 14.849 | −32.014 | 1.00 | 43.20 |
| ATOM | 3460 | O | LYS | B | 858 | 13.438 | 14.753 | −32.217 | 1.00 | 43.70 |
| ATOM | 3461 | CB | LYS | B | 858 | 11.492 | 17.140 | −32.614 | 1.00 | 44.67 |
| ATOM | 3462 | CG | LYS | B | 858 | 10.538 | 18.301 | −32.362 | 1.00 | 46.35 |
| ATOM | 3463 | CD | LYS | B | 858 | 10.463 | 19.170 | −33.610 | 1.00 | 46.33 |
| ATOM | 3464 | CE | LYS | B | 858 | 9.382 | 20.215 | −33.508 | 1.00 | 46.98 |
| ATOM | 3465 | NZ | LYS | B | 858 | 9.214 | 20.892 | −34.816 | 1.00 | 50.99 |
| ATOM | 3466 | N | ILE | B | 859 | 11.367 | 13.855 | −32.265 | 1.00 | 42.00 |
| ATOM | 3467 | CA | ILE | B | 859 | 11.832 | 12.583 | −32.816 | 1.00 | 39.14 |
| ATOM | 3468 | C | ILE | B | 859 | 11.955 | 12.719 | −34.328 | 1.00 | 39.96 |
| ATOM | 3469 | O | ILE | B | 859 | 11.140 | 13.386 | −34.966 | 1.00 | 40.22 |
| ATOM | 3470 | CB | ILE | B | 859 | 10.863 | 11.431 | −32.480 | 1.00 | 37.12 |
| ATOM | 3471 | CG1 | ILE | B | 859 | 10.738 | 11.297 | −30.958 | 1.00 | 37.67 |
| ATOM | 3472 | CG2 | ILE | B | 859 | 11.384 | 10.129 | −33.069 | 1.00 | 34.33 |
| ATOM | 3473 | CD1 | ILE | B | 859 | 9.834 | 10.171 | −30.480 | 1.00 | 33.72 |
| ATOM | 3474 | N | THR | B | 860 | 12.977 | 12.093 | −34.900 | 1.00 | 41.18 |
| ATOM | 3475 | CA | THR | B | 860 | 13.197 | 12.174 | −36.342 | 1.00 | 43.70 |
| ATOM | 3476 | C | THR | B | 860 | 13.596 | 10.844 | −36.971 | 1.00 | 45.83 |
| ATOM | 3477 | O | THR | B | 860 | 14.019 | 9.918 | −36.278 | 1.00 | 47.50 |
| ATOM | 3478 | CB | THR | B | 860 | 14.312 | 13.218 | −36.688 | 1.00 | 42.70 |
| ATOM | 3479 | OG1 | THR | B | 860 | 14.311 | 13.486 | −38.096 | 1.00 | 41.02 |
| ATOM | 3480 | CG2 | THR | B | 860 | 15.685 | 12.683 | −36.317 | 1.00 | 41.73 |
| ATOM | 3481 | N | ASP | B | 861 | 13.463 | 10.772 | −38.294 | 1.00 | 46.92 |
| ATOM | 3482 | CA | ASP | B | 861 | 13.839 | 9.597 | −39.078 | 1.00 | 49.85 |
| ATOM | 3483 | C | ASP | B | 861 | 12.873 | 8.419 | −39.058 | 1.00 | 50.50 |
| ATOM | 3484 | O | ASP | B | 861 | 13.233 | 7.317 | −39.458 | 1.00 | 50.83 |
| ATOM | 3485 | CB | ASP | B | 861 | 15.239 | 9.109 | −38.680 | 1.00 | 50.52 |
| ATOM | 3486 | CG | ASP | B | 861 | 16.070 | 8.688 | −39.883 | 1.00 | 54.77 |
| ATOM | 3487 | OD1 | ASP | B | 861 | 16.304 | 9.533 | −40.764 | 1.00 | 56.69 |
| ATOM | 3488 | OD2 | ASP | B | 861 | 16.495 | 7.518 | −39.959 | 1.00 | 58.38 |
| ATOM | 3489 | N | PHE | B | 862 | 11.649 | 8.633 | −38.600 | 1.00 | 52.16 |
| ATOM | 3490 | CA | PHE | B | 862 | 10.689 | 7.542 | −38.606 | 1.00 | 55.68 |
| ATOM | 3491 | C | PHE | B | 862 | 10.256 | 7.323 | −40.057 | 1.00 | 58.32 |
| ATOM | 3492 | O | PHE | B | 862 | 10.120 | 8.281 | −40.823 | 1.00 | 59.56 |
| ATOM | 3493 | CB | PHE | B | 862 | 9.472 | 7.873 | −37.732 | 1.00 | 55.12 |
| ATOM | 3494 | CG | PHE | B | 862 | 8.874 | 9.233 | −37.995 | 1.00 | 54.59 |
| ATOM | 3495 | CD1 | PHE | B | 862 | 9.153 | 10.303 | −37.149 | 1.00 | 54.37 |
| ATOM | 3496 | CD2 | PHE | B | 862 | 8.038 | 9.445 | −39.085 | 1.00 | 53.46 |
| ATOM | 3497 | CE1 | PHE | B | 862 | 8.610 | 11.560 | −37.385 | 1.00 | 53.72 |
| ATOM | 3498 | CE2 | PHE | B | 862 | 7.489 | 10.700 | −39.331 | 1.00 | 52.07 |
| ATOM | 3499 | CZ | PHE | B | 862 | 7.777 | 11.757 | −38.478 | 1.00 | 53.20 |
| ATOM | 3500 | N | GLY | B | 863 | 10.066 | 6.068 | −40.446 | 1.00 | 60.33 |
| ATOM | 3501 | CA | GLY | B | 863 | 9.646 | 5.797 | −41.809 | 1.00 | 64.81 |
| ATOM | 3502 | C | GLY | B | 863 | 10.679 | 5.163 | −42.725 | 1.00 | 68.14 |
| ATOM | 3503 | O | GLY | B | 863 | 10.377 | 4.174 | −43.393 | 1.00 | 68.10 |
| ATOM | 3504 | N | LEU | B | 864 | 11.887 | 5.726 | −42.768 | 1.00 | 70.85 |
| ATOM | 3505 | CA | LEU | B | 864 | 12.952 | 5.194 | −43.622 | 1.00 | 73.25 |
| ATOM | 3506 | C | LEU | B | 864 | 13.219 | 3.714 | −43.386 | 1.00 | 74.47 |
| ATOM | 3507 | O | LEU | B | 864 | 13.373 | 2.950 | −44.335 | 1.00 | 76.25 |
| ATOM | 3508 | CB | LEU | B | 864 | 14.267 | 5.949 | −43.412 | 1.00 | 73.37 |
| ATOM | 3509 | CG | LEU | B | 864 | 14.399 | 7.414 | −43.815 | 1.00 | 72.75 |
| ATOM | 3510 | CD1 | LEU | B | 864 | 15.853 | 7.800 | −43.685 | 1.00 | 72.55 |
| ATOM | 3511 | CD2 | LEU | B | 864 | 13.925 | 7.633 | −45.237 | 1.00 | 72.97 |
| ATOM | 3512 | N | ALA | B | 865 | 13.291 | 3.319 | −42.121 | 1.00 | 74.99 |
| ATOM | 3513 | CA | ALA | B | 865 | 13.549 | 1.932 | −41.769 | 1.00 | 75.61 |
| ATOM | 3514 | C | ALA | B | 865 | 12.791 | 0.967 | −42.679 | 1.00 | 76.21 |
| ATOM | 3515 | O | ALA | B | 865 | 13.392 | 0.215 | −43.456 | 1.00 | 76.63 |
| ATOM | 3516 | CB | ALA | B | 865 | 13.163 | 1.692 | −40.322 | 1.00 | 76.18 |
| ATOM | 3517 | N | ARG | B | 866 | 11.467 | 1.001 | −42.582 | 1.00 | 75.63 |
| ATOM | 3518 | CA | ARG | B | 866 | 10.615 | 0.127 | −43.377 | 1.00 | 74.69 |
| ATOM | 3519 | C | ARG | B | 866 | 10.806 | 0.377 | −44.866 | 1.00 | 72.85 |
| ATOM | 3520 | O | ARG | B | 866 | 10.788 | −0.555 | −45.672 | 1.00 | 72.17 |
| ATOM | 3521 | CB | ARG | B | 866 | 9.150 | 0.355 | −43.010 | 1.00 | 77.16 |
| ATOM | 3522 | CG | ARG | B | 866 | 8.192 | −0.678 | −43.579 | 1.00 | 80.89 |
| ATOM | 3523 | CD | ARG | B | 866 | 6.766 | −0.351 | −43.162 | 1.00 | 84.67 |
| ATOM | 3524 | NE | ARG | B | 866 | 5.889 | −1.518 | −43.176 | 1.00 | 87.14 |
| ATOM | 3525 | CZ | ARG | B | 866 | 4.665 | −1.535 | −42.656 | 1.00 | 89.52 |
| ATOM | 3526 | NH1 | ARG | B | 866 | 4.169 | −0.441 | −42.081 | 1.00 | 89.56 |
| ATOM | 3527 | NH2 | ARG | B | 866 | 3.942 | −2.648 | −42.698 | 1.00 | 90.37 |
| ATOM | 3528 | N | LEU | B | 867 | 10.999 | 1.639 | −45.229 | 1.00 | 69.43 |
| ATOM | 3529 | CA | LEU | B | 867 | 11.173 | 1.993 | −46.630 | 1.00 | 68.73 |
| ATOM | 3530 | C | LEU | B | 867 | 12.416 | 1.394 | −47.275 | 1.00 | 68.30 |
| ATOM | 3531 | O | LEU | B | 867 | 12.376 | 0.980 | −48.433 | 1.00 | 68.60 |
| ATOM | 3532 | CB | LEU | B | 867 | 11.206 | 3.513 | −46.801 | 1.00 | 67.15 |
| ATOM | 3533 | CG | LEU | B | 867 | 11.287 | 3.964 | −48.258 | 1.00 | 63.74 |
| ATOM | 3534 | CD1 | LEU | B | 867 | 10.063 | 3.458 | −49.008 | 1.00 | 62.81 |
| ATOM | 3535 | CD2 | LEU | B | 867 | 11.384 | 5.474 | −48.331 | 1.00 | 61.46 |
| ATOM | 3536 | N | LEU | B | 868 | 13.516 | 1.348 | −46.532 | 1.00 | 67.70 |
| ATOM | 3537 | CA | LEU | B | 868 | 14.759 | 0.810 | −47.066 | 1.00 | 67.56 |
| ATOM | 3538 | C | LEU | B | 868 | 14.851 | −0.703 | −46.951 | 1.00 | 66.73 |
| ATOM | 3539 | O | LEU | B | 868 | 15.386 | −1.361 | −47.841 | 1.00 | 65.40 |
| ATOM | 3540 | CB | LEU | B | 868 | 15.963 | 1.446 | −46.365 | 1.00 | 69.33 |
| ATOM | 3541 | CG | LEU | B | 868 | 15.984 | 2.976 | −46.258 | 1.00 | 71.54 |
| ATOM | 3542 | CD1 | LEU | B | 868 | 17.337 | 3.418 | −45.705 | 1.00 | 71.31 |
| ATOM | 3543 | CD2 | LEU | B | 868 | 15.719 | 3.613 | −47.617 | 1.00 | 70.98 |
| ATOM | 3544 | N | GLU | B | 869 | 14.329 | −1.263 | −45.865 | 1.00 | 66.47 |
| ATOM | 3545 | CA | GLU | B | 869 | 14.393 | −2.705 | −45.692 | 1.00 | 66.24 |
| ATOM | 3546 | C | GLU | B | 869 | 13.191 | −3.433 | −46.271 | 1.00 | 65.56 |
| ATOM | 3547 | O | GLU | B | 869 | 13.276 | −4.020 | −47.345 | 1.00 | 66.35 |
| ATOM | 3548 | CB | GLU | B | 869 | 14.547 | −3.055 | −44.220 | 1.00 | 67.33 |
| ATOM | 3549 | CG | GLU | B | 869 | 15.784 | −2.462 | −43.592 | 1.00 | 69.61 |
| ATOM | 3550 | CD | GLU | B | 869 | 16.423 | −3.390 | −42.579 | 1.00 | 71.71 |
| ATOM | 3551 | OE1 | GLU | B | 869 | 15.728 | −3.811 | −41.620 | 1.00 | 70.66 |
| ATOM | 3552 | OE2 | GLU | B | 869 | 17.625 | −3.697 | −42.752 | 1.00 | 72.27 |
| ATOM | 3553 | N | GLY | B | 870 | 12.073 | −3.396 | −45.563 | 1.00 | 64.97 |
| ATOM | 3554 | CA | GLY | B | 870 | 10.888 | −4.076 | −46.041 | 1.00 | 64.73 |
| ATOM | 3555 | C | GLY | B | 870 | 10.238 | −4.819 | −44.900 | 1.00 | 65.26 |
| ATOM | 3556 | O | GLY | B | 870 | 10.555 | −4.567 | −43.744 | 1.00 | 64.41 |
| ATOM | 3557 | N | ASP | B | 871 | 9.344 | −5.748 | −45.216 | 1.00 | 66.68 |
| ATOM | 3558 | CA | ASP | B | 871 | 8.647 | −6.501 | −44.182 | 1.00 | 67.47 |
| ATOM | 3559 | C | ASP | B | 871 | 8.975 | −7.982 | −44.162 | 1.00 | 66.00 |
| ATOM | 3560 | O | ASP | B | 871 | 8.345 | −8.747 | −43.434 | 1.00 | 65.97 |
| ATOM | 3561 | CB | ASP | B | 871 | 7.137 | −6.327 | −44.351 | 1.00 | 72.20 |
| ATOM | 3562 | CG | ASP | B | 871 | 6.676 | −4.927 | −44.013 | 1.00 | 76.99 |
| ATOM | 3563 | OD1 | ASP | B | 871 | 6.783 | −4.551 | −42.824 | 1.00 | 80.07 |
| ATOM | 3564 | OD2 | ASP | B | 871 | 6.216 | −4.204 | −44.930 | 1.00 | 78.50 |
| ATOM | 3565 | N | GLU | B | 872 | 9.961 | −8.389 | −44.951 | 1.00 | 64.83 |
| ATOM | 3566 | CA | GLU | B | 872 | 10.333 | −9.794 | −45.018 | 1.00 | 63.94 |
| ATOM | 3567 | C | GLU | B | 872 | 10.666 | −10.338 | −43.631 | 1.00 | 61.57 |
| ATOM | 3568 | O | GLU | B | 872 | 11.580 | −9.857 | −42.963 | 1.00 | 60.56 |
| ATOM | 3569 | CB | GLU | B | 872 | 11.522 | −9.969 | −45.961 | 1.00 | 66.69 |
| ATOM | 3570 | CG | GLU | B | 872 | 11.932 | −11.418 | −46.172 | 1.00 | 71.91 |
| ATOM | 3571 | CD | GLU | B | 872 | 13.047 | −11.569 | −47.202 | 1.00 | 76.05 |
| ATOM | 3572 | OE1 | GLU | B | 872 | 13.408 | −10.553 | −47.842 | 1.00 | 76.98 |
| ATOM | 3573 | OE2 | GLU | B | 872 | 13.557 | −12.702 | −47.378 | 1.00 | 77.59 |
| ATOM | 3574 | N | ALA | B | 873 | 9.914 | −11.342 | −43.202 | 1.00 | 59.27 |
| ATOM | 3575 | CA | ALA | B | 873 | 10.124 | −11.935 | −41.894 | 1.00 | 57.77 |
| ATOM | 3576 | C | ALA | B | 873 | 11.549 | −12.447 | −41.719 | 1.00 | 58.33 |
| ATOM | 3577 | O | ALA | B | 873 | 12.171 | −12.267 | −40.668 | 1.00 | 56.59 |
| ATOM | 3578 | CB | ALA | B | 873 | 9.133 | −13.070 | −41.679 | 1.00 | 55.37 |
| ATOM | 3579 | N | ALA | B | 874 | 12.076 | −13.086 | −42.755 | 1.00 | 59.61 |
| ATOM | 3580 | CA | ALA | B | 874 | 13.418 | −13.640 | −42.666 | 1.00 | 60.12 |
| ATOM | 3581 | C | ALA | B | 874 | 14.500 | −12.681 | −43.134 | 1.00 | 60.09 |
| ATOM | 3582 | O | ALA | B | 874 | 14.395 | −12.075 | −44.203 | 1.00 | 60.48 |
| ATOM | 3583 | CB | ALA | B | 874 | 13.494 | −14.949 | −43.456 | 1.00 | 59.26 |
| ATOM | 3584 | N | TYR | B | 875 | 15.538 | −12.542 | −42.316 | 1.00 | 59.00 |
| ATOM | 3585 | CA | TYR | B | 875 | 16.667 | −11.688 | −42.655 | 1.00 | 58.29 |
| ATOM | 3586 | C | TYR | B | 875 | 17.575 | −12.464 | −43.595 | 1.00 | 58.10 |
| ATOM | 3587 | O | TYR | B | 875 | 17.824 | −13.649 | −43.388 | 1.00 | 57.86 |
| ATOM | 3588 | CB | TYR | B | 875 | 17.487 | −11.336 | −41.414 | 1.00 | 58.23 |
| ATOM | 3589 | CG | TYR | B | 875 | 16.842 | −10.360 | −40.466 | 1.00 | 58.80 |
| ATOM | 3590 | CD1 | TYR | B | 875 | 16.632 | −9.033 | −40.842 | 1.00 | 57.46 |
| ATOM | 3591 | CD2 | TYR | B | 875 | 16.456 | −10.759 | −39.185 | 1.00 | 56.30 |
| ATOM | 3592 | CE1 | TYR | B | 875 | 16.056 | −8.126 | −39.970 | 1.00 | 58.87 |
| ATOM | 3593 | CE2 | TYR | B | 875 | 15.879 | −9.862 | −38.303 | 1.00 | 59.49 |
| ATOM | 3594 | CZ | TYR | B | 875 | 15.680 | −8.543 | −38.701 | 1.00 | 60.51 |
| ATOM | 3595 | OH | TYR | B | 875 | 15.102 | −7.645 | −37.834 | 1.00 | 60.20 |
| ATOM | 3596 | N | ASN | B | 876 | 18.060 | −11.802 | −44.634 | 1.00 | 58.32 |
| ATOM | 3597 | CA | ASN | B | 876 | 18.984 | −12.440 | −45.550 | 1.00 | 58.92 |
| ATOM | 3598 | C | ASN | B | 876 | 20.368 | −12.237 | −44.912 | 1.00 | 59.09 |
| ATOM | 3599 | O | ASN | B | 876 | 20.457 | −11.766 | −43.775 | 1.00 | 57.47 |
| ATOM | 3600 | CB | ASN | B | 876 | 18.879 | −11.805 | −46.943 | 1.00 | 59.97 |
| ATOM | 3601 | CG | ASN | B | 876 | 18.810 | −10.286 | −46.903 | 1.00 | 65.32 |
| ATOM | 3602 | OD1 | ASN | B | 876 | 18.123 | −9.696 | −46.055 | 1.00 | 69.32 |
| ATOM | 3603 | ND2 | ASN | B | 876 | 19.505 | −9.638 | −47.836 | 1.00 | 65.71 |
| ATOM | 3604 | N | ALA | B | 877 | 21.435 | −12.596 | −45.617 | 1.00 | 59.32 |
| ATOM | 3605 | CA | ALA | B | 877 | 22.787 | −12.466 | −45.077 | 1.00 | 59.77 |
| ATOM | 3606 | C | ALA | B | 877 | 23.238 | −11.011 | −44.883 | 1.00 | 61.45 |
| ATOM | 3607 | O | ALA | B | 877 | 24.270 | −10.746 | −44.245 | 1.00 | 59.71 |
| ATOM | 3608 | CB | ALA | B | 877 | 23.765 | −13.201 | −45.983 | 1.00 | 60.47 |
| ATOM | 3609 | N | ASP | B | 878 | 22.459 | −10.079 | −45.434 | 1.00 | 62.48 |
| ATOM | 3610 | CA | ASP | B | 878 | 22.753 | −8.654 | −45.334 | 1.00 | 62.92 |
| ATOM | 3611 | C | ASP | B | 878 | 22.260 | −8.132 | −43.987 | 1.00 | 63.34 |
| ATOM | 3612 | O | ASP | B | 878 | 22.660 | −7.055 | −43.548 | 1.00 | 63.62 |
| ATOM | 3613 | CB | ASP | B | 878 | 22.067 | −7.894 | −46.479 | 1.00 | 62.83 |
| ATOM | 3614 | CG | ASP | B | 878 | 22.688 | −6.519 | −46.737 | 1.00 | 64.73 |
| ATOM | 3615 | OD1 | ASP | B | 878 | 23.921 | −6.442 | −46.947 | 1.00 | 63.55 |
| ATOM | 3616 | OD2 | ASP | B | 878 | 21.938 | −5.516 | −46.745 | 1.00 | 64.59 |
| ATOM | 3617 | N | GLY | B | 879 | 21.386 | −8.902 | −43.340 | 1.00 | 63.87 |
| ATOM | 3618 | CA | GLY | B | 879 | 20.847 | −8.516 | −42.042 | 1.00 | 65.56 |
| ATOM | 3619 | C | GLY | B | 879 | 19.955 | −7.281 | −42.027 | 1.00 | 65.90 |
| ATOM | 3620 | O | GLY | B | 879 | 19.336 | −6.933 | −43.037 | 1.00 | 66.17 |
| ATOM | 3621 | N | GLY | B | 880 | 19.885 | −6.621 | −40.873 | 1.00 | 65.12 |
| ATOM | 3622 | CA | GLY | B | 880 | 19.074 | −5.422 | −40.754 | 1.00 | 64.82 |
| ATOM | 3623 | C | GLY | B | 880 | 19.899 | −4.157 | −40.938 | 1.00 | 64.56 |
| ATOM | 3624 | O | GLY | B | 880 | 21.107 | −4.229 | −41.141 | 1.00 | 64.99 |
| ATOM | 3625 | N | ALA | B | 881 | 19.259 | −2.995 | −40.871 | 1.00 | 63.83 |
| ATOM | 3626 | CA | ALA | B | 881 | 19.978 | −1.736 | −41.025 | 1.00 | 63.13 |
| ATOM | 3627 | C | ALA | B | 881 | 20.131 | −1.016 | −39.686 | 1.00 | 62.11 |
| ATOM | 3628 | O | ALA | B | 881 | 21.023 | −0.188 | −39.522 | 1.00 | 61.84 |
| ATOM | 3629 | CB | ALA | B | 881 | 19.261 | −0.835 | −42.033 | 1.00 | 63.27 |
| ATOM | 3630 | N | MET | B | 882 | 19.270 | −1.334 | −38.726 | 1.00 | 60.73 |
| ATOM | 3631 | CA | MET | B | 882 | 19.346 | −0.692 | −37.422 | 1.00 | 60.66 |
| ATOM | 3632 | C | MET | B | 882 | 20.388 | −1.349 | −36.543 | 1.00 | 57.66 |
| ATOM | 3633 | O | MET | B | 882 | 20.738 | −2.505 | −36.742 | 1.00 | 58.53 |
| ATOM | 3634 | CB | MET | B | 882 | 18.006 | −0.759 | −36.692 | 1.00 | 64.90 |
| ATOM | 3635 | CG | MET | B | 882 | 16.844 | −0.168 | −37.442 | 1.00 | 71.12 |
| ATOM | 3636 | SD | MET | B | 882 | 16.275 | −1.292 | −38.702 | 1.00 | 79.62 |
| ATOM | 3637 | CE | MET | B | 882 | 16.473 | −0.256 | −40.127 | 1.00 | 78.23 |
| ATOM | 3638 | N | PRO | B | 883 | 20.906 | −0.610 | −35.558 | 1.00 | 54.95 |
| ATOM | 3639 | CA | PRO | B | 883 | 21.910 | −1.186 | −34.666 | 1.00 | 54.93 |
| ATOM | 3640 | C | PRO | B | 883 | 21.262 | −2.267 | −33.804 | 1.00 | 54.49 |
| ATOM | 3641 | O | PRO | B | 883 | 20.201 | −2.054 | −33.239 | 1.00 | 55.33 |
| ATOM | 3642 | CB | PRO | B | 883 | 22.392 | 0.022 | −33.869 | 1.00 | 54.23 |
| ATOM | 3643 | CG | PRO | B | 883 | 21.206 | 0.944 | −33.882 | 1.00 | 55.31 |
| ATOM | 3644 | CD | PRO | B | 883 | 20.719 | 0.826 | −35.291 | 1.00 | 53.61 |
| ATOM | 3645 | N | ILE | B | 884 | 21.904 | −3.427 | −33.710 | 1.00 | 55.18 |
| ATOM | 3646 | CA | ILE | B | 884 | 21.363 | −4.549 | −32.949 | 1.00 | 54.75 |
| ATOM | 3647 | C | ILE | B | 884 | 21.109 | −4.230 | −31.481 | 1.00 | 53.77 |
| ATOM | 3648 | O | ILE | B | 884 | 20.193 | −4.778 | −30.871 | 1.00 | 52.49 |
| ATOM | 3649 | CB | ILE | B | 884 | 22.315 | −5.786 | −32.997 | 1.00 | 57.45 |
| ATOM | 3650 | CG1 | ILE | B | 884 | 22.977 | −5.911 | −34.379 | 1.00 | 60.74 |
| ATOM | 3651 | CG2 | ILE | B | 884 | 21.522 | −7.070 | −32.704 | 1.00 | 56.49 |
| ATOM | 3652 | CD1 | ILE | B | 884 | 22.026 | −6.282 | −35.528 | 1.00 | 61.29 |
| ATOM | 3653 | N | LYS | B | 885 | 21.912 | −3.333 | −30.919 | 1.00 | 53.13 |
| ATOM | 3654 | CA | LYS | B | 885 | 21.809 | −3.004 | −29.499 | 1.00 | 51.92 |
| ATOM | 3655 | C | LYS | B | 885 | 20.604 | −2.183 | −29.040 | 1.00 | 50.73 |
| ATOM | 3656 | O | LYS | B | 885 | 20.309 | −2.099 | −27.850 | 1.00 | 50.67 |
| ATOM | 3657 | CB | LYS | B | 885 | 23.118 | −2.350 | −29.052 | 1.00 | 51.96 |
| ATOM | 3658 | CG | LYS | B | 885 | 24.316 | −3.277 | −29.214 | 1.00 | 49.00 |
| ATOM | 3659 | CD | LYS | B | 885 | 25.597 | −2.616 | −28.766 | 1.00 | 52.24 |
| ATOM | 3660 | CE | LYS | B | 885 | 26.798 | −3.482 | −29.122 | 1.00 | 53.83 |
| ATOM | 3661 | NZ | LYS | B | 885 | 28.091 | −2.784 | −28.862 | 1.00 | 56.01 |
| ATOM | 3662 | N | TRP | B | 886 | 19.900 | −1.588 | −29.985 | 1.00 | 48.96 |
| ATOM | 3663 | CA | TRP | B | 886 | 18.728 | −0.797 | −29.667 | 1.00 | 47.71 |
| ATOM | 3664 | C | TRP | B | 886 | 17.494 | −1.600 | −30.055 | 1.00 | 48.21 |
| ATOM | 3665 | O | TRP | B | 886 | 16.363 | −1.142 | −29.899 | 1.00 | 49.83 |
| ATOM | 3666 | CB | TRP | B | 886 | 18.765 | 0.501 | −30.470 | 1.00 | 46.30 |
| ATOM | 3667 | CG | TRP | B | 886 | 19.594 | 1.587 | −29.863 | 1.00 | 46.96 |
| ATOM | 3668 | CD1 | TRP | B | 886 | 19.138 | 2.657 | −29.145 | 1.00 | 45.46 |
| ATOM | 3669 | CD2 | TRP | B | 886 | 21.014 | 1.750 | −29.965 | 1.00 | 47.38 |
| ATOM | 3670 | NE1 | TRP | B | 886 | 20.182 | 3.480 | −28.802 | 1.00 | 45.52 |
| ATOM | 3671 | CE2 | TRP | B | 886 | 21.346 | 2.950 | −29.293 | 1.00 | 46.37 |
| ATOM | 3672 | CE3 | TRP | B | 886 | 22.039 | 1.003 | −30.560 | 1.00 | 48.54 |
| ATOM | 3673 | CZ2 | TRP | B | 886 | 22.661 | 3.422 | −29.202 | 1.00 | 44.45 |
| ATOM | 3674 | CZ3 | TRP | B | 886 | 23.354 | 1.479 | −30.468 | 1.00 | 48.55 |
| ATOM | 3675 | CH2 | TRP | B | 886 | 23.648 | 2.678 | −29.793 | 1.00 | 43.97 |
| ATOM | 3676 | N | MET | B | 887 | 17.731 | −2.814 | −30.536 | 1.00 | 47.39 |
| ATOM | 3677 | CA | MET | B | 887 | 16.689 | −3.702 | −31.038 | 1.00 | 47.29 |
| ATOM | 3678 | C | MET | B | 887 | 16.042 | −4.632 | −30.021 | 1.00 | 46.15 |
| ATOM | 3679 | O | MET | B | 887 | 16.716 | −5.188 | −29.170 | 1.00 | 44.83 |
| ATOM | 3680 | CB | MET | B | 887 | 17.299 | −4.525 | −32.182 | 1.00 | 49.51 |
| ATOM | 3681 | CG | MET | B | 887 | 16.366 | −4.953 | −33.287 | 1.00 | 52.27 |
| ATOM | 3682 | SD | MET | B | 887 | 17.296 | −5.341 | −34.809 | 1.00 | 53.59 |
| ATOM | 3683 | CE | MET | B | 887 | 17.031 | −3.843 | −35.775 | 1.00 | 56.07 |
| ATOM | 3684 | N | ALA | B | 888 | 14.723 | −4.785 | −30.119 | 1.00 | 48.05 |
| ATOM | 3685 | CA | ALA | B | 888 | 13.962 | −5.683 | −29.240 | 1.00 | 48.80 |
| ATOM | 3686 | C | ALA | B | 888 | 14.288 | −7.110 | −29.661 | 1.00 | 49.42 |
| ATOM | 3687 | O | ALA | B | 888 | 14.361 | −7.406 | −30.856 | 1.00 | 49.12 |
| ATOM | 3688 | CB | ALA | B | 888 | 12.460 | −5.432 | −29.386 | 1.00 | 47.59 |
| ATOM | 3689 | N | LEU | B | 889 | 14.472 | −7.988 | −28.681 | 1.00 | 50.17 |
| ATOM | 3690 | CA | LEU | B | 889 | 14.829 | −9.376 | −28.941 | 1.00 | 51.55 |
| ATOM | 3691 | C | LEU | B | 889 | 14.145 | −10.030 | −30.124 | 1.00 | 53.37 |
| ATOM | 3692 | O | LEU | B | 889 | 14.806 | −10.463 | −31.076 | 1.00 | 54.40 |
| ATOM | 3693 | CB | LEU | B | 889 | 14.570 | −10.230 | −27.713 | 1.00 | 52.02 |
| ATOM | 3694 | CG | LEU | B | 889 | 14.987 | −11.690 | −27.885 | 1.00 | 53.15 |
| ATOM | 3695 | CD1 | LEU | B | 889 | 16.443 | −11.748 | −28.320 | 1.00 | 52.50 |
| ATOM | 3696 | CD2 | LEU | B | 889 | 14.777 | −12.445 | −26.571 | 1.00 | 52.22 |
| ATOM | 3697 | N | GLU | B | 890 | 12.822 | −10.114 | −30.067 | 1.00 | 53.20 |
| ATOM | 3698 | CA | GLU | B | 890 | 12.079 | −10.745 | −31.146 | 1.00 | 54.27 |
| ATOM | 3699 | C | GLU | B | 890 | 12.498 | −10.227 | −32.521 | 1.00 | 55.82 |
| ATOM | 3700 | O | GLU | B | 890 | 12.562 | −10.994 | −33.480 | 1.00 | 57.28 |
| ATOM | 3701 | CB | GLU | B | 890 | 10.566 | −10.557 | −30.955 | 1.00 | 52.93 |
| ATOM | 3702 | CG | GLU | B | 890 | 10.094 | −9.116 | −31.001 | 1.00 | 50.27 |
| ATOM | 3703 | CD | GLU | B | 890 | 10.080 | −8.445 | −29.634 | 1.00 | 48.34 |
| ATOM | 3704 | OE1 | GLU | B | 890 | 10.893 | −8.842 | −28.759 | 1.00 | 44.27 |
| ATOM | 3705 | OE2 | GLU | B | 890 | 9.260 | −7.509 | −29.453 | 1.00 | 42.51 |
| ATOM | 3706 | N | CYS | B | 891 | 12.798 | −8.935 | −32.613 | 1.00 | 57.27 |
| ATOM | 3707 | CA | CYS | B | 891 | 13.192 | −8.336 | −33.888 | 1.00 | 58.36 |
| ATOM | 3708 | C | CYS | B | 891 | 14.617 | −8.642 | −34.313 | 1.00 | 58.40 |
| ATOM | 3709 | O | CYS | B | 891 | 15.052 | −8.274 | −35.401 | 1.00 | 56.71 |
| ATOM | 3710 | CB | CYS | B | 891 | 12.972 | −6.826 | −33.852 | 1.00 | 59.30 |
| ATOM | 3711 | SG | CYS | B | 891 | 11.232 | −6.370 | −33.986 | 1.00 | 58.60 |
| ATOM | 3712 | N | ILE | B | 892 | 15.352 | −9.324 | −33.457 | 1.00 | 59.69 |
| ATOM | 3713 | CA | ILE | B | 892 | 16.708 | −9.677 | −33.812 | 1.00 | 60.78 |
| ATOM | 3714 | C | ILE | B | 892 | 16.692 | −10.959 | −34.647 | 1.00 | 60.92 |
| ATOM | 3715 | O | ILE | B | 892 | 17.492 | −11.104 | −35.556 | 1.00 | 61.20 |
| ATOM | 3716 | CB | ILE | B | 892 | 17.584 | −9.872 | −32.549 | 1.00 | 60.34 |
| ATOM | 3717 | CG1 | ILE | B | 892 | 17.844 | −8.516 | −31.898 | 1.00 | 59.88 |
| ATOM | 3718 | CG2 | ILE | B | 892 | 18.892 | −10.551 | −32.910 | 1.00 | 58.41 |
| ATOM | 3719 | CD1 | ILE | B | 892 | 18.713 | −8.585 | −30.683 | 1.00 | 61.44 |
| ATOM | 3720 | N | HIS | B | 893 | 15.766 | −11.873 | −34.350 | 1.00 | 61.38 |
| ATOM | 3721 | CA | HIS | B | 893 | 15.676 | −13.143 | −35.071 | 1.00 | 60.14 |
| ATOM | 3722 | C | HIS | B | 893 | 14.811 | −13.131 | −36.329 | 1.00 | 57.92 |
| ATOM | 3723 | O | HIS | B | 893 | 15.010 | −13.940 | −37.232 | 1.00 | 56.94 |
| ATOM | 3724 | CB | HIS | B | 893 | 15.195 | −14.245 | −34.127 | 1.00 | 61.71 |
| ATOM | 3725 | CG | HIS | B | 893 | 16.205 | −14.626 | −33.092 | 1.00 | 65.26 |
| ATOM | 3726 | ND1 | HIS | B | 893 | 17.326 | −15.375 | −33.384 | 1.00 | 66.05 |
| ATOM | 3727 | CD2 | HIS | B | 893 | 16.239 | −14.400 | −31.756 | 1.00 | 65.76 |
| ATOM | 3728 | CE1 | HIS | B | 893 | 18.006 | −15.596 | −32.272 | 1.00 | 66.79 |
| ATOM | 3729 | NE2 | HIS | B | 893 | 17.368 | −15.016 | −31.270 | 1.00 | 67.66 |
| ATOM | 3730 | N | TYR | B | 894 | 13.850 | −12.218 | −36.379 | 1.00 | 55.44 |
| ATOM | 3731 | CA | TYR | B | 894 | 12.956 | −12.082 | −37.523 | 1.00 | 53.67 |
| ATOM | 3732 | C | TYR | B | 894 | 12.491 | −10.649 | −37.482 | 1.00 | 52.79 |
| ATOM | 3733 | O | TYR | B | 894 | 12.909 | −9.893 | −36.620 | 1.00 | 53.67 |
| ATOM | 3734 | CB | TYR | B | 894 | 11.738 | −13.000 | −37.387 | 1.00 | 53.49 |
| ATOM | 3735 | CG | TYR | B | 894 | 12.051 | −14.469 | −37.473 | 1.00 | 54.03 |
| ATOM | 3736 | CD1 | TYR | B | 894 | 12.231 | −15.091 | −38.706 | 1.00 | 54.75 |
| ATOM | 3737 | CD2 | TYR | B | 894 | 12.223 | −15.228 | −36.318 | 1.00 | 55.06 |
| ATOM | 3738 | CE1 | TYR | B | 894 | 12.580 | −16.438 | −38.784 | 1.00 | 57.69 |
| ATOM | 3739 | CE2 | TYR | B | 894 | 12.574 | −16.571 | −36.381 | 1.00 | 57.19 |
| ATOM | 3740 | CZ | TYR | B | 894 | 12.755 | −17.175 | −37.616 | 1.00 | 59.22 |
| ATOM | 3741 | OH | TYR | B | 894 | 13.136 | −18.504 | −37.679 | 1.00 | 61.11 |
| ATOM | 3742 | N | ARG | B | 895 | 11.623 | −10.269 | −38.405 | 1.00 | 53.26 |
| ATOM | 3743 | CA | ARG | B | 895 | 11.112 | −8.912 | −38.417 | 1.00 | 53.71 |
| ATOM | 3744 | C | ARG | B | 895 | 9.726 | −8.838 | −37.788 | 1.00 | 55.40 |
| ATOM | 3745 | O | ARG | B | 895 | 8.700 | −8.972 | −38.464 | 1.00 | 56.92 |
| ATOM | 3746 | CB | ARG | B | 895 | 11.066 | −8.363 | −39.840 | 1.00 | 52.60 |
| ATOM | 3747 | CG | ARG | B | 895 | 12.425 | −8.042 | −40.416 | 1.00 | 54.61 |
| ATOM | 3748 | CD | ARG | B | 895 | 12.326 | −6.918 | −41.439 | 1.00 | 56.13 |
| ATOM | 3749 | NE | ARG | B | 895 | 13.641 | −6.505 | −41.914 | 1.00 | 58.90 |
| ATOM | 3750 | CZ | ARG | B | 895 | 14.368 | −7.200 | −42.781 | 1.00 | 61.14 |
| ATOM | 3751 | NH1 | ARG | B | 895 | 13.894 | −8.343 | −43.271 | 1.00 | 61.94 |
| ATOM | 3752 | NH2 | ARG | B | 895 | 15.571 | −6.767 | −43.144 | 1.00 | 58.94 |
| ATOM | 3753 | N | ALA | B | 896 | 9.705 | −8.634 | −36.476 | 1.00 | 55.24 |
| ATOM | 3754 | CA | ALA | B | 896 | 8.452 | −8.519 | −35.748 | 1.00 | 53.33 |
| ATOM | 3755 | C | ALA | B | 896 | 8.350 | −7.085 | −35.215 | 1.00 | 51.36 |
| ATOM | 3756 | O | ALA | B | 896 | 8.063 | −6.872 | −34.040 | 1.00 | 51.66 |
| ATOM | 3757 | CB | ALA | B | 896 | 8.419 | −9.536 | −34.598 | 1.00 | 52.72 |
| ATOM | 3758 | N | PHE | B | 897 | 8.592 | −6.104 | −36.086 | 1.00 | 48.95 |
| ATOM | 3759 | CA | PHE | B | 897 | 8.535 | −4.695 | −35.683 | 1.00 | 46.93 |
| ATOM | 3760 | C | PHE | B | 897 | 7.134 | −4.237 | −35.300 | 1.00 | 44.17 |
| ATOM | 3761 | O | PHE | B | 897 | 6.209 | −4.266 | −36.106 | 1.00 | 42.11 |
| ATOM | 3762 | CB | PHE | B | 897 | 9.093 | −3.785 | −36.787 | 1.00 | 47.63 |
| ATOM | 3763 | CG | PHE | B | 897 | 10.572 | −3.928 | −36.993 | 1.00 | 50.51 |
| ATOM | 3764 | CD1 | PHE | B | 897 | 11.076 | −4.474 | −38.172 | 1.00 | 53.25 |
| ATOM | 3765 | CD2 | PHE | B | 897 | 11.465 | −3.567 | −35.988 | 1.00 | 50.87 |
| ATOM | 3766 | CE1 | PHE | B | 897 | 12.451 | −4.662 | −38.342 | 1.00 | 52.04 |
| ATOM | 3767 | CE2 | PHE | B | 897 | 12.835 | −3.750 | −36.148 | 1.00 | 50.84 |
| ATOM | 3768 | CZ | PHE | B | 897 | 13.329 | −4.300 | −37.326 | 1.00 | 52.35 |
| ATOM | 3769 | N | THR | B | 898 | 6.997 | −3.798 | −34.055 | 1.00 | 42.52 |
| ATOM | 3770 | CA | THR | B | 898 | 5.717 | −3.352 | −33.534 | 1.00 | 40.56 |
| ATOM | 3771 | C | THR | B | 898 | 5.916 | −2.231 | −32.543 | 1.00 | 39.82 |
| ATOM | 3772 | O | THR | B | 898 | 7.027 | −1.983 | −32.089 | 1.00 | 38.49 |
| ATOM | 3773 | CB | THR | B | 898 | 5.000 | −4.480 | −32.779 | 1.00 | 39.38 |
| ATOM | 3774 | OG1 | THR | B | 898 | 5.744 | −4.803 | −31.602 | 1.00 | 39.33 |
| ATOM | 3775 | CG2 | THR | B | 898 | 4.881 | −5.711 | −33.638 | 1.00 | 37.90 |
| ATOM | 3776 | N | HIS | B | 899 | 4.826 | −1.558 | −32.198 | 1.00 | 39.68 |
| ATOM | 3777 | CA | HIS | B | 899 | 4.907 | −0.487 | −31.225 | 1.00 | 40.57 |
| ATOM | 3778 | C | HIS | B | 899 | 5.672 | −0.985 | −29.998 | 1.00 | 39.75 |
| ATOM | 3779 | O | HIS | B | 899 | 6.494 | −0.262 | −29.433 | 1.00 | 40.90 |
| ATOM | 3780 | CB | HIS | B | 899 | 3.499 | −0.040 | −30.838 | 1.00 | 41.77 |
| ATOM | 3781 | CG | HIS | B | 899 | 2.711 | 0.476 | −31.994 | 1.00 | 44.38 |
| ATOM | 3782 | ND1 | HIS | B | 899 | 3.136 | 1.539 | −32.765 | 1.00 | 46.58 |
| ATOM | 3783 | CD2 | HIS | B | 899 | 1.560 | 0.042 | −32.554 | 1.00 | 44.66 |
| ATOM | 3784 | CE1 | HIS | B | 899 | 2.283 | 1.734 | −33.752 | 1.00 | 45.15 |
| ATOM | 3785 | NE2 | HIS | B | 899 | 1.317 | 0.838 | −33.648 | 1.00 | 47.56 |
| ATOM | 3786 | N | GLN | B | 900 | 5.420 | −2.227 | −29.604 | 1.00 | 37.28 |
| ATOM | 3787 | CA | GLN | B | 900 | 6.100 | −2.770 | −28.452 | 1.00 | 38.27 |
| ATOM | 3788 | C | GLN | B | 900 | 7.608 | −2.902 | −28.658 | 1.00 | 38.64 |
| ATOM | 3789 | O | GLN | B | 900 | 8.374 | −2.701 | −27.717 | 1.00 | 39.27 |
| ATOM | 3790 | CB | GLN | B | 900 | 5.492 | −4.112 | −28.045 | 1.00 | 38.52 |
| ATOM | 3791 | CG | GLN | B | 900 | 4.165 | −3.987 | −27.302 | 1.00 | 39.82 |
| ATOM | 3792 | CD | GLN | B | 900 | 4.050 | −2.696 | −26.472 | 1.00 | 43.54 |
| ATOM | 3793 | OE1 | GLN | B | 900 | 3.859 | −1.614 | −27.025 | 1.00 | 46.46 |
| ATOM | 3794 | NE2 | GLN | B | 900 | 4.161 | −2.812 | −25.145 | 1.00 | 44.27 |
| ATOM | 3795 | N | SER | B | 901 | 8.048 | −3.226 | −29.872 | 1.00 | 36.36 |
| ATOM | 3796 | CA | SER | B | 901 | 9.482 | −3.340 | −30.110 | 1.00 | 35.93 |
| ATOM | 3797 | C | SER | B | 901 | 10.093 | −1.938 | −30.014 | 1.00 | 37.71 |
| ATOM | 3798 | O | SER | B | 901 | 11.239 | −1.759 | −29.584 | 1.00 | 35.11 |
| ATOM | 3799 | CB | SER | B | 901 | 9.773 | −3.993 | −31.472 | 1.00 | 35.40 |
| ATOM | 3800 | OG | SER | B | 901 | 9.213 | −3.299 | −32.566 | 1.00 | 35.60 |
| ATOM | 3801 | N | ASP | B | 902 | 9.302 | −0.936 | −30.387 | 1.00 | 36.58 |
| ATOM | 3802 | CA | ASP | B | 902 | 9.753 | 0.436 | −30.297 | 1.00 | 35.05 |
| ATOM | 3803 | C | ASP | B | 902 | 9.918 | 0.797 | −28.816 | 1.00 | 35.23 |
| ATOM | 3804 | O | ASP | B | 902 | 10.766 | 1.618 | −28.453 | 1.00 | 31.03 |
| ATOM | 3805 | CB | ASP | B | 902 | 8.738 | 1.368 | −30.961 | 1.00 | 35.87 |
| ATOM | 3806 | CG | ASP | B | 902 | 9.065 | 1.646 | −32.421 | 1.00 | 38.00 |
| ATOM | 3807 | OD1 | ASP | B | 902 | 10.043 | 1.066 | −32.944 | 1.00 | 35.01 |
| ATOM | 3808 | OD2 | ASP | B | 902 | 8.341 | 2.454 | −33.043 | 1.00 | 40.53 |
| ATOM | 3809 | N | VAL | B | 903 | 9.112 | 0.179 | −27.957 | 1.00 | 34.13 |
| ATOM | 3810 | CA | VAL | B | 903 | 9.214 | 0.479 | −26.538 | 1.00 | 36.81 |
| ATOM | 3811 | C | VAL | B | 903 | 10.559 | 0.008 | −26.010 | 1.00 | 37.63 |
| ATOM | 3812 | O | VAL | B | 903 | 11.184 | 0.674 | −25.180 | 1.00 | 37.43 |
| ATOM | 3813 | CB | VAL | B | 903 | 8.075 | −0.178 | −25.727 | 1.00 | 38.45 |
| ATOM | 3814 | CG1 | VAL | B | 903 | 8.380 | −0.100 | −24.229 | 1.00 | 37.24 |
| ATOM | 3815 | CG2 | VAL | B | 903 | 6.763 | 0.533 | −26.026 | 1.00 | 37.24 |
| ATOM | 3816 | N | TRP | B | 904 | 11.005 | −1.144 | −26.498 | 1.00 | 39.08 |
| ATOM | 3817 | CA | TRP | B | 904 | 12.297 | −1.674 | −26.092 | 1.00 | 37.59 |
| ATOM | 3818 | C | TRP | B | 904 | 13.332 | −0.601 | −26.441 | 1.00 | 34.67 |
| ATOM | 3819 | O | TRP | B | 904 | 14.129 | −0.203 | −25.602 | 1.00 | 32.68 |
| ATOM | 3820 | CB | TRP | B | 904 | 12.600 | −2.980 | −26.848 | 1.00 | 40.25 |
| ATOM | 3821 | CG | TRP | B | 904 | 13.943 | −3.588 | −26.523 | 1.00 | 41.50 |
| ATOM | 3822 | CD1 | TRP | B | 904 | 15.160 | −3.031 | −26.742 | 1.00 | 39.65 |
| ATOM | 3823 | CD2 | TRP | B | 904 | 14.190 | −4.857 | −25.891 | 1.00 | 43.17 |
| ATOM | 3824 | NE1 | TRP | B | 904 | 16.153 | −3.861 | −26.285 | 1.00 | 42.57 |
| ATOM | 3825 | CE2 | TRP | B | 904 | 15.588 | −4.991 | −25.759 | 1.00 | 43.45 |
| ATOM | 3826 | CE3 | TRP | B | 904 | 13.364 | −5.892 | −25.424 | 1.00 | 45.15 |
| ATOM | 3827 | CZ2 | TRP | B | 904 | 16.188 | −6.123 | −25.176 | 1.00 | 45.09 |
| ATOM | 3828 | CZ3 | TRP | B | 904 | 13.962 | −7.024 | −24.839 | 1.00 | 45.21 |
| ATOM | 3829 | CH2 | TRP | B | 904 | 15.360 | −7.125 | −24.724 | 1.00 | 44.37 |
| ATOM | 3830 | N | SER | B | 905 | 13.296 | −0.123 | −27.678 | 1.00 | 32.67 |
| ATOM | 3831 | CA | SER | B | 905 | 14.233 | 0.895 | −28.120 | 1.00 | 34.81 |
| ATOM | 3832 | C | SER | B | 905 | 14.182 | 2.102 | −27.190 | 1.00 | 35.70 |
| ATOM | 3833 | O | SER | B | 905 | 15.214 | 2.596 | −26.727 | 1.00 | 35.81 |
| ATOM | 3834 | CB | SER | B | 905 | 13.904 | 1.309 | −29.548 | 1.00 | 35.88 |
| ATOM | 3835 | OG | SER | B | 905 | 13.939 | 0.183 | −30.404 | 1.00 | 34.89 |
| ATOM | 3836 | N | TYR | B | 906 | 12.963 | 2.556 | −26.920 | 1.00 | 37.42 |
| ATOM | 3837 | CA | TYR | B | 906 | 12.712 | 3.679 | −26.032 | 1.00 | 36.19 |
| ATOM | 3838 | C | TYR | B | 906 | 13.517 | 3.478 | −24.763 | 1.00 | 36.71 |
| ATOM | 3839 | O | TYR | B | 906 | 14.208 | 4.378 | −24.304 | 1.00 | 35.97 |
| ATOM | 3840 | CB | TYR | B | 906 | 11.226 | 3.741 | −25.677 | 1.00 | 36.25 |
| ATOM | 3841 | CG | TYR | B | 906 | 10.889 | 4.865 | −24.733 | 1.00 | 35.80 |
| ATOM | 3842 | CD1 | TYR | B | 906 | 10.571 | 6.130 | −25.212 | 1.00 | 33.13 |
| ATOM | 3843 | CD2 | TYR | B | 906 | 10.983 | 4.683 | −23.362 | 1.00 | 37.44 |
| ATOM | 3844 | CE1 | TYR | B | 906 | 10.369 | 7.183 | −24.360 | 1.00 | 36.10 |
| ATOM | 3845 | CE2 | TYR | B | 906 | 10.782 | 5.738 | −22.489 | 1.00 | 40.97 |
| ATOM | 3846 | CZ | TYR | B | 906 | 10.479 | 6.991 | −22.994 | 1.00 | 39.58 |
| ATOM | 3847 | OH | TYR | B | 906 | 10.328 | 8.050 | −22.125 | 1.00 | 39.67 |
| ATOM | 3848 | N | GLY | B | 907 | 13.409 | 2.289 | −24.189 | 1.00 | 36.90 |
| ATOM | 3849 | CA | GLY | B | 907 | 14.150 | 1.995 | −22.982 | 1.00 | 38.61 |
| ATOM | 3850 | C | GLY | B | 907 | 15.642 | 2.197 | −23.171 | 1.00 | 41.80 |
| ATOM | 3851 | O | GLY | B | 907 | 16.285 | 2.870 | −22.362 | 1.00 | 41.26 |
| ATOM | 3852 | N | VAL | B | 908 | 16.196 | 1.616 | −24.238 | 1.00 | 43.21 |
| ATOM | 3853 | CA | VAL | B | 908 | 17.626 | 1.728 | −24.527 | 1.00 | 42.16 |
| ATOM | 3854 | C | VAL | B | 908 | 17.967 | 3.191 | −24.740 | 1.00 | 42.43 |
| ATOM | 3855 | O | VAL | B | 908 | 19.041 | 3.655 | −24.358 | 1.00 | 42.57 |
| ATOM | 3856 | CB | VAL | B | 908 | 18.013 | 0.928 | −25.794 | 1.00 | 43.20 |
| ATOM | 3857 | CG1 | VAL | B | 908 | 19.510 | 1.037 | −26.052 | 1.00 | 42.14 |
| ATOM | 3858 | CG2 | VAL | B | 908 | 17.626 | −0.525 | −25.622 | 1.00 | 42.76 |
| ATOM | 3859 | N | THR | B | 909 | 17.034 | 3.919 | −25.338 | 1.00 | 40.59 |
| ATOM | 3860 | CA | THR | B | 909 | 17.239 | 5.333 | −25.583 | 1.00 | 40.05 |
| ATOM | 3861 | C | THR | B | 909 | 17.318 | 6.168 | −24.318 | 1.00 | 41.07 |
| ATOM | 3862 | O | THR | B | 909 | 18.227 | 6.971 | −24.180 | 1.00 | 44.18 |
| ATOM | 3863 | CB | THR | B | 909 | 16.133 | 5.925 | −26.447 | 1.00 | 38.19 |
| ATOM | 3864 | OG1 | THR | B | 909 | 16.119 | 5.268 | −27.717 | 1.00 | 37.26 |
| ATOM | 3865 | CG2 | THR | B | 909 | 16.375 | 7.413 | −26.658 | 1.00 | 35.17 |
| ATOM | 3866 | N | ILE | B | 910 | 16.383 | 6.006 | −23.390 | 1.00 | 41.77 |
| ATOM | 3867 | CA | ILE | B | 910 | 16.459 | 6.830 | −22.191 | 1.00 | 44.01 |
| ATOM | 3868 | C | ILE | B | 910 | 17.678 | 6.440 | −21.365 | 1.00 | 43.93 |
| ATOM | 3869 | O | ILE | B | 910 | 18.162 | 7.222 | −20.537 | 1.00 | 43.25 |
| ATOM | 3870 | CB | ILE | B | 910 | 15.171 | 6.768 | −21.323 | 1.00 | 44.35 |
| ATOM | 3871 | CG1 | ILE | B | 910 | 15.014 | 5.394 | −20.688 | 1.00 | 45.86 |
| ATOM | 3872 | CG2 | ILE | B | 910 | 13.960 | 7.146 | −22.179 | 1.00 | 44.14 |
| ATOM | 3873 | CD1 | ILE | B | 910 | 13.834 | 5.285 | −19.766 | 1.00 | 45.31 |
| ATOM | 3874 | N | TRP | B | 911 | 18.179 | 5.233 | −21.611 | 1.00 | 43.13 |
| ATOM | 3875 | CA | TRP | B | 911 | 19.369 | 4.746 | −20.927 | 1.00 | 41.24 |
| ATOM | 3876 | C | TRP | B | 911 | 20.542 | 5.605 | −21.424 | 1.00 | 41.42 |
| ATOM | 3877 | O | TRP | B | 911 | 21.375 | 6.032 | −20.629 | 1.00 | 41.37 |
| ATOM | 3878 | CB | TRP | B | 911 | 19.600 | 3.279 | −21.265 | 1.00 | 42.93 |
| ATOM | 3879 | CG | TRP | B | 911 | 20.768 | 2.671 | −20.582 | 1.00 | 46.13 |
| ATOM | 3880 | CD1 | TRP | B | 911 | 20.778 | 2.044 | −19.370 | 1.00 | 48.07 |
| ATOM | 3881 | CD2 | TRP | B | 911 | 22.120 | 2.647 | −21.058 | 1.00 | 47.88 |
| ATOM | 3882 | NE1 | TRP | B | 911 | 22.053 | 1.629 | −19.059 | 1.00 | 48.23 |
| ATOM | 3883 | CE2 | TRP | B | 911 | 22.897 | 1.988 | −20.078 | 1.00 | 48.28 |
| ATOM | 3884 | CE3 | TRP | B | 911 | 22.751 | 3.123 | −22.216 | 1.00 | 46.20 |
| ATOM | 3885 | CZ2 | TRP | B | 911 | 24.274 | 1.792 | −20.221 | 1.00 | 45.73 |
| ATOM | 3886 | CZ3 | TRP | B | 911 | 24.120 | 2.928 | −22.355 | 1.00 | 46.02 |
| ATOM | 3887 | CH2 | TRP | B | 911 | 24.865 | 2.268 | −21.362 | 1.00 | 45.66 |
| ATOM | 3888 | N | GLU | B | 912 | 20.600 | 5.874 | −22.728 | 1.00 | 40.12 |
| ATOM | 3889 | CA | GLU | B | 912 | 21.673 | 6.703 | −23.263 | 1.00 | 42.28 |
| ATOM | 3890 | C | GLU | B | 912 | 21.616 | 8.051 | −22.573 | 1.00 | 43.95 |
| ATOM | 3891 | O | GLU | B | 912 | 22.649 | 8.627 | −22.228 | 1.00 | 45.55 |
| ATOM | 3892 | CB | GLU | B | 912 | 21.524 | 6.955 | −24.759 | 1.00 | 41.44 |
| ATOM | 3893 | CG | GLU | B | 912 | 21.505 | 5.742 | −25.635 | 1.00 | 44.81 |
| ATOM | 3894 | CD | GLU | B | 912 | 21.220 | 6.107 | −27.081 | 1.00 | 47.97 |
| ATOM | 3895 | OE1 | GLU | B | 912 | 22.153 | 6.551 | −27.790 | 1.00 | 44.98 |
| ATOM | 3896 | OE2 | GLU | B | 912 | 20.049 | 5.964 | −27.502 | 1.00 | 50.02 |
| ATOM | 3897 | N | LEU | B | 913 | 20.402 | 8.558 | −22.374 | 1.00 | 44.41 |
| ATOM | 3898 | CA | LEU | B | 913 | 20.235 | 9.857 | −21.734 | 1.00 | 43.18 |
| ATOM | 3899 | C | LEU | B | 913 | 20.652 | 9.823 | −20.270 | 1.00 | 43.99 |
| ATOM | 3900 | O | LEU | B | 913 | 21.414 | 10.675 | −19.823 | 1.00 | 44.57 |
| ATOM | 3901 | CB | LEU | B | 913 | 18.787 | 10.332 | −21.862 | 1.00 | 40.85 |
| ATOM | 3902 | CG | LEU | B | 913 | 18.216 | 10.396 | −23.286 | 1.00 | 41.67 |
| ATOM | 3903 | CD1 | LEU | B | 913 | 16.795 | 10.945 | −23.221 | 1.00 | 39.11 |
| ATOM | 3904 | CD2 | LEU | B | 913 | 19.089 | 11.283 | −24.187 | 1.00 | 38.24 |
| ATOM | 3905 | N | MET | B | 914 | 20.158 | 8.843 | −19.519 | 1.00 | 45.82 |
| ATOM | 3906 | CA | MET | B | 914 | 20.513 | 8.742 | −18.109 | 1.00 | 48.13 |
| ATOM | 3907 | C | MET | B | 914 | 22.023 | 8.596 | −17.917 | 1.00 | 48.88 |
| ATOM | 3908 | O | MET | B | 914 | 22.553 | 8.972 | −16.864 | 1.00 | 48.55 |
| ATOM | 3909 | CB | MET | B | 914 | 19.796 | 7.564 | −17.443 | 1.00 | 50.82 |
| ATOM | 3910 | CG | MET | B | 914 | 18.292 | 7.740 | −17.317 | 1.00 | 52.72 |
| ATOM | 3911 | SD | MET | B | 914 | 17.787 | 9.437 | −16.952 | 1.00 | 53.52 |
| ATOM | 3912 | CE | MET | B | 914 | 16.626 | 9.705 | −18.264 | 1.00 | 52.21 |
| ATOM | 3913 | N | THR | B | 915 | 22.710 | 8.048 | −18.923 | 1.00 | 47.30 |
| ATOM | 3914 | CA | THR | B | 915 | 24.163 | 7.892 | −18.849 | 1.00 | 46.63 |
| ATOM | 3915 | C | THR | B | 915 | 24.875 | 9.052 | −19.562 | 1.00 | 48.58 |
| ATOM | 3916 | O | THR | B | 915 | 26.059 | 8.968 | −19.886 | 1.00 | 48.03 |
| ATOM | 3917 | CB | THR | B | 915 | 24.642 | 6.559 | −19.474 | 1.00 | 44.86 |
| ATOM | 3918 | OG1 | THR | B | 915 | 24.277 | 6.516 | −20.860 | 1.00 | 44.85 |
| ATOM | 3919 | CG2 | THR | B | 915 | 24.043 | 5.373 | −18.737 | 1.00 | 42.30 |
| ATOM | 3920 | N | PHE | B | 916 | 24.148 | 10.135 | −19.809 | 1.00 | 50.49 |
| ATOM | 3921 | CA | PHE | B | 916 | 24.731 | 11.297 | −20.463 | 1.00 | 52.07 |
| ATOM | 3922 | C | PHE | B | 916 | 25.505 | 10.901 | −21.717 | 1.00 | 54.08 |
| ATOM | 3923 | O | PHE | B | 916 | 26.687 | 11.222 | −21.848 | 1.00 | 56.00 |
| ATOM | 3924 | CB | PHE | B | 916 | 25.669 | 12.010 | −19.489 | 1.00 | 52.26 |
| ATOM | 3925 | CG | PHE | B | 916 | 24.972 | 12.617 | −18.305 | 1.00 | 52.03 |
| ATOM | 3926 | CD1 | PHE | B | 916 | 24.174 | 13.747 | −18.452 | 1.00 | 52.39 |
| ATOM | 3927 | CD2 | PHE | B | 916 | 25.128 | 12.076 | −17.040 | 1.00 | 52.11 |
| ATOM | 3928 | CE1 | PHE | B | 916 | 23.547 | 14.329 | −17.356 | 1.00 | 51.26 |
| ATOM | 3929 | CE2 | PHE | B | 916 | 24.502 | 12.654 | −15.938 | 1.00 | 52.35 |
| ATOM | 3930 | CZ | PHE | B | 916 | 23.713 | 13.781 | −16.097 | 1.00 | 50.17 |
| ATOM | 3931 | N | GLY | B | 917 | 24.845 | 10.188 | −22.626 | 1.00 | 54.17 |
| ATOM | 3932 | CA | GLY | B | 917 | 25.489 | 9.772 | −23.863 | 1.00 | 53.58 |
| ATOM | 3933 | C | GLY | B | 917 | 26.130 | 8.392 | −23.882 | 1.00 | 54.28 |
| ATOM | 3934 | O | GLY | B | 917 | 26.799 | 8.047 | −24.858 | 1.00 | 56.32 |
| ATOM | 3935 | N | GLY | B | 918 | 25.925 | 7.596 | −22.834 | 1.00 | 52.63 |
| ATOM | 3936 | CA | GLY | B | 918 | 26.519 | 6.268 | −22.777 | 1.00 | 51.85 |
| ATOM | 3937 | C | GLY | B | 918 | 26.302 | 5.358 | −23.977 | 1.00 | 53.07 |
| ATOM | 3938 | O | GLY | B | 918 | 25.357 | 5.533 | −24.748 | 1.00 | 53.60 |
| ATOM | 3939 | N | LYS | B | 919 | 27.184 | 4.375 | −24.136 | 1.00 | 53.42 |
| ATOM | 3940 | CA | LYS | B | 919 | 27.083 | 3.430 | −25.241 | 1.00 | 53.94 |
| ATOM | 3941 | C | LYS | B | 919 | 26.529 | 2.081 | −24.777 | 1.00 | 54.09 |
| ATOM | 3942 | O | LYS | B | 919 | 27.059 | 1.461 | −23.853 | 1.00 | 53.78 |
| ATOM | 3943 | CB | LYS | B | 919 | 28.451 | 3.224 | −25.898 | 1.00 | 56.15 |
| ATOM | 3944 | CG | LYS | B | 919 | 29.088 | 4.504 | −26.440 | 1.00 | 60.22 |
| ATOM | 3945 | CD | LYS | B | 919 | 30.229 | 4.199 | −27.399 | 1.00 | 62.11 |
| ATOM | 3946 | CE | LYS | B | 919 | 29.703 | 3.490 | −28.644 | 1.00 | 65.81 |
| ATOM | 3947 | NZ | LYS | B | 919 | 30.785 | 3.065 | −29.581 | 1.00 | 68.08 |
| ATOM | 3948 | N | PRO | B | 920 | 25.449 | 1.609 | −25.424 | 1.00 | 53.96 |
| ATOM | 3949 | CA | PRO | B | 920 | 24.779 | 0.340 | −25.122 | 1.00 | 52.93 |
| ATOM | 3950 | C | PRO | B | 920 | 25.730 | −0.832 | −25.235 | 1.00 | 52.53 |
| ATOM | 3951 | O | PRO | B | 920 | 26.390 | −0.994 | −26.254 | 1.00 | 51.03 |
| ATOM | 3952 | CB | PRO | B | 920 | 23.685 | 0.259 | −26.184 | 1.00 | 52.25 |
| ATOM | 3953 | CG | PRO | B | 920 | 23.412 | 1.669 | −26.510 | 1.00 | 54.01 |
| ATOM | 3954 | CD | PRO | B | 920 | 24.783 | 2.283 | −26.550 | 1.00 | 53.55 |
| ATOM | 3955 | N | TYR | B | 921 | 25.774 | −1.658 | −24.197 | 1.00 | 54.38 |
| ATOM | 3956 | CA | TYR | B | 921 | 26.649 | −2.823 | −24.182 | 1.00 | 57.62 |
| ATOM | 3957 | C | TYR | B | 921 | 28.007 | −2.445 | −24.764 | 1.00 | 60.38 |
| ATOM | 3958 | O | TYR | B | 921 | 28.491 | −3.075 | −25.707 | 1.00 | 61.02 |
| ATOM | 3959 | CB | TYR | B | 921 | 26.022 | −3.958 | −24.992 | 1.00 | 56.19 |
| ATOM | 3960 | CG | TYR | B | 921 | 24.546 | −4.115 | −24.735 | 1.00 | 53.82 |
| ATOM | 3961 | CD1 | TYR | B | 921 | 23.612 | −3.483 | −25.551 | 1.00 | 54.08 |
| ATOM | 3962 | CD2 | TYR | B | 921 | 24.084 | −4.862 | −23.658 | 1.00 | 51.52 |
| ATOM | 3963 | CE1 | TYR | B | 921 | 22.257 | −3.588 | −25.303 | 1.00 | 53.70 |
| ATOM | 3964 | CE2 | TYR | B | 921 | 22.730 | −4.975 | −23.396 | 1.00 | 52.18 |
| ATOM | 3965 | CZ | TYR | B | 921 | 21.820 | −4.337 | −24.223 | 1.00 | 53.77 |
| ATOM | 3966 | OH | TYR | B | 921 | 20.474 | −4.454 | −23.983 | 1.00 | 52.26 |
| ATOM | 3967 | N | ASP | B | 922 | 28.605 | −1.409 | −24.184 | 1.00 | 63.62 |
| ATOM | 3968 | CA | ASP | B | 922 | 29.895 | −0.888 | −24.616 | 1.00 | 67.06 |
| ATOM | 3969 | C | ASP | B | 922 | 30.960 | −1.967 | −24.783 | 1.00 | 68.44 |
| ATOM | 3970 | O | ASP | B | 922 | 31.239 | −2.732 | −23.857 | 1.00 | 67.06 |
| ATOM | 3971 | CB | ASP | B | 922 | 30.400 | 0.152 | −23.616 | 1.00 | 69.17 |
| ATOM | 3972 | CG | ASP | B | 922 | 31.481 | 1.037 | −24.200 | 1.00 | 70.95 |
| ATOM | 3973 | OD1 | ASP | B | 922 | 32.337 | 0.521 | −24.957 | 1.00 | 72.34 |
| ATOM | 3974 | OD2 | ASP | B | 922 | 31.478 | 2.250 | −23.894 | 1.00 | 70.84 |
| ATOM | 3975 | N | GLY | B | 923 | 31.556 | −2.008 | −25.971 | 1.00 | 70.59 |
| ATOM | 3976 | CA | GLY | B | 923 | 32.595 | −2.980 | −26.257 | 1.00 | 72.18 |
| ATOM | 3977 | C | GLY | B | 923 | 32.100 | −4.329 | −26.747 | 1.00 | 72.53 |
| ATOM | 3978 | O | GLY | B | 923 | 32.580 | −4.834 | −27.759 | 1.00 | 74.13 |
| ATOM | 3979 | N | ILE | B | 924 | 31.142 | −4.909 | −26.032 | 1.00 | 71.67 |
| ATOM | 3980 | CA | ILE | B | 924 | 30.591 | −6.215 | −26.373 | 1.00 | 70.50 |
| ATOM | 3981 | C | ILE | B | 924 | 30.351 | −6.414 | −27.872 | 1.00 | 71.43 |
| ATOM | 3982 | O | ILE | B | 924 | 29.838 | −5.527 | −28.556 | 1.00 | 71.73 |
| ATOM | 3983 | CB | ILE | B | 924 | 29.278 | −6.462 | −25.597 | 1.00 | 69.48 |
| ATOM | 3984 | CG1 | ILE | B | 924 | 29.545 | −6.319 | −24.091 | 1.00 | 68.05 |
| ATOM | 3985 | CG2 | ILE | B | 924 | 28.729 | −7.850 | −25.917 | 1.00 | 67.40 |
| ATOM | 3986 | CD1 | ILE | B | 924 | 28.299 | −6.301 | −23.212 | 1.00 | 69.45 |
| ATOM | 3987 | N | PRO | B | 925 | 30.737 | −7.594 | −28.398 | 1.00 | 71.75 |
| ATOM | 3988 | CA | PRO | B | 925 | 30.619 | −8.024 | −29.799 | 1.00 | 71.06 |
| ATOM | 3989 | C | PRO | B | 925 | 29.197 | −8.007 | −30.368 | 1.00 | 69.91 |
| ATOM | 3990 | O | PRO | B | 925 | 28.333 | −8.764 | −29.935 | 1.00 | 68.95 |
| ATOM | 3991 | CB | PRO | B | 925 | 31.191 | −9.442 | −29.774 | 1.00 | 71.99 |
| ATOM | 3992 | CG | PRO | B | 925 | 32.182 | −9.390 | −28.661 | 1.00 | 72.05 |
| ATOM | 3993 | CD | PRO | B | 925 | 31.432 | −8.625 | −27.604 | 1.00 | 72.02 |
| ATOM | 3994 | N | THR | B | 926 | 28.977 | −7.158 | −31.361 | 1.00 | 69.43 |
| ATOM | 3995 | CA | THR | B | 926 | 27.675 | −7.030 | −32.002 | 1.00 | 69.48 |
| ATOM | 3996 | C | THR | B | 926 | 26.961 | −8.380 | −32.208 | 1.00 | 69.75 |
| ATOM | 3997 | O | THR | B | 926 | 25.747 | −8.486 | −32.028 | 1.00 | 70.07 |
| ATOM | 3998 | CB | THR | B | 926 | 27.826 | −6.287 | −33.366 | 1.00 | 70.00 |
| ATOM | 3999 | OG1 | THR | B | 926 | 26.543 | −6.131 | −33.980 | 1.00 | 68.48 |
| ATOM | 4000 | CG2 | THR | B | 926 | 28.766 | −7.055 | −34.309 | 1.00 | 70.72 |
| ATOM | 4001 | N | ALA | B | 927 | 27.716 | −9.412 | −32.566 | 1.00 | 69.65 |
| ATOM | 4002 | CA | ALA | B | 927 | 27.140 | −10.732 | −32.804 | 1.00 | 68.51 |
| ATOM | 4003 | C | ALA | B | 927 | 26.683 | −11.403 | −31.510 | 1.00 | 68.30 |
| ATOM | 4004 | O | ALA | B | 927 | 25.807 | −12.270 | −31.526 | 1.00 | 68.58 |
| ATOM | 4005 | CB | ALA | B | 927 | 28.165 | −11.626 | −33.527 | 1.00 | 66.12 |
| ATOM | 4006 | N | GLU | B | 928 | 27.272 | −10.992 | −30.392 | 1.00 | 68.00 |
| ATOM | 4007 | CA | GLU | B | 928 | 26.958 | −11.577 | −29.090 | 1.00 | 68.38 |
| ATOM | 4008 | C | GLU | B | 928 | 25.634 | −11.170 | −28.463 | 1.00 | 66.53 |
| ATOM | 4009 | O | GLU | B | 928 | 25.007 | −11.959 | −27.753 | 1.00 | 64.89 |
| ATOM | 4010 | CB | GLU | B | 928 | 28.075 | −11.270 | −28.088 | 1.00 | 71.51 |
| ATOM | 4011 | CG | GLU | B | 928 | 29.263 | −12.221 | −28.136 | 1.00 | 75.50 |
| ATOM | 4012 | CD | GLU | B | 928 | 30.041 | −12.231 | −26.826 | 1.00 | 79.00 |
| ATOM | 4013 | OE1 | GLU | B | 928 | 30.933 | −13.095 | −26.662 | 1.00 | 81.12 |
| ATOM | 4014 | OE2 | GLU | B | 928 | 29.760 | −11.374 | −25.957 | 1.00 | 79.77 |
| ATOM | 4015 | N | ILE | B | 929 | 25.220 | −9.936 | −28.714 | 1.00 | 64.75 |
| ATOM | 4016 | CA | ILE | B | 929 | 23.987 | −9.415 | −28.147 | 1.00 | 63.35 |
| ATOM | 4017 | C | ILE | B | 929 | 22.858 | −10.446 | −28.077 | 1.00 | 63.03 |
| ATOM | 4018 | O | ILE | B | 929 | 22.337 | −10.734 | −26.998 | 1.00 | 61.05 |
| ATOM | 4019 | CB | ILE | B | 929 | 23.520 | −8.184 | −28.932 | 1.00 | 61.08 |
| ATOM | 4020 | CG1 | ILE | B | 929 | 24.652 | −7.155 | −28.994 | 1.00 | 58.47 |
| ATOM | 4021 | CG2 | ILE | B | 929 | 22.284 | −7.600 | −28.286 | 1.00 | 62.49 |
| ATOM | 4022 | CD1 | ILE | B | 929 | 25.199 | −6.739 | −27.651 | 1.00 | 53.78 |
| ATOM | 4023 | N | PRO | B | 930 | 22.474 | −11.026 | −29.225 | 1.00 | 63.06 |
| ATOM | 4024 | CA | PRO | B | 930 | 21.402 | −12.024 | −29.257 | 1.00 | 63.56 |
| ATOM | 4025 | C | PRO | B | 930 | 21.495 | −13.082 | −28.155 | 1.00 | 64.62 |
| ATOM | 4026 | O | PRO | B | 930 | 20.516 | −13.350 | −27.452 | 1.00 | 64.41 |
| ATOM | 4027 | CB | PRO | B | 930 | 21.541 | −12.617 | −30.652 | 1.00 | 62.01 |
| ATOM | 4028 | CG | PRO | B | 930 | 21.934 | −11.428 | −31.446 | 1.00 | 62.44 |
| ATOM | 4029 | CD | PRO | B | 930 | 22.998 | −10.792 | −30.581 | 1.00 | 61.86 |
| ATOM | 4030 | N | ASP | B | 931 | 22.668 | −13.686 | −28.004 | 1.00 | 65.47 |
| ATOM | 4031 | CA | ASP | B | 931 | 22.839 | −14.703 | −26.982 | 1.00 | 66.27 |
| ATOM | 4032 | C | ASP | B | 931 | 22.656 | −14.072 | −25.616 | 1.00 | 65.47 |
| ATOM | 4033 | O | ASP | B | 931 | 21.839 | −14.537 | −24.821 | 1.00 | 64.75 |
| ATOM | 4034 | CB | ASP | B | 931 | 24.227 | −15.345 | −27.078 | 1.00 | 70.06 |
| ATOM | 4035 | CG | ASP | B | 931 | 24.483 | −16.356 | −25.967 | 1.00 | 73.85 |
| ATOM | 4036 | OD1 | ASP | B | 931 | 23.678 | −17.313 | −25.835 | 1.00 | 75.74 |
| ATOM | 4037 | OD2 | ASP | B | 931 | 25.484 | −16.189 | −25.228 | 1.00 | 74.15 |
| ATOM | 4038 | N | LEU | B | 932 | 23.419 | −13.011 | −25.353 | 1.00 | 65.40 |
| ATOM | 4039 | CA | LEU | B | 932 | 23.349 | −12.298 | −24.080 | 1.00 | 65.25 |
| ATOM | 4040 | C | LEU | B | 932 | 21.902 | −12.006 | −23.678 | 1.00 | 65.94 |
| ATOM | 4041 | O | LEU | B | 932 | 21.452 | −12.390 | −22.594 | 1.00 | 65.04 |
| ATOM | 4042 | CB | LEU | B | 932 | 24.130 | −10.982 | −24.161 | 1.00 | 65.95 |
| ATOM | 4043 | CG | LEU | B | 932 | 25.660 | −11.055 | −24.245 | 1.00 | 68.42 |
| ATOM | 4044 | CD1 | LEU | B | 932 | 26.237 | −9.642 | −24.271 | 1.00 | 69.04 |
| ATOM | 4045 | CD2 | LEU | B | 932 | 26.219 | −11.823 | −23.044 | 1.00 | 68.73 |
| ATOM | 4046 | N | LEU | B | 933 | 21.173 | −11.334 | −24.562 | 1.00 | 65.07 |
| ATOM | 4047 | CA | LEU | B | 933 | 19.787 | −10.989 | −24.298 | 1.00 | 64.62 |
| ATOM | 4048 | C | LEU | B | 933 | 18.923 | −12.152 | −23.810 | 1.00 | 64.97 |
| ATOM | 4049 | O | LEU | B | 933 | 18.110 | −11.980 | −22.899 | 1.00 | 65.95 |
| ATOM | 4050 | CB | LEU | B | 933 | 19.166 | −10.368 | −25.548 | 1.00 | 63.72 |
| ATOM | 4051 | CG | LEU | B | 933 | 19.756 | −9.012 | −25.939 | 1.00 | 63.23 |
| ATOM | 4052 | CD1 | LEU | B | 933 | 19.177 | −8.570 | −27.271 | 1.00 | 61.73 |
| ATOM | 4053 | CD2 | LEU | B | 933 | 19.464 | −7.986 | −24.852 | 1.00 | 61.35 |
| ATOM | 4054 | N | GLU | B | 934 | 19.096 | −13.331 | −24.397 | 1.00 | 64.87 |
| ATOM | 4055 | CA | GLU | B | 934 | 18.294 | −14.487 | −23.997 | 1.00 | 65.11 |
| ATOM | 4056 | C | GLU | B | 934 | 18.805 | −15.154 | −22.730 | 1.00 | 63.75 |
| ATOM | 4057 | O | GLU | B | 934 | 18.163 | −16.050 | −22.199 | 1.00 | 63.55 |
| ATOM | 4058 | CB | GLU | B | 934 | 18.220 | −15.494 | −25.148 | 1.00 | 66.18 |
| ATOM | 4059 | CG | GLU | B | 934 | 17.478 | −14.925 | −26.342 | 1.00 | 71.36 |
| ATOM | 4060 | CD | GLU | B | 934 | 17.763 | −15.642 | −27.649 | 1.00 | 74.08 |
| ATOM | 4061 | OE1 | GLU | B | 934 | 18.950 | −15.765 | −28.029 | 1.00 | 75.67 |
| ATOM | 4062 | OE2 | GLU | B | 934 | 16.790 | −16.066 | −28.306 | 1.00 | 76.95 |
| ATOM | 4063 | N | LYS | B | 935 | 19.958 | −14.705 | −22.249 | 1.00 | 63.26 |
| ATOM | 4064 | CA | LYS | B | 935 | 20.558 | −15.241 | −21.032 | 1.00 | 63.78 |
| ATOM | 4065 | C | LYS | B | 935 | 20.263 | −14.297 | −19.867 | 1.00 | 64.04 |
| ATOM | 4066 | O | LYS | B | 935 | 20.830 | −14.442 | −18.783 | 1.00 | 65.10 |
| ATOM | 4067 | CB | LYS | B | 935 | 22.077 | −15.366 | −21.185 | 1.00 | 64.14 |
| ATOM | 4068 | CG | LYS | B | 935 | 22.556 | −16.374 | −22.206 | 1.00 | 65.76 |
| ATOM | 4069 | CD | LYS | B | 935 | 22.334 | −17.790 | −21.719 | 1.00 | 68.99 |
| ATOM | 4070 | CE | LYS | B | 935 | 23.035 | −18.799 | −22.610 | 1.00 | 70.04 |
| ATOM | 4071 | NZ | LYS | B | 935 | 22.771 | −20.184 | −22.139 | 1.00 | 71.50 |
| ATOM | 4072 | N | GLY | B | 936 | 19.406 | −13.308 | −20.105 | 1.00 | 62.82 |
| ATOM | 4073 | CA | GLY | B | 936 | 19.055 | −12.373 | −19.051 | 1.00 | 61.39 |
| ATOM | 4074 | C | GLY | B | 936 | 19.872 | −11.097 | −18.997 | 1.00 | 60.41 |
| ATOM | 4075 | O | GLY | B | 936 | 19.600 | −10.219 | −18.185 | 1.00 | 60.95 |
| ATOM | 4076 | N | GLU | B | 937 | 20.875 | −10.987 | −19.857 | 1.00 | 59.63 |
| ATOM | 4077 | CA | GLU | B | 937 | 21.729 | −9.797 | −19.903 | 1.00 | 58.26 |
| ATOM | 4078 | C | GLU | B | 937 | 20.903 | −8.522 | −20.150 | 1.00 | 56.06 |
| ATOM | 4079 | O | GLU | B | 937 | 20.057 | −8.496 | −21.038 | 1.00 | 55.96 |
| ATOM | 4080 | CB | GLU | B | 937 | 22.757 | −9.958 | −21.033 | 1.00 | 58.85 |
| ATOM | 4081 | CG | GLU | B | 937 | 23.808 | −8.877 | −21.113 | 1.00 | 61.46 |
| ATOM | 4082 | CD | GLU | B | 937 | 24.884 | −9.045 | −20.069 | 1.00 | 64.22 |
| ATOM | 4083 | OE1 | GLU | B | 937 | 25.719 | −8.129 | −19.921 | 1.00 | 66.78 |
| ATOM | 4084 | OE2 | GLU | B | 937 | 24.900 | −10.099 | −19.401 | 1.00 | 66.18 |
| ATOM | 4085 | N | ARG | B | 938 | 21.135 | −7.474 | −19.366 | 1.00 | 53.07 |
| ATOM | 4086 | CA | ARG | B | 938 | 20.427 | −6.213 | −19.567 | 1.00 | 51.85 |
| ATOM | 4087 | C | ARG | B | 938 | 21.322 | −5.046 | −19.211 | 1.00 | 52.49 |
| ATOM | 4088 | O | ARG | B | 938 | 22.249 | −5.179 | −18.405 | 1.00 | 54.14 |
| ATOM | 4089 | CB | ARG | B | 938 | 19.154 | −6.132 | −18.728 | 1.00 | 49.08 |
| ATOM | 4090 | CG | ARG | B | 938 | 18.047 | −7.023 | −19.212 | 1.00 | 48.98 |
| ATOM | 4091 | CD | ARG | B | 938 | 17.653 | −6.678 | −20.629 | 1.00 | 51.23 |
| ATOM | 4092 | NE | ARG | B | 938 | 17.145 | −7.859 | −21.322 | 1.00 | 56.53 |
| ATOM | 4093 | CZ | ARG | B | 938 | 16.012 | −8.471 | −21.015 | 1.00 | 57.01 |
| ATOM | 4094 | NH1 | ARG | B | 938 | 15.267 | −8.003 | −20.025 | 1.00 | 62.70 |
| ATOM | 4095 | NH2 | ARG | B | 938 | 15.631 | −9.549 | −21.682 | 1.00 | 55.07 |
| ATOM | 4096 | N | LEU | B | 939 | 21.043 | −3.898 | −19.813 | 1.00 | 50.95 |
| ATOM | 4097 | CA | LEU | B | 939 | 21.832 | −2.715 | −19.547 | 1.00 | 51.34 |
| ATOM | 4098 | C | LEU | B | 939 | 21.867 | −2.401 | −18.049 | 1.00 | 51.66 |
| ATOM | 4099 | O | LEU | B | 939 | 20.869 | −2.544 | −17.343 | 1.00 | 50.27 |
| ATOM | 4100 | CB | LEU | B | 939 | 21.282 | −1.534 | −20.349 | 1.00 | 49.96 |
| ATOM | 4101 | CG | LEU | B | 939 | 21.558 | −1.689 | −21.849 | 1.00 | 49.98 |
| ATOM | 4102 | CD1 | LEU | B | 939 | 20.779 | −0.666 | −22.664 | 1.00 | 47.22 |
| ATOM | 4103 | CD2 | LEU | B | 939 | 23.050 | −1.561 | −22.078 | 1.00 | 47.98 |
| ATOM | 4104 | N | PRO | B | 940 | 23.038 | −1.976 | −17.549 | 1.00 | 51.95 |
| ATOM | 4105 | CA | PRO | B | 940 | 23.306 | −1.620 | −16.151 | 1.00 | 51.29 |
| ATOM | 4106 | C | PRO | B | 940 | 22.397 | −0.511 | −15.667 | 1.00 | 51.06 |
| ATOM | 4107 | O | PRO | B | 940 | 21.776 | 0.173 | −16.469 | 1.00 | 52.57 |
| ATOM | 4108 | CB | PRO | B | 940 | 24.751 | −1.146 | −16.184 | 1.00 | 50.42 |
| ATOM | 4109 | CG | PRO | B | 940 | 25.325 | −1.852 | −17.362 | 1.00 | 52.89 |
| ATOM | 4110 | CD | PRO | B | 940 | 24.233 | −1.750 | −18.376 | 1.00 | 50.94 |
| ATOM | 4111 | N | GLN | B | 941 | 22.343 | −0.324 | −14.354 | 1.00 | 50.66 |
| ATOM | 4112 | CA | GLN | B | 941 | 21.535 | 0.725 | −13.760 | 1.00 | 48.49 |
| ATOM | 4113 | C | GLN | B | 941 | 22.339 | 2.010 | −13.777 | 1.00 | 46.91 |
| ATOM | 4114 | O | GLN | B | 941 | 23.409 | 2.089 | −13.200 | 1.00 | 47.29 |
| ATOM | 4115 | CB | GLN | B | 941 | 21.151 | 0.357 | −12.324 | 1.00 | 48.25 |
| ATOM | 4116 | CG | GLN | B | 941 | 20.480 | 1.481 | −11.554 | 1.00 | 49.45 |
| ATOM | 4117 | CD | GLN | B | 941 | 19.811 | 1.011 | −10.271 | 1.00 | 52.87 |
| ATOM | 4118 | OE1 | GLN | B | 941 | 19.261 | 1.815 | −9.512 | 1.00 | 52.38 |
| ATOM | 4119 | NE2 | GLN | B | 941 | 19.842 | −0.296 | −10.026 | 1.00 | 55.49 |
| ATOM | 4120 | N | PRO | B | 942 | 21.838 | 3.038 | −14.460 | 1.00 | 48.21 |
| ATOM | 4121 | CA | PRO | B | 942 | 22.613 | 4.278 | −14.477 | 1.00 | 49.11 |
| ATOM | 4122 | C | PRO | B | 942 | 22.849 | 4.807 | −13.059 | 1.00 | 50.71 |
| ATOM | 4123 | O | PRO | B | 942 | 21.954 | 4.803 | −12.216 | 1.00 | 51.40 |
| ATOM | 4124 | CB | PRO | B | 942 | 21.745 | 5.216 | −15.315 | 1.00 | 46.82 |
| ATOM | 4125 | CG | PRO | B | 942 | 21.020 | 4.271 | −16.223 | 1.00 | 47.86 |
| ATOM | 4126 | CD | PRO | B | 942 | 20.629 | 3.159 | −15.289 | 1.00 | 45.98 |
| ATOM | 4127 | N | PRO | B | 943 | 24.070 | 5.264 | −12.782 | 1.00 | 51.83 |
| ATOM | 4128 | CA | PRO | B | 943 | 24.474 | 5.807 | −11.484 | 1.00 | 51.12 |
| ATOM | 4129 | C | PRO | B | 943 | 23.470 | 6.802 | −10.898 | 1.00 | 51.11 |
| ATOM | 4130 | O | PRO | B | 943 | 23.110 | 6.713 | −9.723 | 1.00 | 51.29 |
| ATOM | 4131 | CB | PRO | B | 943 | 25.803 | 6.482 | −11.805 | 1.00 | 52.65 |
| ATOM | 4132 | CG | PRO | B | 943 | 26.371 | 5.605 | −12.856 | 1.00 | 52.13 |
| ATOM | 4133 | CD | PRO | B | 943 | 25.183 | 5.320 | −13.744 | 1.00 | 52.53 |
| ATOM | 4134 | N | ILE | B | 944 | 23.028 | 7.748 | −11.726 | 1.00 | 50.00 |
| ATOM | 4135 | CA | ILE | B | 944 | 22.099 | 8.792 | −11.299 | 1.00 | 48.49 |
| ATOM | 4136 | C | ILE | B | 944 | 20.658 | 8.315 | −11.174 | 1.00 | 49.21 |
| ATOM | 4137 | O | ILE | B | 944 | 19.785 | 9.043 | −10.681 | 1.00 | 49.19 |
| ATOM | 4138 | CB | ILE | B | 944 | 22.117 | 9.983 | −12.291 | 1.00 | 48.25 |
| ATOM | 4139 | CG1 | ILE | B | 944 | 21.350 | 9.618 | −13.571 | 1.00 | 43.64 |
| ATOM | 4140 | CG2 | ILE | B | 944 | 23.557 | 10.345 | −12.625 | 1.00 | 45.88 |
| ATOM | 4141 | CD1 | ILE | B | 944 | 21.185 | 10.768 | −14.509 | 1.00 | 44.55 |
| ATOM | 4142 | N | CYS | B | 945 | 20.414 | 7.091 | −11.623 | 1.00 | 49.20 |
| ATOM | 4143 | CA | CYS | B | 945 | 19.076 | 6.532 | −11.595 | 1.00 | 50.25 |
| ATOM | 4144 | C | CYS | B | 945 | 18.623 | 6.005 | −10.260 | 1.00 | 50.70 |
| ATOM | 4145 | O | CYS | B | 945 | 19.357 | 5.323 | −9.553 | 1.00 | 53.59 |
| ATOM | 4146 | CB | CYS | B | 945 | 18.933 | 5.413 | −12.630 | 1.00 | 50.33 |
| ATOM | 4147 | SG | CYS | B | 945 | 18.662 | 5.990 | −14.307 | 1.00 | 50.97 |
| ATOM | 4148 | N | THR | B | 946 | 17.388 | 6.339 | −9.930 | 1.00 | 49.35 |
| ATOM | 4149 | CA | THR | B | 946 | 16.765 | 5.883 | −8.710 | 1.00 | 49.02 |
| ATOM | 4150 | C | THR | B | 946 | 16.161 | 4.554 | −9.197 | 1.00 | 48.52 |
| ATOM | 4151 | O | THR | B | 946 | 15.777 | 4.457 | −10.360 | 1.00 | 49.59 |
| ATOM | 4152 | CB | THR | B | 946 | 15.682 | 6.905 | −8.282 | 1.00 | 46.42 |
| ATOM | 4153 | OG1 | THR | B | 946 | 15.473 | 6.829 | −6.872 | 1.00 | 47.27 |
| ATOM | 4154 | CG2 | THR | B | 946 | 14.386 | 6.650 | −9.002 | 1.00 | 43.71 |
| ATOM | 4155 | N | ILE | B | 947 | 16.100 | 3.527 | −8.357 | 1.00 | 48.32 |
| ATOM | 4156 | CA | ILE | B | 947 | 15.546 | 2.252 | −8.821 | 1.00 | 48.32 |
| ATOM | 4157 | C | ILE | B | 947 | 14.151 | 2.434 | −9.422 | 1.00 | 48.84 |
| ATOM | 4158 | O | ILE | B | 947 | 13.685 | 1.600 | −10.196 | 1.00 | 49.30 |
| ATOM | 4159 | CB | ILE | B | 947 | 15.478 | 1.180 | −7.696 | 1.00 | 46.78 |
| ATOM | 4160 | CG1 | ILE | B | 947 | 15.096 | −0.176 | −8.312 | 1.00 | 49.09 |
| ATOM | 4161 | CG2 | ILE | B | 947 | 14.467 | 1.593 | −6.627 | 1.00 | 46.28 |
| ATOM | 4162 | CD1 | ILE | B | 947 | 15.017 | −1.363 | −7.331 | 1.00 | 49.14 |
| ATOM | 4163 | N | ASP | B | 948 | 13.491 | 3.532 | −9.075 | 1.00 | 49.00 |
| ATOM | 4164 | CA | ASP | B | 948 | 12.162 | 3.808 | −9.611 | 1.00 | 50.27 |
| ATOM | 4165 | C | ASP | B | 948 | 12.247 | 3.963 | −11.134 | 1.00 | 49.29 |
| ATOM | 4166 | O | ASP | B | 948 | 11.403 | 3.440 | −11.874 | 1.00 | 48.74 |
| ATOM | 4167 | CB | ASP | B | 948 | 11.602 | 5.098 | −9.002 | 1.00 | 53.06 |
| ATOM | 4168 | CG | ASP | B | 948 | 11.377 | 4.995 | −7.503 | 1.00 | 53.88 |
| ATOM | 4169 | OD1 | ASP | B | 948 | 10.208 | 4.869 | −7.094 | 1.00 | 54.68 |
| ATOM | 4170 | OD2 | ASP | B | 948 | 12.363 | 5.044 | −6.736 | 1.00 | 55.07 |
| ATOM | 4171 | N | VAL | B | 949 | 13.272 | 4.691 | −11.583 | 1.00 | 47.43 |
| ATOM | 4172 | CA | VAL | B | 949 | 13.515 | 4.940 | −13.001 | 1.00 | 45.11 |
| ATOM | 4173 | C | VAL | B | 949 | 14.030 | 3.686 | −13.692 | 1.00 | 46.35 |
| ATOM | 4174 | O | VAL | B | 949 | 13.546 | 3.304 | −14.767 | 1.00 | 46.15 |
| ATOM | 4175 | CB | VAL | B | 949 | 14.576 | 6.043 | −13.208 | 1.00 | 44.06 |
| ATOM | 4176 | CG1 | VAL | B | 949 | 14.835 | 6.254 | −14.696 | 1.00 | 41.72 |
| ATOM | 4177 | CG2 | VAL | B | 949 | 14.128 | 7.325 | −12.565 | 1.00 | 40.72 |
| ATOM | 4178 | N | TYR | B | 950 | 15.019 | 3.050 | −13.068 | 1.00 | 46.25 |
| ATOM | 4179 | CA | TYR | B | 950 | 15.613 | 1.850 | −13.629 | 1.00 | 46.35 |
| ATOM | 4180 | C | TYR | B | 950 | 14.590 | 0.760 | −13.932 | 1.00 | 45.76 |
| ATOM | 4181 | O | TYR | B | 950 | 14.665 | 0.122 | −14.977 | 1.00 | 43.83 |
| ATOM | 4182 | CB | TYR | B | 950 | 16.689 | 1.297 | −12.694 | 1.00 | 46.54 |
| ATOM | 4183 | CG | TYR | B | 950 | 17.528 | 0.203 | −13.325 | 1.00 | 49.48 |
| ATOM | 4184 | CD1 | TYR | B | 950 | 18.209 | 0.422 | −14.524 | 1.00 | 48.95 |
| ATOM | 4185 | CD2 | TYR | B | 950 | 17.649 | −1.050 | −12.721 | 1.00 | 51.79 |
| ATOM | 4186 | CE1 | TYR | B | 950 | 18.986 | −0.571 | −15.105 | 1.00 | 49.83 |
| ATOM | 4187 | CE2 | TYR | B | 950 | 18.428 | −2.057 | −13.294 | 1.00 | 51.29 |
| ATOM | 4188 | CZ | TYR | B | 950 | 19.096 | −1.809 | −14.485 | 1.00 | 51.48 |
| ATOM | 4189 | OH | TYR | B | 950 | 19.891 | −2.785 | −15.041 | 1.00 | 49.38 |
| ATOM | 4190 | N | MET | B | 951 | 13.636 | 0.547 | −13.030 | 1.00 | 46.47 |
| ATOM | 4191 | CA | MET | B | 951 | 12.630 | −0.484 | −13.258 | 1.00 | 47.95 |
| ATOM | 4192 | C | MET | B | 951 | 11.790 | −0.156 | −14.480 | 1.00 | 47.93 |
| ATOM | 4193 | O | MET | B | 951 | 11.274 | −1.059 | −15.152 | 1.00 | 50.04 |
| ATOM | 4194 | CB | MET | B | 951 | 11.741 | −0.679 | −12.026 | 1.00 | 49.33 |
| ATOM | 4195 | CG | MET | B | 951 | 12.514 | −1.169 | −10.798 | 1.00 | 55.37 |
| ATOM | 4196 | SD | MET | B | 951 | 13.753 | −2.470 | −11.188 | 1.00 | 62.22 |
| ATOM | 4197 | CE | MET | B | 951 | 12.677 | −3.817 | −11.608 | 1.00 | 59.62 |
| ATOM | 4198 | N | VAL | B | 952 | 11.650 | 1.128 | −14.784 | 1.00 | 45.24 |
| ATOM | 4199 | CA | VAL | B | 952 | 10.896 | 1.494 | −15.971 | 1.00 | 44.94 |
| ATOM | 4200 | C | VAL | B | 952 | 11.668 | 0.969 | −17.194 | 1.00 | 44.46 |
| ATOM | 4201 | O | VAL | B | 952 | 11.113 | 0.260 | −18.037 | 1.00 | 42.31 |
| ATOM | 4202 | CB | VAL | B | 952 | 10.694 | 3.031 | −16.067 | 1.00 | 44.63 |
| ATOM | 4203 | CG1 | VAL | B | 952 | 10.122 | 3.407 | −17.423 | 1.00 | 40.56 |
| ATOM | 4204 | CG2 | VAL | B | 952 | 9.738 | 3.489 | −14.971 | 1.00 | 42.36 |
| ATOM | 4205 | N | MET | B | 953 | 12.954 | 1.300 | −17.269 | 1.00 | 44.54 |
| ATOM | 4206 | CA | MET | B | 953 | 13.789 | 0.846 | −18.377 | 1.00 | 45.71 |
| ATOM | 4207 | C | MET | B | 953 | 13.760 | −0.677 | −18.498 | 1.00 | 45.34 |
| ATOM | 4208 | O | MET | B | 953 | 13.606 | −1.218 | −19.598 | 1.00 | 44.65 |
| ATOM | 4209 | CB | MET | B | 953 | 15.227 | 1.307 | −18.179 | 1.00 | 46.83 |
| ATOM | 4210 | CG | MET | B | 953 | 15.396 | 2.800 | −18.224 | 1.00 | 52.42 |
| ATOM | 4211 | SD | MET | B | 953 | 16.973 | 3.296 | −17.565 | 1.00 | 57.82 |
| ATOM | 4212 | CE | MET | B | 953 | 16.787 | 5.046 | −17.587 | 1.00 | 57.27 |
| ATOM | 4213 | N | VAL | B | 954 | 13.903 | −1.365 | −17.369 | 1.00 | 43.09 |
| ATOM | 4214 | CA | VAL | B | 954 | 13.891 | −2.815 | −17.375 | 1.00 | 43.36 |
| ATOM | 4215 | C | VAL | B | 954 | 12.588 | −3.343 | −17.961 | 1.00 | 43.58 |
| ATOM | 4216 | O | VAL | B | 954 | 12.605 | −4.189 | −18.857 | 1.00 | 41.66 |
| ATOM | 4217 | CB | VAL | B | 954 | 14.094 | −3.379 | −15.961 | 1.00 | 43.56 |
| ATOM | 4218 | CG1 | VAL | B | 954 | 14.037 | −4.883 | −15.993 | 1.00 | 45.22 |
| ATOM | 4219 | CG2 | VAL | B | 954 | 15.439 | −2.946 | −15.430 | 1.00 | 44.09 |
| ATOM | 4220 | N | LYS | B | 955 | 11.459 | −2.827 | −17.476 | 1.00 | 44.30 |
| ATOM | 4221 | CA | LYS | B | 955 | 10.158 | −3.262 | −17.982 | 1.00 | 44.82 |
| ATOM | 4222 | C | LYS | B | 955 | 10.096 | −3.192 | −19.504 | 1.00 | 44.68 |
| ATOM | 4223 | O | LYS | B | 955 | 9.542 | −4.072 | −20.160 | 1.00 | 46.69 |
| ATOM | 4224 | CB | LYS | B | 955 | 9.041 | −2.410 | −17.391 | 1.00 | 46.61 |
| ATOM | 4225 | CG | LYS | B | 955 | 8.647 | −2.770 | −15.972 | 1.00 | 46.76 |
| ATOM | 4226 | CD | LYS | B | 955 | 7.588 | −1.812 | −15.500 | 1.00 | 47.23 |
| ATOM | 4227 | CE | LYS | B | 955 | 7.121 | −2.128 | −14.119 | 1.00 | 48.72 |
| ATOM | 4228 | NZ | LYS | B | 955 | 6.636 | −0.872 | −13.502 | 1.00 | 52.38 |
| ATOM | 4229 | N | CYS | B | 956 | 10.665 | −2.139 | −20.066 | 1.00 | 43.75 |
| ATOM | 4230 | CA | CYS | B | 956 | 10.682 | −1.978 | −21.509 | 1.00 | 43.94 |
| ATOM | 4231 | C | CYS | B | 956 | 11.396 | −3.138 | −22.195 | 1.00 | 45.19 |
| ATOM | 4232 | O | CYS | B | 956 | 11.205 | −3.365 | −23.397 | 1.00 | 43.93 |
| ATOM | 4233 | CB | CYS | B | 956 | 11.410 | −0.692 | −21.883 | 1.00 | 42.14 |
| ATOM | 4234 | SG | CYS | B | 956 | 10.593 | 0.788 | −21.373 | 1.00 | 41.02 |
| ATOM | 4235 | N | TRP | B | 957 | 12.221 | −3.860 | −21.433 | 1.00 | 44.92 |
| ATOM | 4236 | CA | TRP | B | 957 | 12.995 | −4.957 | −21.995 | 1.00 | 45.41 |
| ATOM | 4237 | C | TRP | B | 957 | 12.543 | −6.352 | −21.592 | 1.00 | 47.76 |
| ATOM | 4238 | O | TRP | B | 957 | 13.328 | −7.295 | −21.641 | 1.00 | 47.21 |
| ATOM | 4239 | CB | TRP | B | 957 | 14.477 | −4.783 | −21.645 | 1.00 | 41.77 |
| ATOM | 4240 | CG | TRP | B | 957 | 15.012 | −3.417 | −21.957 | 1.00 | 38.80 |
| ATOM | 4241 | CD1 | TRP | B | 957 | 14.764 | −2.680 | −23.074 | 1.00 | 38.90 |
| ATOM | 4242 | CD2 | TRP | B | 957 | 15.910 | −2.633 | −21.150 | 1.00 | 37.82 |
| ATOM | 4243 | NE1 | TRP | B | 957 | 15.444 | −1.482 | −23.019 | 1.00 | 39.19 |
| ATOM | 4244 | CE2 | TRP | B | 957 | 16.159 | −1.429 | −21.851 | 1.00 | 37.54 |
| ATOM | 4245 | CE3 | TRP | B | 957 | 16.528 | −2.832 | −19.907 | 1.00 | 36.20 |
| ATOM | 4246 | CZ2 | TRP | B | 957 | 17.004 | −0.425 | −21.353 | 1.00 | 36.54 |
| ATOM | 4247 | CZ3 | TRP | B | 957 | 17.366 | −1.835 | −19.414 | 1.00 | 39.57 |
| ATOM | 4248 | CH2 | TRP | B | 957 | 17.596 | −0.643 | −20.142 | 1.00 | 36.55 |
| ATOM | 4249 | N | MET | B | 958 | 11.289 | −6.491 | −21.185 | 1.00 | 49.98 |
| ATOM | 4250 | CA | MET | B | 958 | 10.793 | −7.810 | −20.828 | 1.00 | 50.86 |
| ATOM | 4251 | C | MET | B | 958 | 10.806 | −8.612 | −22.115 | 1.00 | 50.79 |
| ATOM | 4252 | O | MET | B | 958 | 10.734 | −8.045 | −23.205 | 1.00 | 49.90 |
| ATOM | 4253 | CB | MET | B | 958 | 9.373 | −7.731 | −20.264 | 1.00 | 52.90 |
| ATOM | 4254 | CG | MET | B | 958 | 9.295 | −7.075 | −18.896 | 1.00 | 55.44 |
| ATOM | 4255 | SD | MET | B | 958 | 10.524 | −7.763 | −17.765 | 1.00 | 57.70 |
| ATOM | 4256 | CE | MET | B | 958 | 9.531 | −9.050 | −16.941 | 1.00 | 59.85 |
| ATOM | 4257 | N | ILE | B | 959 | 10.914 | −9.927 | −21.998 | 1.00 | 51.99 |
| ATOM | 4258 | CA | ILE | B | 959 | 10.955 | −10.768 | −23.184 | 1.00 | 53.60 |
| ATOM | 4259 | C | ILE | B | 959 | 9.611 | −10.696 | −23.888 | 1.00 | 52.84 |
| ATOM | 4260 | O | ILE | B | 959 | 9.536 | −10.632 | −25.111 | 1.00 | 52.06 |
| ATOM | 4261 | CB | ILE | B | 959 | 11.251 | −12.232 | −22.823 | 1.00 | 55.50 |
| ATOM | 4262 | CG1 | ILE | B | 959 | 12.621 | −12.335 | −22.135 | 1.00 | 59.93 |
| ATOM | 4263 | CG2 | ILE | B | 959 | 11.235 | −13.079 | −24.090 | 1.00 | 56.33 |
| ATOM | 4264 | CD1 | ILE | B | 959 | 12.904 | −13.710 | −21.482 | 1.00 | 62.42 |
| ATOM | 4265 | N | ASP | B | 960 | 8.551 | −10.695 | −23.093 | 1.00 | 53.61 |
| ATOM | 4266 | CA | ASP | B | 960 | 7.192 | −10.625 | −23.610 | 1.00 | 54.49 |
| ATOM | 4267 | C | ASP | B | 960 | 6.860 | −9.199 | −24.063 | 1.00 | 52.87 |
| ATOM | 4268 | O | ASP | B | 960 | 6.711 | −8.290 | −23.250 | 1.00 | 50.14 |
| ATOM | 4269 | CB | ASP | B | 960 | 6.212 | −11.072 | −22.528 | 1.00 | 57.08 |
| ATOM | 4270 | CG | ASP | B | 960 | 4.851 | −11.400 | −23.081 | 1.00 | 61.48 |
| ATOM | 4271 | OD1 | ASP | B | 960 | 4.233 | −10.509 | −23.706 | 1.00 | 65.36 |
| ATOM | 4272 | OD2 | ASP | B | 960 | 4.395 | −12.550 | −22.891 | 1.00 | 64.23 |
| ATOM | 4273 | N | ALA | B | 961 | 6.745 | −9.020 | −25.371 | 1.00 | 52.25 |
| ATOM | 4274 | CA | ALA | B | 961 | 6.438 | −7.722 | −25.950 | 1.00 | 53.15 |
| ATOM | 4275 | C | ALA | B | 961 | 5.192 | −7.090 | −25.339 | 1.00 | 54.47 |
| ATOM | 4276 | O | ALA | B | 961 | 5.188 | −5.902 | −25.013 | 1.00 | 54.47 |
| ATOM | 4277 | CB | ALA | B | 961 | 6.263 | −7.864 | −27.449 | 1.00 | 49.84 |
| ATOM | 4278 | N | ASP | B | 962 | 4.140 | −7.888 | −25.182 | 1.00 | 56.23 |
| ATOM | 4279 | CA | ASP | B | 962 | 2.888 | −7.393 | −24.618 | 1.00 | 57.94 |
| ATOM | 4280 | C | ASP | B | 962 | 2.943 | −7.036 | −23.135 | 1.00 | 56.89 |
| ATOM | 4281 | O | ASP | B | 962 | 2.045 | −6.358 | −22.632 | 1.00 | 57.51 |
| ATOM | 4282 | CB | ASP | B | 962 | 1.748 | −8.396 | −24.840 | 1.00 | 60.84 |
| ATOM | 4283 | CG | ASP | B | 962 | 1.280 | −8.451 | −26.289 | 1.00 | 64.87 |
| ATOM | 4284 | OD1 | ASP | B | 962 | 1.123 | −7.376 | −26.915 | 1.00 | 66.69 |
| ATOM | 4285 | OD2 | ASP | B | 962 | 1.054 | −9.575 | −26.794 | 1.00 | 67.24 |
| ATOM | 4286 | N | SER | B | 963 | 3.973 | −7.481 | −22.422 | 1.00 | 54.15 |
| ATOM | 4287 | CA | SER | B | 963 | 4.050 | −7.144 | −21.008 | 1.00 | 52.47 |
| ATOM | 4288 | C | SER | B | 963 | 4.973 | −5.954 | −20.761 | 1.00 | 50.73 |
| ATOM | 4289 | O | SER | B | 963 | 5.319 | −5.646 | −19.623 | 1.00 | 51.91 |
| ATOM | 4290 | CB | SER | B | 963 | 4.502 | −8.354 | −20.188 | 1.00 | 53.91 |
| ATOM | 4291 | OG | SER | B | 963 | 5.814 | −8.753 | −20.531 | 1.00 | 56.30 |
| ATOM | 4292 | N | ARG | B | 964 | 5.380 | −5.288 | −21.835 | 1.00 | 48.87 |
| ATOM | 4293 | CA | ARG | B | 964 | 6.235 | −4.115 | −21.710 | 1.00 | 47.43 |
| ATOM | 4294 | C | ARG | B | 964 | 5.327 | −2.916 | −21.536 | 1.00 | 45.64 |
| ATOM | 4295 | O | ARG | B | 964 | 4.202 | −2.898 | −22.036 | 1.00 | 46.22 |
| ATOM | 4296 | CB | ARG | B | 964 | 7.079 | −3.914 | −22.964 | 1.00 | 46.37 |
| ATOM | 4297 | CG | ARG | B | 964 | 8.058 | −5.025 | −23.230 | 1.00 | 47.48 |
| ATOM | 4298 | CD | ARG | B | 964 | 8.697 | −4.888 | −24.604 | 1.00 | 46.80 |
| ATOM | 4299 | NE | ARG | B | 964 | 9.474 | −6.074 | −24.930 | 1.00 | 47.04 |
| ATOM | 4300 | CZ | ARG | B | 964 | 9.889 | −6.388 | −26.149 | 1.00 | 46.77 |
| ATOM | 4301 | NH1 | ARG | B | 964 | 9.609 | −5.600 | −27.174 | 1.00 | 46.76 |
| ATOM | 4302 | NH2 | ARG | B | 964 | 10.564 | −7.507 | −26.343 | 1.00 | 48.41 |
| ATOM | 4303 | N | PRO | B | 965 | 5.800 | −1.890 | −20.831 | 1.00 | 43.80 |
| ATOM | 4304 | CA | PRO | B | 965 | 4.915 | −0.733 | −20.671 | 1.00 | 43.62 |
| ATOM | 4305 | C | PRO | B | 965 | 4.533 | −0.150 | −22.022 | 1.00 | 42.50 |
| ATOM | 4306 | O | PRO | B | 965 | 5.260 | −0.301 | −23.005 | 1.00 | 43.40 |
| ATOM | 4307 | CB | PRO | B | 965 | 5.743 | 0.226 | −19.821 | 1.00 | 42.89 |
| ATOM | 4308 | CG | PRO | B | 965 | 7.161 | −0.156 | −20.152 | 1.00 | 45.29 |
| ATOM | 4309 | CD | PRO | B | 965 | 7.122 | −1.653 | −20.233 | 1.00 | 43.22 |
| ATOM | 4310 | N | LYS | B | 966 | 3.373 | 0.490 | −22.080 | 1.00 | 40.83 |
| ATOM | 4311 | CA | LYS | B | 966 | 2.932 | 1.102 | −23.313 | 1.00 | 40.21 |
| ATOM | 4312 | C | LYS | B | 966 | 3.289 | 2.583 | −23.303 | 1.00 | 39.83 |
| ATOM | 4313 | O | LYS | B | 966 | 3.423 | 3.189 | −22.232 | 1.00 | 38.40 |
| ATOM | 4314 | CB | LYS | B | 966 | 1.446 | 0.861 | −23.491 | 1.00 | 41.77 |
| ATOM | 4315 | CG | LYS | B | 966 | 1.194 | −0.588 | −23.796 | 1.00 | 47.30 |
| ATOM | 4316 | CD | LYS | B | 966 | −0.223 | −0.888 | −24.170 | 1.00 | 51.96 |
| ATOM | 4317 | CE | LYS | B | 966 | −0.392 | −2.395 | −24.327 | 1.00 | 57.53 |
| ATOM | 4318 | NZ | LYS | B | 966 | −1.831 | −2.804 | −24.235 | 1.00 | 61.18 |
| ATOM | 4319 | N | PHE | B | 967 | 3.469 | 3.167 | −24.485 | 1.00 | 38.02 |
| ATOM | 4320 | CA | PHE | B | 967 | 3.866 | 4.569 | −24.547 | 1.00 | 40.61 |
| ATOM | 4321 | C | PHE | B | 967 | 2.998 | 5.514 | −23.741 | 1.00 | 41.52 |
| ATOM | 4322 | O | PHE | B | 967 | 3.509 | 6.479 | −23.180 | 1.00 | 42.24 |
| ATOM | 4323 | CB | PHE | B | 967 | 4.007 | 5.032 | −26.004 | 1.00 | 38.99 |
| ATOM | 4324 | CG | PHE | B | 967 | 5.289 | 4.572 | −26.648 | 1.00 | 38.22 |
| ATOM | 4325 | CD1 | PHE | B | 967 | 5.279 | 3.891 | −27.858 | 1.00 | 37.36 |
| ATOM | 4326 | CD2 | PHE | B | 967 | 6.510 | 4.769 | −26.002 | 1.00 | 37.25 |
| ATOM | 4327 | CE1 | PHE | B | 967 | 6.477 | 3.405 | −28.417 | 1.00 | 42.23 |
| ATOM | 4328 | CE2 | PHE | B | 967 | 7.712 | 4.287 | −26.551 | 1.00 | 39.11 |
| ATOM | 4329 | CZ | PHE | B | 967 | 7.698 | 3.605 | −27.756 | 1.00 | 39.07 |
| ATOM | 4330 | N | ALA | B | 968 | 1.701 | 5.223 | −23.652 | 1.00 | 42.04 |
| ATOM | 4331 | CA | ALA | B | 968 | 0.792 | 6.059 | −22.872 | 1.00 | 44.04 |
| ATOM | 4332 | C | ALA | B | 968 | 1.210 | 6.066 | −21.398 | 1.00 | 46.45 |
| ATOM | 4333 | O | ALA | B | 968 | 1.146 | 7.098 | −20.729 | 1.00 | 46.84 |
| ATOM | 4334 | CB | ALA | B | 968 | −0.650 | 5.549 | −22.998 | 1.00 | 42.69 |
| ATOM | 4335 | N | GLU | B | 969 | 1.640 | 4.913 | −20.895 | 1.00 | 46.92 |
| ATOM | 4336 | CA | GLU | B | 969 | 2.048 | 4.804 | −19.503 | 1.00 | 47.61 |
| ATOM | 4337 | C | GLU | B | 969 | 3.420 | 5.396 | −19.227 | 1.00 | 47.48 |
| ATOM | 4338 | O | GLU | B | 969 | 3.637 | 6.015 | −18.182 | 1.00 | 47.99 |
| ATOM | 4339 | CB | GLU | B | 969 | 2.079 | 3.349 | −19.065 | 1.00 | 51.22 |
| ATOM | 4340 | CG | GLU | B | 969 | 0.851 | 2.539 | −19.384 | 1.00 | 57.69 |
| ATOM | 4341 | CD | GLU | B | 969 | 1.087 | 1.070 | −19.086 | 1.00 | 62.44 |
| ATOM | 4342 | OE1 | GLU | B | 969 | 1.928 | 0.442 | −19.771 | 1.00 | 63.17 |
| ATOM | 4343 | OE2 | GLU | B | 969 | 0.446 | 0.546 | −18.151 | 1.00 | 68.35 |
| ATOM | 4344 | N | LEU | B | 970 | 4.362 | 5.182 | −20.138 | 1.00 | 45.82 |
| ATOM | 4345 | CA | LEU | B | 970 | 5.699 | 5.704 | −19.917 | 1.00 | 44.09 |
| ATOM | 4346 | C | LEU | B | 970 | 5.597 | 7.211 | −19.807 | 1.00 | 44.59 |
| ATOM | 4347 | O | LEU | B | 970 | 6.152 | 7.819 | −18.890 | 1.00 | 45.03 |
| ATOM | 4348 | CB | LEU | B | 970 | 6.645 | 5.276 | −21.050 | 1.00 | 43.43 |
| ATOM | 4349 | CG | LEU | B | 970 | 7.053 | 3.782 | −21.016 | 1.00 | 43.26 |
| ATOM | 4350 | CD1 | LEU | B | 970 | 7.660 | 3.340 | −22.331 | 1.00 | 40.75 |
| ATOM | 4351 | CD2 | LEU | B | 970 | 8.029 | 3.551 | −19.887 | 1.00 | 40.19 |
| ATOM | 4352 | N | ALA | B | 971 | 4.849 | 7.815 | −20.718 | 1.00 | 44.94 |
| ATOM | 4353 | CA | ALA | B | 971 | 4.684 | 9.257 | −20.690 | 1.00 | 45.01 |
| ATOM | 4354 | C | ALA | B | 971 | 4.093 | 9.663 | −19.350 | 1.00 | 46.07 |
| ATOM | 4355 | O | ALA | B | 971 | 4.559 | 10.602 | −18.709 | 1.00 | 48.26 |
| ATOM | 4356 | CB | ALA | B | 971 | 3.782 | 9.699 | −21.806 | 1.00 | 43.14 |
| ATOM | 4357 | N | ALA | B | 972 | 3.075 | 8.939 | −18.919 | 1.00 | 43.91 |
| ATOM | 4358 | CA | ALA | B | 972 | 2.435 | 9.242 | −17.660 | 1.00 | 46.47 |
| ATOM | 4359 | C | ALA | B | 972 | 3.386 | 9.110 | −16.466 | 1.00 | 48.41 |
| ATOM | 4360 | O | ALA | B | 972 | 3.481 | 10.018 | −15.635 | 1.00 | 50.02 |
| ATOM | 4361 | CB | ALA | B | 972 | 1.233 | 8.339 | −17.474 | 1.00 | 46.27 |
| ATOM | 4362 | N | GLU | B | 973 | 4.092 | 7.988 | −16.377 | 1.00 | 49.34 |
| ATOM | 4363 | CA | GLU | B | 973 | 5.003 | 7.768 | −15.259 | 1.00 | 50.14 |
| ATOM | 4364 | C | GLU | B | 973 | 6.160 | 8.769 | −15.233 | 1.00 | 49.00 |
| ATOM | 4365 | O | GLU | B | 973 | 6.488 | 9.309 | −14.182 | 1.00 | 47.51 |
| ATOM | 4366 | CB | GLU | B | 973 | 5.537 | 6.330 | −15.295 | 1.00 | 53.73 |
| ATOM | 4367 | CG | GLU | B | 973 | 6.424 | 5.917 | −14.120 | 1.00 | 58.85 |
| ATOM | 4368 | CD | GLU | B | 973 | 5.733 | 6.039 | −12.764 | 1.00 | 64.87 |
| ATOM | 4369 | OE1 | GLU | B | 973 | 4.555 | 5.625 | −12.644 | 1.00 | 67.25 |
| ATOM | 4370 | OE2 | GLU | B | 973 | 6.374 | 6.540 | −11.808 | 1.00 | 67.54 |
| ATOM | 4371 | N | PHE | B | 974 | 6.784 | 9.026 | −16.376 | 1.00 | 47.15 |
| ATOM | 4372 | CA | PHE | B | 974 | 7.892 | 9.970 | −16.385 | 1.00 | 46.71 |
| ATOM | 4373 | C | PHE | B | 974 | 7.340 | 11.351 | −16.178 | 1.00 | 47.30 |
| ATOM | 4374 | O | PHE | B | 974 | 8.017 | 12.242 | −15.672 | 1.00 | 45.15 |
| ATOM | 4375 | CB | PHE | B | 974 | 8.649 | 9.898 | −17.700 | 1.00 | 44.76 |
| ATOM | 4376 | CG | PHE | B | 974 | 9.648 | 8.794 | −17.742 | 1.00 | 45.39 |
| ATOM | 4377 | CD1 | PHE | B | 974 | 10.790 | 8.851 | −16.952 | 1.00 | 43.84 |
| ATOM | 4378 | CD2 | PHE | B | 974 | 9.433 | 7.669 | −18.533 | 1.00 | 44.99 |
| ATOM | 4379 | CE1 | PHE | B | 974 | 11.705 | 7.800 | −16.946 | 1.00 | 43.56 |
| ATOM | 4380 | CE2 | PHE | B | 974 | 10.343 | 6.612 | −18.531 | 1.00 | 44.73 |
| ATOM | 4381 | CZ | PHE | B | 974 | 11.480 | 6.680 | −17.734 | 1.00 | 43.10 |
| ATOM | 4382 | N | SER | B | 975 | 6.082 | 11.509 | −16.564 | 1.00 | 48.14 |
| ATOM | 4383 | CA | SER | B | 975 | 5.393 | 12.775 | −16.427 | 1.00 | 48.35 |
| ATOM | 4384 | C | SER | B | 975 | 5.302 | 13.184 | −14.953 | 1.00 | 48.95 |
| ATOM | 4385 | O | SER | B | 975 | 5.666 | 14.305 | −14.589 | 1.00 | 49.88 |
| ATOM | 4386 | CB | SER | B | 975 | 3.999 | 12.661 | −17.040 | 1.00 | 48.77 |
| ATOM | 4387 | OG | SER | B | 975 | 3.325 | 13.899 | −17.030 | 1.00 | 52.34 |
| ATOM | 4388 | N | ARG | B | 976 | 4.831 | 12.287 | −14.093 | 1.00 | 47.45 |
| ATOM | 4389 | CA | ARG | B | 976 | 4.732 | 12.658 | −12.697 | 1.00 | 47.19 |
| ATOM | 4390 | C | ARG | B | 976 | 6.105 | 12.708 | −12.029 | 1.00 | 47.91 |
| ATOM | 4391 | O | ARG | B | 976 | 6.298 | 13.433 | −11.055 | 1.00 | 48.07 |
| ATOM | 4392 | CB | ARG | B | 976 | 3.771 | 11.721 | −11.942 | 1.00 | 47.00 |
| ATOM | 4393 | CG | ARG | B | 976 | 4.268 | 10.343 | −11.695 | 1.00 | 48.59 |
| ATOM | 4394 | CD | ARG | B | 976 | 3.392 | 9.578 | −10.696 | 1.00 | 51.30 |
| ATOM | 4395 | NE | ARG | B | 976 | 4.049 | 8.319 | −10.349 | 1.00 | 53.72 |
| ATOM | 4396 | CZ | ARG | B | 976 | 5.139 | 8.240 | −9.593 | 1.00 | 56.06 |
| ATOM | 4397 | NH1 | ARG | B | 976 | 5.697 | 7.066 | −9.337 | 1.00 | 58.55 |
| ATOM | 4398 | NH2 | ARG | B | 976 | 5.652 | 9.337 | −9.056 | 1.00 | 57.80 |
| ATOM | 4399 | N | MET | B | 977 | 7.065 | 11.960 | −12.558 | 1.00 | 49.05 |
| ATOM | 4400 | CA | MET | B | 977 | 8.407 | 11.967 | −11.988 | 1.00 | 51.32 |
| ATOM | 4401 | C | MET | B | 977 | 9.021 | 13.332 | −12.249 | 1.00 | 52.43 |
| ATOM | 4402 | O | MET | B | 977 | 9.887 | 13.796 | −11.503 | 1.00 | 52.50 |
| ATOM | 4403 | CB | MET | B | 977 | 9.269 | 10.854 | −12.604 | 1.00 | 52.10 |
| ATOM | 4404 | CG | MET | B | 977 | 9.056 | 9.489 | −11.939 | 1.00 | 54.48 |
| ATOM | 4405 | SD | MET | B | 977 | 9.708 | 8.090 | −12.871 | 1.00 | 54.18 |
| ATOM | 4406 | CE | MET | B | 977 | 11.338 | 8.258 | −12.532 | 1.00 | 54.40 |
| ATOM | 4407 | N | ALA | B | 978 | 8.548 | 13.981 | −13.309 | 1.00 | 53.20 |
| ATOM | 4408 | CA | ALA | B | 978 | 9.030 | 15.303 | −13.672 | 1.00 | 52.59 |
| ATOM | 4409 | C | ALA | B | 978 | 8.429 | 16.332 | −12.714 | 1.00 | 53.53 |
| ATOM | 4410 | O | ALA | B | 978 | 8.910 | 17.468 | −12.625 | 1.00 | 53.68 |
| ATOM | 4411 | CB | ALA | B | 978 | 8.647 | 15.621 | −15.112 | 1.00 | 49.91 |
| ATOM | 4412 | N | ARG | B | 979 | 7.379 | 15.936 | −11.999 | 1.00 | 53.98 |
| ATOM | 4413 | CA | ARG | B | 979 | 6.749 | 16.836 | −11.042 | 1.00 | 57.90 |
| ATOM | 4414 | C | ARG | B | 979 | 7.590 | 16.920 | −9.767 | 1.00 | 59.18 |
| ATOM | 4415 | O | ARG | B | 979 | 7.417 | 17.834 | −8.970 | 1.00 | 61.14 |
| ATOM | 4416 | CB | ARG | B | 979 | 5.329 | 16.367 | −10.704 | 1.00 | 59.68 |
| ATOM | 4417 | CG | ARG | B | 979 | 4.322 | 16.484 | −11.856 | 1.00 | 61.54 |
| ATOM | 4418 | CD | ARG | B | 979 | 3.022 | 15.725 | −11.544 | 1.00 | 61.64 |
| ATOM | 4419 | NE | ARG | B | 979 | 2.376 | 15.243 | −12.766 | 1.00 | 63.13 |
| ATOM | 4420 | CZ | ARG | B | 979 | 1.427 | 14.309 | −12.809 | 1.00 | 62.83 |
| ATOM | 4421 | NH1 | ARG | B | 979 | 0.989 | 13.740 | −11.690 | 1.00 | 63.57 |
| ATOM | 4422 | NH2 | ARG | B | 979 | 0.931 | 13.924 | −13.980 | 1.00 | 62.38 |
| ATOM | 4423 | N | ASP | B | 980 | 8.496 | 15.964 | −9.583 | 1.00 | 59.01 |
| ATOM | 4424 | CA | ASP | B | 980 | 9.384 | 15.936 | −8.422 | 1.00 | 59.63 |
| ATOM | 4425 | C | ASP | B | 980 | 10.685 | 15.245 | −8.872 | 1.00 | 60.07 |
| ATOM | 4426 | O | ASP | B | 980 | 11.062 | 14.183 | −8.362 | 1.00 | 58.65 |
| ATOM | 4427 | CB | ASP | B | 980 | 8.709 | 15.177 | −7.262 | 1.00 | 60.54 |
| ATOM | 4428 | CG | ASP | B | 980 | 9.456 | 15.334 | −5.922 | 1.00 | 62.11 |
| ATOM | 4429 | OD1 | ASP | B | 980 | 10.255 | 16.288 | −5.765 | 1.00 | 61.29 |
| ATOM | 4430 | OD2 | ASP | B | 980 | 9.226 | 14.502 | −5.015 | 1.00 | 61.48 |
| ATOM | 4431 | N | PRO | B | 981 | 11.392 | 15.865 | −9.837 | 1.00 | 59.64 |
| ATOM | 4432 | CA | PRO | B | 981 | 12.651 | 15.412 | −10.445 | 1.00 | 60.15 |
| ATOM | 4433 | C | PRO | B | 981 | 13.831 | 14.975 | −9.573 | 1.00 | 60.53 |
| ATOM | 4434 | O | PRO | B | 981 | 14.609 | 14.114 | −9.986 | 1.00 | 60.77 |
| ATOM | 4435 | CB | PRO | B | 981 | 13.025 | 16.574 | −11.372 | 1.00 | 58.58 |
| ATOM | 4436 | CG | PRO | B | 981 | 12.393 | 17.753 | −10.726 | 1.00 | 57.47 |
| ATOM | 4437 | CD | PRO | B | 981 | 11.049 | 17.213 | −10.331 | 1.00 | 58.74 |
| ATOM | 4438 | N | GLN | B | 982 | 13.981 | 15.558 | −8.386 | 1.00 | 61.15 |
| ATOM | 4439 | CA | GLN | B | 982 | 15.102 | 15.197 | −7.521 | 1.00 | 60.07 |
| ATOM | 4440 | C | GLN | B | 982 | 14.854 | 13.919 | −6.745 | 1.00 | 57.60 |
| ATOM | 4441 | O | GLN | B | 982 | 15.774 | 13.355 | −6.167 | 1.00 | 56.36 |
| ATOM | 4442 | CB | GLN | B | 982 | 15.427 | 16.339 | −6.559 | 1.00 | 63.51 |
| ATOM | 4443 | CG | GLN | B | 982 | 16.078 | 17.547 | −7.228 | 1.00 | 66.46 |
| ATOM | 4444 | CD | GLN | B | 982 | 16.277 | 18.713 | −6.272 | 1.00 | 69.16 |
| ATOM | 4445 | OE1 | GLN | B | 982 | 15.811 | 18.681 | −5.134 | 1.00 | 72.71 |
| ATOM | 4446 | NE2 | GLN | B | 982 | 16.977 | 19.745 | −6.729 | 1.00 | 70.65 |
| ATOM | 4447 | N | ARG | B | 983 | 13.607 | 13.464 | −6.745 | 1.00 | 56.49 |
| ATOM | 4448 | CA | ARG | B | 983 | 13.221 | 12.230 | −6.062 | 1.00 | 54.51 |
| ATOM | 4449 | C | ARG | B | 983 | 13.456 | 11.011 | −6.955 | 1.00 | 52.63 |
| ATOM | 4450 | O | ARG | B | 983 | 13.445 | 9.870 | −6.489 | 1.00 | 51.88 |
| ATOM | 4451 | CB | ARG | B | 983 | 11.741 | 12.280 | −5.693 | 1.00 | 54.81 |
| ATOM | 4452 | CG | ARG | B | 983 | 11.219 | 11.016 | −5.031 | 1.00 | 56.29 |
| ATOM | 4453 | CD | ARG | B | 983 | 9.712 | 11.054 | −4.892 | 1.00 | 57.50 |
| ATOM | 4454 | NE | ARG | B | 983 | 9.214 | 9.997 | −4.018 | 1.00 | 58.23 |
| ATOM | 4455 | CZ | ARG | B | 983 | 7.924 | 9.735 | −3.834 | 1.00 | 57.38 |
| ATOM | 4456 | NH1 | ARG | B | 983 | 7.003 | 10.452 | −4.469 | 1.00 | 56.60 |
| ATOM | 4457 | NH2 | ARG | B | 983 | 7.556 | 8.765 | −3.011 | 1.00 | 57.50 |
| ATOM | 4458 | N | TYR | B | 984 | 13.673 | 11.257 | −8.241 | 1.00 | 50.73 |
| ATOM | 4459 | CA | TYR | B | 984 | 13.874 | 10.166 | −9.172 | 1.00 | 50.45 |
| ATOM | 4460 | C | TYR | B | 984 | 15.240 | 10.063 | −9.855 | 1.00 | 48.61 |
| ATOM | 4461 | O | TYR | B | 984 | 15.605 | 8.994 | −10.339 | 1.00 | 48.45 |
| ATOM | 4462 | CB | TYR | B | 984 | 12.724 | 10.175 | −10.177 | 1.00 | 49.68 |
| ATOM | 4463 | CG | TYR | B | 984 | 11.415 | 9.906 | −9.468 | 1.00 | 53.31 |
| ATOM | 4464 | CD1 | TYR | B | 984 | 10.591 | 10.950 | −9.047 | 1.00 | 54.83 |
| ATOM | 4465 | CD2 | TYR | B | 984 | 11.049 | 8.606 | −9.118 | 1.00 | 54.60 |
| ATOM | 4466 | CE1 | TYR | B | 984 | 9.435 | 10.703 | −8.289 | 1.00 | 54.57 |
| ATOM | 4467 | CE2 | TYR | B | 984 | 9.902 | 8.353 | −8.361 | 1.00 | 55.09 |
| ATOM | 4468 | CZ | TYR | B | 984 | 9.106 | 9.405 | −7.952 | 1.00 | 54.72 |
| ATOM | 4469 | OH | TYR | B | 984 | 7.984 | 9.146 | −7.207 | 1.00 | 56.77 |
| ATOM | 4470 | N | LEU | B | 985 | 15.996 | 11.157 | −9.878 | 1.00 | 46.87 |
| ATOM | 4471 | CA | LEU | B | 985 | 17.341 | 11.160 | −10.459 | 1.00 | 46.02 |
| ATOM | 4472 | C | LEU | B | 985 | 18.272 | 11.936 | −9.519 | 1.00 | 46.87 |
| ATOM | 4473 | O | LEU | B | 985 | 17.927 | 13.029 | −9.063 | 1.00 | 45.27 |
| ATOM | 4474 | CB | LEU | B | 985 | 17.344 | 11.812 | −11.845 | 1.00 | 44.28 |
| ATOM | 4475 | CG | LEU | B | 985 | 16.651 | 11.099 | −13.001 | 1.00 | 43.30 |
| ATOM | 4476 | CD1 | LEU | B | 985 | 17.075 | 11.770 | −14.289 | 1.00 | 43.28 |
| ATOM | 4477 | CD2 | LEU | B | 985 | 17.024 | 9.617 | −13.037 | 1.00 | 43.77 |
| ATOM | 4478 | N | VAL | B | 986 | 19.443 | 11.372 | −9.226 | 1.00 | 46.59 |
| ATOM | 4479 | CA | VAL | B | 986 | 20.381 | 12.023 | −8.317 | 1.00 | 47.28 |
| ATOM | 4480 | C | VAL | B | 986 | 21.628 | 12.479 | −9.045 | 1.00 | 48.95 |
| ATOM | 4481 | O | VAL | B | 986 | 22.479 | 11.675 | −9.419 | 1.00 | 50.35 |
| ATOM | 4482 | CB | VAL | B | 986 | 20.809 | 11.087 | −7.179 | 1.00 | 49.66 |
| ATOM | 4483 | CG1 | VAL | B | 986 | 21.654 | 11.863 | −6.171 | 1.00 | 48.82 |
| ATOM | 4484 | CG2 | VAL | B | 986 | 19.586 | 10.484 | −6.507 | 1.00 | 48.62 |
| ATOM | 4485 | N | ILE | B | 987 | 21.742 | 13.786 | −9.214 | 1.00 | 49.22 |
| ATOM | 4486 | CA | ILE | B | 987 | 22.854 | 14.377 | −9.927 | 1.00 | 50.44 |
| ATOM | 4487 | C | ILE | B | 987 | 23.496 | 15.472 | −9.084 | 1.00 | 53.00 |
| ATOM | 4488 | O | ILE | B | 987 | 22.797 | 16.347 | −8.568 | 1.00 | 53.10 |
| ATOM | 4489 | CB | ILE | B | 987 | 22.341 | 14.991 | −11.246 | 1.00 | 49.77 |
| ATOM | 4490 | CG1 | ILE | B | 987 | 21.671 | 13.902 | −12.087 | 1.00 | 50.87 |
| ATOM | 4491 | CG2 | ILE | B | 987 | 23.465 | 15.666 | −11.995 | 1.00 | 48.64 |
| ATOM | 4492 | CD1 | ILE | B | 987 | 20.856 | 14.444 | −13.253 | 1.00 | 51.02 |
| ATOM | 4493 | N | GLN | B | 988 | 24.820 | 15.427 | −8.946 | 1.00 | 55.17 |
| ATOM | 4494 | CA | GLN | B | 988 | 25.537 | 16.446 | −8.185 | 1.00 | 56.54 |
| ATOM | 4495 | C | GLN | B | 988 | 25.166 | 17.807 | −8.757 | 1.00 | 58.14 |
| ATOM | 4496 | O | GLN | B | 988 | 25.094 | 17.971 | −9.975 | 1.00 | 57.08 |
| ATOM | 4497 | CB | GLN | B | 988 | 27.047 | 16.245 | −8.300 | 1.00 | 57.24 |
| ATOM | 4498 | CG | GLN | B | 988 | 27.612 | 15.195 | −7.370 | 1.00 | 58.82 |
| ATOM | 4499 | CD | GLN | B | 988 | 29.129 | 15.143 | −7.409 | 1.00 | 60.42 |
| ATOM | 4500 | OE1 | GLN | B | 988 | 29.725 | 14.831 | −8.441 | 1.00 | 63.50 |
| ATOM | 4501 | NE2 | GLN | B | 988 | 29.763 | 15.448 | −6.282 | 1.00 | 62.41 |
| ATOM | 4502 | N | GLY | B | 989 | 24.951 | 18.784 | −7.879 | 1.00 | 59.86 |
| ATOM | 4503 | CA | GLY | B | 989 | 24.562 | 20.106 | −8.332 | 1.00 | 62.53 |
| ATOM | 4504 | C | GLY | B | 989 | 23.067 | 20.003 | −8.486 | 1.00 | 64.69 |
| ATOM | 4505 | O | GLY | B | 989 | 22.426 | 19.366 | −7.661 | 1.00 | 65.23 |
| ATOM | 4506 | N | ASP | B | 990 | 22.494 | 20.611 | −9.516 | 1.00 | 67.44 |
| ATOM | 4507 | CA | ASP | B | 990 | 21.053 | 20.475 | −9.704 | 1.00 | 70.60 |
| ATOM | 4508 | C | ASP | B | 990 | 20.352 | 20.808 | −8.377 | 1.00 | 72.92 |
| ATOM | 4509 | O | ASP | B | 990 | 19.373 | 20.165 | −7.996 | 1.00 | 73.02 |
| ATOM | 4510 | CB | ASP | B | 990 | 20.764 | 19.019 | −10.136 | 1.00 | 68.29 |
| ATOM | 4511 | CG | ASP | B | 990 | 19.376 | 18.821 | −10.727 | 1.00 | 65.16 |
| ATOM | 4512 | OD1 | ASP | B | 990 | 18.904 | 19.688 | −11.493 | 1.00 | 64.82 |
| ATOM | 4513 | OD2 | ASP | B | 990 | 18.764 | 17.771 | −10.433 | 1.00 | 61.33 |
| ATOM | 4514 | N | ALA | B | 991 | 20.862 | 21.814 | −7.675 | 1.00 | 75.66 |
| ATOM | 4515 | CA | ALA | B | 991 | 20.275 | 22.219 | −6.399 | 1.00 | 79.01 |
| ATOM | 4516 | C | ALA | B | 991 | 19.048 | 23.107 | −6.615 | 1.00 | 80.54 |
| ATOM | 4517 | O | ALA | B | 991 | 17.965 | 22.739 | −6.100 | 1.00 | 81.75 |
| ATOM | 4518 | CB | ALA | B | 991 | 21.317 | 22.948 | −5.537 | 1.00 | 77.93 |
| ATOM | 4519 | OXT | ALA | B | 991 | 19.180 | 24.153 | −7.295 | 1.00 | 81.39 |
| ATOM | 4520 | F1 | LIG | C | 1 | −21.453 | 16.649 | −0.604 | 1.00 | 65.92 |
| ATOM | 4521 | C2 | LIG | C | 1 | −21.069 | 17.936 | −0.472 | 1.00 | 62.88 |
| ATOM | 4522 | C3 | LIG | C | 1 | −19.922 | 18.287 | 0.383 | 1.00 | 60.99 |
| ATOM | 4523 | C4 | LIG | C | 1 | −19.435 | 19.703 | 0.395 | 1.00 | 60.35 |
| ATOM | 4524 | C5 | LIG | C | 1 | −20.135 | 20.740 | −0.395 | 1.00 | 61.20 |
| ATOM | 4525 | C6 | LIG | C | 1 | −21.331 | 20.391 | −1.193 | 1.00 | 63.29 |
| ATOM | 4526 | C7 | LIG | C | 1 | −21.800 | 18.979 | −1.237 | 1.00 | 63.70 |
| ATOM | 4527 | C8 | LIG | C | 1 | −18.188 | 20.151 | 1.138 | 1.00 | 58.87 |
| ATOM | 4528 | O9 | LIG | C | 1 | −17.171 | 19.172 | 1.348 | 1.00 | 56.77 |
| ATOM | 4529 | C10 | LIG | C | 1 | −16.358 | 18.640 | 0.332 | 1.00 | 55.56 |
| ATOM | 4530 | C11 | LIG | C | 1 | −15.952 | 17.241 | 0.543 | 1.00 | 56.41 |
| ATOM | 4531 | C12 | LIG | C | 1 | −15.101 | 16.562 | −0.402 | 1.00 | 54.33 |
| ATOM | 4532 | C13 | LIG | C | 1 | −14.603 | 17.275 | −1.616 | 1.00 | 53.77 |
| ATOM | 4533 | C14 | LIG | C | 1 | −15.012 | 18.688 | −1.848 | 1.00 | 55.54 |
| ATOM | 4534 | C15 | LIG | C | 1 | −15.913 | 19.352 | −0.900 | 1.00 | 54.37 |
| ATOM | 4535 | N16 | LIG | C | 1 | −13.762 | 16.701 | −2.549 | 1.00 | 49.69 |
| ATOM | 4536 | C17 | LIG | C | 1 | −13.714 | 15.438 | −3.082 | 1.00 | 50.51 |
| ATOM | 4537 | N18 | LIG | C | 1 | −14.809 | 14.489 | −2.937 | 1.00 | 49.27 |
| ATOM | 4538 | C19 | LIG | C | 1 | −14.740 | 13.219 | −3.533 | 1.00 | 49.13 |
| ATOM | 4539 | N20 | LIG | C | 1 | −13.631 | 12.866 | −4.316 | 1.00 | 48.53 |
| ATOM | 4540 | C21 | LIG | C | 1 | −12.558 | 13.784 | −4.480 | 1.00 | 50.01 |
| ATOM | 4541 | C22 | LIG | C | 1 | −12.550 | 15.113 | −3.834 | 1.00 | 50.59 |
| ATOM | 4542 | S23 | LIG | C | 1 | −11.201 | 16.018 | −4.243 | 1.00 | 54.32 |
| ATOM | 4543 | C24 | LIG | C | 1 | −10.572 | 14.794 | −5.310 | 1.00 | 54.73 |
| ATOM | 4544 | C25 | LIG | C | 1 | −11.377 | 13.652 | −5.309 | 1.00 | 52.56 |
| ATOM | 4545 | CL2 | LIG | C | 1 | −16.406 | 16.290 | 1.907 | 1.00 | 58.80 |
| ATOM | 4546 | C27 | LIG | C | 1 | −8.858 | 16.177 | −6.594 | 1.00 | 59.22 |
| ATOM | 4547 | C26 | LIG | C | 1 | −9.449 | 14.847 | −6.122 | 1.00 | 55.07 |
| ATOM | 4548 | F1 | LIG | D | 1 | 10.853 | 15.135 | −41.543 | 1.00 | 58.61 |
| ATOM | 4549 | C2 | LIG | D | 1 | 11.037 | 13.839 | −41.713 | 1.00 | 54.55 |
| ATOM | 4550 | C3 | LIG | D | 1 | 12.223 | 13.471 | −42.454 | 1.00 | 54.59 |
| ATOM | 4551 | C4 | LIG | D | 1 | 12.685 | 12.058 | −42.421 | 1.00 | 55.40 |
| ATOM | 4552 | C5 | LIG | D | 1 | 11.870 | 11.031 | −41.722 | 1.00 | 55.68 |
| ATOM | 4553 | C6 | LIG | D | 1 | 10.584 | 11.399 | −41.086 | 1.00 | 55.06 |
| ATOM | 4554 | C7 | LIG | D | 1 | 10.166 | 12.828 | −41.075 | 1.00 | 55.50 |
| ATOM | 4555 | C8 | LIG | D | 1 | 14.013 | 11.667 | −43.046 | 1.00 | 55.59 |
| ATOM | 4556 | O9 | LIG | D | 1 | 14.925 | 12.754 | −43.284 | 1.00 | 54.46 |
| ATOM | 4557 | C10 | LIG | D | 1 | 15.752 | 13.298 | −42.283 | 1.00 | 54.12 |
| ATOM | 4558 | C11 | LIG | D | 1 | 16.205 | 14.668 | −42.540 | 1.00 | 54.63 |
| ATOM | 4559 | C12 | LIG | D | 1 | 17.138 | 15.313 | −41.638 | 1.00 | 54.53 |
| ATOM | 4560 | C13 | LIG | D | 1 | 17.592 | 14.617 | −40.390 | 1.00 | 54.63 |
| ATOM | 4561 | C14 | LIG | D | 1 | 17.151 | 13.224 | −40.122 | 1.00 | 54.98 |
| ATOM | 4562 | C15 | LIG | D | 1 | 16.212 | 12.590 | −41.043 | 1.00 | 53.54 |
| ATOM | 4563 | N16 | LIG | D | 1 | 18.380 | 15.217 | −39.444 | 1.00 | 52.13 |
| ATOM | 4564 | C17 | LIG | D | 1 | 18.311 | 16.478 | −38.879 | 1.00 | 52.92 |
| ATOM | 4565 | N18 | LIG | D | 1 | 17.153 | 17.343 | −38.997 | 1.00 | 52.67 |
| ATOM | 4566 | C19 | LIG | D | 1 | 17.111 | 18.603 | −38.359 | 1.00 | 52.27 |
| ATOM | 4567 | N20 | LIG | D | 1 | 18.197 | 19.019 | −37.573 | 1.00 | 52.85 |
| ATOM | 4568 | C21 | LIG | D | 1 | 19.370 | 18.189 | −37.471 | 1.00 | 53.46 |
| ATOM | 4569 | C22 | LIG | D | 1 | 19.449 | 16.879 | −38.137 | 1.00 | 52.71 |
| ATOM | 4570 | S23 | LIG | D | 1 | 20.838 | 16.039 | −37.751 | 1.00 | 55.09 |
| ATOM | 4571 | C24 | LIG | D | 1 | 21.417 | 17.235 | −36.656 | 1.00 | 55.61 |
| ATOM | 4572 | C25 | LIG | D | 1 | 20.602 | 18.381 | −36.703 | 1.00 | 54.95 |
| ATOM | 4573 | CL2 | LIG | D | 1 | 15.733 | 15.611 | −43.921 | 1.00 | 56.18 |
| ATOM | 4574 | C27 | LIG | D | 1 | 23.036 | 15.744 | −35.342 | 1.00 | 59.16 |
| ATOM | 4575 | C26 | LIG | D | 1 | 22.474 | 17.108 | −35.765 | 1.00 | 54.82 |
| ATOM | 4576 | O | HOH | E | 1 | −7.599 | 8.623 | −13.123 | 1.00 | 62.46 |
| ATOM | 4577 | O | HOH | E | 2 | 24.517 | 23.513 | −28.035 | 1.00 | 64.27 |
| ATOM | 4578 | O | HOH | E | 3 | −20.296 | 13.666 | 15.285 | 1.00 | 63.01 |
| ATOM | 4579 | O | HOH | E | 4 | −22.374 | 8.176 | −7.192 | 1.00 | 49.42 |
| ATOM | 4580 | O | HOH | E | 5 | −29.264 | 30.402 | −15.340 | 1.00 | 37.27 |
| ATOM | 4581 | O | HOH | E | 6 | −15.548 | 26.298 | −3.002 | 1.00 | 60.28 |
| ATOM | 4582 | O | HOH | E | 7 | −16.488 | 46.164 | −1.672 | 1.00 | 37.96 |
| ATOM | 4583 | O | HOH | E | 8 | −23.337 | 42.864 | −22.019 | 1.00 | 54.63 |
| ATOM | 4584 | O | HOH | E | 9 | 11.610 | 18.211 | −56.805 | 1.00 | 64.90 |
| ATOM | 4585 | O | HOH | E | 10 | 16.106 | 5.158 | −39.459 | 1.00 | 71.20 |
| ATOM | 4586 | O | HOH | E | 11 | 24.422 | −1.900 | −32.376 | 1.00 | 37.06 |
| ATOM | 4587 | O | HOH | E | 12 | 18.940 | −3.777 | −26.629 | 1.00 | 37.73 |
| ATOM | 4588 | O | HOH | E | 13 | 2.846 | 1.640 | −26.716 | 1.00 | 48.90 |
| ATOM | 4589 | O | HOH | E | 14 | 22.115 | 6.710 | −30.625 | 1.00 | 46.15 |
| ATOM | 4590 | O | HOH | E | 15 | 27.166 | −0.416 | −20.681 | 1.00 | 52.66 |
| ATOM | 4591 | O | HOH | E | 16 | 27.370 | 0.625 | −28.338 | 1.00 | 48.54 |
| ATOM | 4592 | O | HOH | E | 17 | 8.879 | −10.943 | −20.472 | 1.00 | 43.79 |
| ATOM | 4593 | O | HOH | E | 18 | 22.979 | 20.646 | −12.884 | 1.00 | 55.83 |
| ATOM | 4594 | O | HOH | E | 19 | −23.835 | 38.304 | −2.575 | 1.00 | 46.63 |
| ATOM | 4595 | O | HOH | E | 20 | 3.858 | 19.802 | −44.299 | 1.00 | 51.25 |
| ATOM | 4596 | O | HOH | E | 21 | 6.565 | 20.312 | −34.549 | 1.00 | 67.07 |
| ATOM | 4597 | O | HOH | E | 22 | 6.762 | 22.561 | −32.466 | 1.00 | 63.76 |
| ATOM | 4598 | O | HOH | E | 23 | 17.087 | 2.388 | −32.802 | 1.00 | 50.29 |
| ATOM | 4599 | O | HOH | E | 24 | 19.085 | −5.539 | −28.680 | 1.00 | 47.97 |
| ATOM | 4600 | O | HOH | E | 25 | 8.303 | −6.214 | −39.578 | 1.00 | 53.88 |
| ATOM | 4601 | O | HOH | E | 26 | 24.363 | 7.903 | −14.629 | 1.00 | 43.74 |
| ATOM | 4602 | O | HOH | E | 27 | −27.032 | 11.186 | 2.400 | 1.00 | 74.22 |
| ATOM | 4603 | O | HOH | E | 28 | −12.942 | 20.542 | −5.335 | 1.00 | 44.65 |
| ATOM | 4604 | O | HOH | E | 29 | −7.825 | 34.064 | −9.696 | 1.00 | 36.01 |
| ATOM | 4605 | O | HOH | E | 30 | 6.161 | 13.446 | −8.135 | 1.00 | 48.06 |
| ATOM | 4606 | O | HOH | E | 31 | 4.895 | 21.272 | −54.439 | 1.00 | 37.34 |
| ATOM | 4607 | O | HOH | E | 32 | 14.560 | 16.150 | −38.934 | 1.00 | 49.73 |
| ATOM | 4608 | O | HOH | E | 33 | −17.191 | 16.196 | −2.787 | 1.00 | 49.94 |
| ATOM | 4609 | O | HOH | E | 34 | −12.431 | 16.753 | −33.087 | 1.00 | 50.01 |
| ATOM | 4610 | O | HOH | E | 35 | 19.316 | 15.273 | −8.990 | 1.00 | 41.76 |
| ATOM | 4611 | O | HOH | E | 36 | 22.919 | −2.802 | −12.770 | 1.00 | 32.09 |
| ATOM | 4612 | O | HOH | E | 37 | −20.465 | 42.538 | −22.926 | 1.00 | 48.88 |
| ATOM | 4613 | O | HOH | E | 38 | 24.551 | −2.864 | −35.433 | 1.00 | 38.92 |
| ATOM | 4614 | O | HOH | E | 39 | 10.469 | 2.554 | −40.581 | 1.00 | 54.18 |
| ATOM | 4615 | O | HOH | E | 40 | 4.881 | 19.023 | −24.625 | 1.00 | 58.60 |
| ATOM | 4616 | O | HOH | E | 41 | 18.317 | 10.202 | −53.601 | 1.00 | 71.11 |
| ATOM | 4617 | O | HOH | E | 42 | −13.865 | 22.064 | 10.271 | 1.00 | 57.28 |
| ATOM | 4618 | O | HOH | E | 43 | −35.587 | 21.497 | 3.030 | 1.00 | 36.88 |
| ATOM | 4619 | O | HOH | E | 44 | −26.803 | 14.899 | −5.791 | 1.00 | 40.87 |
| ATOM | 4620 | O | HOH | E | 45 | −24.968 | 8.964 | −9.528 | 1.00 | 60.43 |
| ATOM | 4621 | O | HOH | E | 46 | −18.782 | 34.826 | −10.499 | 1.00 | 37.91 |
| ATOM | 4622 | O | HOH | E | 47 | −15.030 | 29.928 | −8.619 | 1.00 | 52.85 |
| ATOM | 4623 | O | HOH | E | 48 | −19.234 | 30.472 | −9.478 | 1.00 | 49.50 |
| ATOM | 4624 | O | HOH | E | 49 | −10.976 | 11.240 | 12.729 | 1.00 | 52.40 |
| ATOM | 4625 | O | HOH | E | 50 | 16.214 | −4.944 | −38.622 | 1.00 | 53.39 |
| ATOM | 4626 | O | HOH | E | 51 | −14.354 | 32.711 | −8.242 | 1.00 | 47.04 |
| ATOM | 4627 | O | HOH | E | 52 | −13.127 | 35.963 | −19.946 | 1.00 | 44.74 |
| ATOM | 4628 | O | HOH | E | 53 | −26.260 | 30.450 | −26.299 | 1.00 | 46.29 |
| ATOM | 4629 | O | HOH | E | 54 | 9.344 | 23.549 | −34.825 | 1.00 | 60.48 |
| ATOM | 4630 | O | HOH | E | 55 | 12.830 | 1.506 | −32.483 | 1.00 | 44.17 |
| END | | | | | | | | | | |

### REFERENCES

1. Johnson, L. N., Noble, M. E. M & Owen, D. J. (1996). Active and inactive protein kinases: structural basis for regulation. Cell 85, 149-158.
2. Cox, S., Radzio-Andzelm, E. & Taylor, S. S. (1994). Domain movements in protein kinases. Curr. Opin. Struct. Biol. 4, 893-901.
3. Schindler, T., Bornmann, W., Pellicena, P., Miller, W. T., Clarkson, B., Kuriyan, J. (2000). Structural Mechanism for Sti-571 Inhibition of Abelson Tyrosine Kinase. Science 289, 1857-9.
4. Knighton, D. R., et al., & Sowadski, J. M. (1991). Crystal structure of the catalytic subunit of cyclic adenosine monophosphate-dependent protein kinase. Science 253, 407-413.
5. Mohammadi, M., Schlessinger, J. & Hubbard, S. R. (1996). Structure of the FGF receptor tyrosine kinase domain reveals a novel autoinhibitory mechanism. Cell 86, 577-87.
6. Wybenga-Groot, L. E., Baskin, B., Ong, S. H., Tong, J., Pawson, T. & Sicheri, F. (2001). Structural Basis for Autoinhibition of the EphB2 Receptor Tyrosine Kinase by the Unphosphorylated Juxtamembrane Region. Cell 106, 745-757.
7. Hubbard, S. R. (1997). Crystal structure of the activated insulin receptor tyrosine kinase in complex with peptide substrate and ATP analog. Embo J. 16, 5572-5581.
8. Hubbard, S. R., Wei, L., Ellis, L. & Hendrickson, W. A. (1994). Crystal structure of the tyrosine kinase domain of the human insulin receptor. Nature 372, 746-754.
9. McTigue, M. A., et al., & Appelt, K. (1999). Crystal structure of the kinase domain of human vascular endothelial growth factor receptor 2: a key enzyme in angiogenesis. Structure 7, 319-330.
10. Xu, W., Doshi, A., Lei, M., Eck, M. J. & Harrison S C (1999) Crystal structures of c-Src reveal features of its autoinhibitory mechanism. Mol. Cell 5, 629-38.

What is claimed is:

1. An ErbB4 kinase domain in liganded crystalline form, comprising the amino acid sequence of SEQ ID NO: 1 and having the structural coordinates of Table 2.

2. A liganded ErbB4 kinase domain as claimed in claim 1, wherein the crystalline form has lattice constants of a=63.95 Å, b=63.95 Å, c=163.95 Å, α=90°, β=90°, and γ=90°.

3. A liganded ErbB4 kinase domain in crystalline form as claimed in claim 1, wherein said crystalline form has a space group of P4₃.

4. A method of ErbB4 inhibitor design, comprising:
generating a three dimensional computer model which represents ErbB4 kinase domain in liganded form, said kinase domain described by the amino acid sequence of SEQ ID NO: 1 and having the structural coordinates of Table 2;
evaluating compounds as potential ErbB4 inhibitors using said model; and
selecting compounds for further testing based on said evaluation.

5. A method of ErbB4 inhibitor design, comprising:
generating a three dimensional computer model which represents a ErbB4 kinase domain in liganded form, said kinase domain described by the amino acid sequence of SEQ ID NO: 1 and having the structural coordinates of Table 2;
evaluating compounds as potential ErbB4 inhibitors using said model;
wherein said evaluation comprises identifying compounds capable of at least one of the following ErbB4 kinase domain/compound interactions:
(iv) one or more interactions with amino acid residues of the ErbB4 kinase domain hinge region;
(v) one or more interactions with amino acid residues of the ErbB4 kinase domain adenine pocket,
(iii) one or more interactions with amino acid residues of the ErbB4 kinase sugar pocket,
(iv) one or more interactions with amino acid residues of the ErbB4 kinase domain back pocket, and
(v) one or more interactions with amino acid residues of the ErbB4 kinase domain solvent interface; and
selecting compounds for further testing based on said evaluation.

6. A method of ErbB4 inhibitor design, comprising:
generating a three dimensional computer model which represents a ErbB4 kinase domain in liganded form, said kinase domain described by the amino acid sequence of SEQ ID NO: 1 and having the structural coordinates of Table 2;
evaluating compounds as potential ErbB4 inhibitors using said model; wherein said evaluation comprises identifying compounds capable of at least one of the following ErbB4 kinase domain/compound interactions:
(i) one or more interactions with amino acid residues 796, 797, 798, 799, and 800;
(ii) one or more interactions with amino acid residues 724, 749, and 850;
(iii) one or more interactions with amino acid residues 848, 860, 803, 847, 732, and 725;
(iv) one or more interactions with amino acid residues 732, 749, 751, 796, 861, 860, 772, 781, 783, 794, 796, and 862; and
(v) one or more interactions with residues 801, 802, 803, 806, and 810; and
selecting compounds for further testing based on said evaluation.

7. A method of treating a disorder characterized by inappropriate ErbB4 activity in a mammal, comprising: administering to said mammal a therapeutically effective amount of a compound that can form a complex with a ErbB4 kinase domain thereby resulting in a ErbB4 kinase domain in liganded form, said kinase domain in liganded form being described by the amino acid sequence of SEQ ID NO: 1 and the structural coordinates of Table 2, wherein said complex is characterized by at least one of the following ErbB4 kinase domain/compound interactions:
(i) one or more interactions with amino acid residues of the ErbB4 kinase domain hinge region;
(ii) one or more interactions with amino acid residues of the ErbB4 kinase domain adenine pocket,
(iii) one or more interactions with amino acid residues of the ErbB4 kinase sugar pocket and phosphate region,
(iv) one or more interactions with amino acid residues of the ErbB4 kinase domain back pocket, and
(v) one or more interactions with amino acid residues of the ErbB4 kinase domain solvent interface.

8. A method of inhibiting ErbB4 in a mammal, comprising: administering to said mammal a therapeutically effective amount of a compound that can form a complex with a ErbB4 kinase domain thereby resulting in a ErbB4 kinase domain in liganded form, said kinase domain in liganded form being described by the amino acid sequence of SEQ ID NO: 1 and the structural coordinates of Table 2, wherein said complex is characterized by at least one of the following ErbB4 kinase domain/compound interactions:
(i) one or more interactions with amino acid residues of the ErbB4 kinase domain hinge region;
(ii) one or more interactions with amino acid residues of the ErbB4 kinase domain adenine pocket,
(iii) one or more interactions with amino acid residues of the ErbB4 kinase sugar pocket and phosphate region,
(iv) one or more interactions with amino acid residues of the ErbB4 kinase domain back pocket, and
(v) one or more interactions with amino acid residues of the ErbB4 kinase domain solvent interface.

9. An ErbB4 kinase domain/inhibitor complex, comprising: an ErbB4 kinase domain form being described by the amino acid sequence of SEQ ID NO: 1 and the structural coordinates of Table 2 and a compound that can form a complex with the ErbB4 kinase domain said complex is characterized by at least one of the following ErbB4 kinase domain/compound interactions:
(i) one or more interactions with amino acid residues of the ErbB4 kinase domain hinge region;
(ii) one or more interactions with amino acid residues of the ErbB4 kinase domain adenine pocket,
(iii) one or more interactions with amino acid residues of the ErbB4 kinase sugar pocket and phosphate region,
(iv) one or more interactions with amino acid residues of the ErbB4 kinase domain back pocket, and
(v) one or more interactions with amino acid residues of the ErbB4 kinase domain solvent interface.


SEQUENCE LISTING (2 sequences)

SEQ ID NO: 1 - protein, 364 aa, homo sapien
MKKGHHHHHHGLETELVEPLTPSGTAPNQAQLRILKETELKRVKVLGSGAFGTVYKGIWVPEGETVKIPVAIKILNETTGPKANVEFMDEALIMASMDHPHLVRLLGVCLSPTIQLVTQLMPHGCLLEYVHEHKDNIGSQLLLNWCVQIAKGMMYLEERRLVHRDLAARNVLVKSPNHVKITDFGLARLLEGDEKEYNADGGKMPIKWMALECIHYRKFTHQSDVWSYGVTIWELMTFGGKPYDGIPTREIPDLLEKGERLPQPPICTIDVYMVMVKCWMIDADSRPKFKELAAEFSRMARDPQRYLVIQGDDRMKLPSPNDSKFFQNLLDEEDLEDMMDAEEYLVPQAFNIPPPIYTSRARID

SEQ ID NO: 2 - DNA, 966 bp, homo sapien
atgaaaaaaggtcatcatcatcatcatcatggtttggaaacagagttggtggaaccattaactcccagtggcacagcacccaatcaagctcaacttcgtattttgaaagaaactgagctgaagagggtaaaagtccttggctcaggtgcttttggaacggtttataaaggtatttgggtacctgaaggagaaactgtgaagattcctgtggctattaagattcttaatgagacaactggtcccaaggcaaatgtggagttcatggatgaagctctgatcatggcaagtatggatcatccacacctagtccggttgctgggtgtgtgtctgagcccaaccatccagctggttactcaacttatgccccatggctgcctgttggagtatgtccacgagcacaaggataacattggatcacaactgctgcttaactggtgtgtccagatagctaagggaatgatgtacctggaagaaagacgactcgttcatcgggatttggcagcccgtaatgtcttagtgaaatctccaaaccatgtgaaaatcacagattttgggctagccagactcttggaaggagatgaaaaagagtacaatgctgatggaggaaagatgccaattaaatggatggctctggagtgtatacattacaggaaattcacccatcagagtgacgtttggagctatggagttactatatgggaactgatgacctttggaggaaaaccctatgatggaattccaacgcgagaaatccctgatttattagagaaaggagaacgtttgcctcagcctcccatctgcactattgacgtttacatggtcatggtcaaatgttggatgattgatgctgacagtagacctaaatttaaggaactggctgctgagttttcaaggatggctcgagaccctcaaagatacctagttattcagggtgatgatcgtatgaagcttcccagtccaaattga